SEQUENCE LISTING (82 sequences)

SEQ ID NO: 1 - protein, 19 aa, Artificial Sequence
  Synthetic Construct
KLALKLALKALKLAALKLA

SEQ ID NO: 2 - protein, 18 aa, Artificial Sequence
  Synthetic Construct
KLALKLALKALKAALKLA

SEQ ID NO: 3 - protein, 18 aa, Artificial Sequence
  Synthetic Construct
KLALKLALKALKAALKLA

SEQ ID NO: 4 - protein, 18 aa, Artificial Sequence
  Synthetic Construct
ALKLAAKLAKLALKLALK

SEQ ID NO: 5 - protein, 15 aa, Artificial Sequence
  Synthetic Construct
LKLLKKLLKKLLKLL

SEQ ID NO: 6 - protein, 16 aa, Artificial Sequence
  Synthetic Construct
KLALKLALKALKAALK

SEQ ID NO: 7 - protein, 20 aa, Artificial Sequence
  Synthetic Construct
KLALKLALKALKAALKLALK

SEQ ID NO: 8 - protein, 18 aa, Artificial Sequence
  Synthetic Construct
KLAWKLALKALKAALKLA

SEQ ID NO: 9 - protein, 18 aa, Artificial Sequence
  Synthetic Construct
KLAWKLALKALKAAWKLA

SEQ ID NO: 10 - protein, 18 aa, Artificial Sequence
  Synthetic Construct
KLAWKLAWKALKAAWKLA

SEQ ID NO: 11 - protein, 15 aa, Artificial Sequence
  Synthetic Construct
LKLLKKLLKKLLKLL

SEQ ID NO: 12 - protein, 15 aa, Artificial Sequence
  Synthetic Construct
LKLLKKLLKKLLKLL

SEQ ID NO: 13 - protein, 18 aa, Artificial Sequence
  Synthetic Construct
KALAALLKKAAKLLAALK

SEQ ID NO: 14 - protein, 18 aa, Artificial Sequence
  Synthetic Construct
KALAALLKKLAKLLAALK

SEQ ID NO: 15 - protein, 257 aa, Artificial Sequence
  Synthetic Construct
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 16 - DNA, 771 bp, Artificial Sequence
  Synthetic Construct
atgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaaca

SEQ ID NO: 17 - protein, 6 aa, Artificial Sequence
  Synthetic Construct
  MOD_RES (1)..(1): ACETYLATION
ADTPPV

SEQ ID NO: 18 - protein, 6 aa, Artificial Sequence
  Synthetic Construct
  MOD_RES (1)..(1): ACETYLATION
SHAVSS

SEQ ID NO: 19 - protein, 241 aa, Artificial Sequence
  Synthetic Construct
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 20 - protein, 120 aa, Artificial Sequence
  Synthetic Construct
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 21 - protein, 270 aa, Artificial Sequence
  Synthetic Construct
MVTFATILQVNKVMSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 22 - protein, 119 aa, Artificial Sequence [sequence omitted]

SEQ ID NO: 23 - protein, 210 aa, Artificial Sequence
  Synthetic Construct
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 24 - protein, 130 aa, Artificial Sequence
  Synthetic Construct
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 25 - protein, 247 aa, Artificial Sequence
  Synthetic Construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 26 - protein, 262 aa, Artificial Sequence
  Synthetic Construct
MQSREEEWFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 27 - protein, 329 aa, Artificial Sequence
  Synthetic Construct
MCGATSFLHECTRLILVTTQNAEFLQKGLQVHTCFGVYPHASVWHDCASQKKGCAVYLHVSVEFNKLIPENGFIKFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 28 - protein, 255 aa, Artificial Sequence
  Synthetic Construct
MFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 29 - protein, 276 aa, Artificial Sequence
  Synthetic Construct
MLCAISLCARVRKLRSAGRCGKFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 30 - protein, 119 aa, Artificial Sequence
  Synthetic Construct
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 31 - protein, 120 aa, Artificial Sequence
  Synthetic Construct
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 32 - protein, 121 aa, Artificial Sequence
  Synthetic Construct
SSSHPIFHRGEFSVCDSVSVWVGDKTTATDIKGKEVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 33 - protein, 123 aa, Artificial Sequence
  Synthetic Construct
SSSHPIFHRGEFSVCDSVSVWVTAADKKTAVDMSGGTVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 34 - protein, 120 aa, Artificial Sequence
  Synthetic Construct
SSSHPIAHRGEFSVCDSVSVWVGDKTTATDIKGKEVMVLGEVNINNSVFKQYFFETKCRDPNPVDSGCRGIDSKHWNSYCTTTATFVKALTMDGKQAAWRFIAIDTACVCVLSRKAVRRA

SEQ ID NO: 35 - protein, 120 aa, Artificial Sequence
  Synthetic Construct
SSSHPIAHRGEFSVCDSVSVWVGDKTTATDIKGKEVMVLGEVNIANSVFKQYFFETKCRDPNPVDSGCRGIDSKHWNSYCTTTHTFVKALTMDGKQAAWRFIAIDTACVCVLSRKAVRRA

SEQ ID NO: 36 - protein, 120 aa, Artificial Sequence
  Synthetic Construct
SSSHPIAHRGEFSVCDSVSVWVGDKTTATDIAGAAVMVLGEVNINNSVFKQYFFETKCRDPNPVDSGCRGIDSKHWNSYCTTTATFVKALTMDGKQAAWRFIAIDTACVCVLSRKAVRRA

SEQ ID NO: 37 - protein, 120 aa, Artificial Sequence
  Synthetic Construct
SSSHPIAHRGEFSVCDSVSVWVGDKTTATDIAGAAVMVLGEVNIANSVFKQYFFETKCRDPNPVDSGCRGIDSKHWNSYCTTTHTFVKALTMDGKQAAWRFIAIDTACVCVLSRKAVRRA

SEQ ID NO: 38 - protein, 115 aa, Artificial Sequence
  Synthetic Construct
SSSHPIFHVCDSVSVWVGDKTTATDIAGAAVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 39 - protein, 120 aa, Artificial Sequence
  Synthetic Construct
SSSHPIFHRGEYSVCDSESLWVTDKSSAIDIAGAQVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 40 - protein, 120 aa, Artificial Sequence
  Synthetic Construct
SSSHPIFHRGEYSVCDSESLWVTDKSSAIDIRGHQVTVLGEIKTGNSPVKQYFYETRCKEARPVKNGCRGIDDAAWASQCKTSQTYVRALTSESKKRIGWRWIRIDTSCVCALSRKIGRT

SEQ ID NO: 41 - protein, 120 aa, Artificial Sequence
  Synthetic Construct
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSAAIGRT

SEQ ID NO: 42 - protein, 120 aa, Artificial Sequence
  Synthetic Construct
SSSHPIFHRGEYSVCDSESLWVTDKSSAIDIAGAQVTVLGEIKTGNSPVKQYFYETRCKEARPVKNGCRGIDDAAWASQCKTSQTYVRALTSESKKRIGWRWIRIDTSCVCALSAAIGRT

SEQ ID NO: 43 - protein, 121 aa, Artificial Sequence
  Synthetic Construct
SSSHPIFHRGEFSVCDSVSVWVGDKTTATDIAGAAVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 44 - protein, 123 aa, Artificial Sequence
  Synthetic Construct
SSSHPIFHRGEFSVCDSVSVWVTAADKKTAVDMAGAAVMVLGEVNINNSVFKQYFFETKCNPMGYTKEGCRGIDSKHWNSQCRTTQSYVRALTMDSKKRIGWRFIRIDTACVCVLSRKAVRRA

SEQ ID NO: 45 - protein, 120 aa, Artificial Sequence
  Synthetic Construct
SSSHPIFHRGEYSVCDSESLWVTDKSSAIDIAGAAVTVLGEIKTGNSPVKQYFYETRCKEARPVKNGCRGIDDKHWNSQCKTSQTYVRALTSESKKRIGWRWIRIDTSCVCALSRKIGRT

SEQ ID NO: 46 - protein, 120 aa, Artificial Sequence
  Synthetic Construct
SSSHPIFHRGEYSVCDSESLWVTDKSSAIDIRGHQVTVLGEIKTGNSPVKQYFYETRCKESRPVKNGCRGIDDKHWNSQCKTSQTYVRALTSESKKRIGWEWIRIDTSCVCALSRKIGRT

SEQ ID NO: 47 - protein, 120 aa, Artificial Sequence
  Synthetic Construct
SSSHPIFHRGEYSVCDSESLWVTDKSSAIDIRGHQVTVLGEIKTGNSPVKQYFYETRCKEARPVKNGCRGIDDKHWNSQCKTSQTYVRALTSESKKRIGWRWIRIDTSCVCALSRKIART

SEQ ID NO: 48 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIAIDTSCVCALSRKIGRT

SEQ ID NO: 49 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 50 - protein, 119 aa, Artificial Sequence
  Synthetic Construct
YAEHKSHRGEASVCASESLWVTDKSSAIDIRGHQVTVLGEIKTGNSPVKQYFYETRCKEARPVKNGCAGIDDKHWNSQCKTSQTYVAALTSENNKLVGWRWIRIDTSCVCALSAAIGRT

SEQ ID NO: 51 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCAGIDDKHWNSQCKTSQTYVAALTSENNKLVGWRWIRIDTSCVCALSAAIGRT

SEQ ID NO: 52 - protein, 119 aa, Artificial Sequence
  Synthetic Construct
YAEHKSHRGEASVCASESLWVTDKSSAIDIRGHQVTVLGEIKTGNSPVKQYFYETRCKEARPVKNGCRGIDDKHWNSQCKTSQTYVAALTSENNKLVGWRWIRIDTSCVCALSAAIGRT

SEQ ID NO: 53 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCRGIDDKHWNSQCKTSQTYVAALTSENNKLVGWRWIRIDTSCVCALSAAIGRT

SEQ ID NO: 54 - protein, 119 aa, Artificial Sequence
  Synthetic Construct
YAEHKSHRGEASVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 55 - protein, 119 aa, Artificial Sequence
  Synthetic Construct
YAEHKSHRGEYSVCASESLWVTDKSSAIDIRGHQVTVLGEIKTGNSPVKQAFYETRCKEARPVKNGCRGIDDKHWNSQCKTSQTYVRALTSENNKLVGWRWIAIDTSCVCALSRKIGRT

SEQ ID NO: 56 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIAIDTSCVCALSAAIGRT

SEQ ID NO: 57 - protein, 119 aa, Artificial Sequence
  Synthetic Construct
YAEHKSHRGEYSVCASESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 58 - protein, 119 aa, Artificial Sequence
  Synthetic Construct
YAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSAAIGRT

SEQ ID NO: 59 - protein, 119 aa, Artificial Sequence
  Synthetic Construct
YAEHKSHRGEYSVCASESLWVTDKSSAIDIAGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 60 - protein, 119 aa, Artificial Sequence
  Synthetic Construct
YAEHKSHRGEYSVCASESLWVTDKSSAIDIAGHQVTVLGAIKTGNSPVKQYFYETRCKEARPVKNGCRGIDDKHWNSQCKTSQTYVRALTSENNKLVGWRWIRIDTSCVCALSRKIGRT

SEQ ID NO: 61 - protein, 200 aa, Artificial Sequence
  Synthetic Construct
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 62 - protein, 195 aa, Artificial Sequence
  Synthetic Construct
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 63 - protein, 153 aa, Artificial Sequence
  Synthetic Construct
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 64 - protein, 137 aa, Artificial Sequence
  Synthetic Construct
MITPTVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 65 - protein, 158 aa, Artificial Sequence
  Synthetic Construct
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 66 - protein, 180 aa, Artificial Sequence
  Synthetic Construct
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 67 - protein, 183 aa, Artificial Sequence
  Synthetic Construct
MGIPMGKSMLVLLTFLAFASCCIAAYRPSETLCGGELVDTLQFVCGDRGFYFRLPGRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 68 - protein, 236 aa, Artificial Sequence
  Synthetic Construct
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 69 - DNA, 368 bp, Artificial Sequence
  Synthetic Construct
gatcttcctcccacccaatcttccacagaggtgaattctctgtctgtgactctgtctctgtctgggttggtgataagaccactgctaccgacatcaagggtaaggaagtcatggttttgggtgaagttaacattaacaactccgttttcaagcaatacttcttcgaaactaagtgtagagatccaaacccagttgactctggttgtagaggtatcgattccaagcactggaactcttactgtaccactacccacactttcgtcaaggctttgactatggacggtaagcaagctgcctggagattcatcagaattgacaccgcttgtgtctgtgttttgtctagaaaggctgttagaagagcttgataa

SEQ ID NO: 70 - DNA, 811 bp, Artificial Sequence
  Synthetic Construct
atggttacttttgccacgatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacat

SEQ ID NO: 71 - DNA, 631 bp, Artificial Sequence
  Synthetic Construct
atgctccctctcccctcatgctccctccccatcctcctccttttcctcctccccagtgtgccaattgagtcccaacccccaccctcaacattgcccccttttctggcccctgagtgggaccttctctccccccgagtagtcctgtctaggggtgcccctgctgggccccctctgctcttcctgctggaggctggggcctttcgggagtcagcaggtgccccggccaaccgcagccggcgtggggtgagcgaaactgcaccagcgagtcgtcggggtgagctggctgtgtgcgatgcagtcagtggctgggtgacagaccgccggaccgctgtggacttgcgtgggcgcgaggtggaggtgttgggcgaggtgcctgcagctggcggcagtcccctccgccagtacttctttgaaacccgctgcaaggctgataacgctgaggaaggtggcccgggggcaggtggagggggctgccggggagtggacaggaggcactgggtatctgagtgcaaggccaagcagtcctatgtgcgggcattgaccgctgatgcccagggccgtgtgggctggcgatggattcgaattgacactgcctgcgtctgcacactcctcagccggactggccgggcct

SEQ ID NO: 72 - DNA, 784 bp, Artificial Sequence
  Synthetic Construct
acagagccgggaagaggaatggttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 73 - DNA, 987 bp, Artificial Sequence
  Synthetic Construct
atgtgtggagccaccagttttctccatgagtgcacaaggttaatccttgttactactcagaatgctgagtttctacagaaagggttgcaggtccacacatgttttggcgtctacccacacgcttctgtatggcatgactgtgcatcccagaagaagggctgtgctgtgtacctccacgtttcagtggaatttaacaaactgatccctgaaaatggtttcataaagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 74 - DNA, 765 bp, Artificial Sequence
  Synthetic Construct
atgttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 75 - DNA, 741 bp, Artificial Sequence
  Synthetic Construct
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 76 - DNA, 723 bp, Artificial Sequence
  Synthetic Construct
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcc

SEQ ID NO: 77 - DNA, 600 bp, Artificial Sequence
  Synthetic Construct
atggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatg

SEQ ID NO: 78 - DNA, 459 bp, Artificial Sequence
  Synthetic Construct
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatg

SEQ ID NO: 79 - DNA, 411 bp, Artificial Sequence
  Synthetic Construct
atgattacacctacagtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatg

SEQ ID NO: 80 - DNA, 474 bp, Artificial Sequence
  Synthetic Construct
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaag

SEQ ID NO: 81 - DNA, 540 bp, Artificial Sequence
  Synthetic Construct
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaag

SEQ ID NO: 82 - DNA, 708 bp, Artificial Sequence
  Synthetic Construct
atggtttccccagacccccaaattatcgtggtggcccccgagaccgaactcgcgtctatgcaagtccaacgcactgaggacggggtaaccattatccagatattttgggtgggccgcaaaggcgagctacttagacgcaccccggtgagctcggccatgcagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaag

(19) United States
(12) Patent Application Publication
(10) Pub. No.: US 2022/0054640 A1
(43) Pub. Date: Feb. 24, 2022

(54) COMPOSITIONS AND METHODS FOR THE DELIVERY OF THERAPEUTIC AGENTS ACROSS THE ROUND WINDOW MEMBRANE

(71) Applicant: Decibel Therapeutics, Inc., Boston, MA (US)

(72) Inventors: Qi-Ying HU, Needham, MA (US); John LEE, Arlington, MA (US); Paul H. LIEBESNY, Boston, MA (US); Changsuk MOON, Brookline, MA (US); Yong REN, Sharon, MA (US)

(21) Appl. No.: 17/413,223

(22) PCT Filed: Dec. 11, 2019
(86) PCT No.: PCT/US19/65804

§ 371 Date: Jun. 11, 2021

Related U.S. Application Data
(60) Provisional application No. 62/778,082, filed on Dec. 11, 2018.
(60) Provisional application No. 62/886,857, filed on Aug. 14, 2019.

(51) Int. Cl. A61K 47/42; A61K 9/00; A61K 45/06

(52) U.S. Cl. CPC A61K 47/42; A61K 45/06; A61K 9/0046

(57) ABSTRACT

The invention provides compositions and methods containing alpha-helical polypeptides as permeation enhancers for effectuating the passage of therapeutic agents into the inner ear by passage through the round window membrane. Examples of permeation-enhancing peptides useful in conjunction with the compositions and methods described herein are facially amphipathic, positively charged peptides. The permeation-enhancing peptides of the disclosure may be used to effectuate the entry of a variety of therapeutic agents into the inner ear, such as proteins, antibodies, nucleic acids, viral vectors, and nanoparticles, among others. Upon accessing the inner ear, the therapeutic agents may migrate to a particular site at which they may exert their biological effect. Exemplary conditions that may be treated/or prevented using the pharmaceutical compositions described herein are, without limitation, otic diseases, such as ceruminosis, ear pruritus, otitis externa, otalgia, tinnitus, vestibular dysfunction, ear fullness, hearing loss, Meniere's disease, auditory neuropathy, and sensorineural hearing loss, among others.

### SEQUENCE LISTING

The instant application contains a Sequence Listing which has been submitted electronically in ASCII format and is hereby incorporated by reference in its entirety. Said ASCII copy, created Dec. 11, 2019 is named 51124-047WO3_Sequence_Listing_12.11.19_ST25.txt and is 92,404 bytes in size.

### FIELD OF THE INVENTION

The invention relates to the field of therapeutic treatment, such as of otic diseases and conditions in human patients.

### BACKGROUND OF THE INVENTION

The inner ear represents an environment in which a variety of important cells and tissue systems reside. Among the cells that are resident in this niche are sensory hair cells, which are specialized mechanosensory receptors found in vertebrate auditory, vestibular and lateral line organs that transduce vibratory and acoustic stimuli into the sensations of hearing and balance. Sensorineural hearing loss typically occurs when hair cells are damaged from infection, noise exposure, ototoxins, and age-related decline. Worldwide, 1.3 billion humans suffer some form of hearing loss, while 360 million suffer debilitating hearing loss as a direct result of the absence of these sensory hair cells. Numerous well-known drugs are known to have ototoxic effects, including aminoglycoside antibiotics, cisplatin, loop diuretics, antimalarial sesquiterpene lactone endoperoxides, antimalarial quinines, salicylates, and interferon polypeptides. However, delivery therapeutic agents into the inner ear where they may exert a beneficial phenotype remains a challenge due to the difficulty associated with penetrating the epithelial barrier of the round window membrane. There exists a need for improved pharmaceutical compositions capable of effectuating the passage of therapeutic agents across the round window membrane.

### SUMMARY OF THE INVENTION

The present disclosure relates to compositions and methods for the otic delivery of therapeutic agents to subjects in need thereof, such as human subjects. Particularly, the compositions and methods described herein can be used to effectuate the passage of therapeutic agents across the round window membrane of a subject into the inner ear, where the therapeutic agents may exert a beneficial effect. The compositions and methods described herein solve a challenging biological problem, as the administration of therapeutic agents—particularly large substances, such as proteins, viral vectors, nucleic acids, and nanoparticles, among others—across the round window membrane has been a difficult barrier to overcome. The compositions and methods described herein provide important clinical benefits, as these can be used not only to deliver therapeutic agents across the round window membrane and into the inner ear, but also sustain high, therapeutically effective concentrations of these agents in vivo for long periods of time.

In a first aspect, the invention features a pharmaceutical composition formulated for otic administration to a human patient. The pharmaceutical composition may contain a therapeutic agent and a permeation enhancer. In some embodiments, the permeation enhancer is a polypeptide, such as an alpha-helical, facially amphipathic polypeptide. The polypeptide may have a pl of at least 7.0, such as a pl of from about 7.0 to about 12.0 or higher, as described herein, and may have, e.g., a net positive charge at physiological pH. In some embodiments, the polypeptide has a molecular weight of from about 1,000 Da to about 3,500 Da (e.g., a molecular weight of from about 1,100 Da to about 3,400 Da, from about 1,200 Da to about 3,300 Da, from about 1,300 Da to about 3,200 Da, from about 1,400 Da to about 3,100 Da, from about 1,500 Da to about 3,000 Da, from about 1,500 Da to about 2,900 Da, from about 1,500 Da to about 2,800 Da, from about 1,500 Da to about 2,600 Da, from about 1,500 Da to about 2,500 Da, from about 1,500 Da to about 2,400 Da, from about 1,600 Da to about 2,300 Da, from about 1,700 Da to about 2,200 Da, from about 1,800 Da to about 2,100 Da, from about 1,900 Da to about 2,100 Da, or from about 1,950 Da to about 2,050 Da).

In some embodiments, the polypeptide has a molecular weight of about 1,000 Da. In some embodiments, the polypeptide has a molecular weight of about 1,050 Da. In some embodiments, the polypeptide has a molecular weight of about 1,100 Da. In some embodiments, the polypeptide has a molecular weight of about 1,150 Da. In some embodiments, the polypeptide has a molecular weight of about 1,200 Da. In some embodiments, the polypeptide has a molecular weight of about 1,250 Da. In some embodiments, the polypeptide has a molecular weight of about 1,300 Da. In some embodiments, the polypeptide has a molecular weight of about 1,350 Da. In some embodiments, the polypeptide has a molecular weight of about 1,400 Da. In some embodiments, the polypeptide has a molecular weight of about 1,450 Da. In some embodiments, the polypeptide has a molecular weight of about 1,500 Da. In some embodiments, the polypeptide has a molecular weight of about 1,550 Da. In some embodiments, the polypeptide has a molecular weight of about 1,600 Da. In some embodiments, the polypeptide has a molecular weight of about 1,650 Da. In some embodiments, the polypeptide has a molecular weight of about 1,700 Da. In some embodiments, the polypeptide has a molecular weight of about 1,750 Da. In some embodiments, the polypeptide has a molecular weight of about 1,800 Da. In some embodiments, the polypeptide has a molecular weight of about 1,850 Da. In some embodiments, the polypeptide has a molecular weight of about 1,900 Da. In some embodiments, the polypeptide has a molecular weight of about 1,950 Da. In some embodiments, the polypeptide has a molecular weight of about 2,000 Da. In some embodiments, the polypeptide has a molecular weight of about 2,050 Da. In some embodiments, the polypeptide has a molecular weight of about 2,100 Da. In some embodiments, the polypeptide has a molecular weight of about 2,150 Da. In some embodiments, the polypeptide has a molecular weight of about 2,200 Da. In some embodiments, the polypeptide has a molecular weight of about 2,250 Da. In some embodiments, the polypeptide has a molecular weight of about 2,300 Da. In some embodiments, the polypeptide has a molecular weight of about 2,350 Da. In some embodiments, the polypeptide has a molecular weight of about 2,400 Da. In some embodiments, the polypeptide has a molecular weight of about 2,450 Da. In some embodiments, the polypeptide has a molecular weight of about 2,500 Da. In some embodiments, the polypeptide has a molecular weight of about 2,550 Da. In some embodiments, the polypeptide has a molecular weight of about 2,600 Da. In some embodiments, the polypeptide has a molecular weight of about 2,650 Da. In some embodiments, the polypeptide has a molecular weight of about 2,700 Da. In some embodiments, the polypeptide has a molecular weight of about 2,750 Da. In some embodiments, the polypeptide has a molecular weight of about 2,800 Da. In some embodiments, the polypeptide has a molecular weight of about 2,850 Da. In some embodiments, the polypeptide has a molecular weight of about 2,900 Da. In some embodiments, the polypeptide has a molecular weight of about 2,950 Da. In some embodiments, the polypeptide has a molecular weight of about 3,000 Da. In some embodiments, the polypeptide has a molecular weight of about 3,050 Da. In some embodiments, the polypeptide has a molecular weight of about 3,100 Da. In some embodiments, the polypeptide has a molecular weight of about 3,150 Da. In some embodiments, the polypeptide has a molecular weight of about 3,200 Da. In some embodiments, the polypeptide has a molecular weight of about 3,250 Da. In some embodiments, the polypeptide has a molecular weight of about 3,300 Da. In some embodiments, the polypeptide has a molecular weight of about 3,350 Da. In some embodiments, the polypeptide has a molecular weight of about 3,400 Da. In some embodiments, the polypeptide has a molecular weight of about 3,450 Da. In some embodiments, the polypeptide has a molecular weight of about 3,500 Da.

In some embodiments, the polypeptide has a molecular weight of about 1,800 Da. In some embodiments, the polypeptide has a molecular weight of about 1,810 Da. In some embodiments, the polypeptide has a molecular weight of about 1,820 Da. In some embodiments, the polypeptide has a molecular weight of about 1,830 Da. In some embodiments, the polypeptide has a molecular weight of about 1,840 Da. In some embodiments, the polypeptide has a molecular weight of about 1,850 Da. In some embodiments, the polypeptide has a molecular weight of about 1,860 Da. In some embodiments, the polypeptide has a molecular weight of about 1,870 Da. In some embodiments, the polypeptide has a molecular weight of about 1,880 Da. In some embodiments, the polypeptide has a molecular weight of about 1,890 Da. In some embodiments, the polypeptide has a molecular weight of about 1,900 Da. In some embodiments, the polypeptide has a molecular weight of about 1,910 Da. In some embodiments, the polypeptide has a molecular weight of about 1,920 Da. In some embodiments, the polypeptide has a molecular weight of about 1,930 Da. In some embodiments, the polypeptide has a molecular weight of about 1,940 Da. In some embodiments, the polypeptide has a molecular weight of about 1,950 Da. In some embodiments, the polypeptide has a molecular weight of about 1,960 Da. In some embodiments, the polypeptide has a molecular weight of about 1,970 Da. In some embodiments, the polypeptide has a molecular weight of about 1,980 Da. In some embodiments, the polypeptide has a molecular weight of about 1,990 Da. In some embodiments, the polypeptide has a molecular weight of about 2,000 Da. In some embodiments, the polypeptide has a molecular weight of about 2,010 Da. In some embodiments, the polypeptide has a molecular weight of about 2,020 Da. In some embodiments, the polypeptide has a molecular weight of about 2,030 Da. In some embodiments, the polypeptide has a molecular weight of about 2,040 Da. In some embodiments, the polypeptide has a molecular weight of about 2,050 Da. In some embodiments, the polypeptide has a molecular weight of about 2,060 Da. In some embodiments, the polypeptide has a molecular weight of about 2,070 Da. In some embodiments, the polypeptide has a molecular weight of about 2,080 Da. In some embodiments, the polypeptide has a molecular weight of about 2,090 Da. In some embodiments, the polypeptide has a molecular weight of about 2,100 Da.

In some embodiments, the polypeptide contains one or more regions represented, from N-terminus to C-terminus, by formula (I)

X¹—X²—X² (I)

wherein each X¹ independently represents an amino acid containing a cationic side chain at physiological pH;

each X² independently represents an amino acid containing a hydrophobic side chain; and

each “—” independently represents a peptide bond or a peptide bond isostere;

or a retro-inverso peptide thereof.

In some embodiments of formula (I), each X¹ independently represents an amino acid containing a lysine or arginine side chain; and each X² independently represents an amino acid containing an alanine, leucine, isoleucine, valine, methionine, tryptophan, phenylalanine, cysteine, or tyrosine side chain.

In some embodiments of formula (I), each X¹ independently represents an amino acid containing a lysine or arginine side chain; and each X² independently represents an amino acid containing an alanine, leucine, isoleucine, valine, or tryptophan side chain.

In some embodiments of formula (I), each X¹ independently represents an amino acid containing a lysine side chain; and each X² independently represents an amino acid containing an alanine, leucine, or tryptophan side chain.

In some embodiments, the polypeptide contains from 2 to 10 of the regions represented by formula (I), such as 2 regions represented by formula (I), 3 regions represented by formula (I), 4 regions represented by formula (I), 5 regions represented by formula (I), 6 regions represented by formula (I), 7 regions represented by formula (I), 8 regions represented by formula (I), 9 regions represented by formula (1), or 10 regions represented by formula (I). In some embodiments, the polypeptide contains from 3 to 7 of the regions represented by formula (I), such as 3 regions represented by formula (I), 4 regions represented by formula (I), 5 regions represented by formula (I), 6 regions represented by formula (I), or 7 regions represented by formula (I). In some embodiments, the polypeptide contains 5 of the regions represented by formula (I).

In some embodiments, each of the regions represented by formula (I) are consecutive or separated by up to two amino acid residues.

In some embodiments, the polypeptide contains one or more regions represented, from N-terminus to C-terminus, by formula (II)

X³—X⁴—X⁵—X⁴ (II)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain; and

each X⁵ independently represents an amino acid containing an alanine side chain;

or a retro-inverso peptide thereof.

In some embodiments of formula (II), each X³ independently represents an amino acid containing a lysine or arginine side chain; each X⁴ independently represents an amino acid containing a leucine, isoleucine, valine, or tryptophan side chain; each X⁵ independently represents an amino acid containing an alanine side chain; and each “—” independently represents a peptide bond or a peptide bond isostere.

In some embodiments of formula (II), each X³ independently represents an amino acid containing a lysine side chain; each X⁴ independently represents an amino acid containing a leucine or tryptophan side chain; and each X⁵ independently represents an amino acid containing an alanine side chain.

In some embodiments, the polypeptide contains from 1 to 10 of the regions represented by formula (II), such as 1 region represented by formula (II), 2 regions represented by formula (II), 3 regions represented by formula (II), 4 regions represented by formula (II), 5 regions represented by formula (II), 6 regions represented by formula (II), 7 regions represented by formula (II), 8 regions represented by formula (II), 9 regions represented by formula (II), or 10 regions represented by formula (II). In some embodiments, the polypeptide contains from 2 to 5 of the regions represented by formula (II), such as 2 regions represented by formula (II), 3 regions represented by formula (II), 4 regions represented by formula (II), or 5 regions represented by formula (II). In some embodiments, the polypeptide contains 3 regions represented by formula (II).

In some embodiments, the polypeptide contains one or more regions represented, from N-terminus to C-terminus, by formula (III)

X³— X⁴—X⁵—X⁴—X³—X⁴—X⁵—X⁴ (III)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain; and

each “—” independently represents a peptide bond or a peptide bond isostere;

or a retro-inverso peptide thereof.

In some embodiments of formula (III), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments of formula (III), each X³ independently represents an amino acid containing a lysine side chain; each X⁴ independently represents an amino acid containing a leucine or tryptophan side chain; and each X⁵ independently represents an amino acid containing an alanine side chain.

In some embodiments, the polypeptide contains from 1 to 5 of the regions represented by formula (III), such as 1 region represented by formula (III), 2 regions represented by formula (III), 3 regions represented by formula (III), 4 regions represented by formula (III), or 5 regions represented by formula (III). In some embodiments, the polypeptide contains one region represented by formula (III).

In some embodiments, the polypeptide contains a region represented, from N-terminus to C-terminus, by formula (IV)

[X³—X⁴— X⁵— X⁴]ₙ—[X³—X⁴—X⁵]ₘ (IV)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5 (i.e., 1, 2, 3, 4, or 5);

m represents an integer from 1 to 5; and

each “—” independently represents a peptide bond or a peptide bond isostere

or a retro-inverso peptide thereof.

In some embodiment of formula (IV), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiment of formula (IV), each X³ independently represents an amino acid containing a lysine side chain; each X⁴ independently represents an amino acid containing a leucine or tryptophan side chain; and each X⁵ independently represents an amino acid containing an alanine side chain.

In some embodiment of formula (IV), n represents an integer from 2 to 4. For example, in some embodiments, n is 2. In some embodiment of formula (IV), m represents an integer from 1 to 3. In some embodiments, m is 1.

In some embodiments, the polypeptide contains a region represented, from N-terminus to C-terminus, by formula (V)

[X³—X⁴—X⁵—X⁴]ₙ—[X³—X⁴— X⁵]ₘ—[X³—X⁴— X⁵— X⁵—X⁴]_{q} (V)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5;

m represents an integer from 1 to 5;

q represents an integer from 1 to 5; and

each “—” independently represents a peptide bond or a peptide bond isostere;

or a retro-inverso peptide thereof.

In some embodiments of formula (V), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments of formula (V), each X³ independently represents an amino acid containing a lysine side chain; each X⁴ independently represents an amino acid containing a leucine or tryptophan side chain; and each X⁵ independently represents an amino acid containing an alanine side chain.

In some embodiments of formula (V), n represents an integer from 2 to 4. In some embodiments of formula (V), n is 2. In some embodiments of formula (V), m represents an integer from 1 to 3. In some embodiments of formula (V), m is 1. In some embodiments of formula (V), q represents an integer from 1 to 3. In some embodiments of formula (V), q is 1.

In some embodiments, the polypeptide contains a region represented, from N-terminus to C-terminus, by formula (VI)

[X³—X⁴—X⁵—X⁴]ₙ—[X³—X⁴— X⁵]ₘ—[X³—X⁴— X⁵— X⁵— X⁴]_{q}—[X³—X⁴—X⁵]ᵣ (VI)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5;

m represents an integer from 1 to 5;

q represents an integer from 1 to 5;

r represents an integer from 1 to 5; and

each “—” independently represents a peptide bond or a peptide bond isostere;

or a retro-inverso peptide thereof.

In some embodiments of formula (VI), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments of formula (VI), each X³ independently represents an amino acid containing a lysine side chain; each X⁴ independently represents an amino acid containing a leucine or tryptophan side chain; and each X⁵ independently represents an amino acid containing an alanine side chain.

In some embodiments of formula (VI), n represents an integer from 2 to 4. In some embodiments of formula (VI), n is 2. In some embodiments of formula (VI), m represents an integer from 1 to 3. In some embodiments of formula (VI), m is 1. In some embodiments of formula (VI), q represents an integer from 1 to 3. In some embodiments of formula (VI), q is 1. In some embodiments of formula (VI), r represents an integer from 1 to 3. In some embodiments of formula (VI), r is 1.

In some embodiments of any of the above formulas, one or more of each “—” is a peptide bond isostere, such as a peptide bond isostere selected from:

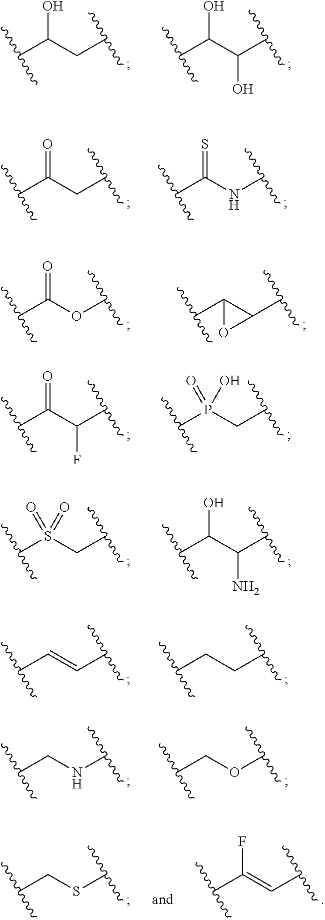

In some embodiments of any of the above formulas, the polypeptide contains one or more intramolecular crosslinks. The intramolecular crosslinks may stabilize a secondary structure of the polypeptide, such as an alpha-helical structure. In some embodiments, the one or more intramolecular crosslinks are ionic in nature, such as a salt bridge. In some embodiments, the one or more intramolecular crosslinks are formed of covalent bonds. For example, in some embodiments, the one or more intramolecular crosslinks are selected from:

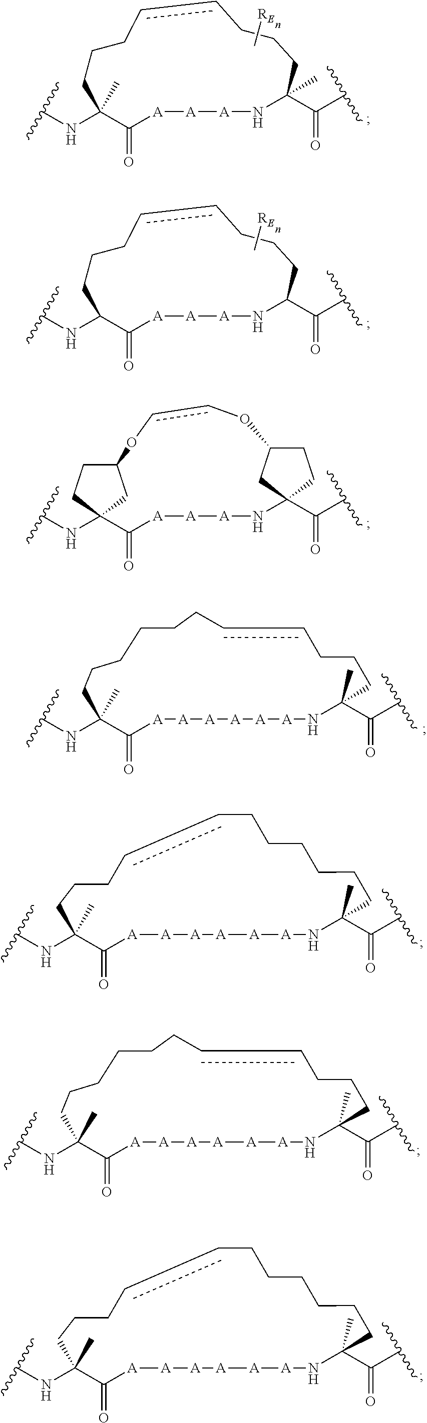

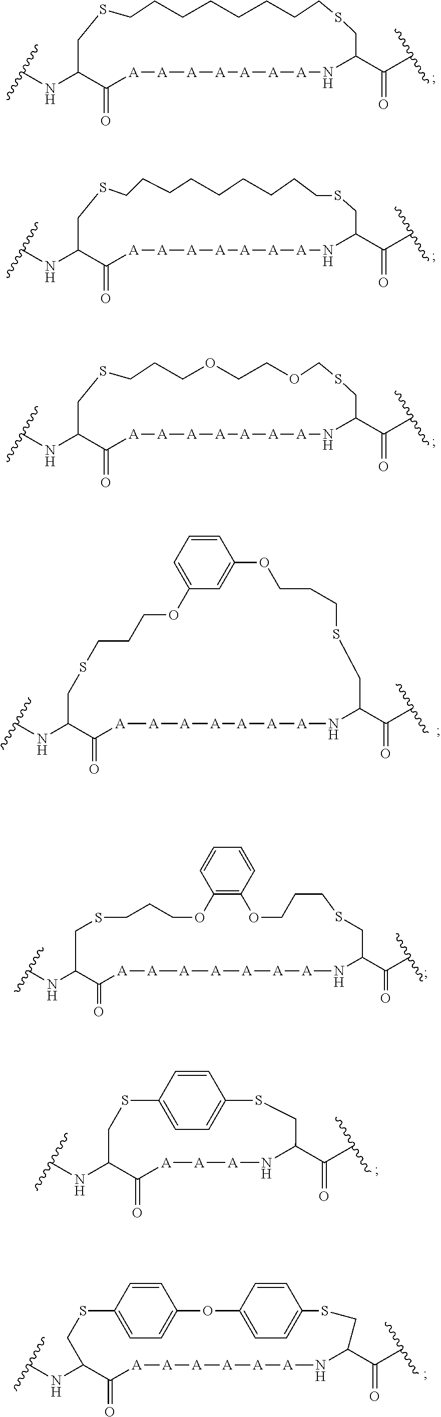

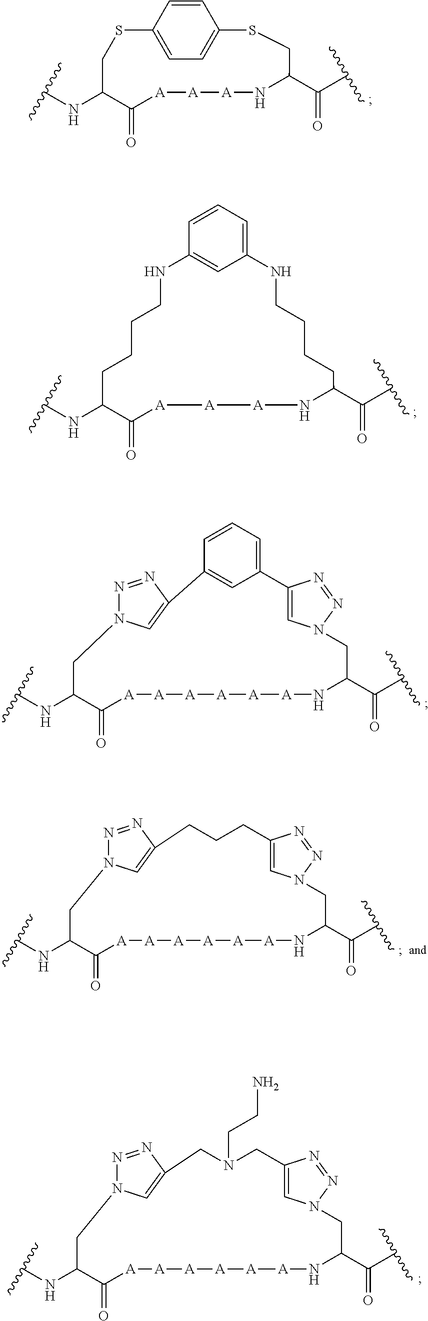

wherein each “A” represents an individual amino acid residue within the polypeptide.

In some embodiments, the polypeptide is cyclized from N-terminus to C-terminus.

In some embodiments, the polypeptide is from 10 to 30 amino acid residues in length, such as 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, or 30 amino acids in length. In some embodiments, the polypeptide is from 14 to 26 amino acids in length, such as 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, or 26 amino acids in length. In some embodiments, the polypeptide is from 15 to 20 amino acids in length, such as 15 amino acids in length, 16 amino acids in length, 17 amino acids in length, 18 amino acids in length, 19 amino acids in length, or 20 amino acids in length. In some embodiments, the polypeptide is 19 amino acids in length.

In some embodiments, the polypeptide has an isoelectric point (pl) of from about 8 to about 13. For example, the polypeptide may have an isoelectric point of about 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, or 13. Particularly, in some embodiments, the polypeptide has an isoelectric point of about 8.0, 8.01, 8.02, 8.03, 8.04, 8.05, 8.06, 8.07, 8.08, 8.09, 8.10, 8.11, 8.12, 8.13, 8.14, 8.15, 8.16, 8.17, 8.18, 8.19, 8.20, 8.21, 8.22, 8.23, 8.24, 8.25, 8.26, 8.27, 8.28, 8.29, 8.30, 8.31, 8.32, 8.33, 8.34, 8.35, 8.36, 8.37, 8.38, 8.39, 8.40, 8.41, 8.42, 8.43, 8.44, 8.45, 8.46, 8.47, 8.48, 8.49, 8.50, 8.51, 8.52, 8.53, 8.54, 8.55, 8.56, 8.57, 8.58, 8.59, 8.60, 8.61, 8.62, 8.63, 8.64, 8.65, 8.66, 8.67, 8.68, 8.69, 8.70, 8.71, 8.72, 8.73, 8.74, 8.75, 8.76, 8.77, 8.78, 8.79, 8.80, 8.81, 8.82, 8.83, 8.84, 8.85, 8.86, 8.87, 8.88, 8.89, 8.90, 8.91, 8.92, 8.93, 8.94, 8.95, 8.96, 8.97, 8.98, 8.99, 9.0, 9.01, 8.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, 12.0, 12.01, 8.02, 12.03, 12.04, 12.05, 12.06, 12.07, 12.08, 12.09, 12.10, 12.11, 12.12, 12.13, 12.14, 12.15, 12.16, 12.17, 12.18, 12.19, 12.20, 12.21, 12.22, 12.23, 12.24, 12.25, 12.26, 12.27, 12.28, 12.29, 12.30, 12.31, 12.32, 12.33, 12.34, 12.35, 12.36, 12.37, 12.38, 12.39, 12.40, 12.41, 12.42, 12.43, 12.44, 12.45, 12.46, 12.47, 12.48, 12.49, 12.50, 12.51, 12.52, 12.53, 12.54, 12.55, 12.56, 12.57, 12.58, 12.59, 12.60, 12.61, 12.62, 12.63, 12.64, 12.65, 12.66, 12.67, 12.68, 12.69, 12.70, 12.71, 12.72, 12.73, 12.74, 12.75, 12.76, 12.77, 12.78, 12.79, 12.80, 12.81, 12.82, 12.83, 12.84, 12.85, 12.86, 12.87, 12.88, 12.89, 12.90, 12.91, 12.92, 12.93, 12.94, 12.95, 12.96, 12.97, 12.98, 12.99, or 13.0.

In some embodiments, the polypeptide has a pl of from about 8.5 to about 12.5, such as a pl of 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, or 12.5 (e.g., a pl of 8.50, 8.51, 8.52, 8.53, 8.54, 8.55, 8.56, 8.57, 8.58, 8.59, 8.60, 8.61, 8.62, 8.63, 8.64, 8.65, 8.66, 8.67, 8.68, 8.69, 8.70, 8.71, 8.72, 8.73, 8.74, 8.75, 8.76, 8.77, 8.78, 8.79, 8.80, 8.81, 8.82, 8.83, 8.84, 8.85, 8.86, 8.87, 8.88, 8.89, 8.90, 8.91, 8.92, 8.93, 8.94, 8.95, 8.96, 8.97, 8.98, 8.99, 9.0, 9.01, 8.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, 12.0, 12.01, 8.02, 12.03, 12.04, 12.05, 12.06, 12.07, 12.08, 12.09, 12.10, 12.11, 12.12, 12.13, 12.14, 12.15, 12.16, 12.17, 12.18, 12.19, 12.20, 12.21, 12.22, 12.23, 12.24, 12.25, 12.26, 12.27, 12.28, 12.29, 12.30, 12.31, 12.32, 12.33, 12.34, 12.35, 12.36, 12.37, 12.38, 12.39, 12.40, 12.41, 12.42, 12.43, 12.44, 12.45, 12.46, 12.47, 12.48, 12.49, or 12.50).

In some embodiments, the polypeptide has a pl of from about 9 to about 12, such as a pl of 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, or 12.5 (e.g., a pl of 9.0, 9.01, 8.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, or 12.0).

In some embodiments, the polypeptide has a pl of from about to about 9.5 to about 11.5, such as a pl of 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, or 11.5 (e.g., a pl of 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, or 11.50).

In some embodiments, the polypeptide has a pl of from about 10 to about 11, such as a pl of 10, 10.1, 10.2, 10.3, 10.4, 10.5, 10.6, 10.7, 10.8, 10.9, or 11 (e.g., a pl of 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, or 11.0).

In some embodiments, the polypeptide has a pl of from about 10.3 to about 10.9, such as a pl of 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, or 10.90. In some embodiments, the polypeptide has a pl of about 10.6.

In some embodiments, the polypeptide has an alpha-helicity of at least about 50%, as assessed, for example, by circular dichroism. The polypeptide may have, for example, an alpha-helicity of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments, the polypeptide has an alpha-helicity of at least about 55%, at least about 60%, at least about 65%, at least about 70%, at least about 75%, or more. In some embodiments, the polypeptide has an alpha-helicity of from about 55% to about 85%, from about 51% to about 84%, from about 52% to about 83%, from about 53% to about 82%, from about 54% to about 81%, from about 55% to about 80%, from about 60% to about 75%, of from about 60% to about 70%. In some embodiments, the polypeptide has an alpha-helicity of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the polypeptide has an alpha-helicity of from about 61% to about 68%, such as an alpha-helicity of 61%, 62%, 63%, 64%, 65%, 66%, 67%, or 68%.

In some embodiments, the polypeptide represented by formula (VII)

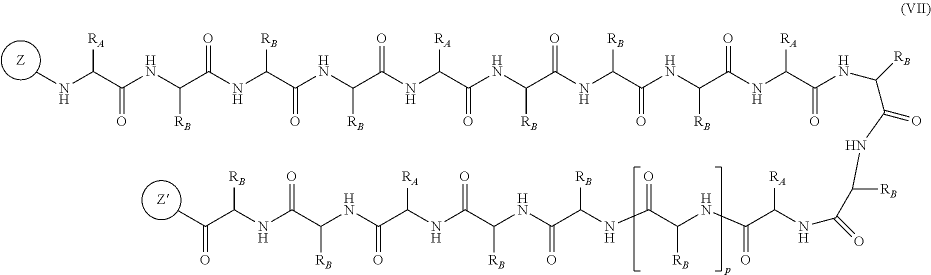

wherein each R_{A} is independently selected from:

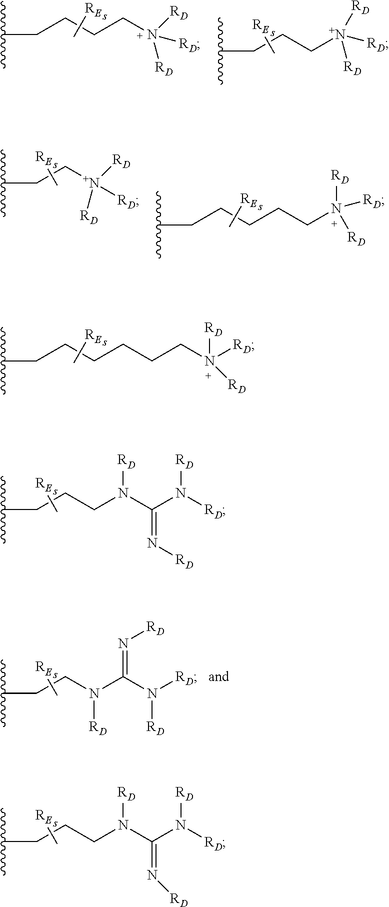

each R_{B} is independently selected from:

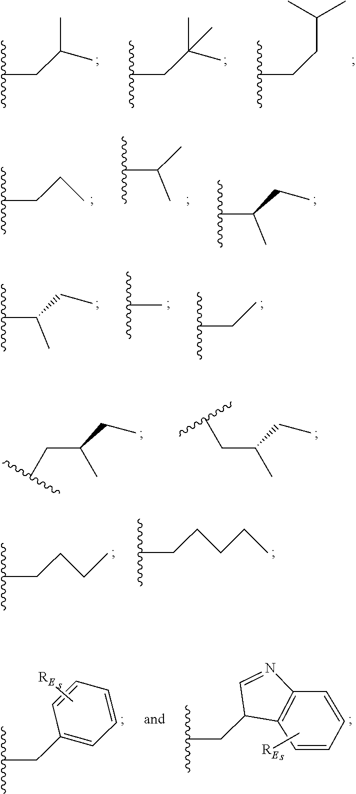

each R_{D} is independently hydrogen or an optionally substituted C₁-C₆ alkyl group;

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

p is an integer from 0 to 3;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂;

or a retro-inverso peptide thereof.

In some embodiments, the polypeptide is represented by formula (VIII)

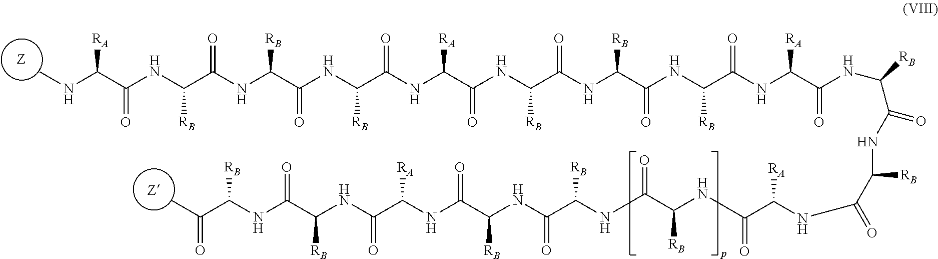

wherein each of R^{A}, R^{B}, R^{D}, R^{E}, p, s, Z, and Z′ are as defined for formula (VII);

or a retro-inverso peptide thereof.

In some embodiments, the polypeptide is represented by formula (IX)

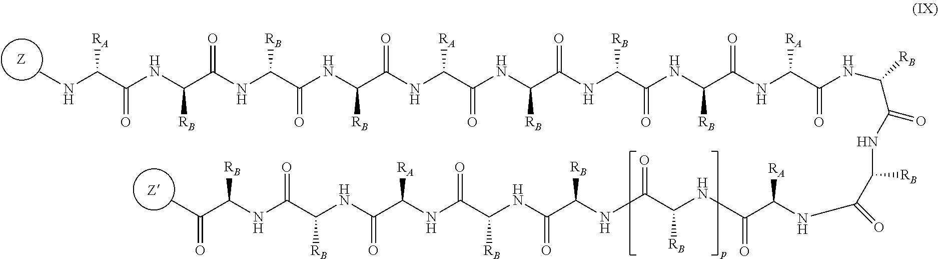

wherein each of R^{A}, R^{B}, R^{D}, R^{E}, p, s, Z, and Z′ are as defined for formula (VII);

or a retro-inverso peptide thereof.

In some embodiments, the polypeptide is represented by formula (X)

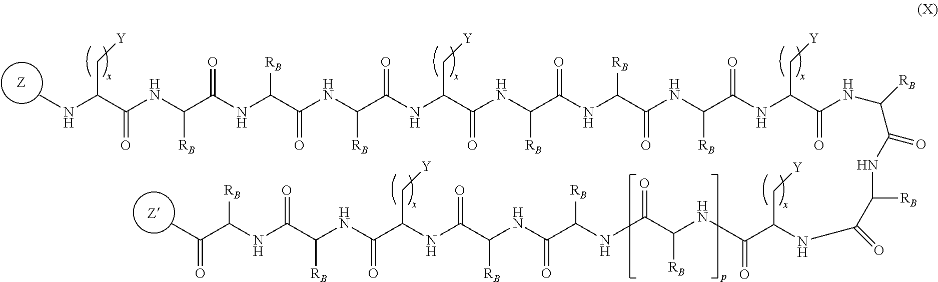

wherein each Y is independently an optionally substituted amino group or an optionally substituted guanidinium group;

each R_{B} is independently selected from:

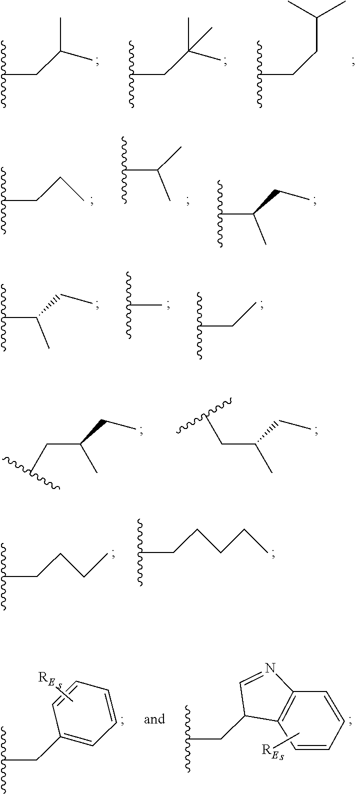

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

x is an integer from 3 to 5;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂ or a retro-inverso peptide thereof.

In some embodiments, the polypeptide is represented by formula (XI)

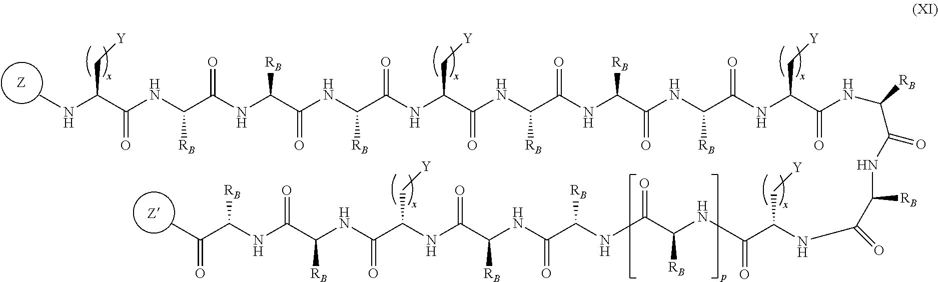

wherein each of Y, R^{B}, R^{E}, x, s, Z, and Z′ are as defined for formula (X);

or a retro-inverso peptide thereof.

In some embodiments, the polypeptide is represented by formula (XII)

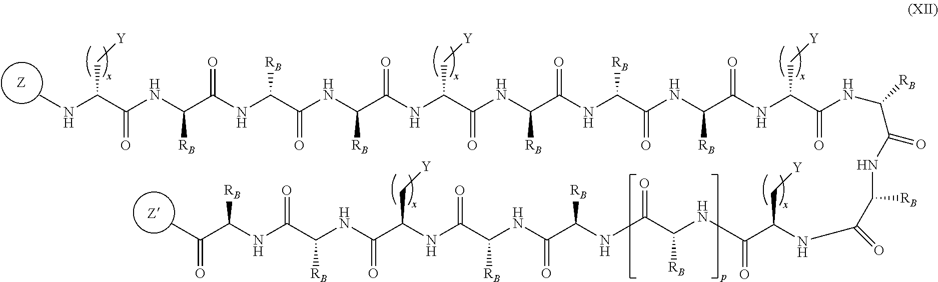

wherein each of Y, R^{B}, R^{E}, x, s, Z, and Z′ are as defined for formula (X);

or a retro-inverso peptide thereof.

In some embodiments, the polypeptide is represented by formula (XIII)

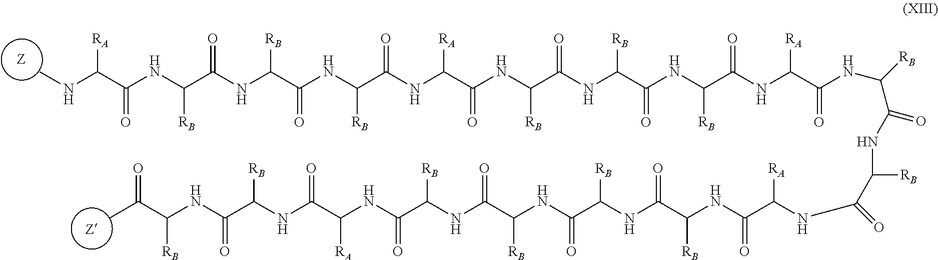

wherein each R_{A} is independently selected from:

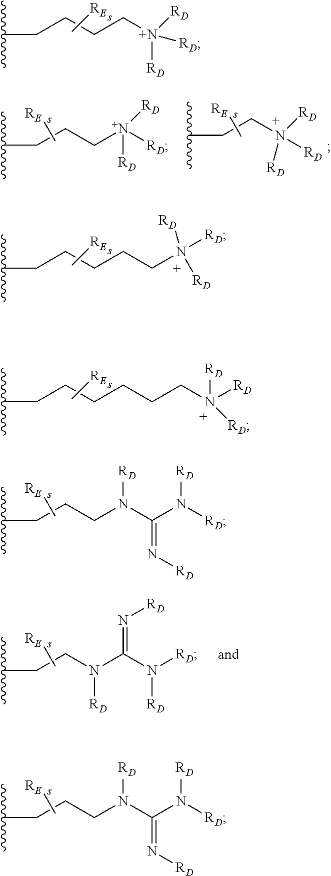

each R_{B} is independently selected from:

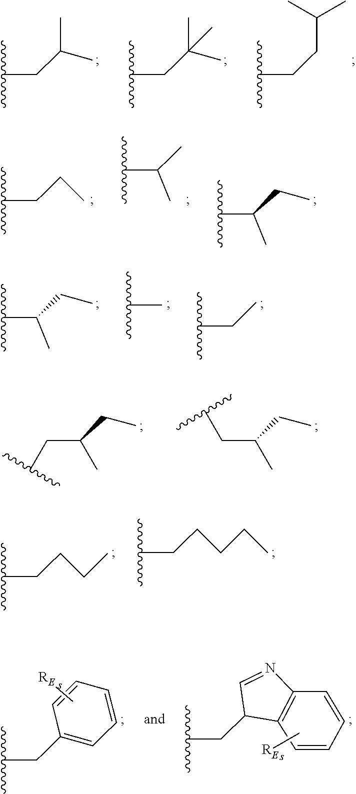

each R_{D} is independently hydrogen or an optionally substituted C₁-C₆ alkyl group;

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂;

or a retro-inverso peptide thereof.

In some embodiments, the polypeptide is represented by formula (XIV)

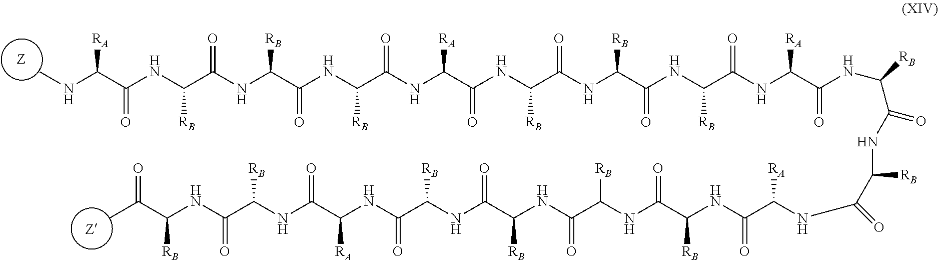

wherein each of R^{A}, R^{B}, R^{D}, R^{E}, s, Z, and Z′ are as defined for formula (XIII);

or a retro-inverso peptide thereof.

In some embodiments, the polypeptide is represented by formula (XV)

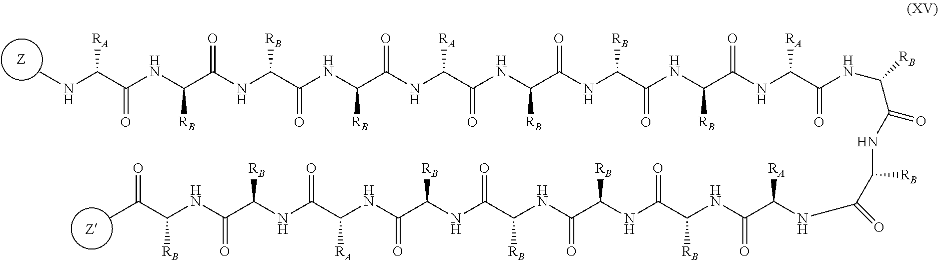

wherein each of R^{A}, R^{B}, R^{D}, R^{E}, s, Z, and Z′ are as defined for formula (XIII);

or a retro-inverso peptide thereof.

In some embodiments, the polypeptide is represented by formula (XVI)

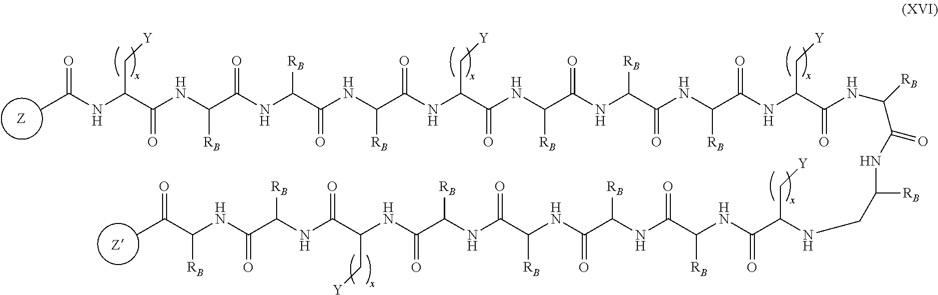

wherein each Y is independently an optionally substituted amino group or an optionally substituted guanidinium group;

each R_{B} is independently selected from:

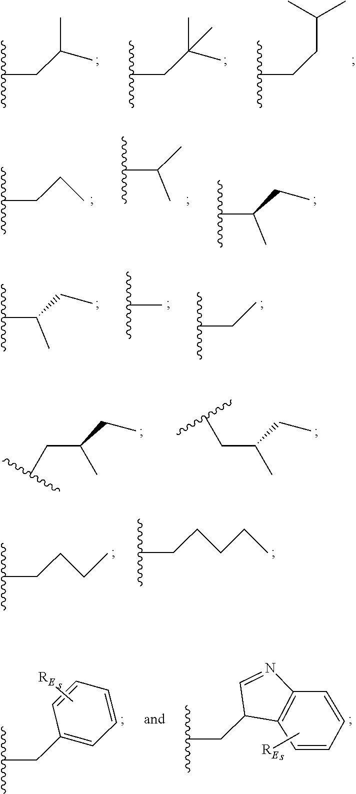

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

x is an integer from 3 to 5;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂;

or a retro-inverso peptide thereof.

In some embodiments, the polypeptide is represented by formula (XVII)

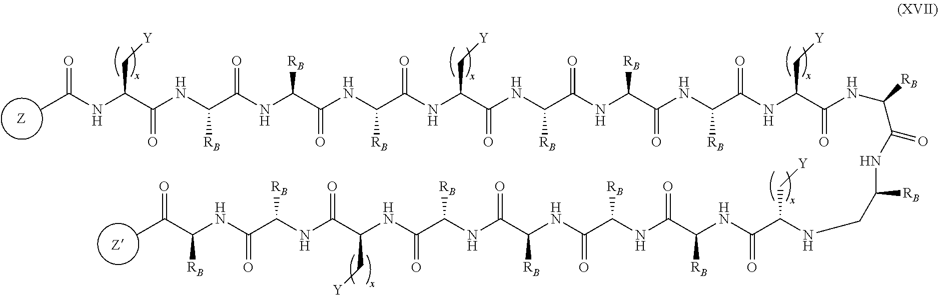

wherein each of Y, R^{B}, R^{E}, x, s, Z, and Z′ are as defined for formula (XVI);

or a retro-inverso peptide thereof.

In some embodiments, the polypeptide is represented by formula (XVIII)

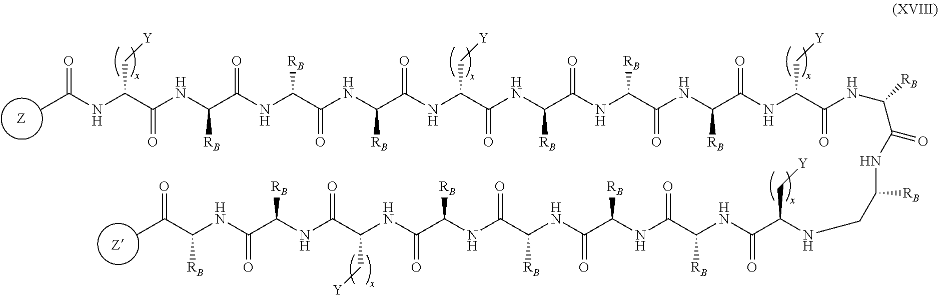

wherein each of Y, R^{B}, R^{E}, x, s, Z, and Z′ are as defined for formula (XVI);

or a retro-inverso peptide thereof.

In some embodiments, the polypeptide is represented by formula (XIX)

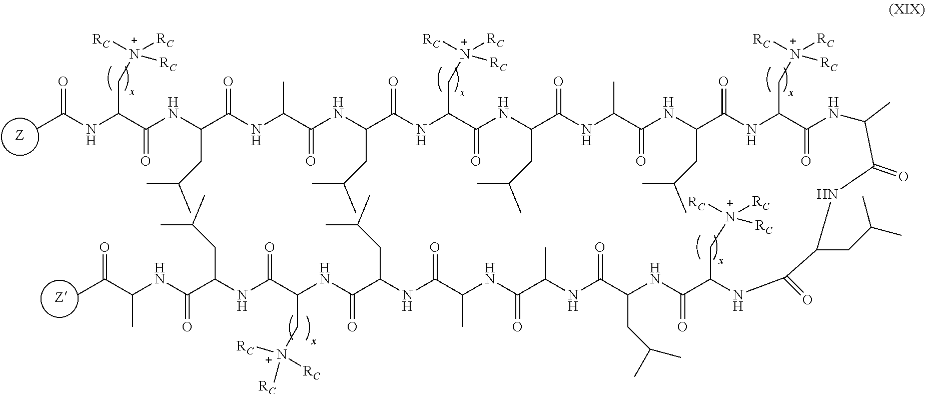

wherein each R_{C} is independently hydrogen or optionally substituted C₁-C₆ alkyl;

x is an integer from 3 to 5; such as 4;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂;

or a retro-inverso peptide thereof.

In some embodiments, the polypeptide is represented by formula (XX)

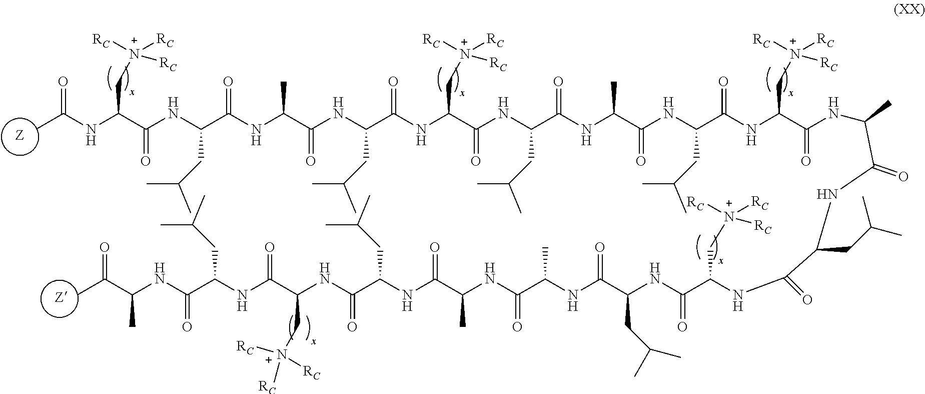

wherein R_{C}, x, Z, and Z′ are as defined for formula (XIX);

or a retro-inverso peptide thereof.

In some embodiments, the polypeptide is represented by formula (XXI)

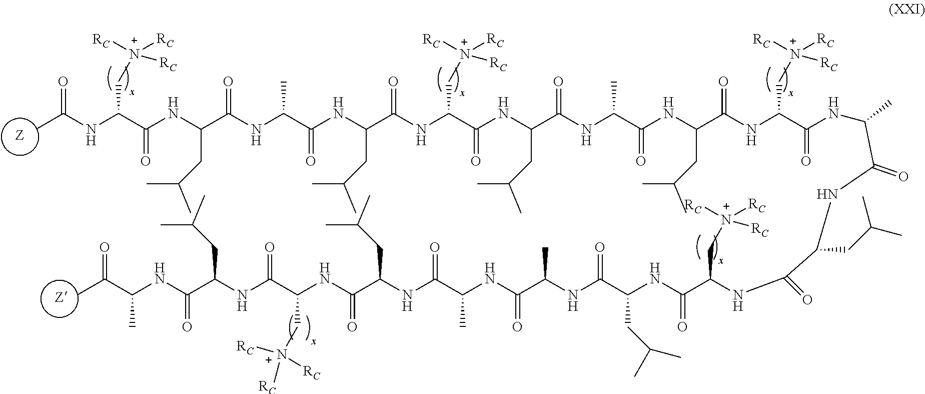

wherein R_{C}, x, Z, and Z′ are as defined for formula (XIX); or a retro-inverso peptide thereof.

In some embodiments, the polypeptide is represented by formula (XXII)

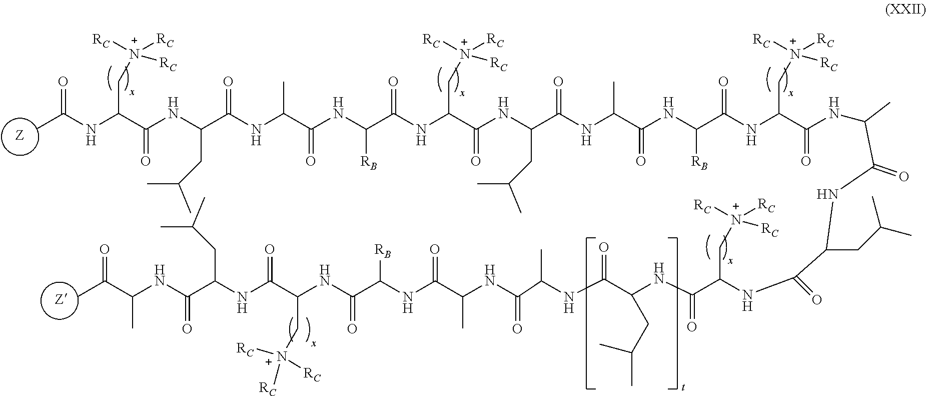

wherein each R_{C} is independently hydrogen or optionally substituted C₁-C₆ alkyl;

x is an integer from 3 to 5; such as 4;

t is 0 or 1;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂;

or a retro-inverso peptide thereof.

In some embodiments, the polypeptide is represented by formula (XXIII)

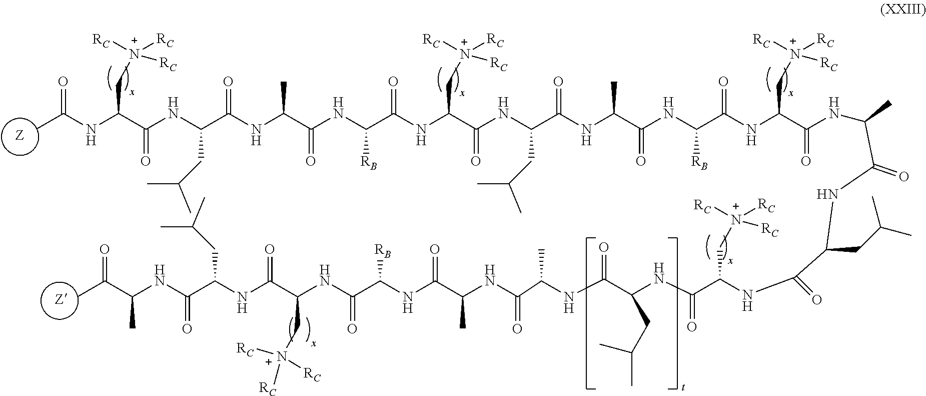

wherein R_{C}, x, t, Z, and Z′ are as defined for formula (XXII);

or a retro-inverso peptide thereof.

In some embodiments, the polypeptide is represented by formula (XXIV)

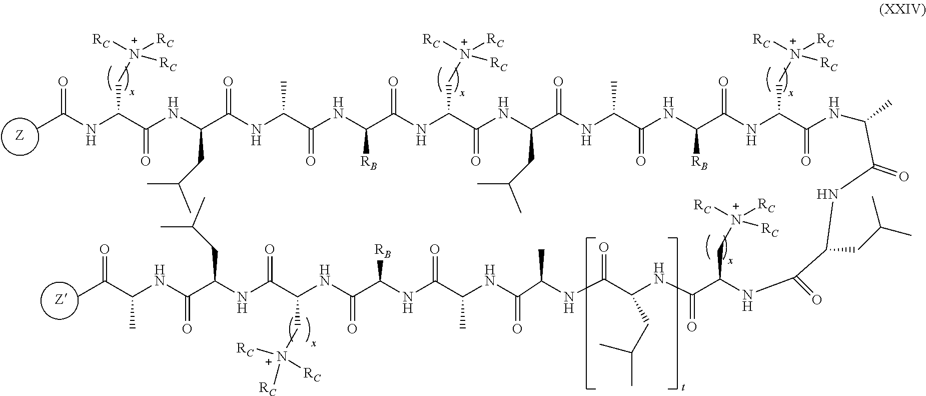

wherein R_{C}, x, t, Z, and Z′ are as defined for formula (XXII);

or a retro-inverso peptide thereof.

In some embodiments, the polypeptide is represented by formula (XXV)

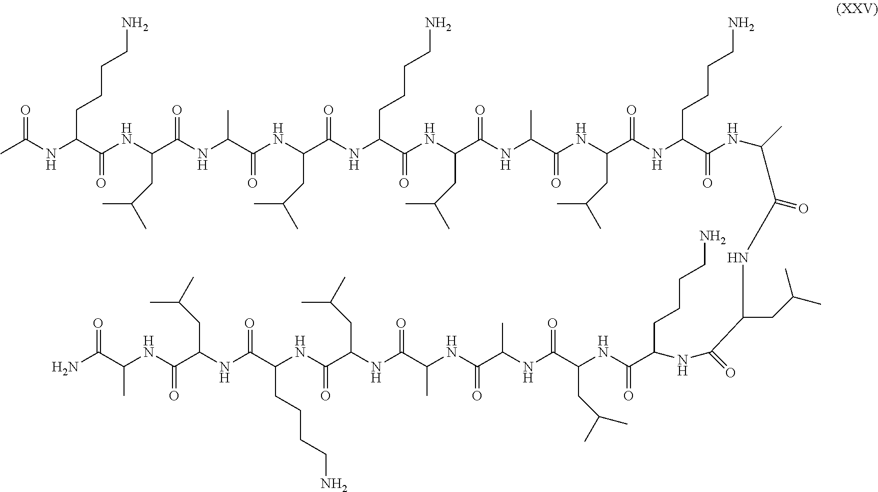 [SMILES: CC(=O)NC(CCCCN)C(=O)NC(CC(C)C)C(=O)NC(C)C(=O)NC(CC(C)C)C(=O)NC(CCCCN)C(=O)NC(CC(C)C)C(=O)NC(C)C(=O)NC(CC(C)C)C(=O)NC(CCCCN)C(=O)NC(C)C(=O)NC(CC(C)C)C(=O)NC(CCCCN)C(=O)NC(CC(C)C)C(=O)NC(C)C(=O)NC(C)C(=O)NC(CC(C)C)C(=O)NC(CCCCN)C(=O)NC(CC(C)C)C(=O)NC(C)C(N)=O]

or a pharmaceutically acceptable salt thereof;

or a retro-inverso peptide thereof.

In some embodiments, the polypeptide is represented by formula (XXVI)

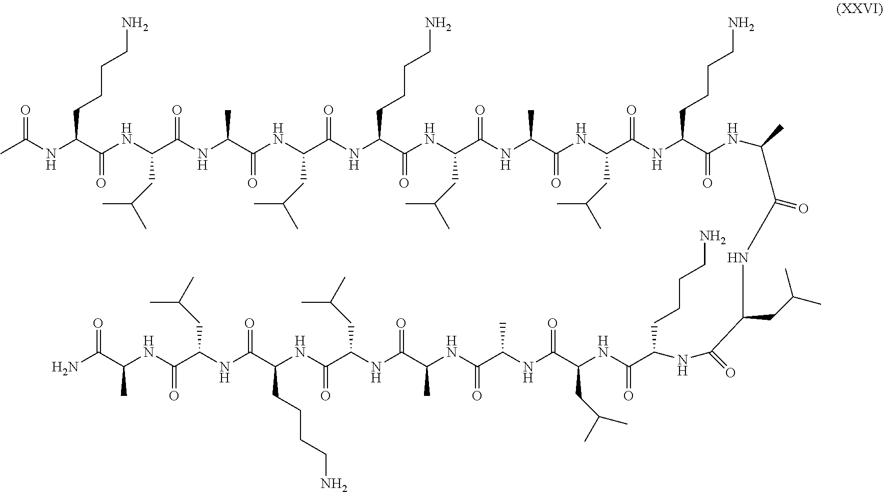 [SMILES: CC(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](C)C(=O)N[C@@H](CC(C)C)C(=O)N[C@@H](CCCCN)C(=O)N[C@@H](CC(C)C)C(=O)N[C@@H](C)C(N)=O]

or a pharmaceutically acceptable salt thereof;

or a retro-inverso peptide thereof.

In some embodiments, the polypeptide is represented by formula (XXVII)

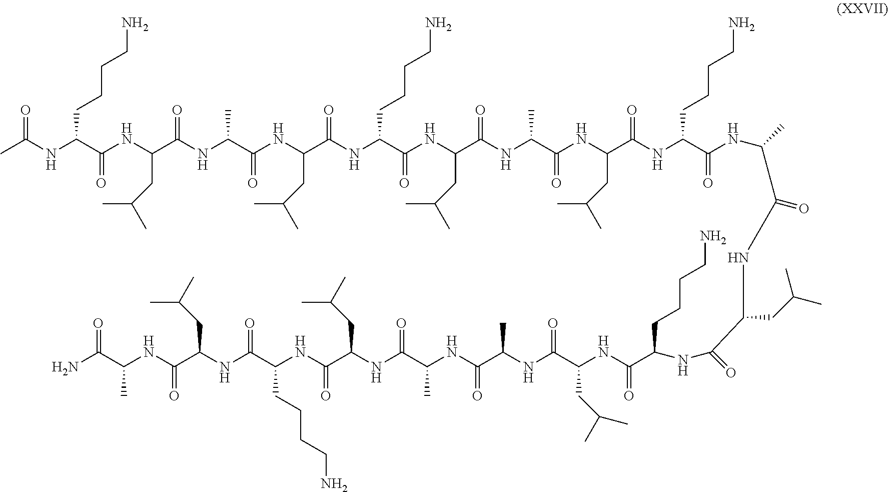 [SMILES: CC(=O)N[C@H](CCCCN)C(=O)NC(CC(C)C)C(=O)N[C@H](C)C(=O)NC(CC(C)C)C(=O)N[C@H](CCCCN)C(=O)NC(CC(C)C)C(=O)N[C@H](C)C(=O)NC(CC(C)C)C(=O)N[C@H](CCCCN)C(=O)N[C@H](C)C(=O)N[C@H](CC(C)C)C(=O)N[C@H](CCCCN)C(=O)N[C@H](CC(C)C)C(=O)N[C@H](C)C(=O)N[C@H](C)C(=O)N[C@H](CC(C)C)C(=O)N[C@H](CCCCN)C(=O)N[C@H](CC(C)C)C(=O)N[C@H](C)C(N)=O]

or a pharmaceutically acceptable salt thereof;

or a retro-inverso peptide thereof.

In some embodiments, the polypeptide contains a region having an amino acid sequence that is at least 85% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, polypeptide has an amino acid sequence that is at least 90% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, the polypeptide has an amino acid sequence that is at least 95% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to five amino acid substitutions (e.g., by one amino acid substitution, by two amino acid substitutions, by three amino acid substitutions, by four amino acid substitutions, by five amino acid substitutions, or by zero amino acid substitutions).

In some embodiments, the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to three amino acid substitutions.

In some embodiments, the amino acid substitutions are conservative amino acid substitutions.

In some embodiments, the polypeptide has an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, the polypeptide is present within the pharmaceutical composition at a concentration of from about 0.001% w/v to about 50% w/v. In some embodiments, the polypeptide is present within the pharmaceutical composition at a concentration of from about 0.01% w/v to about 10% w/v. In some embodiments, the polypeptide is present within the pharmaceutical composition at a concentration of from about 0.1% w/v to about 5% w/v. In some embodiments, the polypeptide is present within the pharmaceutical composition at a concentration of from about 0.1% w/v to about 1% w/v. In some embodiments, the polypeptide is present within the pharmaceutical composition at a concentration of about 1% w/v. In some embodiments, the polypeptide is present within the pharmaceutical composition at a concentration of from about 0.1% w/v to about 1% w/v. In some embodiments, the polypeptide is present within the pharmaceutical composition at a concentration of from about 0.1% w/v to about 0.25% w/v. In some embodiments, the polypeptide is present within the pharmaceutical composition at a concentration of about 0.1% w/v. In some embodiments, the polypeptide is present within the pharmaceutical composition at a concentration of about 0.25% w/v.

In some embodiments, the therapeutic agent is present within the pharmaceutical composition at a concentration of from about 0.001% w/v to about 50% w/v. In some embodiments, the therapeutic agent is present within the pharmaceutical composition at a concentration of from about 0.01% w/v to about 10% w/v. In some embodiments, the therapeutic agent is present within the pharmaceutical composition at a concentration of from about 0.1% w/v to about 5% w/v. In some embodiments, the therapeutic agent is present within the pharmaceutical composition at a concentration of about 1% w/v.

In some embodiments, the pharmaceutical composition further contains a gelling agent. In some embodiments, the gelling agent is hyaluronan, hyaluronic acid, a polyoxyethylene-polyoxypropylene block copolymer, poly(lactic-co-glycolic) acid, polylactic acid, polycaprolactone, alginic acid or a salt thereof, polyethylene glycol, a cellulose, a cellulose ether, agar-agar, gelatin, glucomannan, galactomannan (e.g., locust bean gum or tara gum), xanthan gum, guar gum, chitosan, pectin, starch, tragacanth, carrageenan, polyvinylpyrrolidone, polyvinyl alcohol, paraffin, polyethoxylated sorbitan monolaurate, petrolatum, silicates, fibroin, gellan, CARBOPOL 940®, polyoxamines, lecithin gels, polysorbate-80, (poly)aniline derivatives, xyloglucane, collagen, silicon dioxide, tyloxapol, Cremophor, aluminum magnesium silicate, sodium stearate, bladderwrack, bentonite, eratonia, chondrus, dextrose, furcellaran, Ghatti gum, hectorite, lactose, sucrose, sucralose, maltodextrin, mannitol, sorbitol, honey, maize starch, wheat starch, rice starch, potato starch, oxypolygelatin, polygeline, sterculia gum, propylene carbonate, methyl vinyl ether/maleic anhydride copolymer, poly(methoxyethyl methacrylate), and poly(methoxyethoxyethyl methacrylate), and combinations thereof. In some embodiments, the gelling agent is a polyoxyethylene-polyoxypropylene block copolymer, alginic acid or a pharmaceutically acceptable salt thereof, collagen, hyaluronic acid or a pharmaceutically acceptable salt thereof, gelatin, or fibroin. In some embodiments, the polyoxyethylene-polyoxypropylene block copolymer is poloxamer 407. In some embodiments, the polyoxyethylene-polyoxypropylene block copolymer is poloxamer 188.

In some embodiments, the polyoxyethylene-polyoxypropylene block copolymer is present within the pharmaceutical composition at a concentration of from about 0.001% w/v to about 50% w/v. In some embodiments, the polyoxyethylene-polyoxypropylene block copolymer is present within the pharmaceutical composition at a concentration of from about 0.01% w/v to about 40% w/v. In some embodiments, the polyoxyethylene-polyoxypropylene block copolymer is present within the pharmaceutical composition at a concentration of from about 1% w/v to about 30% w/v. In some embodiments, the polyoxyethylene-polyoxypropylene block copolymer is present within the pharmaceutical composition at a concentration of about 20% w/v.

In some embodiments, upon administration (for example, upon intratympanic or transtympanic administration, such as intratympanic or transtympanic injection) to a mammalian subject, the therapeutic agent is delivered across the round window membrane of the subject. In some embodiments, upon administration (for example, upon intratympanic or transtympanic administration, such as intratympanic or transtympanic injection) to a mammalian subject, the therapeutic agent remains present within perilymph of the subject for at least from about 1 hour to about 6 weeks following the administration to the subject (e.g., at least about 1 hour, about 12 hours, about 24 hours, about 1 week, about 2 weeks, about 3 weeks, about 4 weeks, about 5 weeks, about 6 weeks, or more). In some embodiments, upon administration (for example, upon intratympanic or transtympanic administration, such as intratympanic or transtympanic injection) to a mammalian subject, the therapeutic agent remains present within perilymph of the subject for at least from about 4 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 8 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 12 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 24 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 48 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 72 hours to about 6 weeks following the administration to the subject. In some embodiments, upon administration (for example, upon intratympanic or transtympanic administration, such as intratympanic or transtympanic injection) to a mammalian subject, the therapeutic agent remains present within perilymph of the subject for at least from about 96 hours to about 6 weeks following the administration to the subject.

In some embodiments, the pharmaceutical composition contains a unit dosage form having a volume of from about 50 μL to about 1 mL. In some embodiments, the pharmaceutical composition contains a unit dosage form having a volume of from 100 μL to about 1 mL, from about 200 μL to about 1 mL, from about 300 μL to 0.8 mL, from about 200 μL to about 0.8 mL, from about 100 μL to about 0.5 mL, from about 200 μL to about 0.5 mL, from about 0.5 mL to about 1.0 mL, from about 0.5 mL to about 0.8 mL, or from about 0.8 mL to about 1.0 mL.

In some embodiments, the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, or a WNT modulator. In some embodiments, the therapeutic agent is a TrkB receptor agonist antibody. In some embodiments, the therapeutic agent is a TrkC receptor agonist antibody. In some embodiments, the therapeutic agent is a Wnt modulator. In some embodiments, the therapeutic agent is an Atoh1 modulator (e.g., an Atoh1 polypeptide or a nucleic acid vector engineered to express Atoh1, e.g., human Atoh1 (Hath1)).

In some embodiments, the therapeutic agent is a neurotrophin. In some embodiments, the neurotrophin is selected from neurotrophin-3 (NT-3), nerve growth factor (NGF), brain-derived neurotrophic factor (BDNF), ciliary neurotrophic factor (CNTF), a pan-neurotrophin (e.g., PNT-1) a chimeric neurotrophin, glial cell-line derived neurotrophic factor (GDNF), neurotrophin-4 (NT-4), fibroblast growth factor (FGF), insulin-like growth factor (IGF), epidermal growth factor (EGF), platelet-derived growth factor (PGF), mesencephalic astrocyte-derived neurotrophic factor (MANF), cerebral dopamine neurotrophic factor (CDNF), and combinations thereof.

In some embodiments, the neurotrophin is NT-3. In some embodiments, the NT-3 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 15. In some embodiments, the NT-3 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 21. In some embodiments, the NT-3 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 22. In some embodiments, the NT-3 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 15. In some embodiments, the NT-3 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 21. In some embodiments, the NT-3 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 22. In some embodiments, the NT-3 has an amino acid sequence having at least 95% (e.g., at least 95%, 96%, 97%, 98%, 99%, 99.9%, 99.99%, or 100% sequence identity) to the amino acid sequence of SEQ ID NO: 15. In some embodiments, the NT-3 has an amino acid sequence having at least 95% (e.g., at least 95%, 96%, 97%, 98%, 99%, 99.9%, 99.99%, or 100% sequence identity) to the amino acid sequence of SEQ ID NO: 21.

In some embodiments, the NT-3 has an amino acid sequence having at least 95% (e.g., at least 95%, 96%, 97%, 98%, 99%, 99.9%, 99.99%, or 100% sequence identity) to the amino acid sequence of SEQ ID NO: 22. In some embodiments, the NT-3 has an amino acid sequence that differs from the amino acid sequence of SEQ ID NO: 15, SEQ ID NO: 21, or SEQ ID NO: 22 by only one or more conservative amino acid substitutions, such as by up to 25, up to 20, up to 15, up to 10, or up to 5 conservative amino acid substitutions. In some embodiments, the NT-3 is an NT-3 variant having an amino acid sequence with one or more (e.g., 1, 2, 3, 4, 5, 6, 7, 8, 9, 10 or more) amino acid substitutions relative to the amino acid sequence of SEQ ID NO: 15, SEQ ID NO: 21, or SEQ ID NO: 22. In some embodiments, the NT-3 variant has the sequence of any one of SEQ ID NOs: 48-60. In some embodiments, the NT-3 variant has the sequence of SEQ ID NO: 49. In some embodiments, the NT-3 variant has the sequence of SEQ ID NO: 57. In some embodiments, the NT-3 variant has the sequence of SEQ ID NO: 58. In some embodiments, the NT-3 is an NT-3 variant having an amino acid sequence with one or more (e.g., 1, 2, 3, 4, 5, 6, 7, 8, 9, 10 or more) amino acid substitutions relative to the amino acid sequence of SEQ ID NO: 15, SEQ ID NO: 21, or SEQ ID NO: 22 and/or one or more (e.g., 1, 2, 3, 4, 5, 6, 7, 8, 9, 10 or more) deleted amino acids relative to the amino acid sequence of SEQ ID NO: 15, SEQ ID NO: 21, or SEQ ID NO: 22. In some embodiments, the NT-3 is encoded by a nucleic acid having the sequence of SEQ ID NO: 16 or SEQ ID NO: 70.

In some embodiments, the neurotrophin is NGF. In some embodiments, the NGF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 19. In some embodiments, the NGF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 20. In some embodiments, the NGF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 19. In some embodiments, the NGF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 20. In some embodiments, the NGF has an amino acid sequence having at least 95% (e.g., at least 95%, 96%, 97%, 98%, 99%, 99.9%, 99.99%, or 100% sequence identity) to the amino acid sequence of SEQ ID NO: 19. In some embodiments, the NGF has an amino acid sequence having at least 95% (e.g., at least 95%, 96%, 97%, 98%, 99%, 99.9%, 99.99%, or 100% sequence identity) to the amino acid sequence of SEQ ID NO: 20. In some embodiments, the NGF has an amino acid sequence that differs from the amino acid sequence of SEQ ID NO: 19 or SEQ ID NO:20 by only one or more conservative amino acid substitutions, such as by up to 25, up to 20, up to 15, up to 10, or up to 5 conservative amino acid substitutions. In some embodiments, the NGF is an NGF variant having an amino acid sequence with one or more (e.g., 1, 2, 3, 4, 5, 6, 7, 8, 9, 10 or more) amino acid substitutions relative to the amino acid sequence of SEQ ID NO: 19 or SEQ ID NO: 20. In some embodiments, the NGF variant has the sequence any one of SEQ ID NOs: 34-37. In some embodiments, the NGF is an NGF variant having an amino acid sequence with one or more (e.g., 1, 2, 3, 4, 5, 6, 7, 8, 9, 10 or more) amino acid substitutions relative to the amino acid sequence of SEQ ID NO: 19 or SEQ ID NO: 20 and/or one or more (e.g., 1, 2, 3, 4, 5, 6, 7, 8, 9, 10 or more) deleted amino acids relative to the amino acid sequence of SEQ ID NO: 19 or SEQ ID NO: 20. In some embodiments, the NGF variant has the sequence of SEQ ID NO: 38. In some embodiments, the NGF is encoded by a nucleic acid having the sequence of SEQ ID NO: 69 or SEQ ID NO: 76.

In some embodiments, the neurotrophin is NT-4. In some embodiments, the NT-4 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 23. In some embodiments, the NT-4 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 24. In some embodiments, the NT-4 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 23. In some embodiments, the NT-4 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 24. In some embodiments, the NT-4 has an amino acid sequence having at least 95% (e.g., at least 95%, 96%, 97%, 98%, 99%, 99.9%, 99.99%, or 100% sequence identity) to the amino acid sequence of SEQ ID NO: 23. In some embodiments, the NT-4 has an amino acid sequence having at least 95% (e.g., at least 95%, 96%, 97%, 98%, 99%, 99.9%, 99.99%, or 100% sequence identity) to the amino acid sequence of SEQ ID NO: 24. In some embodiments, the NT-4 has an amino acid sequence that differs from the amino acid sequence of SEQ ID NO: 23 or SEQ ID NO:24 by only one or more conservative amino acid substitutions, such as by up to 25, up to 20, up to 15, up to 10, or up to 5 conservative amino acid substitutions. In some embodiments, the NT-4 is an NT-4 variant having an amino acid sequence with one or more (e.g., 1, 2, 3, 4, 5, 6, 7, 8, 9, 10 or more) amino acid substitutions relative to the amino acid sequence of SEQ ID NO: 23 or SEQ ID NO: 24. In some embodiments, the NT-4 is an NT-4 variant having an amino acid sequence with one or more (e.g., 1, 2, 3, 4, 5, 6, 7, 8, 9, 10 or more) amino acid substitutions relative to the amino acid sequence of SEQ ID NO: 23 or SEQ ID NO: 24 and/or one or more (e.g., 1, 2, 3, 4, 5, 6, 7, 8, 9, 10 or more) deleted amino acids relative to the amino acid sequence of SEQ ID NO: 23 or SEQ ID NO: 24. In some embodiments, the NT-4 is encoded by a nucleic acid having the sequence of SEQ ID NO: 71.

In some embodiments, the neurotrophin is BDNF. In some embodiments, the BDNF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 25. In some embodiments, the BDNF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of any one of SEQ ID NOs: 26-29. In some embodiments, the BDNF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 30. In some embodiments, the BDNF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 25. In some embodiments, the BDNF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of any one of SEQ ID NOs: 26-29. In some embodiments, the BDNF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 30. In some embodiments, the BDNF has an amino acid sequence having at least 95% (e.g., at least 95%, 96%, 97%, 98%, 99%, 99.9%, 99.99%, or 100% sequence identity) to the amino acid sequence of SEQ ID NO: 25. In some embodiments, the BDNF has an amino acid sequence having at least 95% (e.g., at least 95%, 96%, 97%, 98%, 99%, 99.9%, 99.99%, or 100% sequence identity) to the amino acid sequence of any one of SEQ ID NOs: 26-29. In some embodiments, the BDNF has an amino acid sequence having at least 95% (e.g., at least 95%, 96%, 97%, 98%, 99%, 99.9%, 99.99%, or 100% sequence identity) to the amino acid sequence of SEQ ID NO: 30. In some embodiments, the BDNF has an amino acid sequence that differs from the amino acid sequence of any one of SEQ ID NOs: 25-30 by only one or more conservative amino acid substitutions, such as by up to 25, up to 20, up to 15, up to 10, or up to 5 conservative amino acid substitutions. In some embodiments, the BDNF is a BDNF variant having an amino acid sequence with one or more (e.g., 1, 2, 3, 4, 5, 6, 7, 8, 9, 10 or more) amino acid substitutions relative to the amino acid sequence of any one of SEQ ID NOs: 25-30. In some embodiments, the BDNF is a BDNF variant having an amino acid sequence with one or more (e.g., 1, 2, 3, 4, 5, 6, 7, 8, 9, 10 or more) amino acid substitutions relative to the amino acid sequence of any one of SEQ ID NOs: 25-30 and/or one or more (e.g., 1, 2, 3, 4, 5, 6, 7, 8, 9, 10 or more) deleted amino acids relative to the amino acid sequence of any one of SEQ ID NOs: 25-30. In some embodiments, the BDNF is encoded by a nucleic acid having the sequence of any one of SEQ ID NOs: 72-75.

In some embodiments, the neurotrophin is a pan-neurotrophin. In some embodiments, the pan-neurotrophin is PNT-1. In some embodiments, the PNT-1 has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 31. In some embodiments, the PNT-1 has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 31. In some embodiments, the PNT-1 has an amino acid sequence having at least 95% (e.g., at least 95%, 96%, 97%, 98%, 99%, 99.9%, 99.99%, or 100% sequence identity) to the amino acid sequence of SEQ ID NO: 31. In some embodiments, the PNT-1 has an amino acid sequence that differs from the amino acid sequence of SEQ ID NO: 31 by only one or more conservative amino acid substitutions, such as by up to 25, up to 20, up to 15, up to 10, or up to 5 conservative amino acid substitutions. In some embodiments, the PNT-1 is a PNT-1 variant having an amino acid sequence with one or more (e.g., 1, 2, 3, 4, 5, 6, 7, 8, 9, 10 or more) amino acid substitutions relative to the amino acid sequence of SEQ ID NO: 31. In some embodiments, the PNT-1 variant has the sequence any one of SEQ ID NOs: 39-42 or 45-47. In some embodiments, the PNT-1 is a PNT-1 variant having an amino acid sequence with one or more (e.g., 1, 2, 3, 4, 5, 6, 7, 8, 9, 10 or more) amino acid substitutions relative to the amino acid sequence of SEQ ID NO: 31 and/or one or more (e.g., 1, 2, 3, 4, 5, 6, 7, 8, 9, 10 or more) deleted amino acids relative to the amino acid sequence of SEQ ID NO: 31.

In some embodiments, the neurotrophin is a chimeric neurotrophin. In some embodiments, the chimeric neurotrophin is an NGF/BDNF chimera. In some embodiments, the chimeric neurotrophin has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 32. In some embodiments, the chimeric neurotrophin has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 33. In some embodiments, the chimeric neurotrophin has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 43. In some embodiments, the chimeric neurotrophin has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 44. In some embodiments, the chimeric neurotrophin has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 32. In some embodiments, the chimeric neurotrophin has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 33. In some embodiments, the chimeric neurotrophin has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 43. In some embodiments, the chimeric neurotrophin has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 44. In some embodiments, the chimeric neurotrophin has an amino acid sequence having at least 95% (e.g., at least 95%, 96%, 97%, 98%, 99%, 99.9%, 99.99%, or 100% sequence identity) to the amino acid sequence of SEQ ID NO: 32. In some embodiments, the chimeric neurotrophin has an amino acid sequence having at least 95% (e.g., at least 95%, 96%, 97%, 98%, 99%, 99.9%, 99.99%, or 100% sequence identity) to the amino acid sequence of SEQ ID NO: 33. In some embodiments, the chimeric neurotrophin has an amino acid sequence having at least 95% (e.g., at least 95%, 96%, 97%, 98%, 99%, 99.9%, 99.99%, or 100% sequence identity) to the amino acid sequence of SEQ ID NO: 43. In some embodiments, the chimeric neurotrophin has an amino acid sequence having at least 95% (e.g., at least 95%, 96%, 97%, 98%, 99%, 99.9%, 99.99%, or 100% sequence identity) to the amino acid sequence of SEQ ID NO: 44. In some embodiments, the chimeric neurotrophin has an amino acid sequence that differs from the amino acid sequence of any one of SEQ ID NOs: 32, 33, 43, or 44 by only one or more conservative amino acid substitutions, such as by up to 25, up to 20, up to 15, up to 10, or up to 5 conservative amino acid substitutions. In some embodiments, the chimeric neurotrophin is a chimeric neurotrophin variant having an amino acid sequence with one or more (e.g., 1, 2, 3, 4, 5, 6, 7, 8, 9, 10 or more) amino acid substitutions relative to the amino acid sequence of any one of SEQ ID NOs: 32, 33, 43, or 44. In some embodiments, the chimeric neurotrophin is a chimeric neurotrophin variant having an amino acid sequence with one or more (e.g., 1, 2, 3, 4, 5, 6, 7, 8, 9, 10 or more) amino acid substitutions relative to the amino acid sequence of any one of SEQ ID NOs: 32, 33, 43, or 44 and/or one or more (e.g., 1, 2, 3, 4, 5, 6, 7, 8, 9, 10 or more) deleted amino acids relative to the amino acid sequence of any one of SEQ ID NOs: 32, 33, 43, or 44.

In some embodiments, the neurotrophin is CNTF. In some embodiments, the CNTF has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 61. In some embodiments, the CNTF has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 61. In some embodiments, the CNTF has an amino acid sequence having at least 95% (e.g., at least 95%, 96%, 97%, 98%, 99%, 99.9%, 99.99%, or 100% sequence identity) to the amino acid sequence of SEQ ID NO: 61. In some embodiments, the CNTF has an amino acid sequence that differs from the amino acid sequence of SEQ ID NO: 61 by only one or more conservative amino acid substitutions, such as by up to 25, up to 20, up to 15, up to 10, or up to 5 conservative amino acid substitutions. In some embodiments, the CNTF is a CNTF variant having an amino acid sequence with one or more (e.g., 1, 2, 3, 4, 5, 6, 7, 8, 9, 10 or more) amino acid substitutions relative to the amino acid sequence of SEQ ID NO: 61. In some embodiments, the CNTF is a CNTF variant having an amino acid sequence with one or more (e.g., 1, 2, 3, 4, 5, 6, 7, 8, 9, 10 or more) amino acid substitutions relative to the amino acid sequence of SEQ ID NO: 61 and/or one or more (e.g., 1, 2, 3, 4, 5, 6, 7, 8, 9, 10 or more) deleted amino acids relative to the amino acid sequence of SEQ ID NO: 61. In some embodiments, the CNTF is encoded by a nucleic acid having the sequence of SEQ ID NO: 77.

In some embodiments, the neurotrophin is IGF (e.g., IGF1 or IGF2). In some embodiments, the IGF is IGF1 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 62. In some embodiments, the IGF is IGF1 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 63. In some embodiments, the IGF is IGF1 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 64 or SEQ ID NO: 65. In some embodiments, the IGF is IGF2 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 66. In some embodiments, the IGF is IGF2 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 67. In some embodiments, the IGF is IGF2 and has an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 68. In some embodiments, the IGF is IGF1 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 62. In some embodiments, the IGF is IGF1 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 63. In some embodiments, the IGF is IGF1 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 64 or SEQ ID NO: 65. In some embodiments, the IGF is IGF2 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 66. In some embodiments, the IGF is IGF2 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 67. In some embodiments, the IGF is IGF2 and has an amino acid sequence having at least 90% sequence identity (e.g., at least 90%, 91%, 92%, 93%, 94%, 95%, 96%, 97%, 98%, 99%, 99.9%, 99.99%, or 100% sequence identity) to the amino acid sequence of SEQ ID NO: 68. In some embodiments, the IGF is IGF1 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 62. In some embodiments, the IGF is IGF1 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 63. In some embodiments, the IGF is IGF1 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 64 or SEQ ID NO: 65. In some embodiments, the IGF is IGF2 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 66. In some embodiments, the IGF is IGF2 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 67. In some embodiments, the IGF is IGF2 and has an amino acid sequence having at least 95% sequence identity (e.g., at least 95%, 96%, 97%, 98%, 99%, 99.9%, 99.99%, or 100% sequence identity) to the amino acid sequence of SEQ ID NO: 68. In some embodiments, the IGF is IGF1 and has an amino acid sequence that differs from the amino acid sequence of any one of SEQ ID NOs: 62-65 by only one or more conservative amino acid substitutions, such as by up to 25, up to 20, up to 15, up to 10, or up to 5 conservative amino acid substitutions. In some embodiments, the IGF is IGF2 and has an amino acid sequence that differs from the amino acid sequence of any one of SEQ ID NOs: 66-68 by only one or more conservative amino acid substitutions, such as by up to 25, up to 20, up to 15, up to 10, or up to 5 conservative amino acid substitutions. In some embodiments, the IGF is an IGF1 variant having an amino acid sequence with one or more (e.g., 1, 2, 3, 4, 5, 6, 7, 8, 9, 10 or more) amino acid substitutions relative to the amino acid sequence of any one of SEQ ID NOs: 62-65. In some embodiments, the IGF is an IGF2 variant having an amino acid sequence with one or more (e.g., 1, 2, 3, 4, 5, 6, 7, 8, 9, 10 or more) amino acid substitutions relative to the amino acid sequence of any one of SEQ ID NOs: 66-68. In some embodiments, the IGF is an IGF1 variant having an amino acid sequence with one or more (e.g., 1, 2, 3, 4, 5, 6, 7, 8, 9, 10 or more) amino acid substitutions relative to the amino acid sequence of any one of SEQ ID NOs: 62-65 and/or one or more (e.g., 1, 2, 3, 4, 5, 6, 7, 8, 9, 10 or more) deleted amino acids relative to the amino acid sequence of any one of SEQ ID NOs: 62-65. In some embodiments, the IGF is an IGF2 variant having an amino acid sequence with one or more (e.g., 1, 2, 3, 4, 5, 6, 7, 8, 9, 10 or more) amino acid substitutions relative to the amino acid sequence of any one of SEQ ID NOs: 66-68 and/or one or more (e.g., 1, 2, 3, 4, 5, 6, 7, 8, 9, 10 or more) deleted amino acids relative to the amino acid sequence of any one of SEQ ID NOs: 66-68. In some embodiments, the IGF is IGF1 and is encoded by a nucleic acid having the sequence of any one of SEQ ID NOs: 78-80. In some embodiments, the IGF is IGF1 and is encoded by a nucleic acid having the sequence of SEQ ID NO: 81 or SEQ ID NO: 82.

In some embodiments, the therapeutic agent is a glial cell line-derived neurotrophic factor family ligand, a neuropoietic cytokine, an anti-inflammatory cytokine, a neuroprotection agent, growth differentiation factor 11, erythropoietin (EPO), granulocyte-colony stimulating factor, granulocyte-macrophage colony stimulating factor, growth differentiation factor-9, thrombopoietin, transforming growth factor alpha (TGF-α), stromal cell-derived factor 1, myostatin (growth differentiation factor 8), parathyroid hormone, parathyroid hormone related peptide, interleukin 1 receptor antagonist, fibroblast growth factor 18, high-mobility group protein 2, glucocorticoid receptor, fibroblast growth factor 9, hepatocyte growth factor, or a TGFβ-superfamily protein.

In some embodiments, the therapeutic agent is a glial cell line-derived neurotrophic factor family ligand selected from glial cell line-derived neurotrophic factor (GDNF), neurturin, artemin, and persephin.

In some embodiments, the therapeutic agent is a neuropoietic cytokine selected from interleukin-6, interleukin-11, inteleukin-27, leukemia inhibitory factor, CNTF, cardiotrophin 1, neuropoietin, cardiotrophin-like cytokine, and fibroblast growth factor 2.

In some embodiments, the therapeutic agent is an anti-inflammatory cytokine selected from interleukin-4 and interleukin-10.

In some embodiments, the therapeutic agent is a neuroprotection agent selected from neuregulin-1, vascular endothelial growth factor (VEGF), sodium thiosulfate, and N-acetyl cysteine.

In some embodiments, the therapeutic agent is a TGFβ-superfamily protein selected from TGFβ, TGFβ3, BMP2, and BMP7.

In some embodiments, the therapeutic agent is a nucleic acid vector. In some embodiments, the nucleic acid vector is a plasmid, cosmid, artificial chromosome, or viral vector. In some embodiments, the viral vector is an adeno-associated virus (AAV), adenovirus, lentivirus, retrovirus, poxvirus, baculovirus, herpes simplex virus, or a vaccinia virus. In some embodiments, the viral vector is an AAV vector. In some embodiments, the serotype of the AAV vector is selected from the group consisting of AAV1, AAV2, AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, and PHP.S. In some embodiments, the serotype of the AAV vector is AAV1.

In some embodiments, the serotype of the AAV vector isAAV2. In some embodiments, the serotype of the AAV vector is 7m8. In some embodiments, the AAV vector has a mutation at one or more surface-exposed tyrosine residues on capsid proteins, such as Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704 (Y704F), Tyr730 to Phe730 (Y730F), and Tyr 733 to Phe733 (Y733F). In some embodiments, the nucleic acid vector (e.g., AAV vector) is engineered to express a protein described herein (e.g., a neurotrophin, such as NT-3, NGF, NT-4, BDNF, CNTF, IGF, PNT-1, or a chimeric neurotrophin, or Atoh1). In some embodiments, the nucleic acid vector includes the sequence of any one of SEQ ID NOs: 16 or 61-68.

In some embodiments, the therapeutic agent is an antibody or antigen-binding fragment thereof.

The antibody or antigen-binding fragment thereof may be, for example, a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFv.

In some embodiments, the therapeutic agent is a liposome, vesicle, synthetic vesicle, exosome, synthetic exosome, dendrimer, or nanoparticle.

In some embodiments, the therapeutic agent is a small molecule, such as a small molecule that is not naturally round window membrane-penetrant.

In some embodiments, the therapeutic agent is an interfering RNA, such as a short interfering RNA (siRNA), a short hairpin RNA (shRNA), or a micro RNA (miRNA).

In some embodiments, the pharmaceutical composition is a gel at normal human body temperature.

In some embodiments, the gel has a dynamic viscosity of at about 100 cP to about 1,000,000 cP, such as a dynamic viscosity of about 100 cP, 200 cP, 300 cP, 400 cP, 500 cP, 600 cP, 700 cP, 800 cP, 900 cP, 1,000 cP, 2,000 cP, 3,000 cP, 4,000 cP, 5,000 cP, 6,000 cP, 7,000 cP, 8,000 cP, 9,000 cP, 10,000 cP, 20,000 cP, 30,000 cP, 40,000 cP, 50,000 cP, 60,000 cP, 70,000 cP, 80,000 cP, 90,000 cP, 100,000 cP, 200,000 cP, 300,000 cP, 400,000 cP, 500,000 cP, 600,000 cP, 700,000 cP, 800,000 cP, 900,000 cP, or 1,000,000 cP.

In some embodiments, the pharmaceutical composition further contains a pharmaceutically acceptable liquid solvent, such as water.

In some embodiments, the pharmaceutical composition contains one or more agents selected from an antimicrobial agent, an arylcycloalkylamine, an elipticine derivative, an anti-apoptotic agent, a c-JNK inhibitor, an antioxidant, an NSAID, an analgesic, a neuroprotection agent, a glutamate modulator, an interleukin 1 modulator, an interleukin-1 antagonist, a corticosteroid, an anti-TNF agent, a calcineurin inhibitor, an IKK inhibitor, an interleukin inhibitor, a platelet activating factor antagonist, a TNF-α converting enzyme (TACE) inhibitor, a Toll-like receptor inhibitor, an autoimmune agent, an IL-1 modulator, an RNA interference agent, an aquaporin modulator, an estrogen-related receptor beta modulator, a GAP junction protein, a vasopressin receptor modulator, a NMDA receptor modulator, an ENaC receptor modulator, an osmotic diuretic, a progesterone receptor, a prostaglandin, a cytotoxic agent, a cytoprotective agent, anti-intercellular adhesion molecule-1 antibody, an Atoh1 modulator (e.g., an Atoh1 polypeptide or a nucleic acid vector engineered to express Atoh1, e.g., human Atoh1 (Hath1)), a Math1 modulator, a BRN-3 modulator, a carbamate, an estrogen receptor, a fatty acid, a gamma-secretase inhibitor, a glutamate-receptor modulator, a neurotrophic agent, salicylic acid, nicotine, a retinoblastoma protein modulator, an ion channel blocker, a thyroid hormone receptor modulator, a TRPV modulator, an adenosine modulator, a KCNQ modulator, a P2X modulator, a CNS modulating agent, an anticholinergic, an antihistamine, a GABA receptor modulator, a neurotransmitter reuptake inhibitor, a thyrotropin-releasing hormone, a free radical modulator, a metal atom chelator, a mitochondrial modulator, a nitric oxide synthase modulator, a sirtuin modulator, a purinergic receptor modulator, a truncated TrkC or TrkB antagonist, a truncated TrkC or TrkB isoform, a nucleic acid polymer antagonist, a small molecule antagonist, a polypeptide antagonist, a non-natural TrkC or TrkB agonist, a neurotrophin variant, a WNT modulator, a glycogen synthase kinase 3 (GSK3) inhibitor (e.g., a GSK3β and/or GSK3α inhibitor), a protein kinase C beta modulator, a repulsive guidance molecule a (RGMa) inhibitor, a neogenin inhibitor, a SK2 channel activator, a BK channel activator, a sphingosine-1-phosphate receptor modulator, a stemness driver, a differentiation inhibitor, an N-Methyl-D-Aspartate (NMDA) receptor antagonist, a histone deacetylase (HDAC) inhibitor, a proteasome inhibitor, an EZH2/HMT inhibitor, a notch inhibitor, ebselen, ancrod, an α-amino-3-hydroxy-5-methyl-4-isoxazolepropionic acid (AMPA) glutamate-positive allosteric modulator, D-methionine, an antagonist of histamine type 4 receptors, a chemotherapeutic accumulation reducer, choline ester, plant alkaloid, reversible cholinesterase inhibitor, acetylcholine release promoter, anti-adrenergy, a sympathomimetic, an antineoplastic agent, R(+)-N-propargyl-1-aminoindan, and R-azasetron besylate. In another aspect, the invention features a method of delivering a therapeutic agent across the round window membrane of a subject (e.g., a mammalian subject, such as a human subject) by providing to the subject (e.g., by way of direct administration to the subject) a therapeutically effective amount of the pharmaceutical composition of any of the above aspects or embodiments of the invention. In some embodiments, the pharmaceutical composition is administered to or near the round window membrane. In some embodiments, the pharmaceutical composition is administered intratympanically ortranstympanically. In some embodiments, the method is used to treat an otic disease, such as ceruminosis or ceruminosis associated with an otic disease or condition, ear pruritus, otitis externa, otalgia, tinnitus, vestibular dysfunction (e.g., vertigo, dizziness, or loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

In another aspect, the invention features a method of treating a subject (e.g., a mammalian subject, such as a human subject) having or at risk of developing hearing loss (e.g., sensorineural hearing loss, deafness, or auditory neuropathy) by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In some embodiments of any of the above aspects, the hearing loss is genetic hearing loss. In some embodiments, the genetic hearing loss is autosomal dominant hearing loss, autosomal recessive hearing loss, or X-linked hearing loss.

In some embodiments of any of the above aspects, the hearing loss is acquired hearing loss. In some embodiments, the acquired hearing loss is noise-induced hearing loss, age-related hearing loss, disease or infection-related hearing loss, head trauma-related hearing loss, or ototoxic drug-induced hearing loss.

In another aspect, the invention features a method of treating a subject (e.g., a mammalian subject, such as a human subject) having or at risk of developing vestibular dysfunction by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In some embodiments of any of the above aspects, the vestibular dysfunction is vertigo, dizziness, or loss of balance.

In another aspect, the invention features a method of promoting hair cell regeneration in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of increasing the number of supporting cells (e.g., cochlear and/or vestibular supporting cells, e.g., increasing supporting cell proliferation) in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of promoting SGN regeneration in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of preventing or reducing ototoxic drug-induced hair cell damage or death in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of preventing or reducing ototoxic drug-induced SGN damage or death in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In some embodiments of any of the above aspects, the ototoxic drug is selected from the group including aminoglycosides (e.g., gentamycin, neomycin, streptomycin, tobramycin, kanamycin, vancomycin, and amikacin), antineoplastic drugs (e.g., platinum-containing chemotherapeutic agents, such as cisplatin, carboplatin, and oxaliplatin), ethacrynic acid, furosemide, salicylates (e.g., aspirin, particularly at high doses), and quinine.

In another aspect, the invention features a method of treating a subject (e.g., a mammalian subject, such as a human subject) having or at risk of developing tinnitus by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of preventing or reducing hair cell damage or death in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of preventing or reducing SGN damage or death in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of increasing hair cell survival in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In another aspect, the invention features a method of increasing SGN survival in a subject (e.g., a mammalian subject, such as a human subject) in need thereof by administering to the subject a therapeutically effective amount of the pharmaceutical composition of any of the above aspects or embodiments of the invention, such as embodiments in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, a neuroprotection agent, or a WNT modulator (e.g., embodiments in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., a NGF/BDNF chimera), and combinations thereof).

In some embodiments of any of the above aspects, the hair cell is a cochlear hair cell. In some embodiments of any of the above aspects, the cochlear hair cell is an inner hair cell. In some embodiments of any of the above aspects, the cochlear hair cell is an outer hair cell. In some embodiments of any of the above aspects, the hair cell is a vestibular hair cell. In some embodiments of any of the above aspects, the hair cell is a mammalian hair cell. In some embodiments, the mammalian hair cell is a human hair cell. In some embodiments of any of the above aspects, the SGN is a mammalian SGN. In some embodiments of any of the above aspects, the mammalian SGN is a human SGN.

In some embodiments of any of the above aspects, the method further includes evaluating the hearing of the subject prior to administering the pharmaceutical composition (e.g., evaluating hearing using standard tests, such as audiometry, auditory brainstem response (ABR), electrochocleography (ECOG), or otoacoustic emissions).

In some embodiments of any of the above aspects, the method further includes evaluating the hearing of the subject after administering the pharmaceutical composition (e.g., evaluating hearing using standard tests, such as audiometry, ABR, ECOG, or otoacoustic emissions).

In some embodiments of any of the above aspects, the method further includes evaluating the vestibular function of the subject prior to administering the pharmaceutical composition (e.g., evaluating vestibular function using standard tests, such as electronystagmogram (ENG) or videonystagmogram (VNG), posturography, rotary-chair testing, ECOG, vestibular evoked myogenic potentials (VEMP), or specialized clinical balance tests).

In some embodiments of any of the above aspects, the method further includes evaluating the vestibular function of the subject after administering the pharmaceutical composition (e.g., evaluating vestibular function using standard tests, such as ENG or VNG, posturography, rotary-chair testing, ECOG, VEMP, or specialized clinical balance tests).

In some embodiments of any of the above aspects, the pharmaceutical composition is locally administered. In some embodiments, the pharmaceutical composition is administered to the ear of the subject (e.g., administered to the inner ear or middle ear, e.g., into the perilymph or endolymph, such as through the oval window, round window, semicircular canals, or horizontal canal, e.g., administered by transtympanic or intratympanic injection).

In some embodiments of any of the above aspects, the pharmaceutical composition is administered in an amount sufficient to prevent or reduce hearing loss, prevent or reduce vestibular dysfunction, prevent or reduce tinnitus, delay the development of hearing loss, delay the development of vestibular dysfunction, slow the progression of hearing loss, slow the progression of vestibular dysfunction, improve hearing, improve vestibular function, improve hair cell function, prevent or reduce hair cell damage, prevent, slow, or reduce hair cell death, promote or increase hair cell survival, increase hair cell numbers, increase supporting cell numbers, promote or induce hair cell regeneration, improve SGN function, prevent or reduce SGN damage, prevent, slow, or reduce SGN death, promote or increase SGN survival, increase SGN numbers, promote or induce SGN regeneration, preserve ribbon synapses, promote or increase ribbon synapse formation, maintain the connections between hair cells and SGNs, or increase or restore the connections between hair cells and SGNs.

In an additional aspect, the invention features a kit containing the pharmaceutical composition of any of the above aspects or embodiments of the invention. In some embodiments, the kit contains a package insert instructing a user of the kit to administer the pharmaceutical composition to a subject (e.g., a mammalian subject, such as a human subject) in need thereof. In some embodiments, the subject is suffering from an otic disease, such as ceruminosis or ceruminosis associated with an otic disease or condition, ear pruritus, otitis externa, otalgia, tinnitus, vestibular dysfunction (e.g., vertigo, dizziness, or loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), auto immune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof. In some embodiments, the package insert instructs a user of the kit to administer the pharmaceutical composition to the subject in order to prevent or mitigate sensory hair cell death in the subject.

The invention is also described by the enumerated items below.

1. A pharmaceutical composition formulated for otic administration to a human patient, the pharmaceutical composition comprising a therapeutic agent and a permeation enhancer, wherein the permeation enhancer is an alpha-helical, facially amphipathic polypeptide having a molecular weight of from about 1,000 Da to about 3,500 Da and a pl of at least 7.0.

2. The pharmaceutical composition of item 1, wherein polypeptide comprises one or more regions represented, from N-terminus to C-terminus, by formula (I)

X¹—X²—X² (I)
wherein each X¹ independently represents an amino acid comprising a cationic side chain at physiological pH;
each X² independently represents an amino acid comprising a hydrophobic side chain; and
each “—” independently represents a peptide bond or a peptide bond isostere.

3. The pharmaceutical composition of item 2, wherein:
each X¹ independently represents an amino acid comprising a lysine or arginine side chain; and
each X² independently represents an amino acid comprising an alanine, leucine, isoleucine, valine, methionine, tryptophan, phenylalanine, cysteine, or tyrosine side chain.

4. The pharmaceutical composition of item 3, wherein:
each X¹ independently represents an amino acid comprising a lysine or arginine side chain; and
each X² independently represents an amino acid comprising an alanine, leucine, isoleucine, valine, or tryptophan side chain.

5. The pharmaceutical composition of item 4, wherein:
each X¹ independently represents an amino acid comprising a lysine side chain; and
each X² independently represents an amino acid comprising an alanine, leucine, or tryptophan side chain.

6. The pharmaceutical composition of any one of items 2-5, wherein the polypeptide comprises from 2 to 10 of the regions represented by formula (I).

7. The pharmaceutical composition of item 6, wherein the polypeptide comprises from 3 to 7 of the regions represented by formula (I).

8. The pharmaceutical composition of item 7, wherein the polypeptide comprises 5 of the regions represented by formula (I).

9. The pharmaceutical composition of any one of items 2-8, wherein the regions represented by formula (I) are consecutive or separated by up to two amino acid residues.

10. The pharmaceutical composition of item 1, wherein the polypeptide comprises one or more regions represented, from N-terminus to C-terminus, by formula (II)

X³—X⁴—X⁵—X⁴ (II)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain; and
each X⁵ independently represents an amino acid comprising an alanine side chain.

11. The pharmaceutical composition of item 10, wherein:
each X³ independently represents an amino acid comprising a lysine or arginine side chain;
each X⁴ independently represents an amino acid comprising a leucine, isoleucine, valine, or tryptophan side chain;
each X⁵ independently represents an amino acid comprising an alanine side chain; and
each “—” independently represents a peptide bond or a peptide bond isostere.

12. The pharmaceutical composition of item 11, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

13. The pharmaceutical composition of any one of items 10-12, wherein the polypeptide comprises from 1 to 10 of the regions represented by formula (II).

14. The pharmaceutical composition of item 13, wherein the polypeptide comprises from 2 to 5 of the regions represented by formula (II).

15. The pharmaceutical composition of item 14, wherein the polypeptide comprises 3 of the regions represented by formula (II).

16. The pharmaceutical composition of item 1, wherein the polypeptide comprises one or more regions represented, from N-terminus to C-terminus, by formula (III)

X³—X⁴—X⁵—X⁴—X³—X⁴—X⁵—X⁴ (III)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain; and
each “—” independently represents a peptide bond or a peptide bond isostere.

17. The pharmaceutical composition of item 16, wherein:
each X³ independently represents an amino acid comprising a lysine or arginine side chain;
each X⁴ independently represents an amino acid comprising a leucine, isoleucine, valine, or tryptophan side chain; and
each X⁵ independently represents an amino acid comprising an alanine side chain.

18. The pharmaceutical composition of item 17, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

19. The pharmaceutical composition of any one of items 16-18, wherein the polypeptide comprises from 1 to 5 of the regions represented by formula (III).

20. The pharmaceutical composition of item 19, wherein the polypeptide comprises one region represented by formula (III).

21. The pharmaceutical composition of item 1, wherein the polypeptide comprises a region represented, from N-terminus to C-terminus, by formula (IV)

[X³—X⁴— X⁵— X⁴]ₙ—[X³—X⁴—X⁵]ₘ (IV)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain;
n represents an integer from 1 to 5;
m represents an integer from 1 to 5; and
each “—” independently represents a peptide bond or a peptide bond isostere.

22. The pharmaceutical composition of item 21, wherein:
each X³ independently represents an amino acid comprising a lysine or arginine side chain;
each X⁴ independently represents an amino acid comprising a leucine, isoleucine, valine, or tryptophan side chain; and
each X⁵ independently represents an amino acid comprising an alanine side chain.

23. The pharmaceutical composition of item 22, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

24. The pharmaceutical composition of any one of items 21-23, wherein n represents an integer from 2 to 4.

25. The pharmaceutical composition of item 24, wherein n is 2.

26. The pharmaceutical composition of any one of items 21-25, wherein m represents an integer from 1 to 3.

27. The pharmaceutical composition of item 26, wherein m is 1.

28. The pharmaceutical composition of item 1, wherein the polypeptide comprises a region represented, from N-terminus to C-terminus, by formula (V)

[X³—X⁴— X⁵— X⁴]ₙ—[X³—X⁴— X⁵]ₘ—[X³—X⁴— X⁵— X⁵—X⁴]_{q} (V)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain;
n represents an integer from 1 to 5;
m represents an integer from 1 to 5;
q represents an integer from 1 to 5; and
each “—” independently represents a peptide bond or a peptide bond isostere.

29. The pharmaceutical composition of item 28, wherein:
each X³ independently represents an amino acid comprising a lysine or arginine side chain;
each X⁴ independently represents an amino acid comprising a leucine, isoleucine, valine, or tryptophan side chain; and
each X⁵ independently represents an amino acid comprising an alanine side chain.

30. The pharmaceutical composition of item 29, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

31. The pharmaceutical composition of any one of items 28-30, wherein n represents an integer from 2 to 4.

32. The pharmaceutical composition of item 31, wherein n is 2.

33. The pharmaceutical composition of any one of items 28-32, wherein m represents an integer from 1 to 3.

34. The pharmaceutical composition of item 33, wherein m is 1.

35. The pharmaceutical composition of any one of items 28-34, wherein q represents an integer from 1 to 3.

36. The pharmaceutical composition of item 35, wherein q is 1.

37. The pharmaceutical composition of item 1, wherein the polypeptide comprises a region represented, from N-terminus to C-terminus, by formula (VI)

[X³—X⁴— X⁵— X⁴]ₙ—[X³—X⁴— X⁵]ₘ—[X³—X⁴— X⁵—X⁵—X⁴]_{q}—[X³—X⁴—X⁵]ᵣ (VI)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain;
n represents an integer from 1 to 5;
m represents an integer from 1 to 5;
q represents an integer from 1 to 5;
r represents an integer from 1 to 5; and
each “—” independently represents a peptide bond or a peptide bond isostere.

38. The pharmaceutical composition of item 37, wherein:
each X³ independently represents an amino acid comprising a lysine or arginine side chain;
each X⁴ independently represents an amino acid comprising a leucine, isoleucine, valine, or tryptophan side chain; and
each X⁵ independently represents an amino acid comprising an alanine side chain.

39. The pharmaceutical composition of item 38, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

40. The pharmaceutical composition of any one of items 37-39, wherein n represents an integer from 2 to 4.

41. The pharmaceutical composition of item 40, wherein n is 2.

42. The pharmaceutical composition of any one of items 37-41, wherein m represents an integer from 1 to 3.

43. The pharmaceutical composition of item 42, wherein m is 1.

44. The pharmaceutical composition of any one of items 37-43, wherein q represents an integer from 1 to 3.

45. The pharmaceutical composition of item 44, wherein q is 1.

46. The pharmaceutical composition of any one of items 37-45, wherein r represents an integer from 1 to 3.

47. The pharmaceutical composition of item 46, wherein r is 1.

48. The pharmaceutical composition of any one of items 2-47, wherein one or more of each “—” is a peptide bond isostere selected from:

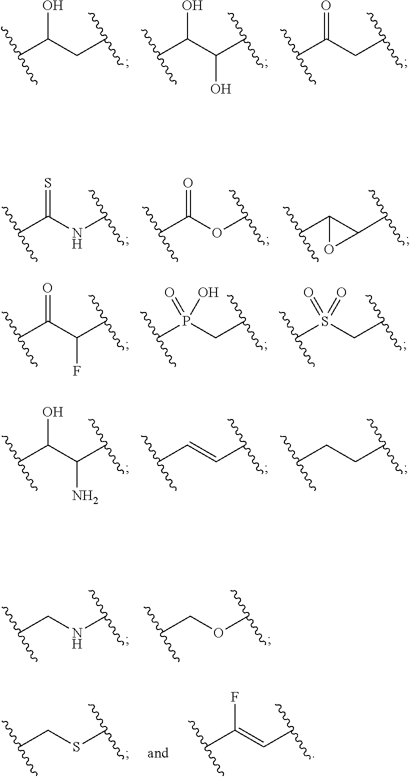

49. The pharmaceutical composition of any one of items 2-48, wherein the polypeptide comprises one or more intramolecular crosslinks.

50. The pharmaceutical composition of item 49, wherein the one or more intramolecular crosslinks stabilize an alpha-helical structure.

51. The pharmaceutical composition of item 49 or 50, wherein the polypeptide comprises one or more electrostatic intramolecular crosslinks.

52. The pharmaceutical composition of item 49 or 50, wherein the polypeptide comprises one or more covalent intramolecular crosslinks.

53. The pharmaceutical composition of item 52, wherein the one or more covalent intramolecular crosslinks are selected from:

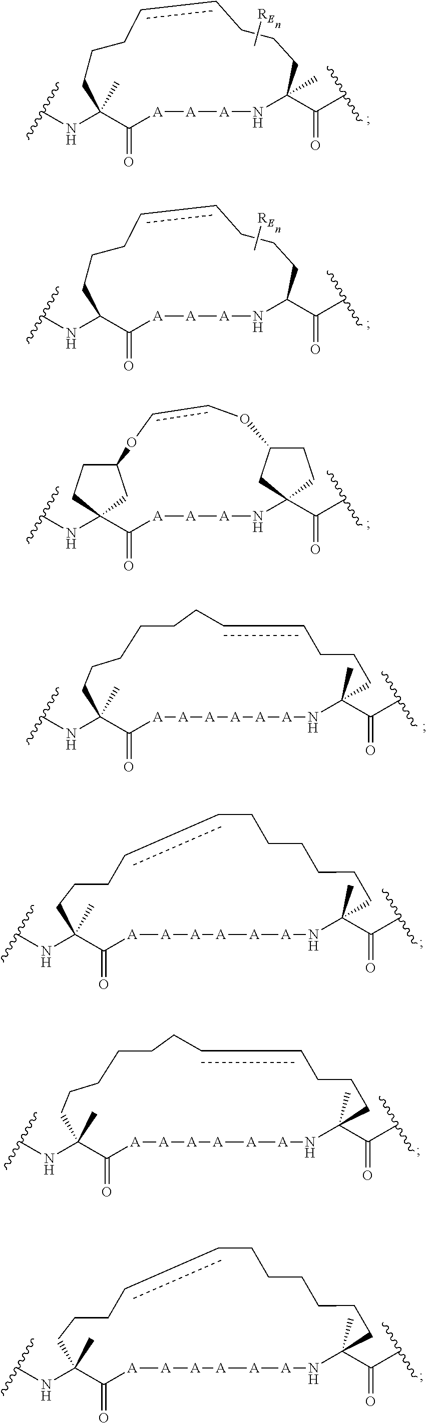

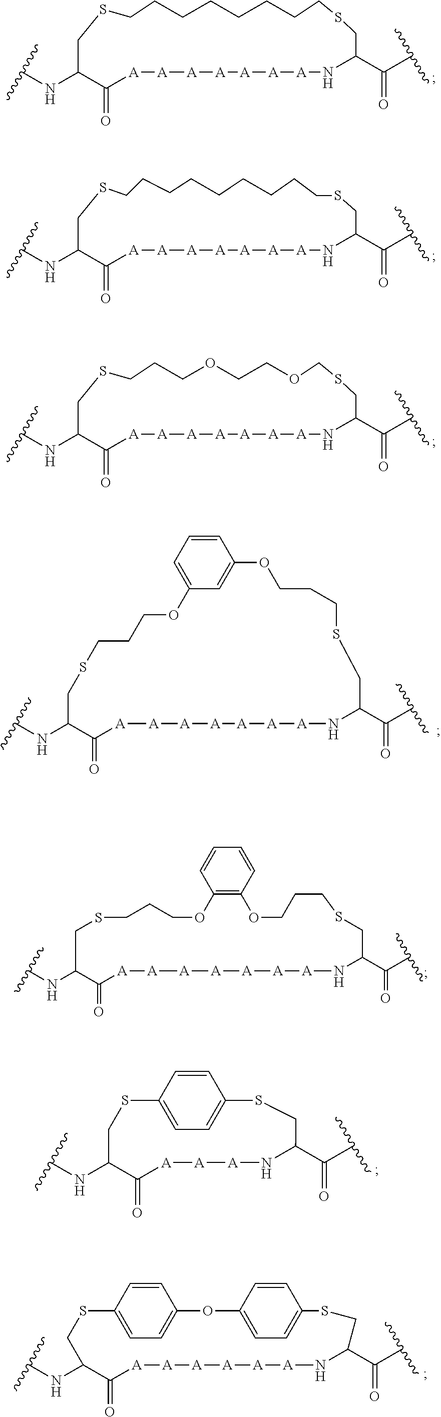

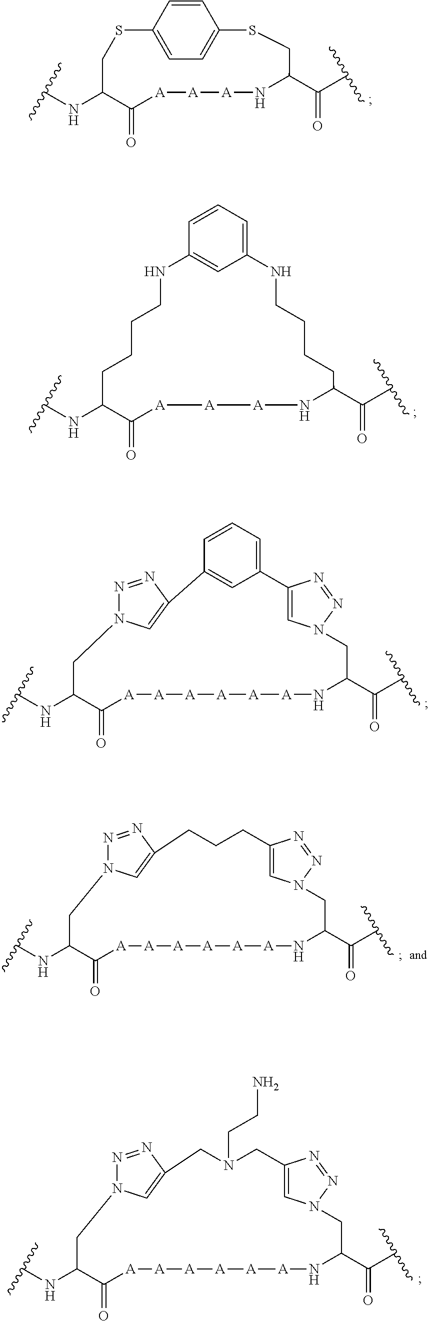

wherein each “A” represents an individual amino acid within the polypeptide.

54. The pharmaceutical composition of any one of items 1-53, wherein the polypeptide is cyclized from N-terminus to C-terminus.

55. The pharmaceutical composition of any one of items 1-54, wherein the polypeptide is from 10 to 30 amino acid residues in length.

56. The pharmaceutical composition of item 55, wherein the polypeptide is from 14 to 26 amino acid residues in length.

57. The pharmaceutical composition of item 56, wherein the polypeptide is from 15 to 20 amino acid residues in length.

58. The pharmaceutical composition of item 57, wherein the polypeptide is 19 amino acid residues in length.

59. The pharmaceutical composition of any one of items 1-58, wherein the polypeptide has an isoelectric point (pl) of from about 8 to about 13.

60. The pharmaceutical composition of item 59, wherein the polypeptide has a pl of from about 8.5 to about 12.5.

61. The pharmaceutical composition of item 60, wherein the polypeptide has a pl of from about 9 to about 12.

62. The pharmaceutical composition of item 61, wherein the polypeptide has a pl of from about 9.5 to about 11.5.

63. The pharmaceutical composition of item 62, wherein the polypeptide has a pl of from about 10 to about 11.

64. The pharmaceutical composition of item 63, wherein the polypeptide has a pl of from about 10.1 to about 10.9.

65. The pharmaceutical composition of item 64, wherein the polypeptide has a pl of about 10.6.

66. The pharmaceutical composition of any one of items 1-65, wherein the polypeptide has a molecular weight of from about 1,400 Da to about 2,800 Da.

67. The pharmaceutical composition of item 66, wherein the polypeptide has a molecular weight of from about 1,450 Da to about 2,750 Da.

68. The pharmaceutical composition of item 67, wherein the polypeptide has a molecular weight of from about 1,600 to about 2,500 Da.

69. The pharmaceutical composition of item 68, wherein the polypeptide has a molecular weight of from about 1,700 to about 2,400 Da.

70. The pharmaceutical composition of item 69, wherein the polypeptide has a molecular weight of from about 1,800 to about 2,100 Da.

71. The pharmaceutical composition of item 70, wherein the polypeptide has a molecular weight of from about 1,900 to about 2,050 Da.

72. The pharmaceutical composition of item 71, wherein the polypeptide has a molecular weight of about 1,990 Da.

73. The pharmaceutical composition of any one of items 1-72, wherein the polypeptide has at least about 50% alpha-helicity as assessed by circular dichroism.

74. The pharmaceutical composition of item 73, wherein the polypeptide has at least about 55% alpha-helicity as assessed by circular dichroism.

75. The pharmaceutical composition of item 74, wherein the polypeptide has at least about 60% alpha-helicity as assessed by circular dichroism.

76. The pharmaceutical composition of item 75, wherein the polypeptide has at least about 65% alpha-helicity as assessed by circular dichroism.

77. The pharmaceutical composition of any one of items 1-72, wherein the polypeptide has from about 50% to about 85% alpha-helicity as assessed by circular dichroism.

78. The pharmaceutical composition of item 77, wherein the polypeptide has from about 55% to about 80% alpha-helicity as assessed by circular dichroism.

79. The pharmaceutical composition of item 78, wherein the polypeptide has from about 60% to about 75% alpha-helicity as assessed by circular dichroism.

80. The pharmaceutical composition of item 78, wherein the polypeptide has from about 60% to about 70% alpha-helicity as assessed by circular dichroism.

81. The pharmaceutical composition of item 80, wherein the polypeptide has from about 61% to about 68% alpha-helicity as assessed by circular dichroism.

82. The pharmaceutical composition of any one of items 1, 55-69, and 73-81, wherein the polypeptide is represented by formula (VII)

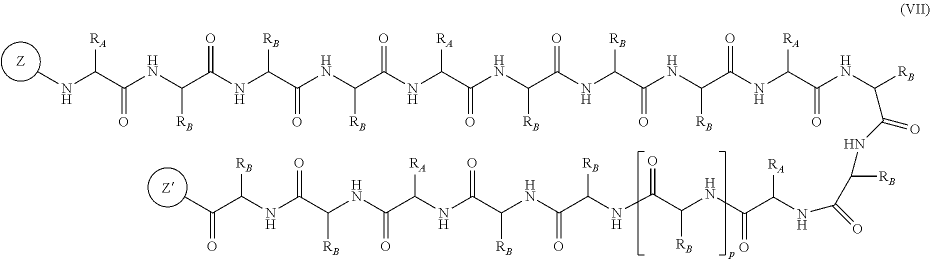

wherein each R_{A} is independently selected from:

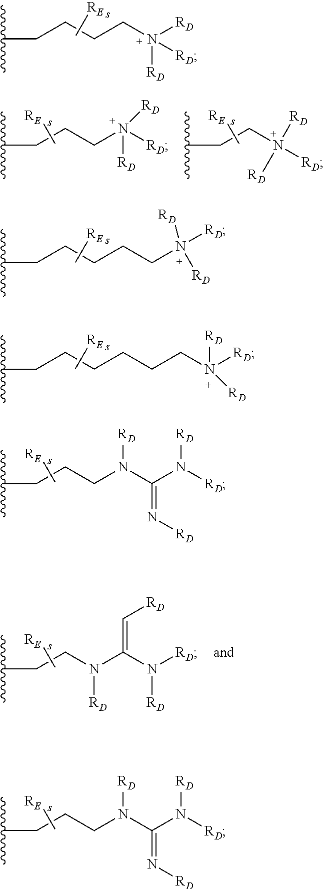

each R_{B} is independently selected from:

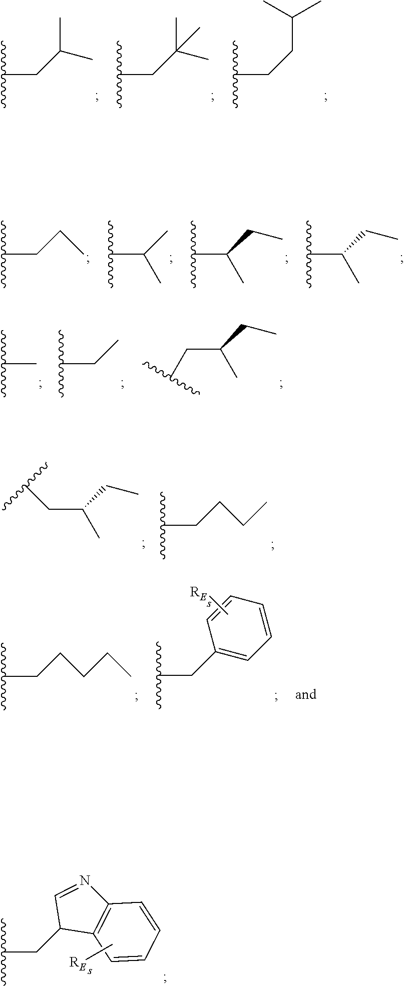

each R_{D} is independently hydrogen or an optionally substituted C₁-C₆ alkyl group;
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
p is an integer from 0 to 3;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

83. The pharmaceutical composition of item 82, wherein the polypeptide is represented by formula (VIII)

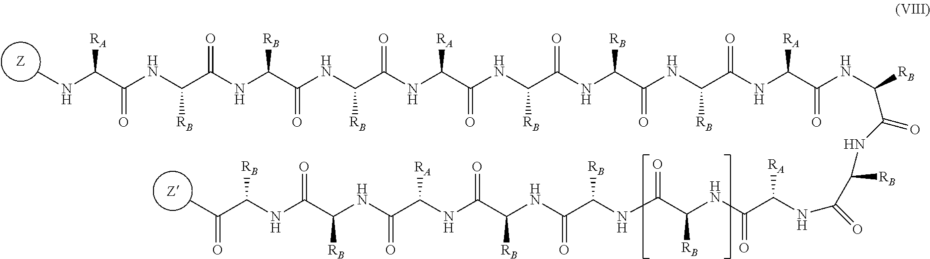

wherein each of R^{A}, R^{B}, R^{D}, R^{E}, p, s, Z, and Z′ are as defined for formula (VII).

84. The pharmaceutical composition of item 82, wherein the polypeptide is represented by formula (IX)

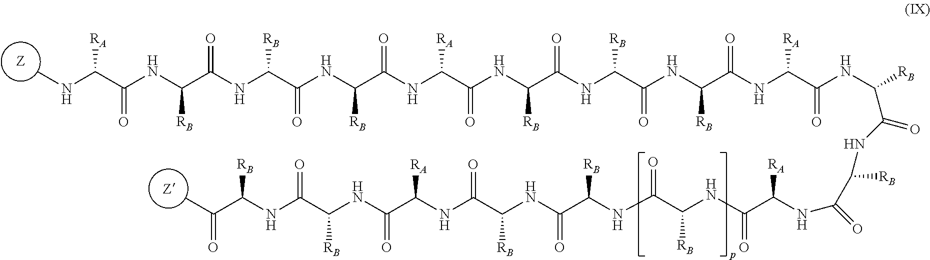

wherein each of R^{A}, R^{B}, R^{D}, R^{E}, p, s, Z, and Z′ are as defined for formula (VII).

85. The pharmaceutical composition of any one of items 1, 55-69, and 73-81, wherein the polypeptide is represented by formula (X)

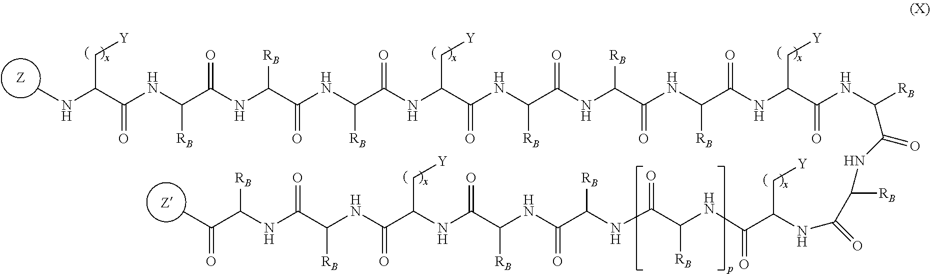

wherein each Y is independently an optionally substituted amino group or an optionally substituted guanidinium group;
each R_{B} is independently selected from:

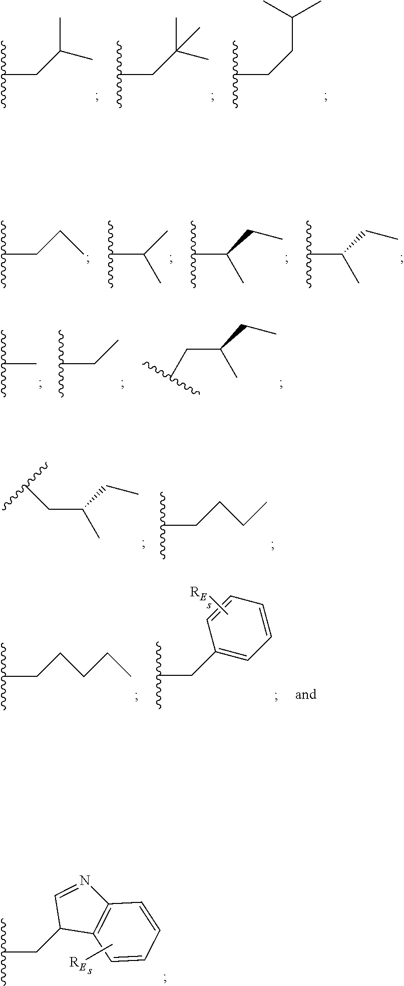

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
x is an integer from 3 to 5;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

86. The pharmaceutical composition of item 85, wherein the polypeptide is represented by formula (XI)

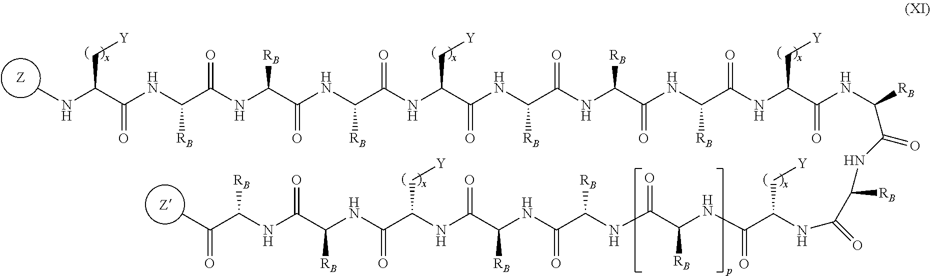

wherein each of Y, R^{B}, R^{E}, x, s, Z, and Z′ are as defined for formula (X).

87. The pharmaceutical composition of item 85, wherein the polypeptide is represented by formula (XII)

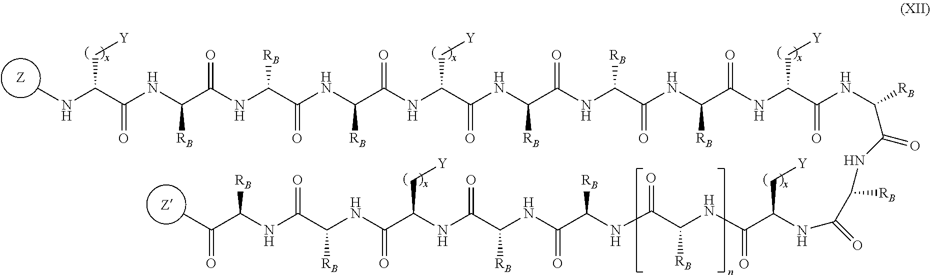

wherein each of Y, R_{B}, R^{E}, X, s, Z, and Z′ are as defined for formula (X).

88. The pharmaceutical composition of any one of items 1, 55-69, and 73-81, wherein the polypeptide is represented by formula (XIII)

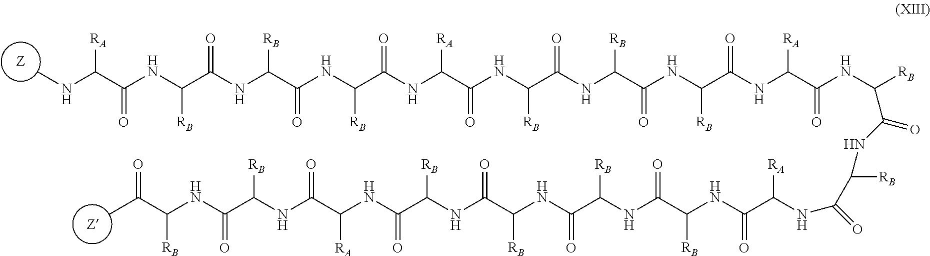

wherein each R_{A} is independently selected from:

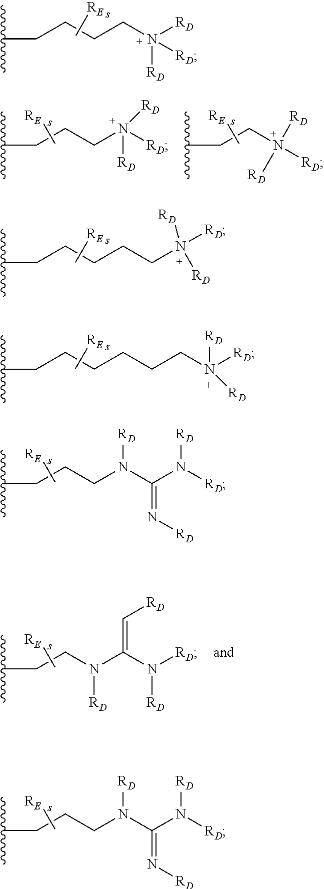

each R_{B} is independently selected from:

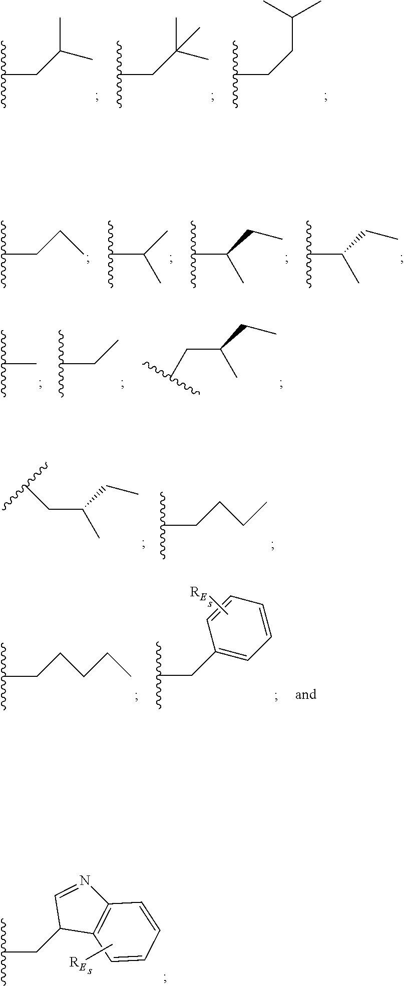

each R_{D} is independently hydrogen or an optionally substituted C₁-C₆ alkyl group;
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

89. The pharmaceutical composition of item 88, wherein the polypeptide is represented by formula (XIV)

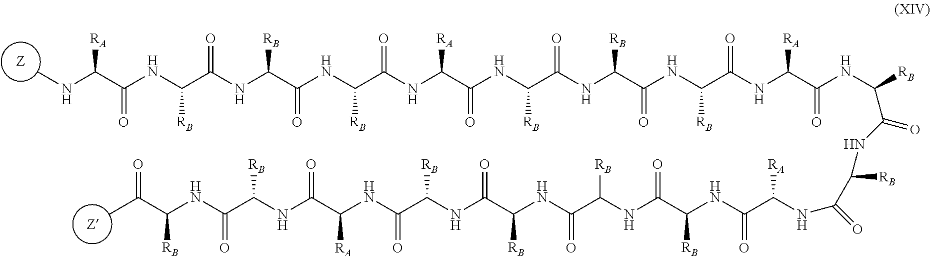

wherein each of R_{A}, R_{B}, R_{D}, R_{E}, s, Z, and Z′ are as defined for formula (XIII).

90. The pharmaceutical composition of item 88, wherein the polypeptide is represented by formula (XV)

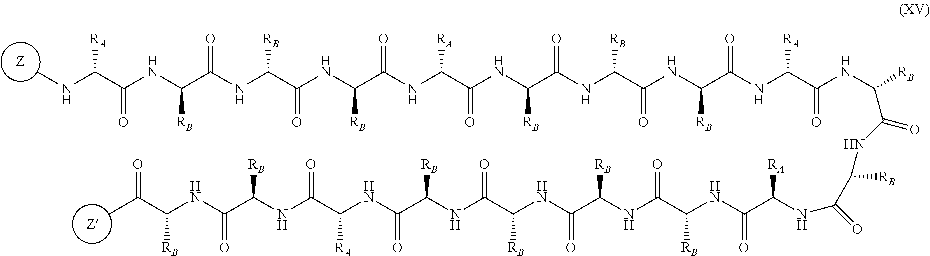

wherein each of R^{A}, R^{B}, R^{D}, R^{E}, s, Z, and Z′ are as defined for formula (XIII).

91. The pharmaceutical composition of any one of items 1, 55-69, and 73-81, wherein the polypeptide is represented by formula (XVI)

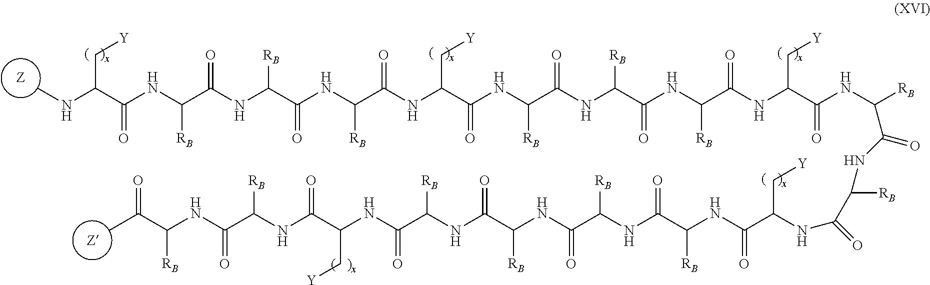

wherein each Y is independently an optionally substituted amino group or an optionally substituted guanidinium group;
each R_{B} is independently selected from:

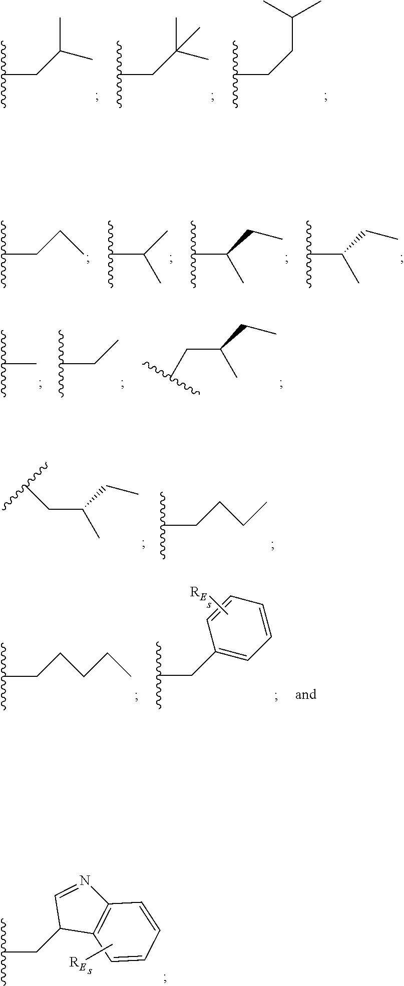

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
x is an integer from 3 to 5;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

92. The pharmaceutical composition of item 91, wherein the polypeptide is represented by formula (XVII)

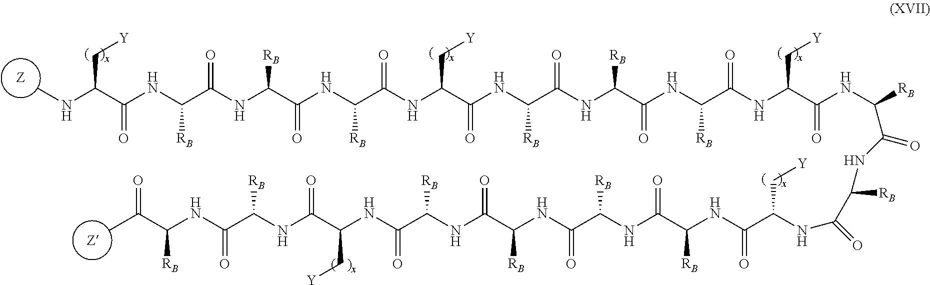

wherein each of Y, R^{B}, R^{E}, x, s, Z, and Z′ are as defined for formula (XVI).

93. The pharmaceutical composition of item 91, wherein the polypeptide is represented by formula (XVIII)

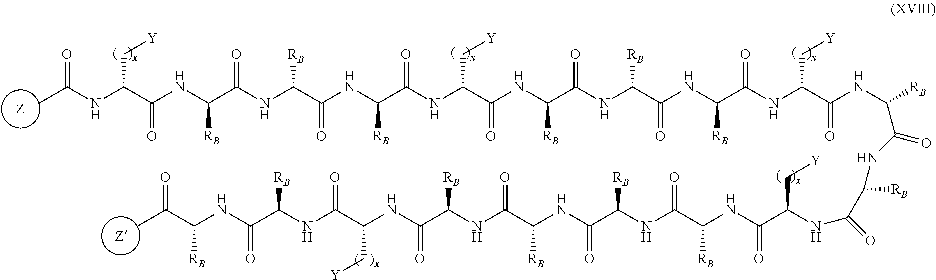

wherein each of Y, R^{B}, R^{E}, x, s, Z, and Z′ are as defined for formula (XVI).

94. The pharmaceutical composition of any one of items 1, 55-69, and 73-81, wherein the polypeptide is represented by formula (XIX)

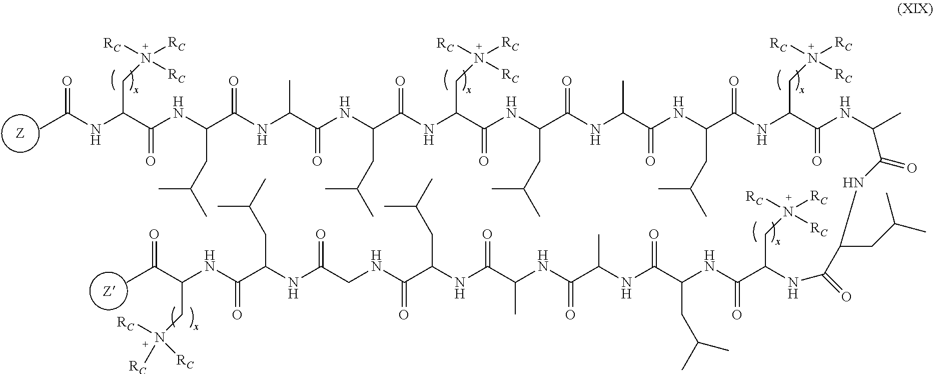

wherein each R_{C} is independently hydrogen or optionally substituted C₁-C₆ alkyl;
x is an integer from 3 to 5; optionally wherein x is 4;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

95. The pharmaceutical composition of item 94, wherein the polypeptide is represented by formula (XX)

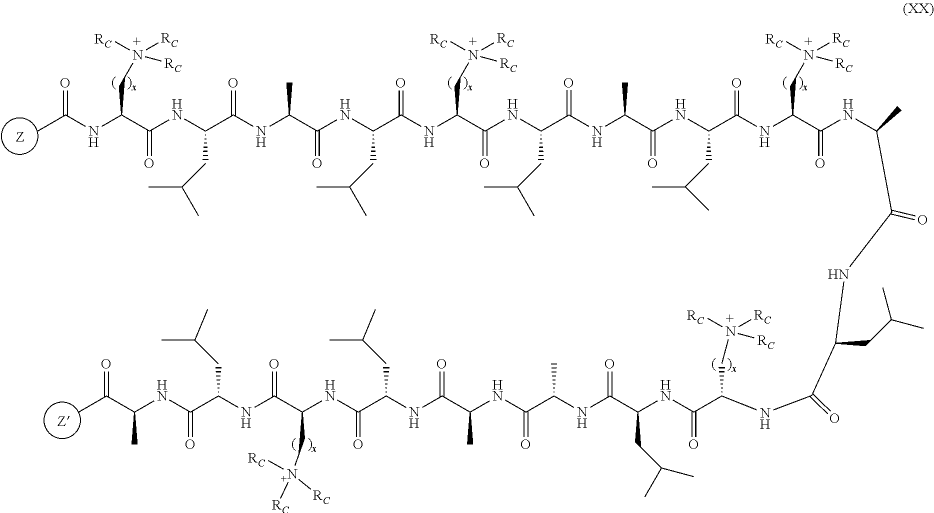

wherein R_{C}, x, Z, and Z′ are as defined for formula (XIX).

96. The pharmaceutical composition of item 94, wherein the polypeptide is represented by formula (XXI)

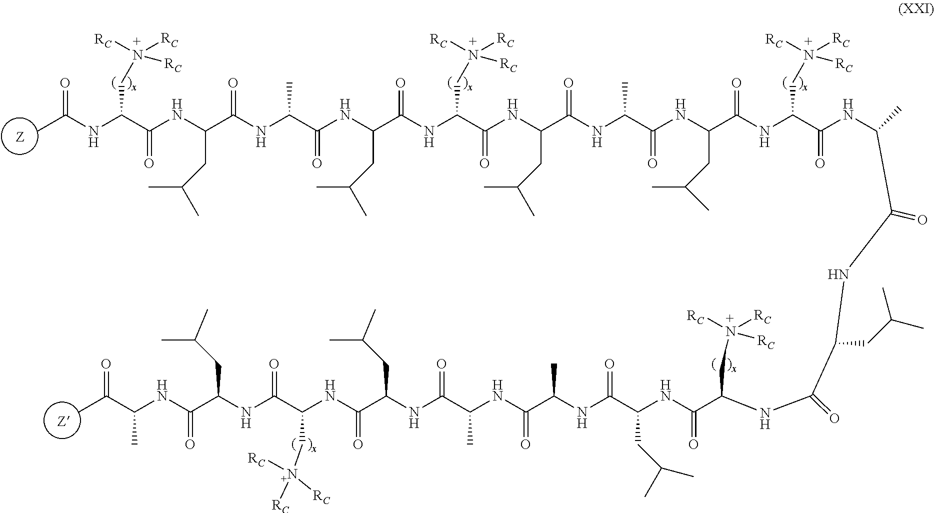

wherein R_{C}, x, Z, and Z′ are as defined for formula (XIX).

97. The pharmaceutical composition of any one of items 1, 55-69, and 73-81, wherein the polypeptide is represented by formula (XXII)

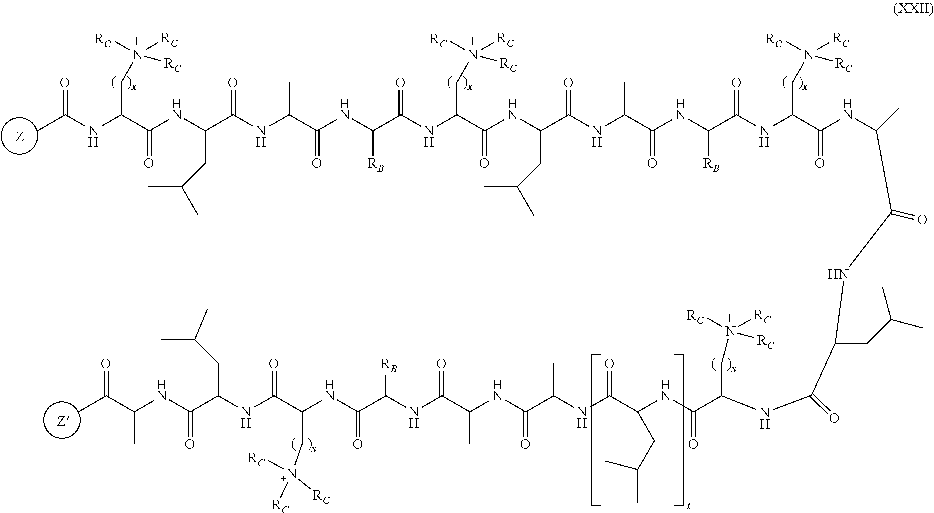

wherein each R_{C} is independently hydrogen or optionally substituted C₁-C₆ alkyl;
x is an integer from 3 to 5; optionally wherein x is 4;
t is 0 or 1;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

98. The pharmaceutical composition of item 97, wherein the polypeptide is represented by formula (XXIII)

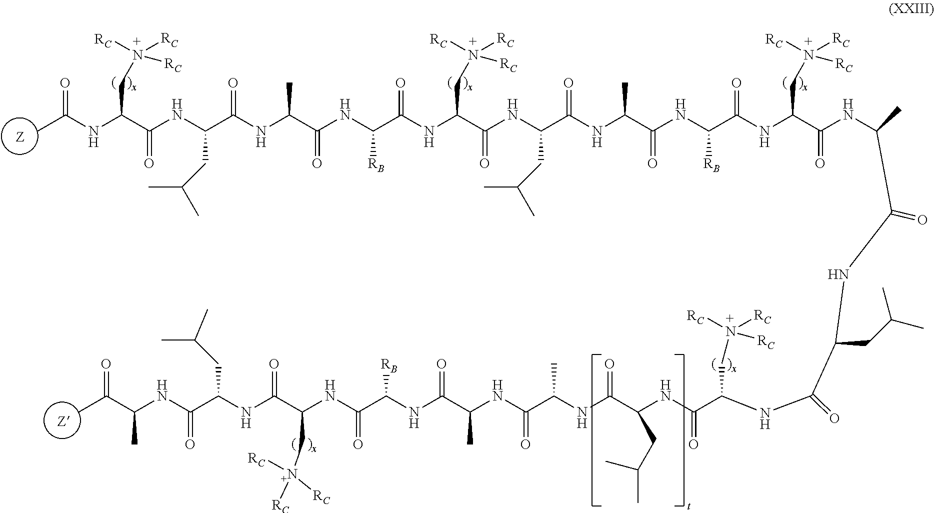

wherein R_{C}, x, t, Z, and Z′ are as defined for formula (XXII).

99. The pharmaceutical composition of item 97, wherein the polypeptide is represented by formula (XXIV)

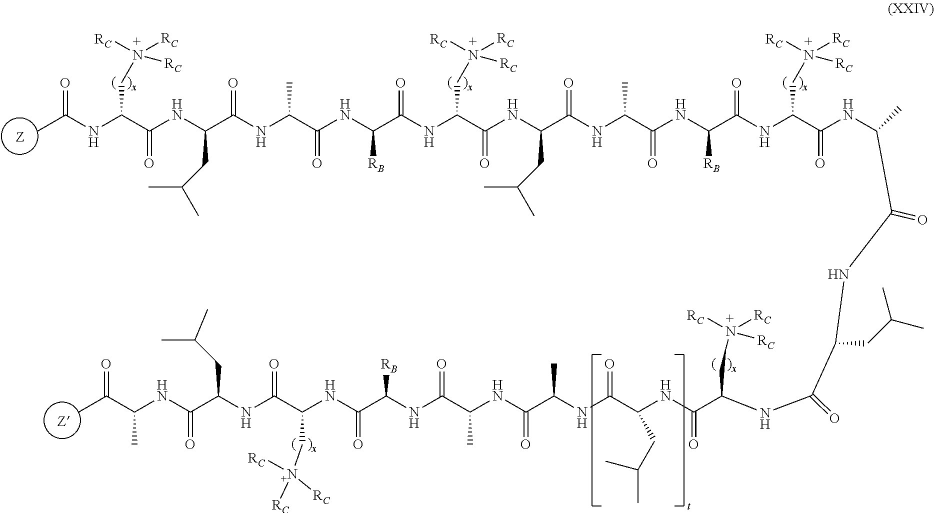

wherein R_{C}, x, t, Z, and Z′ are as defined for formula (XXII).

100. The pharmaceutical composition of item 1, wherein the polypeptide is represented by formula (XXV)

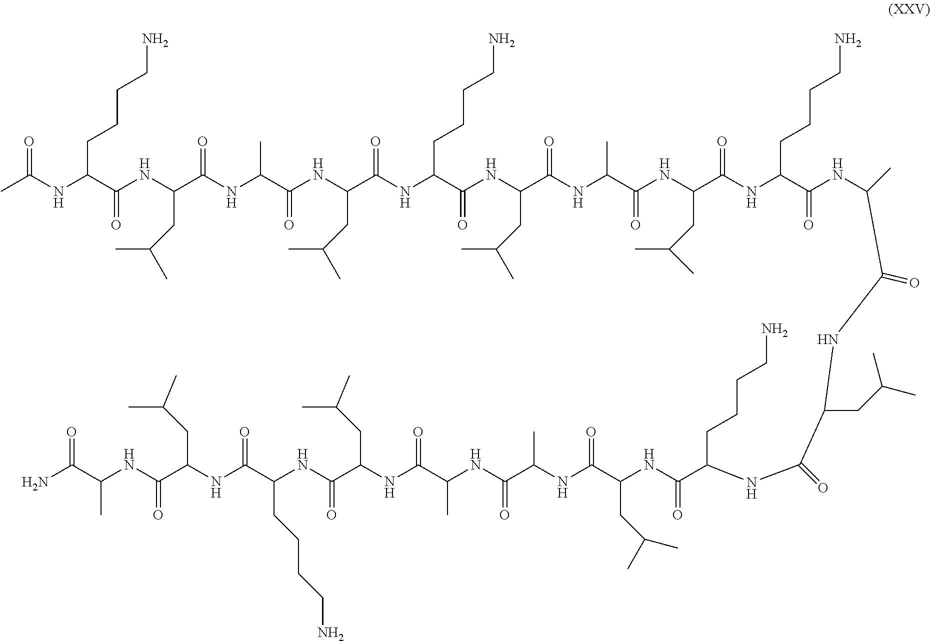 [SMILES: CC(=O)NC(CCCCN)C(=O)NC(CC(C)C)C(=O)NC(C)C(=O)NC(CC(C)C)C(=O)NC(CCCCN)C(=O)NC(CC(C)C)C(=O)NC(C)C(=O)NC(CC(C)C)C(=O)NC(CCCCN)C(=O)NC(C)C(=O)NC(CC(C)C)C(=O)NC(CCCCN)C(=O)NC(CC(C)C)C(=O)NC(C)C(=O)NC(C)C(=O)NC(CC(C)C)C(=O)NC(CCCCN)C(=O)NC(CC(C)C)C(=O)NC(C)C(N)=O]

or a pharmaceutically acceptable salt thereof.

101. The pharmaceutical composition of item 100, wherein the polypeptide is represented by formula (XXVI)

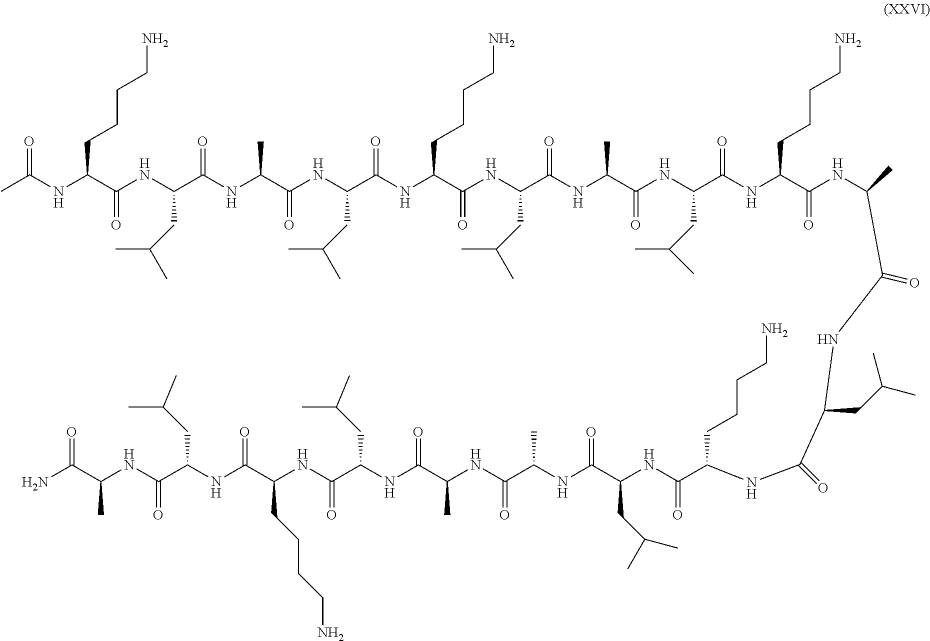 [SMILES: CC(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](C)C(=O)N[C@@H](CC(C)C)C(=O)N[C@@H](CCCCN)C(=O)N[C@@H](CC(C)C)C(=O)N[C@@H](C)C(N)=O]

or a pharmaceutically acceptable salt thereof.

102. The pharmaceutical composition of item 100, wherein the polypeptide is represented by formula (XXVII)

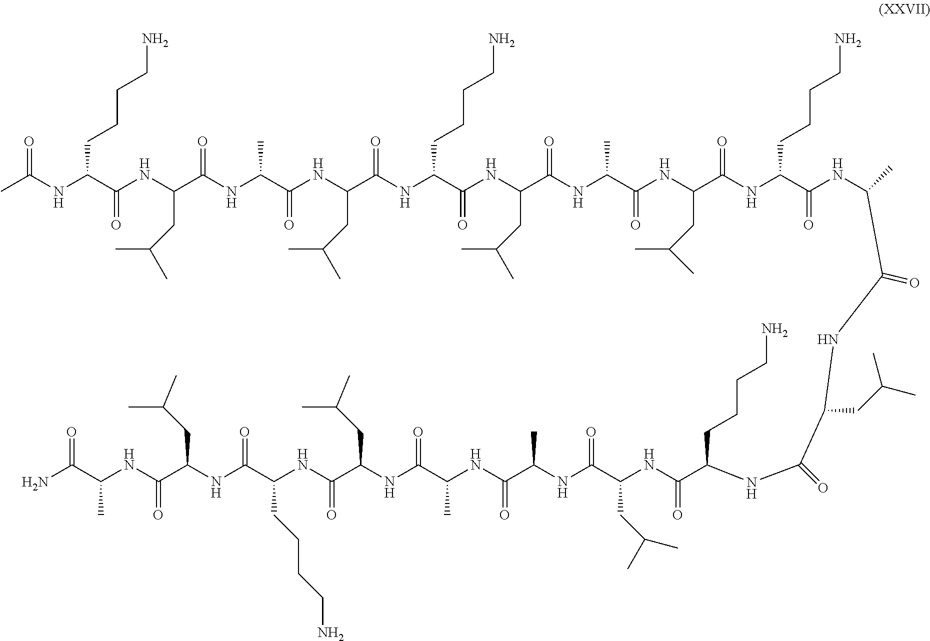 [SMILES: CC(=O)N[C@H](CCCCN)C(=O)NC(CC(C)C)C(=O)N[C@H](C)C(=O)NC(CC(C)C)C(=O)N[C@H](CCCCN)C(=O)NC(CC(C)C)C(=O)N[C@H](C)C(=O)NC(CC(C)C)C(=O)N[C@H](CCCCN)C(=O)N[C@H](C)C(=O)N[C@H](CC(C)C)C(=O)N[C@H](CCCCN)C(=O)N[C@H](CC(C)C)C(=O)N[C@H](C)C(=O)N[C@H](C)C(=O)N[C@H](CC(C)C)C(=O)N[C@H](CCCCN)C(=O)N[C@H](CC(C)C)C(=O)N[C@H](C)C(N)=O]

or a pharmaceutically acceptable salt thereof.

103. The pharmaceutical composition of any one of items 1, 55-69, and 73-81, wherein the polypeptide comprises a region having an amino acid sequence that is at least 85% identical to an amino acid sequence selected from:

(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

104. The pharmaceutical composition of item 103, wherein the polypeptide has an amino acid sequence that is at least 90% identical to an amino acid sequence selected from:

(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

105. The pharmaceutical composition of item 104, wherein the polypeptide has an amino acid sequence that is at least 95% identical to an amino acid sequence selected from:

(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

106. The pharmaceutical composition of any one of items 1, 55-69, and 73-81, wherein the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to five amino acid substitutions.

107. The pharmaceutical composition of item 106, wherein the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to three amino acid substitutions.

108. The pharmaceutical composition of item 106, wherein the amino acid substitutions are conservative amino acid substitutions.

109. The pharmaceutical composition of item 1, wherein the polypeptide has an amino acid sequence selected from:

(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

110. The pharmaceutical composition of any one of items 1-109, wherein the polypeptide is present within the pharmaceutical composition at a concentration of from about 0.001% w/v to about 50% w/v.

111. The pharmaceutical composition of item 110, wherein the polypeptide is present within the pharmaceutical composition at a concentration of from about 0.01% w/v to about 10% w/v.

112. The pharmaceutical composition of item 110, wherein the polypeptide is present within the pharmaceutical composition at a concentration of from about 0.1% w/v to about 5% w/v.

113. The pharmaceutical composition of item 112, wherein the polypeptide is present within the pharmaceutical composition at a concentration of about 1% w/v.

114. The pharmaceutical composition of any one of items 1-113, wherein the therapeutic agent is present within the pharmaceutical composition at a concentration of from about 0.001% w/v to about 50% w/v.

115. The pharmaceutical composition of item 114, wherein the therapeutic agent is present within the pharmaceutical composition at a concentration of from about 0.01% w/v to about 10% w/v.

116. The pharmaceutical composition of item 115, wherein the therapeutic agent is present within the pharmaceutical composition at a concentration of from about 0.1% w/v to about 5% w/v.

117. The pharmaceutical composition of item 116, wherein the therapeutic agent is present within the pharmaceutical composition at a concentration of from about 0.1% w/v to about 1% w/v.

118. The pharmaceutical composition of item 117, wherein the therapeutic agent is present within the pharmaceutical composition at a concentration of from about 0.1% w/v to about 0.25% w/v.

119. The pharmaceutical composition of any one of items 1-118, wherein the pharmaceutical composition further comprises a gelling agent.

120. The pharmaceutical composition of item 119, wherein the gelling agent is selected from the group consisting of hyaluronan, hyaluronic acid, a polyoxyethylene-polyoxypropylene block copolymer, poly(lactic-co-glycolic) acid, polylactic acid, polycaprolactone, alginic acid or a salt thereof, polyethylene glycol, a cellulose, a cellulose ether, agar-agar, gelatin, glucomannan, galactomannan (e.g., locust bean gum or tara gum), xanthan gum, guar gum, chitosan, pectin, starch, tragacanth, carrageenan, polyvinylpyrrolidone, polyvinyl alcohol, paraffin, polyethoxylated sorbitan monolaurate, petrolatum, silicates, fibroin, gellan, CARBOPOL 940®, polyoxamines, lecithin gels, polysorbate-80, (poly)aniline derivatives, xyloglucane, collagen, silicon dioxide, tyloxapol, Cremophor, aluminum magnesium silicate, sodium stearate, bladderwrack, bentonite, eratonia, chondrus, dextrose, furcellaran, Ghatti gum, hectorite, lactose, sucrose, sucralose, maltodextrin, mannitol, sorbitol, honey, maize starch, wheat starch, rice starch, potato starch, oxypolygelatin, polygeline, sterculia gum, propylene carbonate, methyl vinyl ether/maleic anhydride copolymer, poly(methoxyethyl methacrylate), and poly(methoxyethoxyethyl methacrylate), and combinations thereof.

121. The pharmaceutical composition of item 119 or 120, wherein the gelling agent is selected from the group consisting of a polyoxyethylene-polyoxypropylene block copolymer, alginic acid or a pharmaceutically acceptable salt thereof, collagen, hyaluronic acid or a pharmaceutically acceptable salt thereof, gelatin, and fibroin.

122. The pharmaceutical composition of item 120 or 121, wherein the polyoxyethylene-polyoxypropylene block copolymer is poloxamer 407.

123. The pharmaceutical composition of item 120 or 121, wherein the polyoxyethylene-polyoxypropylene block copolymer is poloxamer 188.

124. The pharmaceutical composition of any one of items 120-123, wherein the polyoxyethylene-polyoxypropylene block copolymer is present within the pharmaceutical composition at a concentration of from about 0.001% w/v to about 50% w/v.

125. The pharmaceutical composition of item 124, wherein the polyoxyethylene-polyoxypropylene block copolymer is present within the pharmaceutical composition at a concentration of from about 0.01% w/v to about 40% w/v.

126. The pharmaceutical composition of item 125, wherein the polyoxyethylene-polyoxypropylene block copolymer is present within the pharmaceutical composition at a concentration of from about 1% w/v to about 30% w/v.

127. The pharmaceutical composition of item 124, wherein the polyoxyethylene-polyoxypropylene block copolymer is present within the pharmaceutical composition at a concentration of about 20% w/v.

128. The pharmaceutical composition of any one of items 1-127, wherein upon intratympanic or transtympanic administration to a mammalian subject, the therapeutic agent is delivered across the round window membrane of the subject.

129. The pharmaceutical composition of item 128, wherein upon intratympanic or transtympanic administration to a mammalian subject, the therapeutic agent remains present within perilymph of the subject for at least from about 1 hour to about 6 weeks following the administration to the subject.

130. The pharmaceutical composition of item 129, wherein upon intratympanic or transtympanic administration to a mammalian subject, the therapeutic agent remains present within perilymph of the subject for from about 4 hours to about 72 hours following the administration to the subject.

131. The pharmaceutical composition of item 130, wherein upon intratympanic or transtympanic administration to a mammalian subject, the therapeutic agent remains present within perilymph of the subject for from about 8 hours to about 48 hours following the administration to the subject.

132. The pharmaceutical composition of item 131, wherein upon intratympanic or transtympanic administration to a mammalian subject, the therapeutic agent remains present within perilymph of the subject for from about 12 hours to about 24 hours following the administration to the subject.

133. The pharmaceutical composition of item 132, wherein upon intratympanic or transtympanic administration to a mammalian subject, the therapeutic agent remains present within perilymph of the subject for about 16 hours following the administration to the subject.

134. The pharmaceutical composition of any one of items 1-133, wherein the pharmaceutical composition comprises a unit dosage form having a volume of from about 50 μL to about 1 mL.

135. The pharmaceutical composition of item 134, wherein the pharmaceutical composition comprises a unit dosage form having a volume of from 100 μL to about 1 mL, from about 200 μL to about 1 mL, from about 300 μL to 0.8 mL, from about 200 μL to about 0.8 mL, from about 100 μL to about 0.5 mL, from about 200 μL to about 0.5 mL, from about 0.5 mL to about 1.0 mL, from about 0.5 mL to about 0.8 mL, or from about 0.8 mL to about 1.0 mL.

136. The pharmaceutical composition of any one of items 1-135, wherein the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, or a WNT modulator.

137. The pharmaceutical composition of item 136, wherein the therapeutic agent is a TrkB receptor agonist antibody.

138. The pharmaceutical composition of item 136, wherein the therapeutic agent is a TrkC receptor agonist antibody.

139. The pharmaceutical composition of item 136, wherein the therapeutic agent is an Atoh1 modulator.

140. The pharmaceutical composition of item 136 wherein the therapeutic agent is a WNT modulator.

141. The pharmaceutical composition of item 136, wherein the therapeutic agent is a neurotrophin selected from neurotrophin-3 (NT-3), nerve growth factor (NGF), brain-derived neurotrophic factor (BDNF), ciliary neurotrophic factor (CNTF), glial cell-line derived neurotrophic factor (GDNF), neurotrophin-4 (NT-4), fibroblast growth factor (FGF), insulin-like growth factor (IGF), epidermal growth factor (EGF), platelet-derived growth factor (PGF), mesencephalic astrocyte-derived neurotrophic factor (MANF), cerebral dopamine neurotrophic factor (CDNF), a pan-neurotrophic factor, a chimeric neurotrophic factor, and combinations thereof.

142. The pharmaceutical composition of item 141, wherein the neurotrophic factor is NT-3.

143. The pharmaceutical composition of item 142, wherein the NT-3 has an amino acid sequence having at least 85% sequence identity to the amino acid sequence of SEQ ID NO: 15, SEQ ID NO: 21, or SEQ ID NO: 22.

144. The pharmaceutical composition of item 143, wherein the NT-3 has an amino acid sequence having at least 90% sequence identity to the amino acid sequence of SEQ ID NO: 15, SEQ ID NO: 21, or SEQ ID NO: 22.

145. The pharmaceutical composition of item 144, wherein the NT-3 has an amino acid sequence having at least 95% sequence identity to the amino acid sequence of SEQ ID NO: 15, SEQ ID NO: 21, or SEQ ID NO: 22.

146. The pharmaceutical composition of item 145, wherein the NT-3 has the amino acid sequence of SEQ ID NO: 15.

147. The pharmaceutical composition of item 145, wherein the NT-3 has the amino acid sequence of SEQ ID NO: 21.

148. The pharmaceutical composition of item 145, wherein the NT-3 has the amino acid sequence of SEQ ID NO: 22.

149. The pharmaceutical composition of item 142, wherein the NT-3 is an NT-3 variant having one or more amino acid substitutions relative to amino acid sequence of SEQ ID NO: 22.

150. The pharmaceutical composition of item 149, wherein the NT-3 variant has the amino acid sequence of any one of SEQ ID NOs: 48-60.

151. The pharmaceutical composition of any one of items 1-135, wherein the therapeutic agent is a glial cell line-derived neurotrophic factor family ligand, a neuropoietic cytokine, an anti-inflammatory cytokine, a neuroprotection agent, growth differentiation factor 11, erythropoietin (EPO), granulocyte-colony stimulating factor, granulocyte-macrophage colony stimulating factor, growth differentiation factor-9, thrombopoietin, transforming growth factor alpha (TGF-α), stromal cell-derived factor 1, myostatin, parathyroid hormone, parathyroid hormone related peptide, interleukin 1 receptor antagonist, fibroblast growth factor 18, high-mobility group protein 2, glucocorticoid receptor, fibroblast growth factor 9, hepatocyte growth factor, or a TGFβ-superfamily protein.

152. The pharmaceutical composition of item 151, wherein the therapeutic agent is a glial cell line-derived neurotrophic factor family ligand selected from glial cell line-derived neurotrophic factor (GDNF), neurturin, artemin, and persephin.

153. The pharmaceutical composition of item 151, wherein the therapeutic agent is a neuropoietic cytokine selected from interleukin-6, interleukin-11, inteleukin-27, leukemia inhibitory factor, CNTF, cardiotrophin 1, neuropoietin, cardiotrophin-like cytokine, and fibroblast growth factor 2.

154. The pharmaceutical composition of item 151, wherein the therapeutic agent is an anti-inflammatory cytokine selected from interleukin-4 and interleukin-10.

155. The pharmaceutical composition of item 151, wherein the therapeutic agent is a neuroprotection agent selected from neuregulin-1, vascular endothelial growth factor (VEGF), sodium thiosulfate, and N-acetyl cysteine.

156. The pharmaceutical composition of item 151, wherein the therapeutic agent is a TGFβ-superfamily protein selected from TGFβ, TGFβ3, BMP2, and BMP7.

157. The pharmaceutical composition of any one of items 1-135, wherein the therapeutic agent is a nucleic acid vector.

158. The pharmaceutical composition of item 157, wherein the nucleic acid vector is a plasmid, cosmid, artificial chromosome, or viral vector.

159. The pharmaceutical composition of item 158, wherein the viral vector is an adeno-associated virus (AAV), adenovirus, lentivirus, retrovirus, poxvirus, baculovirus, herpes simplex virus, or a vaccinia virus.

160. The pharmaceutical composition of item 159, wherein the viral vector is an AAV.

161. The pharmaceutical composition of item 160, wherein the serotype of the AAV vector is selected from the group consisting of AAV1, AAV2, AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, and PHP.S, preferably AAV1.

162. The pharmaceutical composition of any one of items 1-135, wherein the therapeutic agent is an antibody or antigen-binding fragment thereof.

163. The pharmaceutical composition of item 162, wherein the antibody or antigen-binding fragment thereof is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFv.

164. The pharmaceutical composition of any one of items 1-135, wherein the therapeutic agent is encapsulated within a liposome, vesicle, synthetic vesicle, exosome, synthetic exosome, dendrimer, or nanoparticle.

165. The pharmaceutical composition of any one of items 1-135, wherein the therapeutic agent is a small molecule, optionally wherein the small molecule is one that is not naturally round window membrane-penetrant.

166. The pharmaceutical composition of any one of items 1-135, wherein the therapeutic agent is an interfering RNA.

167. The pharmaceutical composition of item 166, wherein the interfering RNA is a short interfering RNA (siRNA), a short hairpin RNA (shRNA), or a micro RNA (miRNA).

168. The pharmaceutical composition of any one of items 1-167, wherein the pharmaceutical composition is a gel at normal human body temperature.

169. The pharmaceutical composition of item 168, wherein the gel has a dynamic viscosity of at about 100 cP to about 1,000,000 cP.

170. The pharmaceutical composition of any one of items 1-169, wherein the pharmaceutical composition further comprises a pharmaceutically acceptable liquid solvent.

171. The pharmaceutical composition of item 170, wherein the pharmaceutically acceptable liquid solvent is water.

172. The pharmaceutical composition of any one of items 1-169, wherein the pharmaceutical composition comprises one or more agents selected from an antimicrobial agent, an arylcycloalkylamine, an elipticine derivative, an anti-apoptotic agent, a c-JNK inhibitor, an antioxidant, an NSAID, an analgesic, a neuroprotection agent, a glutamate modulator, an interleukin 1 modulator, an interleukin-1 antagonist, a corticosteroid, an anti-TNF agent, a calcineurin inhibitor, an IKK inhibitor, an interleukin inhibitor, a platelet activating factor antagonist, a TNF-α converting enzyme (TACE) inhibitor, a Toll-like receptor inhibitor, an autoimmune agent, an IL-1 modulator, an RNA interference agent, an aquaporin modulator, an estrogen-related receptor beta modulator, a GAP junction protein, a vasopressin receptor modulator, an NMDA receptor modulator, an ENaC receptor modulator, an osmotic diuretic, a progesterone receptor, a prostaglandin, a cytotoxic agent, a cytoprotective agent, an anti-intercellular adhesion molecule-1 antibody, an Atoh1 modulator, a Math1 modulator, a BRN-3 modulator, a carbamate, an estrogen receptor, a fatty acid, a gamma-secretase inhibitor, a glutamate-receptor modulator, a neurotrophic agent, salicylic acid, nicotine, a retinoblastoma protein modulator, an ion channel blocker, a thyroid hormone receptor modulator, a TRPV modulator, an adenosine modulator, a KCNQ modulator, a P2X modulator, a CNS modulating agent, an anticholinergic, an antihistamine, a GABA receptor modulator, a neurotransmitter reuptake inhibitor, a thyrotropin-releasing hormone, a free radical modulator, a metal atom chelator, a mitochondrial modulator, a nitric oxide synthase modulator, a sirtuin modulator, a purinergic receptor modulator, a truncated TrkC or TrkB antagonist, a truncated TrkC or TrkB isoform, a nucleic acid polymer antagonist, a small molecule antagonist, a polypeptide antagonist, a non-natural TrkC or TrkB agonist, a neurotrophin variant, a WNT modulator, a glycogen synthase kinase inhibitor, a protein kinase C beta modulator, a repulsive guidance molecule a (RGMa) inhibitor, a neogenin inhibitor, a SK2 channel activator, a BK channel activator, a sphingosine-1-phosphate receptor modulator, a stemness driver, a differentiation inhibitor, an N-Methyl-D-Aspartate (NMDA) receptor antagonist, a histone deacetylase (HDAC) inhibitor, a proteasome inhibitor, an EZH2/HMT inhibitor, a notch inhibitor, ebselen, ancrod, an α-amino-3-hydroxy-5-methyl-4-isoxazolepropionic acid (AMPA) glutamate-positive allosteric modulator, D-methionine, an antagonist of histamine type 4 receptors, a chemotherapeutic accumulation reducer, choline ester, plant alkaloid, reversible cholinesterase inhibitor, acetylcholine release promoter, anti-adrenergy, a sympathomimetic, an antineoplastic agent, R(+)-N-propargyl-1-aminoindan, and R-azasetron besylate.

173. A method of delivering a therapeutic agent across the round window membrane of a subject, the method comprising administering to the subject a therapeutically effective amount of the pharmaceutical composition of any one of items 1-172.

174. The method of item 173, wherein the pharmaceutical composition is administered to or near the round window membrane.

175. The method of item 173 or 174, wherein the pharmaceutical composition is administered intratympanically or transtympanically.

176. The method of any one of items 173-175, wherein the method is used to treat an otic disease.

177. The method of item 176, wherein the otic disease is ceruminosis or ceruminosis associated with an otic disease or condition, ear pruritus, otitis externa, otalgia, tinnitus, vestibular dysfunction, vertigo, dizziness, loss of balance, ear fullness, hearing loss, Meniere's disease, sensorineural hearing loss, noise-induced hearing loss, age-related hearing loss (presbycusis), ototoxic drug-induced hearing loss, hearing loss related to head trauma, hearing loss related to infection, autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

178. A method of treating a subject having or at risk of developing hearing loss, comprising administering to the subject a therapeutically effective amount of the pharmaceutical composition of any one of items 1-172.

179. The method of item 178, wherein the hearing loss is genetic hearing loss.

180. The method of item 179, wherein the genetic hearing loss is autosomal dominant hearing loss, autosomal recessive hearing loss, orX-linked hearing loss.

181. The method of item 178, wherein the hearing loss is acquired hearing loss.

182. The method of item 181, wherein the acquired hearing loss is noise-induced hearing loss, age-related hearing loss, disease or infection-related hearing loss, head trauma-related hearing loss, or ototoxic drug-induced hearing loss.

183. A method of treating a subject having or at risk of developing vestibular dysfunction, comprising administering to the subject a therapeutically effective amount of the pharmaceutical composition of any one of items 1-172.

184. The method of item 183, wherein the vestibular dysfunction is vertigo, dizziness, or imbalance.

185. A method of promoting hair cell regeneration in a subject in need thereof, comprising administering to the subject a therapeutically effective amount of the pharmaceutical composition of any one of items 1-172.

186. A method of promoting SGN regeneration in a subject in need thereof, comprising administering to the subject a therapeutically effective amount of the pharmaceutical composition of any one of items 1-172.

187. A method of preventing or reducing ototoxic drug-induced hair cell damage or death in a subject in need thereof, comprising administering to the subject a therapeutically effective amount of the pharmaceutical composition of any one of items 1-172.

188. A method of preventing or reducing ototoxic drug-induced SGN damage or death, comprising administering to the subject a therapeutically effective amount of the pharmaceutical composition of any one of items 1-172.

189. The method of item 182, 187, or 188, wherein the ototoxic drug is selected from the group consisting of aminoglycosides, antineoplastic drugs, ethacrynic acid, furosemide, salicylates, and quinine.

190. A method of treating a subject having or at risk of developing tinnitus, comprising administering to the subject a therapeutically effective amount of the pharmaceutical composition of any one of items 1-172.

191. A method of preventing or reducing hair cell damage or death in a subject in need thereof, comprising administering to the subject a therapeutically effective amount of the pharmaceutical composition of any one of items 1-172.

192. A method of preventing or reducing SGN damage or death in a subject in need thereof, comprising administering to the subject a therapeutically effective amount of the pharmaceutical composition of any one of items 1-172.

193. A method of increasing hair cell survival in a subject in need thereof, comprising administering to the subject a therapeutically effective amount of the pharmaceutical composition of any one of items 1-172.

194. A method of increasing SGN survival in a subject in need thereof, comprising administering to the subject a therapeutically effective amount of the pharmaceutical composition of any one of items 1-172.

195. A method of increasing the number of supporting cells subject in need thereof, comprising administering to the subject a therapeutically effective amount of the pharmaceutical composition of any one of items 1-172.

196. The method of any one of items 176-195, wherein the method further comprises evaluating the hearing of the subject prior to administering the pharmaceutical composition.

197. The method of any one of items 176-196, wherein the method further comprises evaluating the hearing of the subject after administering the pharmaceutical composition.

198. The method of any one of items 176-197, wherein the method further comprises evaluating the vestibular function of the subject prior to administering the pharmaceutical composition.

199. The method of any one of items 176-198, wherein the method further comprises evaluating the vestibular function of the subject after administering the pharmaceutical composition.

200. The method of any one of items 178-199, wherein the pharmaceutical composition is locally administered.

201. The method of any one of items 176-200, wherein the pharmaceutical composition is administered in an amount sufficient to prevent or reduce hearing loss, prevent or reduce vestibular dysfunction, prevent or reduce tinnitus, delay the development of hearing loss, delay the development of vestibular dysfunction, slow the progression of hearing loss, slow the progression of vestibular dysfunction, improve hearing, improve vestibular function, improve hair cell function, prevent or reduce hair cell damage, prevent, slow, or reduce hair cell death, promote or increase hair cell survival, increase supporting cell numbers, increase hair cell numbers, promote or induce hair cell regeneration, improve SGN function, prevent or reduce SGN damage, prevent, slow, or reduce SGN death, promote or increase SGN survival, increase SGN numbers, promote or induce SGN regeneration, preserve ribbon synapses, promote or increase ribbon synapse formation, maintain the connections between hair cells and SGNs, or increase or restore the connections between hair cells and SGNs.

202. The method of any one of items 173-201, wherein the subject is a mammalian subject.

203. The method of item 202, wherein the mammalian subject is a human subject.

204. A kit comprising the pharmaceutical composition of any one of items 1-172.

205. The kit of item 204, wherein the kit further comprises a package insert instructing a user of the kit to administer the pharmaceutical composition to a subject in need thereof.

206. The kit of item 205, wherein the subject is a mammalian subject.

207. The kit of item 206, wherein the mammalian subject is a human subject.

### Definitions

As used herein, the term “about” refers to a value that is within 10% above or below the value being described. For instance, a value of “about 5 μL” refers to a quantity that is from 4.5 μL to 5.5 μL.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1, below.

TABLE 1
Representative physicochemical properties of
naturally-occurring amino acids

| | | | | Electrostatic | |
|---|---|---|---|---|---|
| | 3 | 1 | Side | character at | |
| Amino | Letter | Letter | chain | physiological | Steric |
| Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic | Asp | D | polar | anionic | intermediate |
| acid | | | | | |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenyl- | Phe | F | nonpolar | neutral | large |
| alanine | | | | | |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) 0 and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

The formal charge of an amino acid residue at a particular pH, such as physiological pH (7.4), can be determined using the Henderson-Hasselbalch equation, pH=pKa+log [A−]/[HA], as applied to the side-chain functional group of the amino acid of interest, wherein “HA” designates the protonated form of the side-chain substituent and “A-” designates the deprotonated form of the side-chain substituent. It will be appreciated by one of skill in the art that the Henderson-Hasselbalch equation may be applied multiple times to the same amino acid for those that contain side-chains that undergo more than one ionization at the pH of interest (e.g., pH of 7.4), such as those that contain a phosphate substituent, among others. The formal charge of an amino acid as described herein refers to the charge of the predominant form (i.e., the form present in the highest quantity at chemical equilibrium) of the amino acid side chain substituent (e.g., “HA” or “A-”) as determined by the Henderson-Hasselbalch equation.

As used herein, the term “hydrophobic side-chain” refers to an amino acid side chain that exhibits low solubility in water relative due to, e.g., the steric or electronic properties of the chemical moieties present within the side chain. Amino acids containing hydrophobic side-chains include those containing unsaturated aliphatic hydrocarbons, such as valine, leucine, isoleucine, proline, and methionine, as well as amino acids containing aromatic ring systems that are electrostatically neutral at physiological pH, such as tryptophan, phenylalanine, and tyrosine.

As used herein, the terms “neurotrophin-3” and its abbreviation, “NT-3,” are used interchangeably and refer to the protein growth factor that exerts growth or survival activating effects on various neurons of the central and/or peripheral nervous system, as well as to the nucleic acid encoding the protein. The terms “neurotrophin-3” and its abbreviation, “NT-3,” refer not only to wild-type forms of NT-3, but also to variants of wild-type NT-3 proteins that retain (or improve upon) the neuronal growth or survival activating properties of wild-type NT-3, as well as to nucleic acids encoding such variants. The amino acid sequence and corresponding cDNA sequence of a wild-type form of human NT-3 are provided herein as SEQ ID NOs: 15 and 16, which correspond to UniProtKB Accession No. P20783 and to CCDS 8538.1, respectively. These sequences are shown in Table 2, below. The amino acid sequence of isoform 2 of wild-type human NT-3 and its corresponding cDNA sequence (SEQ ID NOs: 21 and 62), the amino acid sequence of the mature form of wild-type human NT-3 (SEQ ID NO: 22), and amino acid sequences of variants of the mature form of wild-type human NT-3 (SEQ ID NOs: 48-60) are provided in Table 3, herein.

TABLE 2
Amino acid and cDNA nucleic
acid sequences of wild-type human NT-3

| SEQ | |
|---|---|
| ID NO. | Sequence |
| 15 | MSILFYVIFLAYLRGIQGNNMDQRSLPEDSLNSLIIKLIQA |
| | DILKNKLSKQMVDVKENYQSTLPKAEAPREPERGGPAKSAF |
| | QPVIAMDTELLRQQRRYNSPRVLLSDSTPLEPPPLYLMEDY |
| | VGSPVVANRTSRRKRYAEHKSHRGEYSVCDSESLWVTDKSS |
| | AIDIRGHQVTVLGEIKTGNSPVKQYFYETRCKEARPVKNGC |
| | RGIDDKHWNSQCKTSQTYVRALTSENNKLVGWRWIRIDTSC |
| | VCALSRKIGRT |
| 16 | ATGTCCATCTTGTTTTATGTGATATTTCTCGCTTATCTCCG |
| | TGGCATCCAAGGTAACAACATGGATCAAAGGAGTTTGCCAG |
| | AAGACTCGCTCAATTCCCTCATTATTAAGCTGATCCAGGCA |
| | GATATTTTGAAAAACAAGCTCTCCAAGCAGATGGTGGACGT |
| | TAAGGAAAATTACCAGAGCACCCTGCCCAAAGCTGAGGCTC |
| | CCCGAGAGCCGGAGCGGGGAGGGCCCGCCAAGTCAGCATTC |
| | CAGCCGGTGATTGCAATGGACACCGAACTGCTGCGACAACA |
| | GAGACGCTACAACTCACCGCGGGTCCTGCTGAGCGACAGCA |
| | CCCCCTTGGAGCCCCCGCCCTTGTATCTCATGGAGGATTAC |
| | GTGGGCAGCCCCGTGGTGGCGAACAGAACATCACGGCGGAA |
| | ACGGTACGCGGAGCATAAGAGTCACCGAGGGGAGTACTCGG |
| | TATGTGACAGTGAGAGTCTGTGGGTGACCGACAAGTCATCG |
| | GCCATCGACATTCGGGGACACCAGGTCACGGTGCTGGGGGA |
| | GATCAAAACGGGCAACTCTCCCGTCAAACAATATTTTTATG |
| | AAACGCGATGTAAGGAAGCCAGGCCGGTCAAAAACGGTTGC |
| | AGGGGTATTGATGATAAACACTGGAACTCTCAGTGCAAAAC |
| | ATCCCAAACCTACGTCCGAGCACTGACTTCAGAGAACAATA |
| | AACTCGTGGGCTGGCGGTGGATACGGATAGACACGTCCTGT |
| | GTGTGTGCCTTGTCGAGAAAAATCGGAAGAACA |

The terms “neurotrophin-3” and its abbreviation, “NT-3,” as used herein include, for example, forms of the human NT-3 protein that have an amino acid sequence that is at least 85% identical to the amino acid sequence of SEQ ID NOs: 15, 21, or 22 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 15, 21, or 22) and/or forms of the human NT-3 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type NT-3 protein. Similarly, the terms “neurotrophin-3” and its abbreviation, “NT-3,” as used herein include, for example, forms of the human NT-3 gene that correspond to a cDNA having a nucleic acid sequence that is at least 85% identical to the nucleic acid sequence of SEQ ID NO: 16 or 62 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 16 or 62).

As used herein, the term “dose” refers to the quantity of a therapeutic agent, such as a neurotrophin described herein (e.g., NT-3) that is administered to a subject for the treatment of a disorder or condition, such as to treat or prevent sensory hair cell loss). A therapeutic agent as described herein may be administered in the form of a pharmaceutical composition in a single dose or in multiple doses for the treatment of a particular indication. In each case, the pharmaceutical composition agent may be administered using one or more unit dosage forms. For instance, a single dose of 1 mg of a therapeutic agent may be administered using, e.g., two 0.5 mg unit dosage forms of the therapeutic agent, four 0.25 mg unit dosage forms of the therapeutic agent, one single 1 mg unit dosage form of the therapeutic agent, and the like.

As used herein, the term “endogenous” describes a molecule (e.g., a metabolite, polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a small molecule, polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “interfering RNA” refers to a RNA, such as a short interfering RNA (siRNA), micro RNA (miRNA), or short hairpin RNA (shRNA) that suppresses the expression of a target RNA transcript, for example, by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having a neurological disorder described herein, in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” means a mixture containing a therapeutic compound to be administered to a patient, such as a mammal, e.g., a human, in order to prevent, treat or control a particular disease or condition affecting the mammal, such as a neurological disorder described herein.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of therapeutic treatment, the terms “provide” and “providing” refer to the delivery of a therapeutic agent to a subject (e.g., a mammalian subject, such as a human) in need of treatment, such as a subject experiencing or at risk of developing a neurological disorder described herein. A therapeutic agent may be provided to a subject in need thereof, for instance, by direct administration of the therapeutic agent to the subject, or by administration of a prodrug that is converted in vivo to the therapeutic agent upon administration of the prodrug to the subject. Exemplary prodrugs include, without limitation, esters, phosphates, and other chemical functionalities susceptible to hydrolysis upon administration to a subject. Prodrugs include those known in the art, such as those described, for instance, in Vig et al., Adv. Drug Deliv. Rev. 65:1370-1385 (2013), and Huttunen et al., Pharmacol. Rev. 63:750-771 (2011), the disclosures of each of which are incorporated herein by reference in their entirety.

As used herein, the term “sample” refers to a specimen (e.g., blood, blood component (e.g., serum or plasma), urine, saliva, amniotic fluid, cerebrospinal fluid, tissue (e.g., placental or myometrial), pancreatic fluid, chorionic villus sample, and cells) isolated from a patient.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 μM and 100 nM). A ligand that does not exhibit specific binding to a protein or a domain thereof will exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are used interchangeably and refer to an organism, such as a mammal (e.g., a human) that receives therapy for the treatment or prevention of a disease or condition described herein, for example, for an otic disease or condition, such as ear pruritus, otitis externa, otalgia, tinnitus, vestibular dysfunction (e.g., vertigo, dizziness, loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., hearing, such as hearing measured using standard clinical tests) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating sensorineural hearing loss or vestibular dysfunction, it is an amount of the pharmaceutical composition sufficient to achieve a treatment response as compared to the response obtained without administration of the pharmaceutical composition. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a pharmaceutical composition of the present disclosure is an amount which results in a beneficial or desired result in a subject as compared to a control. As defined herein, a therapeutically effective amount of a pharmaceutical composition of the present disclosure may be readily determined by one of ordinary skill by routine methods known in the art. Dosage regimen may be adjusted to provide the optimum therapeutic response.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear (e.g., transtympanic or intratympanic injection), and administration to a mucous membrane of the subject, wherein the administration is intended to have a local and not a systemic effect.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “spiral ganglion neuron” and “SGN” refer to a type of specialized cell in the inner ear that innervates cochlear hair cells. There are two main classes of SGNs: Type I SGNs, which innervate inner hair cells and transmit sound information to the central nervous system (CNS), and Type II SGNs, which innervate outer hair cells and are not required for the transmission of sound information to the CNS.

As used herein, the term “vestibular hair cell” refers to group of specialized cells in the inner ear that are involved in sensing movement and contribute to the sense of balance and spatial orientation.

Vestibular hair cells are located in the semicircular canals and otolith organs (e.g., utricle and saccule) of the inner ear. Damage to vestibular hair cells and genetic mutations that disrupt vestibular hair cell function are implicated in vestibular dysfunction such as vertigo and imbalance disorders.

As used herein, the term “wild-type” refers to a genotype with the highest frequency for a particular gene in a given organism.

As used herein in the context of a neurological disorder, the terms “treat” or “treatment” refer to therapeutic treatment, in which the object is to slow, delay, or halt the progression or development of a neurological disorder, e.g., in a human subject.

As used herein, the term “alkyl” refers to monovalent, optionally branched alkyl groups, such as those having from 1 to 6 carbon atoms, or more. This term is exemplified by groups such as methyl, ethyl, n-propyl, isopropyl, n-butyl, isobutyl, tert-butyl, n-hexyl and the like.

As used herein, the terms “lower alkyl” and “C₁-C₆ alkyl” refer to alkyl groups having from 1 to 6 carbon atoms.

As used herein, the term “aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenanthrenyl and the like.

As used herein, the terms “aralkyl” and “aryl alkyl” are used interchangeably and refer to an alkyl group containing an aryl moiety. Similarly, the terms “aryl lower alkyl” and the like refer to lower alkyl groups containing an aryl moiety.

As used herein, the term “alkyl aryl” refers to alkyl groups having an aryl substituent, including benzyl, phenethyl and the like.

As used herein, the term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, I,3,4-oxadiazolyl,1,3,4-triazinyl, 1,2,3-triazinyl, benzofuryl, [2,3-dihydrojbenzofuryl, isobenzofuryl, benzothienyl, benzotriazolyl, isobenzothienyl, indolyl, isoindolyl, 3H-indolyl, benzimidazolyl, imidazo[I,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “alkyl heteroaryl” refers to alkyl groups having a heteroaryl substituent, including 2-furylmethyl, 2-thienylmethyl, 2-(1H-indol-3-yl)ethyl and the like.

As used herein, the term “lower alkenyl” refers to alkenyl groups preferably having from 2 to 6 carbon atoms and having at least 1 or 2 sites of alkenyl unsaturation. Exemplary alkenyl groups are ethenyl (—CH═CH₂), n-2-propenyl (allyl, —CH₂CH═CH₂) and the like.

As used herein, the term “alkenyl aryl” refers to alkenyl groups having an aryl substituent, including 2-phenylvinyl and the like.

As used herein, the term “alkenyl heteroaryl” refers to alkenyl groups having a heteroaryl substituent, including 2-(3-pyridinyl)vinyl and the like.

As used herein, the term “lower alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

As used herein, the term “alkynyl aryl” refers to alkynyl groups having an aryl substituent, including phenylethynyl and the like.

As used herein, the term “alkynyl heteroaryl” refers to alkynyl groups having a heteroaryl substituent, including 2-thienylethynyl and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “lower cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

As used herein, the term “heterocycloalkyl” refers to a cycloalkyl group in which one or more ring carbon atoms are replaced with a heteroatom, such as a nitrogen atom, an oxygen atom, a sulfur atom, and the like. Exemplary heterocycloalkyl groups are pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the term “alkyl cycloalkyl” refers to alkyl groups having a cycloalkyl substituent, including cyclohexylmethyl, cyclopentylpropyl, and the like.

As used herein, the term “alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

As used herein, the term “carboxy” refers to the group —C(O)OH.

As used herein, the term “alkyl carboxy” refers to C₁-C₅-alkyl groups having a carboxy substituent, including 2-carboxyethyl and the like.

As used herein, the term “acyl” refers to the group —C(O)R, wherein R may be, for example, C₁-C₆-alkyl, aryl, heteroaryl, C₁-C₆-alkyl aryl, or C₁-C₆-alkyl heteroaryl, among other substituents. The term “C₁-C₆ acyl” refers to an acyl group containing from 1 to 6 carbon atoms.

As used herein, the term “acyloxy” refers to the group —OC(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “alkoxy” refers to the group —O—R, wherein R is, for example, an optionally substituted alkyl group, such as an optionally substituted C₁-C₆-alkyl, aryl, heteroaryl, C₁-C₆-alkyl aryl, or C₁-C₆-alkyl heteroaryl, among other substituents. Exemplary alkoxy groups include by way of example, methoxy, ethoxy, phenoxy, and the like.

As used herein, the term “alkoxycarbonyl” refers to the group —C(O)OR, wherein R is, for example, hydrogen, C₁-C₆-alkyl, aryl, heteroaryl, C₁-C₆-alkyl aryl, or C₁-C₆-alkyl heteroaryl, among other possible substituents.

As used herein, the term “alkyl alkoxycarbonyl” refers to alkyl groups having an alkoxycarbonyl substituent, including 2-(benzyloxycarbonyl)ethyl and the like.

As used herein, the term “aminocarbonyl” refers to the group —C(O)NRR′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl aminocarbonyl” refers to alkyl groups having an aminocarbonyl substituent, including 2-(dimethylaminocarbonyl)ethyl and the like.

As used herein, the term “acylamino” refers to the group —NRC(O)R′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl acylamino” refers to alkyl groups having an acylamino substituent, including 2-(propionylamino)ethyl and the like.

As used herein, the term “ureido” refers to the group —NRC(O)NR′R″, wherein each of R, R′, and R″ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary ureido groups further include moieties in which R′ and R″, together with the nitrogen atom to which they are attached, form a 3-8-membered heterocycloalkyl ring.

As used herein, the term “alkyl ureido” refers to alkyl groups having an ureido substituent, including 2-(N′-methylureido)ethyl and the like.

As used herein, the term “amino” refers to the group —NRR′, wherein each of R and R′ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary amino groups further include moieties in which R and R′, together with the nitrogen atom to which they are attached, can form a 3-8-membered heterocycloalkyl ring.

As used herein, the term “alkyl amino” refers to alkyl groups having an amino substituent, including 2-(1-pyrrolidinyl)ethyl and the like.

As used herein, the term “ammonium” refers to a positively charged group —N*RR′R″, wherein each of R, R′, and R″ may independently be, for example, C₁-C₆-alkyl, C₁-C₆-alkyl aryl, C₁-C₆-alkyl heteroaryl, cycloalkyl, or heterocycloalkyl, among other substituents. Exemplary ammonium groups further include moieties in which R and R′, together with the nitrogen atom to which they are attached, form a 3-8-membered heterocycloalkyl ring.

As used herein, the term “halogen” refers to fluoro, chloro, bromo and iodo atoms.

As used herein, the term “sulfonyloxy” refers to a group —OSO₂—R wherein R is selected from hydrogen, C₁-C₆-alkyl, C₁-C₆-alkyl substituted with halogens, e.g., an —OSO₂—CF₃ group, aryl, heteroaryl, C₁-C₆-alkyl aryl, and C₁-C₆-alkyl heteroaryl.

As used herein, the term “alkyl sulfonyloxy” refers to alkyl groups having a sulfonyloxy substituent, including 2-(methylsulfonyloxy)ethyl and the like.

As used herein, the term “sulfonyl” refers to group “—SO₂—R” wherein R is selected from hydrogen, aryl, heteroaryl, C₁-C₆-alkyl, C₁-C₆-alkyl substituted with halogens, e.g., an —SO₂—CF₃ group, C₁-C₆-alkyl aryl or C₁-C₆-alkyl heteroaryl.

As used herein, the term “alkyl sulfonyl” refers to alkyl groups having a sulfonyl substituent, including 2-(methylsulfonyl)ethyl and the like.

As used herein, the term “sulfinyl” refers to a group “—S(O)—R” wherein R is selected from hydrogen, C₁-C₆-alkyl, C₁-C₆-alkyl substituted with halogens, e.g., a —SO—CF₃ group, aryl, heteroaryl, C₁-C₆-alkyl aryl or C₁-C₆-alkyl heteroaryl.

As used herein, the term “alkyl sulfinyl” refers to C₁-C₅-alkyl groups having a sulfinyl substituent, including 2-(methylsulfinyl)ethyl and the like.

As used herein, the term “sulfanyl” refers to groups —S—R, wherein R is, for example, alkyl, aryl, heteroaryl, C₁-C₆-alkyl aryl, or C₁-C₆-alkyl heteroaryl, among other substituents. Exemplary sulfanyl groups are methylsulfanyl, ethylsulfanyl, and the like.

As used herein, the term “alkyl sulfanyl” refers to alkyl groups having a sulfanyl substituent, including 2-(ethylsulfanyl)ethyl and the like.

As used herein, the term “sulfonylamino” refers to a group —NRSO₂—R′, wherein each of R and R′ may independently be hydrogen, C₁-C₆-alkyl, aryl, heteroaryl, C₁-C₆-alkyl aryl, or C₁-C₆-alkyl heteroaryl, among other substituents.

As used herein, the term “alkyl sulfonylamino” refers to alkyl groups having a sulfonylamino substituent, including 2-(ethylsulfonylamino)ethyl and the like.

As used herein, the term “optionally substituted” refers to the optional inclusion of one or more chemical functional groups, such as those set forth above, in an indicated moiety. Unless otherwise constrained by the definition of the individual substituent, the above set out groups, like “alkyl”, “alkenyl”, “alkynyl”, “aryl” and “heteroaryl” etc. groups can optionally be substituted, for example, with one or more substituents, as valency permits, such as a substituent selected from alkyl (e.g., C₁-C₆-alkyl), alkenyl (e.g., C₂-C₆-alkenyl), alkynyl (e.g., C₂-C₆-alkynyl), cycloalkyl, heterocycloalkyl, alkyl aryl (e.g., C₁-C₆-alkyl aryl), alkyl heteroaryl (e.g., C₁-C₆-alkyl heteroaryl, alkyl cycloalkyl (e.g., C₁-C₆-alkyl cycloalkyl), alkyl heterocyloalyl (e.g., C₁-C₆-alkyl heterocycloalkyl), amino, ammonium, acyl, acyloxy, acylamino, aminocarbonyl, alkoxycarbonyl, ureido, aryl, heteroaryl, sulfinyl, sulfonyl, alkoxy, sulfanyl, halogen, carboxy, trihalomethyl, cyano, hydroxy, mercapto, nitro, and the like. In some embodiments, the substitution is one in which neighboring substituents have undergone ring closure, such as situations in which vicinal functional substituents are involved, thus forming, e.g., lactams, lactones, cyclic anhydrides, acetals, thioacetals, and aminals, among others.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR, R′, R″ *Z—, wherein each of R, R′, and R″ may independently be, for example, hydrogen, alkyl, benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

The structural compositions described herein also include the tautomers, geometrical isomers (e.g., E/Z isomers and cis/trans isomers), enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

As used herein, chemical structural formulas that do not depict the stereochemical configuration of a compound having one or more stereocenters will be interpreted as encompassing any one of the stereoisomers of the indicated compound, or a mixture of one or more such stereoisomers (e.g., any one of the enantiomers or diastereomers of the indicated compound, or a mixture of the enantiomers (e.g., a racemic mixture) or a mixture of the diastereomers). As used herein, chemical structural formulas that do specifically depict the stereochemical configuration of a compound having one or more stereocenters will be interpreted as referring to the substantially pure form of the particular stereoisomer shown. “Substantially pure” forms refer to compounds having a purity of greater than 85%, such as a purity of from 85% to 99%, 85% to 99.9%, 85% to 99.99%, or 85% to 100%, such as a purity of 85%, 86%, 87%, 88%, 89%, 90%, 91%, 92%, 93%, 94%, 95%, 96%, 97%, 98%, 99%, 99.9%, 99.99%, 99.999%, or 100%, as assessed, for example, using chromatography and nuclear magnetic resonance techniques known in the art.

Unless otherwise indicated, all amino acid sequences using three-letter code or one-letter code are depicted in the N-terminal to C-terminal direction.

As used herein in the context of an amino acid sequence, capital letters designate the naturally-occurring “L” form of the indicated amino acid (i.e., using the one-letter code set forth in Table 1, herein). Lowercase letters denote the “D” form of the indicated amino acid (i.e., using the same one-letter code set forth in Table 1, herein).

As used herein, the terms “amino acid,” “amino acid monomer,” and “amino acid residue” are used interchangeably and refer to naturally-occurring alpha-amino acid molecules, such as those set forth in Table 1, herein. The terms “amino acid,” “amino acid monomer,” and “amino acid residue” additionally refer to variants of the amino acid molecules set forth in Table 1, such as beta-amino acids and gamma-amino acids, as well as amino acids in which the side chain is located on the amino nitrogen (such as the amino acid monomers that comprise peptoids, described, for example, in Kwon and K_{D} dedk, J. Am. Chem. Soc. 129:1508-1509 (2007), the disclosure of which is incorporated herein by reference). The terms “amino acid,” “amino acid monomer,” and “amino acid residue” additionally refer to variants of the amino acid molecules set forth in Table 1 in which the amino nitrogen atom is modified, for example, by way of N-alkylation (e.g., N-methylation). Examples of N-methylated amino acids are described in Chatterjee et al., Acc. Chem. Res. 41:1331-1342 (2008), the disclosure of which is incorporated herein by reference). The terms amino acid,” “amino acid monomer,” and “amino acid residue” additionally refer to amino acid variants that are configured so as to be joined to a neighboring amino acid by way of a peptide bond isostere.

As used herein the terms “peptide” and “polypeptide” are used interchangeably and refer to polymers containing repeating amino acid molecules covalently bound to one another by way of amide bonds (also referred to as peptide bonds) or peptide bond isosteres).

As used herein, the term “peptide bond isostere” refers to a variant of a peptide bond that mimics the steric and/or electronic properties of a conventional —NH—C(O)— amide bond. Exemplary amino acid isosteres that may be used in conjunction with the compositions and methods described herein are described, e.g., in Choudhary et al., Chembiochem 12:1801-1807 (2011), the disclosure of which is incorporated herein by reference. Examples of peptide bond isosteres that may be incorporated into the permeation enhancers described herein are:

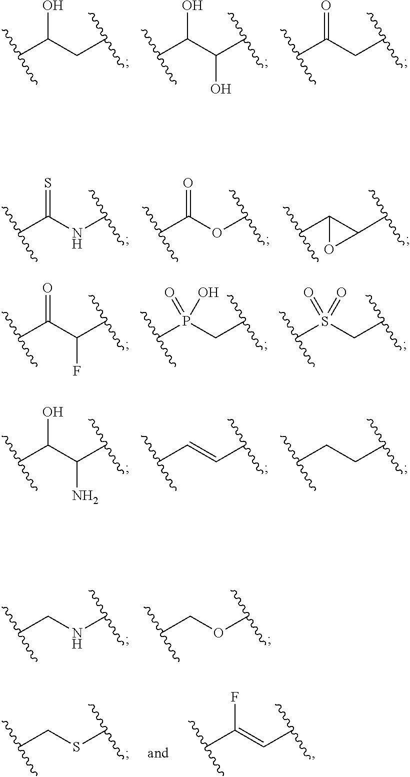

among others.

As used herein, the term “facially amphipathic” refers to a peptide, such as an alpha-helical peptide, that contains one plane or face having hydrophobic amino acids, and another plane or face having hydrophilic amino acids. An example of a facially amphipathic peptide is shown in FIG. 23, which is a helical wheel diagram for the peptide represented by SEQ ID NO: 1, herein. As shown in FIG. 23, this peptide contains a hydrophobic region comprised primarily of leucine and alanine residues (lower and left portions of FIG. 23) and a hydrophilic region containing several positively charged lysine residues (upper right portion of FIG. 23). Facially amphipathic peptides can be designed by placing amino acid residues in alternating positions within the primary sequence of a polypeptide so as to create hydrophobic and hydrophilic faces on the resulting peptide. In the context of an alpha-helix, the spatial location of these amino acids along the helix can be predicted using helical wheel methods known in the art, such as those described in Eisenberg et al., Nature 229:371-374 (1982), the disclosure of which is incorporated herein by reference.

As used herein, the term “retro-inverso” refers to a variant of a parent peptide in which the amino acid residues of the parent peptide are (i) inverted in stereochemical configuration at all chiral centers and (ii) arranged in the opposite direction relative to that of the parent sequence. For example, the retro-inverso sequence of the peptide KLA (from N-terminus to C-terminus), comprised of all L amino acids, is alk (from N-terminus to C-terminus), comprised of all D-amino acids. As used herein, the term “antibody” (Ab) refers to an immunoglobulin molecule that specifically binds to, or is immunologically reactive with, a particular antigen, and includes polyclonal, monoclonal, genetically engineered, and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rIgG, and scFv fragments. In some embodiments, two or more portions of an immunoglobulin molecule are covalently bound to one another, e.g., via an amide bond, a thioether bond, a carbon-carbon bond, a disulfide bridge, or by a linker, such as a linker described herein or known in the art. Antibodies also include antibody-like protein scaffolds, such as the tenth fibronectin type III domain (¹⁰Fn3), which contains BC, DE, and FG structural loops similar in structure and solvent accessibility to antibody complementarity-determining regions (CDRs). The tertiary structure of the ¹⁰Fn3 domain resembles that of the variable region of the IgG heavy chain, and one of skill in the art can graft, e.g., the CDRs of a reference antibody onto the fibronectin scaffold by replacing residues of the BC, DE, and FG loops of ¹⁰Fn3 with residues from the CDR-H1, CDR-H2, or CDR-H3 regions, respectively, of the reference antibody.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated CDR; and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “bispecific antibodies” refers to monoclonal, often human or humanized antibodies that have binding specificities for at least two different antigens.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, 1985, Science 229(4719): 1202-7; Oi et al, 1986, BioTechniques 4:214-221; Gillies et al, 1985, J. Immunol. Methods 125:191-202; U.S. Pat. Nos. 5,807,715; 4,816,567; and 4,816,397; incorporated herein by reference.

As used herein, the term “complementarity-determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may comprising modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference).

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al, mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized antibodies” refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Exemplary multi-specific molecules have been reviewed by K_{D} ntermann, 2012, mAbs 4(2):182-197, Yazaki et al, 2013, Protein Engineering, Design & Selection 26(3):187-193, and Grote et al, 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

The term “intratympanic,” as used herein in reference to a route of administration, means delivery to the round window by injection or infusion through an ear canal with a temporarily removed or lifted tympanic membrane or through a port created through an auditory bulla into the middle ear of a subject.

The term “transtympanic,” as used herein, in reference to a route of administration, means delivery to the round window by injection or infusion across tympanic membrane. A transtympanic injection may be performed directly through the tympanic membrane or through a tube embedded in the tympanic membrane (e.g., through a tympanostomy tube or grommet).

### BRIEF DESCRIPTION OF THE FIGURES

FIG. 1 is a schematic diagram illustrating the experimental setup of the in vitro membrane penetration experiments described in Example 1, below.

FIG. 2 is a graph showing the calculated round window membrane permeability (Papp) of NT-3, atenolol, metoprolol, and lucifer yellow. Hollow squares denote Papp values obtained in artificial perilymph (AP+) solution. Squares filled with “X” denote values obtained upon 30 minutes of pre-treatment with EDTA. Triangles denote values obtained in 20% poloxamer 407.

FIG. 3 is a graph showing NT-3 concentration as a function of time in perilymph from n=5 guinea pig subjects upon administration of a formulation of 1 mg/ml NT-3 in 20% w/v poloxamer 407. NT-3 concentration was assessed in a perilymph sample obtained using a pipette.

FIG. 4 is a graph showing NT-3 concentration as a function of time in perilymph from n=5 guinea pig subjects upon administration of a formulation of 1 mg/ml NT-3 in poloxamer 407 (20% w/v) with 2% benzyl alcohol as a permeation enhancer.

FIG. 5 is a graph showing NT-3 concentration as a function of time in perilymph from n=5 guinea pig subjects upon administration of a formulation of 1 mg/ml NT-3 in poloxamer 407 (20% w/v) with 2× phosphate buffered saline (PBS) in an attempt to increase osmolarity and enhance permeation.

FIG. 6 is a graph showing NT-3 concentration as a function of time in perilymph from n=5 guinea pig subjects upon administration of a formulation of 1 mg/ml NT-3 in poloxamer 407 (20% w/v) with 1% EDTA as a permeation enhancer.

FIG. 7 is a graph showing NT-3 concentration as a function of time in perilymph from n=5 guinea pig subjects upon administration of a formulation of 1 mg/ml NT-3 in poloxamer 407 (20% w/v) with 2× PBS for elevated osmolarity and 1% EDTA in an attempt to enhance permeation.

FIG. 8 is a graph showing NT-3 concentration as a function of time in perilymph from n=5 guinea pig subjects upon administration of a formulation of 1 mg/ml NT-3 and 0.5% sodium caprate as a permeation enhancer.

FIG. 9 is a graph showing NT-3 concentration as a function of time in perilymph from n=5 guinea pig subjects upon administration of a formulation of 1 mg/ml NT-3 and 2% dipotassium glycyrrhizinate as a permeation enhancer.

FIG. 10 is a graph showing NT-3 concentration as a function of time in perilymph from n=5 guinea pig subjects upon administration of a formulation of 1 mg/ml NT-3 and 1% of peptide Ac-ADTPPV (SEQ ID NO: 17) as a permeation enhancer.

FIG. 11 is a graph showing NT-3 concentration as a function of time in perilymph from n=5 guinea pig subjects upon administration of a formulation of 1 mg/ml NT-3 and 1% of peptide Ac-SHAVSS (SEQ ID NO: 18) as a permeation enhancer.

FIG. 12 is a graph showing NT-3 concentration as a function of time in perilymph from n=5 guinea pig subjects upon administration of a formulation of 1 mg/ml NT-3 in poloxamer 407 (20% w/v) with 1% of the peptide represented by formula (XVI), herein, as a permeation enhancer. Robust, sustained NT-3 exposure was observed in guinea pig cochlear perilymph, as evidenced by the consistent, elevated NT-3 concentration recorded at each time point in the assay.

FIGS. 13A and 13B show a side-by-side comparison of the NT-3 exposure observed as a function of time in perilymph from n=5 guinea pig subjects upon administration of a formulation of 1 mg/ml NT-3 in poloxamer 407 (20% w/v) (FIG. 15A) and upon administration of 1 mg/ml NT-3 in poloxamer 407 (20% w/v) with 1% of the peptide represented by formula (XVI), herein, as a permeation enhancer (FIG. 15B). A significant increase in cochlear NT-3 exposure was obtained upon inclusion of the peptide of formula (XVI).

FIG. 14 is a graph showing a comparison of the cochlear NT-3 exposure observed in n=5 guinea pig subjects upon administration of 0.1 mg/ml NT-3 in 20% (w/v) poloxamer 407 (i) with 1% of the peptide represented by formula (XVI) (circles); (ii) with 0.5% (w/v) sodium caprate (squares); and (iii) in tris buffer (triangles).

FIG. 15 is a graph showing a comparison of the cochlear NT-3 exposure observed in n=5 guinea pig subjects upon intratympanic administration of 0.1 mg/ml NT-3 in 20% (w/v) poloxamer 407 (i) with 1% of the peptide represented by formula (XVI) (circles represent two independent trials); (ii) with 0.5% (w/v) sodium caprate (squares); and (iii) in PBS (triangles).

FIG. 16 is a graph demonstrating the storage stability of NT-3 formulations in various excipients as assessed by the TrkC-binding affinity of the protein in an ELISA assay (expressed as a ratio of 460 nm/530 nm readouts along the y-axis). From top to bottom, along the figure legend: NT-3 standard, freshly used; 20% poloxamer 407 (w/v), freshly used; peptide represented by formula (XVI), frozen immediately after preparation and subsequently thawed; 1 mg/ml NT-3 alone after 2 days at 4° C.; 1 mg/ml NT-3 frozen immediately after preparation and subsequently thawed; 1 mg/ml NT-3 and 1% peptide represented by formula (XVI), frozen immediately after preparation and subsequently thawed; 1 mg/ml NT-3 in 1% peptide represented by formula (XVI), frozen within 8 hours of preparation and subsequently thawed; 1 mg/ml NT-3 in 1% peptide represented by formula (XVI), frozen within 24 hours of preparation and subsequently thawed; 1 mg/ml NT-3 in 1% peptide represented by formula (XVI), frozen within 48 hours of preparation and subsequently thawed. In FIG. 16, Pep is KLALKLALKALKLAALKLA.TFA.

FIG. 17 is a graph showing IgG1 concentration as a function of time in perilymph from n=5 guinea pig subjects upon administration of a formulation of 5 mg/mL IgG1 in poloxamer 407 (20%), with 1% peptide represented by formula (XVI), herein, as a permeation enhancer. Robust, sustained IgG1 exposure was observed in guinea pig cochlear perilymph, as evidenced by the consistent, elevated IgG1 concentration recorded at each time point in the assay. FIG. 18 is a schematic diagram illustrating the experimental setup of the in vitro membrane penetration experiments designed to evaluate the permeability of NT-3 with 1% of the peptide represented by formula (XVI). Tight and adherens junctions were evaluated after 3.5 hrs of incubation with NT-3 and 1% of the peptide represented by formula (XVI) at 37° C.

FIG. 19 is a series of images showing immunohistochemical staining for cell junction proteins after 3.5 hours of incubation with NT-3 in vehicle (control condition). Adherens junctions appeared normal after 3.5 hours of incubation with NT-3 in vehicle,

FIG. 20 is a series of images showing immunohistochemical staining for tight junction proteins after 3.5 hours of incubation with NT-3 and 1% of the peptide represented by formula (XVI). After incubation with NT-3 and 1% of the peptide permeabilizer, gaps were observed between cells (asterisks in merged image on bottom right).

FIG. 21 is a graph showing the flux profiles for NT-3 in vehicle with a 3 μm pore filter (control) and NT-3 and 1% of the peptide represented by formula (XVI).

FIG. 22 is a graph showing the apparent permeability (Papp) of NT-3 in different conditions (vehicle with 0.4 μm pore filter, EDTA with 0.4 μm pore filter, vehicle with 3 μm pore filter, and 1% of the peptide represented by formula (XVI)). 1% of the peptide represented by formula (XVI) increased NT-3 permeability.

FIG. 23 is a helical wheel diagram for the peptide represented by SEQ ID NO: 1, herein. As shown in FIG. 23, this peptide contains a hydrophobic region comprised primarily of leucine and alanine residues (lower and left portions of FIG. 23) and a hydrophilic region containing several positively charged lysine residues (upper right portion of FIG. 23). Facially amphipathic peptides can be designed by placing amino acid residues in alternating positions within the primary sequence of a polypeptide so as to create hydrophobic and hydrophilic faces on the resulting peptide. In the context of an alpha-helix, the spatial location of these amino acids along the helix can be predicted using helical wheel methods known in the art, such as those described in Eisenberg et al., Nature 229:371-374 (1982), the disclosure of which is incorporated herein by reference.

FIG. 24A is a series of images evaluating tight junction integrity in the control guinea pig RWM after 1 hour in ex vivo culture. Actin, occludin, beta-catenin, or alpha-catenin staining was used.

FIG. 24B is a series of images evaluating tight junction integrity in the guinea pig RWM after application of 1% (w/v) KLALKLALKALKLAALKLA.TFA in ex vivo culture. Actin, occludin, beta-catenin, or alpha-catenin staining was used.

FIG. 25A is a series of images evaluating tight junction integrity in the control guinea pig RWM after 1 hour in ex vivo culture. Actin, ZO-1, beta-catenin, or alpha-catenin staining was used.

FIG. 25B is a series of images evaluating tight junction integrity in the guinea pig RWM after application of 0.1% (w/v) KLALKLALKALKLAALKLA.HCl in ex vivo culture. Actin, ZO-1, beta-catenin, or alpha-catenin staining was used.

FIG. 26A is a series of images evaluating tight junction integrity in the control guinea pig RWM after 1 hour in ex vivo culture. Actin, ZO-1, or alpha-catenin staining was used.

FIG. 26B is a series of images evaluating tight junction integrity in the guinea pig RWM after application of 0.1% (w/v) KLALKLALKALKLAALKLA.HCl in ex vivo culture. Actin, ZO-1, or alpha-catenin staining was used.

FIG. 26C is a series of images evaluating tight junction integrity in the guinea pig RWM after application of 0.1% (w/v) KLALKLALKALKLAALKLA.HCl in ex vivo culture with subsequent 75 minutes of incubation in DMEM without the KLALKLALKALKLAALKLA peptide. Actin, ZO-1, or alpha-catenin staining was used.

FIG. 27A is a series of images evaluating tight junction integrity in the control guinea pig RWM in vivo. Actin, ZO-1, or alpha-catenin staining was used.

FIG. 27B is a series of images evaluating tight junction integrity in the guinea pig RWM 21 days post dosing with 0.1% (w/v) KLALKLALKALKLAALKLA.HCl in vivo. Actin, ZO-1, or alpha-catenin staining was used.

FIG. 28A is a chart showing ABR thresholds measured in guinea pig ears at Day 0 and Day 7 post-dosing with 10 μL of 0.1% (w/v) KLALKLALKALKLAALKLA.TFA in aqueous hyaluronan (1% w/v) with His buffer (pH6).

FIG. 28B is a chart showing ABR thresholds measured in guinea pig ears at Day 0 and Day 7 post-dosing with 10 μL of 0.25% (w/v) KLALKLALKALKLAALKLA.HCl in aqueous hyaluronan (1% w/v) with His buffer (pH6).

FIG. 28C is a chart showing ABR thresholds measured in guinea pig ears at Day 0 and Day 7 post-dosing with 10 μL of 0.25% (w/v) KLALKLALKALKLAALKLA.HAc in aqueous hyaluronan (1% w/v) with His buffer (pH6).

FIG. 28D is a chart showing ABR thresholds measured in guinea pig ears at Day 14 post-dosing with 1 μL of 1% (w/v) KLALKLALKALKLAALKLA.HCl in aqueous Poloxamer 407 (20% w/v) with His buffer (pH6). The ABR hearing test at Day 14 post-dosing showed no change relative to Day 0.

FIG. 29 is a chart showing the NT3 levels in perilymph collected from cynomolgus monkeys at 3 h, 4 h, 6 h, and 7 h post-dosing.

### DETAILED DESCRIPTION

The compositions and methods described herein can be used to deliver therapeutic agents into the inner ear of a subject (e.g., a mammalian subject, such as a human), where they may exert a beneficial effect. For example, using the compositions and methods described herein, a therapeutic agent may be delivered to the inner ear of a patient suffering from or at risk of developing one or more of a variety of pathological conditions. The pharmaceutical compositions described herein contain a permeation-enhancing peptide capable of effectuating the passage of one or more therapeutic agents across the round window membrane. The delivery of therapeutic agents through the round window membrane and into the inner ear is a longstanding challenge, particularly for larger molecular weight agents, such as proteins, antibodies, nucleic acids, viral vectors, and nanoparticles, among others. The compositions and methods described herein thus solve an important clinical problem, as the permeation-enhancing peptides described herein can be used not only to engender the passage of therapeutic agents across the round window membrane, but also to promote the sustained residence of such therapeutic agents in the inner ear for extended periods of time.

The present disclosure is based, in part, on the discovery that a series of positively charged, alpha-helical, facially amphipathic peptides can be used to promote the entry of therapeutic agents through the round window membrane and into the inner ear. Without being limited by mechanism, the permeation-enhancing peptides described herein may modulate tight junctions between epithelial cells of the round window membrane, enabling the penetration of therapeutic agents through this barrier.

Upon accessing the inner ear, the therapeutic agents may migrate to a particular site at which they may exert their biological effect. Exemplary conditions that may be treated and/or prevented using the pharmaceutical compositions that contain these therapeutic agents are, without limitation, otic diseases, such as ceruminosis or ceruminosis associated with an otic disease or condition, ear pruritus, otitis externa, otalgia, tinnitus, vestibular dysfunction (e.g., vertigo, dizziness, or loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof. The pharmaceutical compositions described herein may additionally be used to prevent or mitigate sensory hair cell death. Particular embodiments of the pharmaceutical composition that can be used for this purpose include those in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, or a WNT modulator. For example, pharmaceutical compositions that are particularly suited for this indication are those in which the therapeutic agent is a neurotrophin selected from NT-3, NGF, BDNF, CNTF, GDNF, NT-4, FGF, IGF, EGF, PGF, MANF, CDNF, a pan-neurotrophin (PNT-1), a chimeric neurotrophin (e.g., an NGF/BDNF chimera), or a combination thereof.

The sections that follow provide a description of the structural features of the permeation-enhancing peptides that may be incorporated into the pharmaceutical compositions of the present disclosure, as well as therapeutic agents that may be co-formulated with such peptides and exemplary pathologies that may be treated, prevented, and/or mitigated using these pharmaceutical compositions.

### Permeation-Enhancing Peptides

### Polypeptide Structure

Peptides that may be used in conjunction with the compositions and methods described herein for the delivery of one or more therapeutic agents across the round window membrane and into the inner ear include those that contain one or more regions represented, from N-terminus to C-terminus, by formula (I)

X¹—X²—X² (I)

wherein each X¹ independently represents an amino acid containing a cationic side chain at physiological pH;

each X² independently represents an amino acid containing a hydrophobic side chain; and

each “—” independently represents a peptide bond or a peptide bond isostere.

In some embodiments of formula (I), each X¹ independently represents an amino acid containing a lysine or arginine side chain; and each X² independently represents an amino acid containing an alanine, leucine, isoleucine, valine, methionine, tryptophan, phenylalanine, cysteine, or tyrosine side chain.

In some embodiments of formula (I), each X¹ independently represents an amino acid containing a lysine or arginine side chain; and each X² independently represents an amino acid containing an alanine, leucine, isoleucine, valine, or tryptophan side chain.

In some embodiments of formula (I), each X¹ independently represents an amino acid containing a lysine side chain; and each X² independently represents an amino acid containing an alanine, leucine, or tryptophan side chain.

In some embodiments, the polypeptide contains from 2 to 10 of the regions represented by formula (I), such as 2 regions represented by formula (I), 3 regions represented by formula (I), 4 regions represented by formula (I), 5 regions represented by formula (I), 6 regions represented by formula (I), 7 regions represented by formula (I), 8 regions represented by formula (I), 9 regions represented by formula (1), or 10 regions represented by formula (I). In some embodiments, the polypeptide contains from 3 to 7 of the regions represented by formula (I), such as 3 regions represented by formula (I), 4 regions represented by formula (I), 5 regions represented by formula (I), 6 regions represented by formula (I), or 7 regions represented by formula (I). In some embodiments, the polypeptide contains 5 of the regions represented by formula (I).

In some embodiments, each of the regions represented by formula (I) are consecutive or separated by up to two amino acid residues.

In some embodiments, the polypeptide contains one or more regions represented, from N-terminus to C-terminus, by formula (II)

X³—X⁴—X⁵—X⁴ (II)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain; and

each X⁵ independently represents an amino acid containing an alanine side chain.

In some embodiments of formula (II), each X³ independently represents an amino acid containing a lysine or arginine side chain; each X⁴ independently represents an amino acid containing a leucine, isoleucine, valine, or tryptophan side chain; each X⁵ independently represents an amino acid containing an alanine side chain; and each “—” independently represents a peptide bond or a peptide bond isostere.

In some embodiments of formula (II), each X³ independently represents an amino acid containing a lysine side chain; each X⁴ independently represents an amino acid containing a leucine or tryptophan side chain; and each X⁵ independently represents an amino acid containing an alanine side chain.

In some embodiments, the polypeptide contains from 1 to 10 of the regions represented by formula (II), such as 1 region represented by formula (II), 2 regions represented by formula (II), 3 regions represented by formula (II), 4 regions represented by formula (II), 5 regions represented by formula (II), 6 regions represented by formula (II), 7 regions represented by formula (II), 8 regions represented by formula (II), 9 regions represented by formula (II), or 10 regions represented by formula (II). In some embodiments, the polypeptide contains from 2 to 5 of the regions represented by formula (II), such as 2 regions represented by formula (II), 3 regions represented by formula (II), 4 regions represented by formula (II), or 5 regions represented by formula (II). In some embodiments, the polypeptide contains 3 regions represented by formula (II).

In some embodiments, the polypeptide contains one or more regions represented, from N-terminus to C-terminus, by formula (III)

X³—X⁴—X⁵—X⁴—X³—X⁴—X⁵—X⁴ (III)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain; and

each “—” independently represents a peptide bond or a peptide bond isostere.

In some embodiments of formula (III), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments formula (III), each X³ independently represents an amino acid containing a lysine side chain; each X⁴ independently represents an amino acid containing a leucine or tryptophan side chain; and each X⁵ independently represents an amino acid containing an alanine side chain.

In some embodiments, the polypeptide contains from 1 to 5 of the regions represented by formula (III), such as 1 region represented by formula (III), 2 regions represented by formula (III), 3 regions represented by formula (III), 4 regions represented by formula (III), or 5 regions represented by formula (III). In some embodiments, the polypeptide contains one region represented by formula (III).

In some embodiments, the polypeptide contains a region represented, from N-terminus to C-terminus, by formula (IV)

[X³—X⁴— X⁵— X⁴]ₙ—[X³—X⁴—X⁵]ₘ (IV)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5;

m represents an integer from 1 to 5; and

each “—” independently represents a peptide bond or a peptide bond isostere.

In some embodiment of formula (IV), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiment of formula (IV), each X³ independently represents an amino acid containing a lysine side chain; each X⁴ independently represents an amino acid containing a leucine or tryptophan side chain; and each X⁵ independently represents an amino acid containing an alanine side chain.

In some embodiment of formula (IV), n represents an integer from 2 to 4. For example, in some embodiments, n is 2. In some embodiment of formula (IV), m represents an integer from 1 to 3. In some embodiments, m is 1.

In some embodiments, the polypeptide contains a region represented, from N-terminus to C-terminus, by formula (V)

[X³—X⁴—X⁵—X⁴]ₙ—[X³—X⁴— X⁵]ₘ—[X³—X⁴— X⁵— X⁵—X⁴]_{q} (V)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5;

m represents an integer from 1 to 5;

q represents an integer from 1 to 5; and

each “—” independently represents a peptide bond or a peptide bond isostere.

In some embodiments of formula (V), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments of formula (V), each X³ independently represents an amino acid containing a lysine side chain; each X⁴ independently represents an amino acid containing a leucine or tryptophan side chain; and each X⁵ independently represents an amino acid containing an alanine side chain.

In some embodiments of formula (V), n represents an integer from 2 to 4. In some embodiments In some embodiments of formula (V), n is 2. In some embodiments of formula (V), m represents an integer from 1 to 3. In some embodiments of formula (V), m is 1. In some embodiments of formula (V), q represents an integer from 1 to 3. In some embodiments of formula (V), q is 1.

In some embodiments, the polypeptide contains a region represented, from N-terminus to C-terminus, by formula (VI)

[X³—X⁴— X⁵— X⁴]ₙ—[X³—X⁴— X⁵]ₘ—[X³—X⁴— X⁵— X⁵— X⁴]_{q}—[X³—X⁴—X⁵]ᵣ (VI)

wherein each X³ independently represents an amino acid containing a lysine or arginine side chain;

each X⁴ independently represents an amino acid containing a leucine, isoleucine, valine, methionine, tryptophan, phenylalanine, or tyrosine side chain;

each X⁵ independently represents an amino acid containing an alanine side chain;

n represents an integer from 1 to 5;

m represents an integer from 1 to 5;

q represents an integer from 1 to 5;

r represents an integer from 1 to 5; and

each “—” independently represents a peptide bond or a peptide bond isostere.

In some embodiments of formula (VI), each X³ independently represents an amino acid containing a lysine or arginine side chain; each X⁴ independently represents an amino acid containing a leucine, isoleucine, valine, or tryptophan side chain; and each X⁵ independently represents an amino acid containing an alanine side chain.

In some embodiments of formula (VI), each X³ independently represents an amino acid containing a lysine side chain; each X⁴ independently represents an amino acid containing a leucine or tryptophan side chain; and each X⁵ independently represents an amino acid containing an alanine side chain.

In some embodiments of formula (VI), n represents an integer from 2 to 4. In some embodiments of formula (VI), n is 2. In some embodiments of formula (VI), m represents an integer from 1 to 3. In some embodiments of formula (VI), m is 1. In some embodiments of formula (VI), q represents an integer from 1 to 3. In some embodiments of formula (VI), q is 1. In some embodiments of formula (VI), r represents an integer from 1 to 3. In some embodiments of formula (VI), r is 1.

In some embodiments, the polypeptide represented by formula (VII)

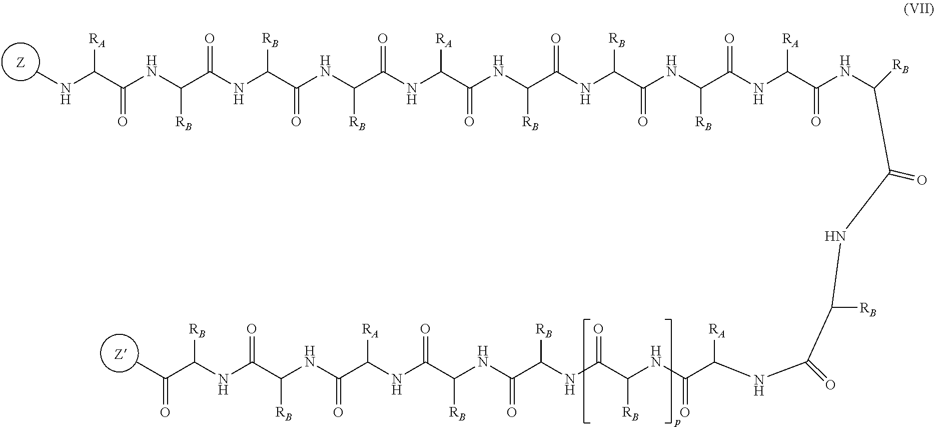

wherein each R_{A} is independently selected from:

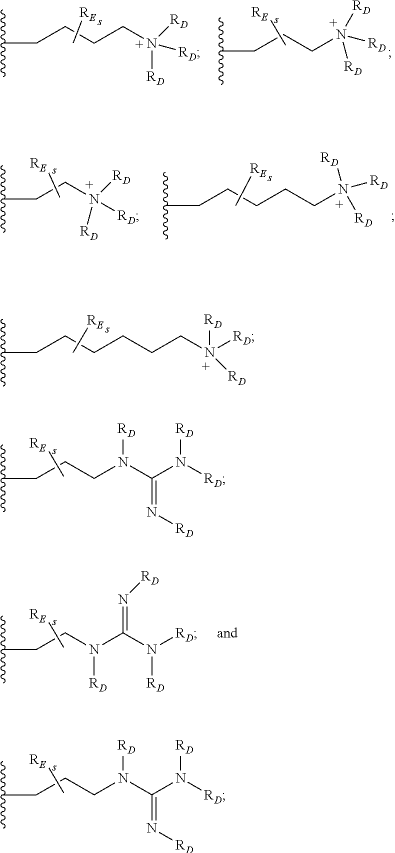

each R_{B} is independently selected from:

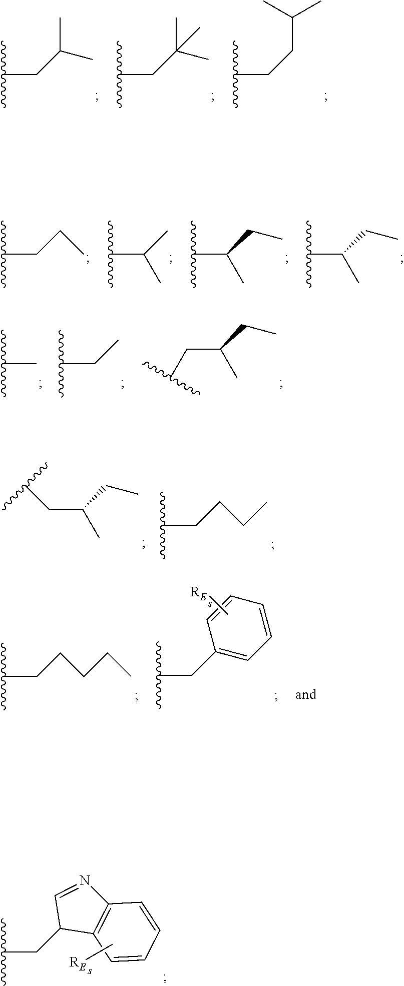

each R_{D} is independently hydrogen or an optionally substituted C₁-C₆ alkyl group;

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

p is an integer from 0 to 3;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (VIII)

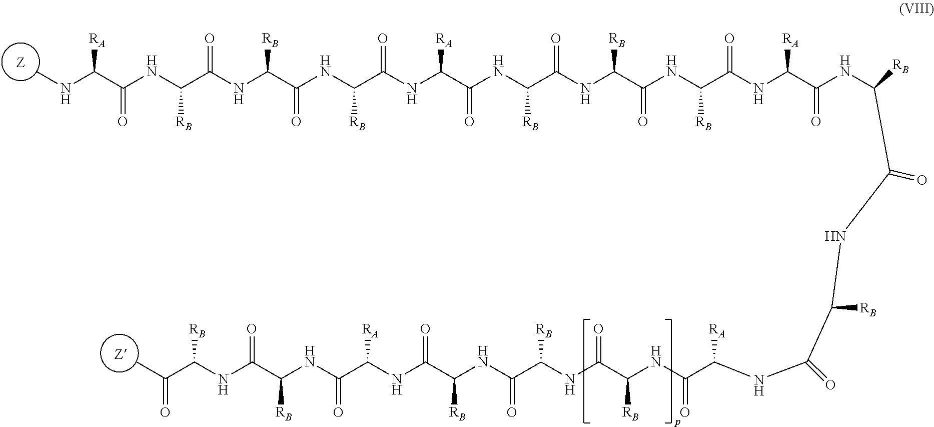

wherein each of R^{A}, R^{B}, R^{D}, R^{E}, p, s, Z, and Z′ are as defined for formula (VII).

In some embodiments, the polypeptide is represented by formula (IX)

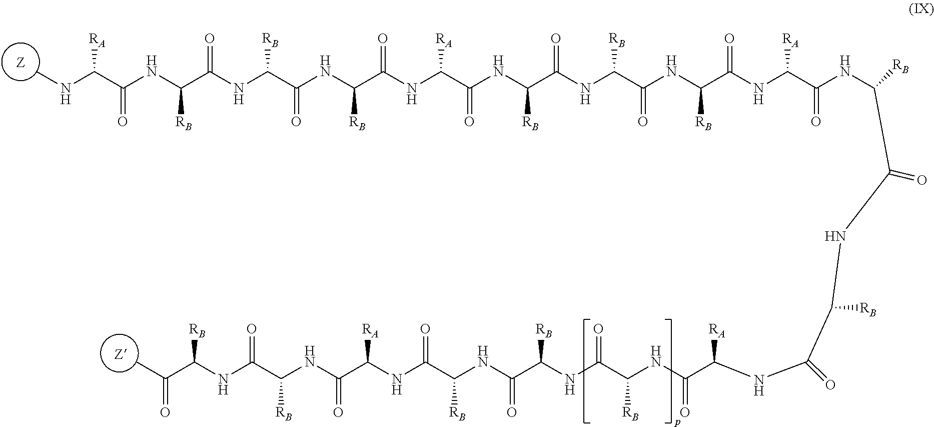

wherein each of R^{A}, R^{B}, R^{D}, R^{E}, p, s, Z, and Z′ are as defined for formula (VII).

In some embodiments, the polypeptide is represented by formula (X)

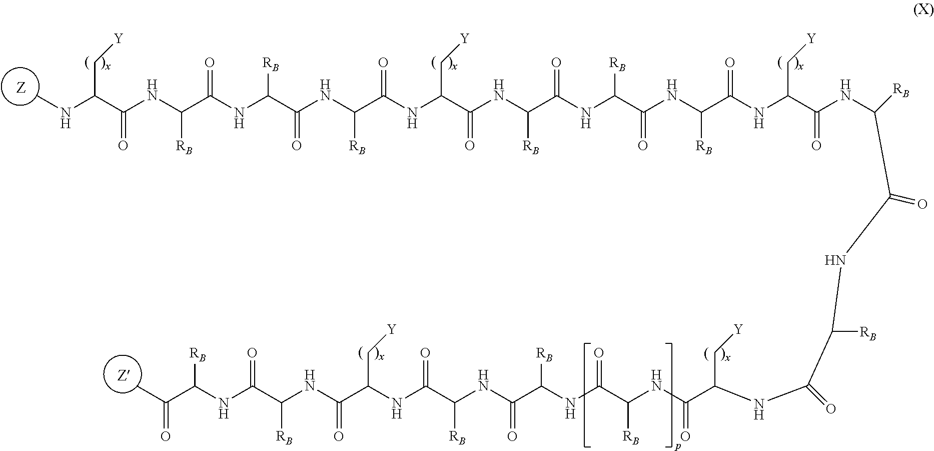

wherein each Y is independently an optionally substituted amino group or an optionally substituted guanidinium group;

each R_{B} is independently selected from:

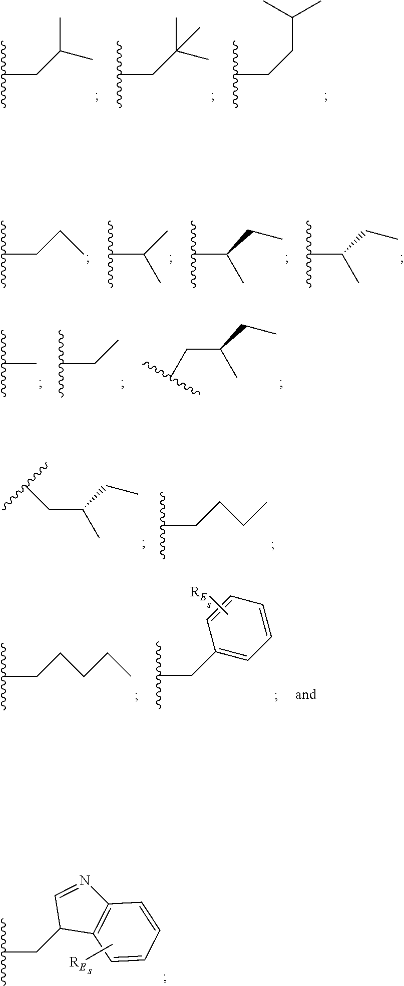

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

x is an integer from 3 to 5;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (XI)

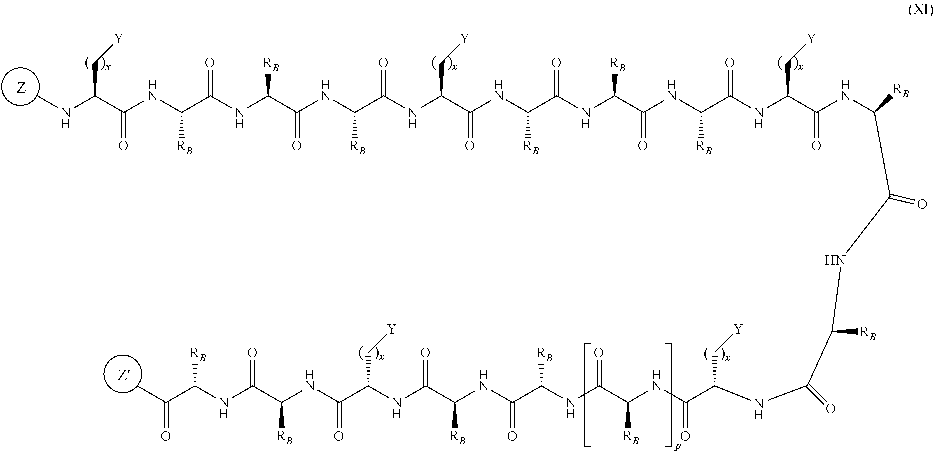

wherein each of Y, R^{B}, R^{E}, x, s, Z, and Z′ are as defined for formula (X).

In some embodiments, the polypeptide is represented by formula (XII)

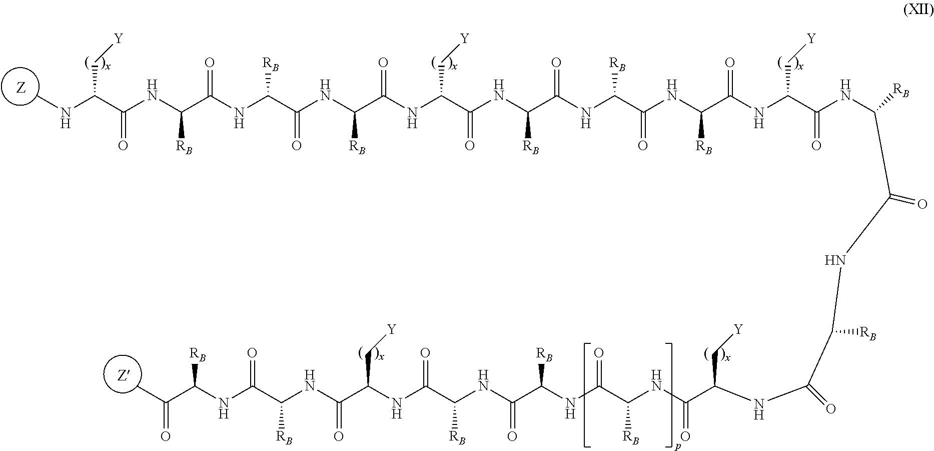

wherein each of Y, R^{B}, R^{E}, x, s, Z, and Z′ are as defined for formula (X).

In some embodiments, the polypeptide is represented by formula (XIII)

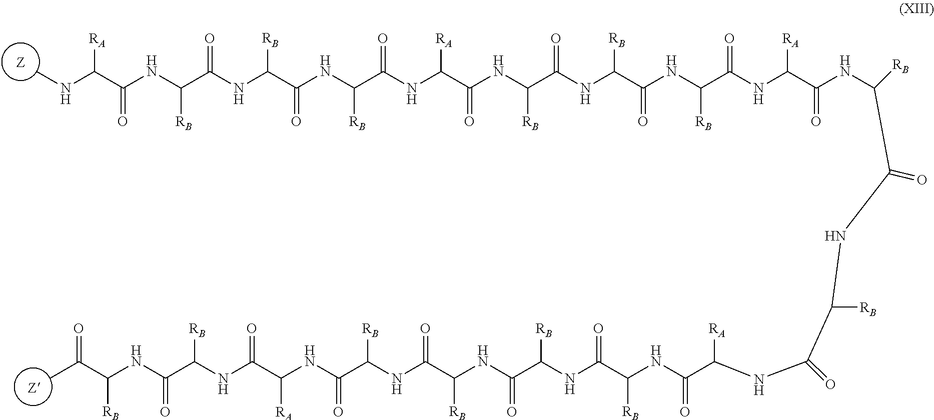

wherein each R_{A} is independently selected from:

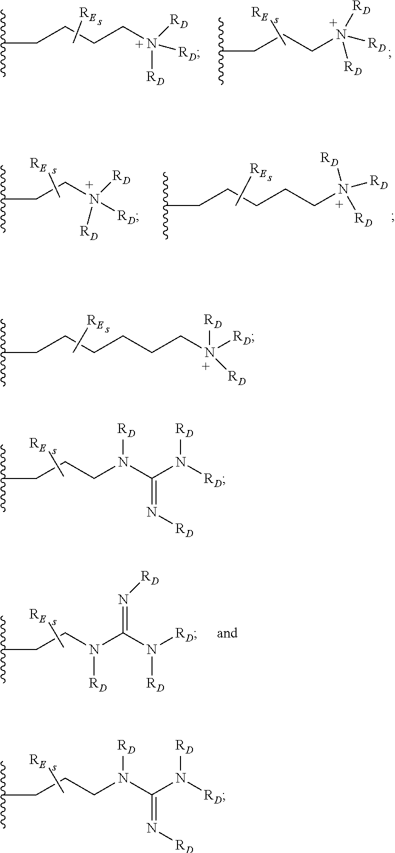

each R_{B} is independently selected from:

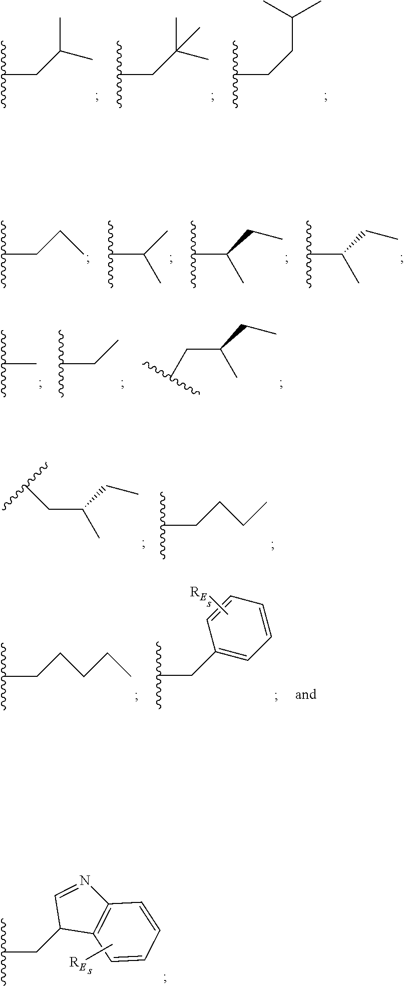

each R_{D} is independently hydrogen or an optionally substituted C₁-C₆ alkyl group;

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (XIV)

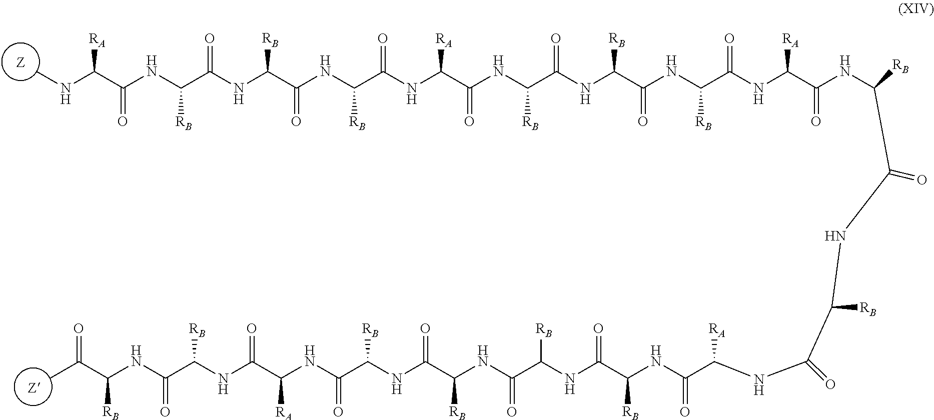

wherein each of R^{A}, R^{B}, R^{D}, R^{E}, s, Z, and Z′ are as defined for formula (XIII).

In some embodiments, the polypeptide is represented by formula (XV)

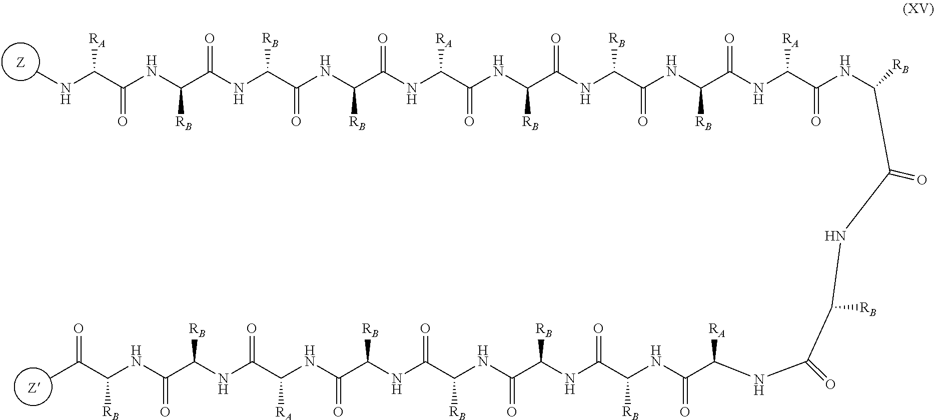

wherein each of R^{A}, R^{B}, R^{D}, R^{E}, s, Z, and Z′ are as defined for formula (XIII).

In some embodiments, the polypeptide is represented by formula (XVI)

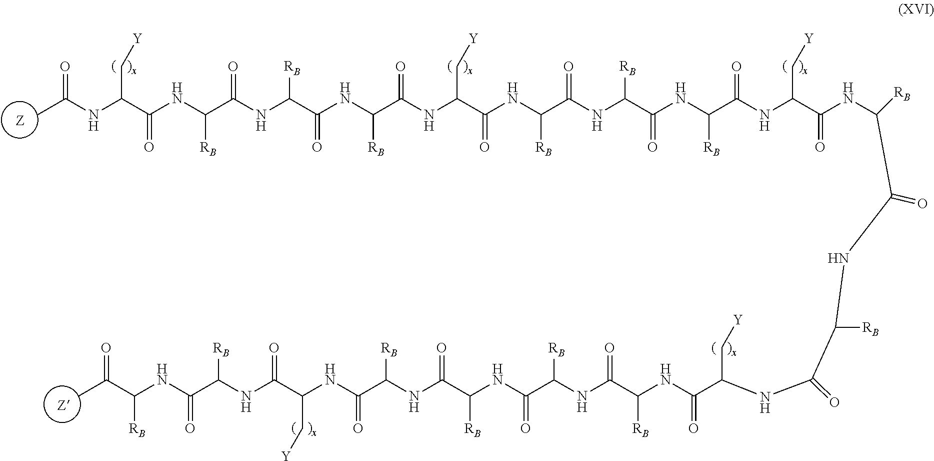

wherein each Y is independently an optionally substituted amino group or an optionally substituted guanidinium group;

each R_{B} is independently selected from:

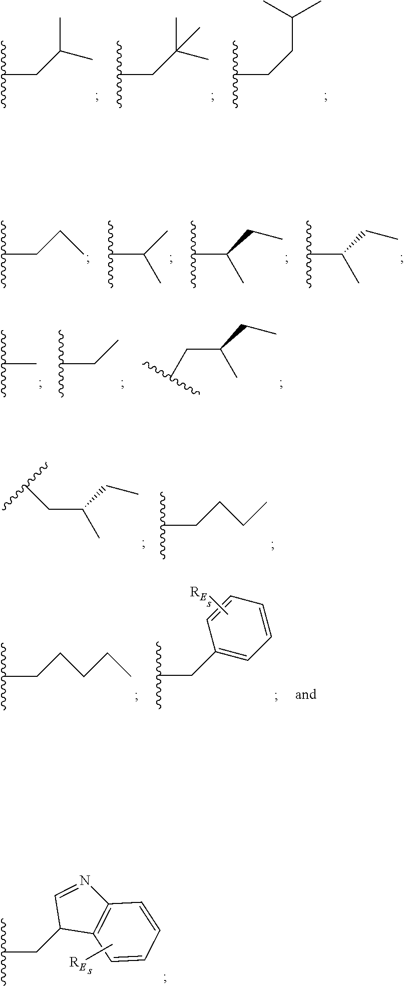

each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;

x is an integer from 3 to 5;

s is an integer from 0 to 5;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (XVII)

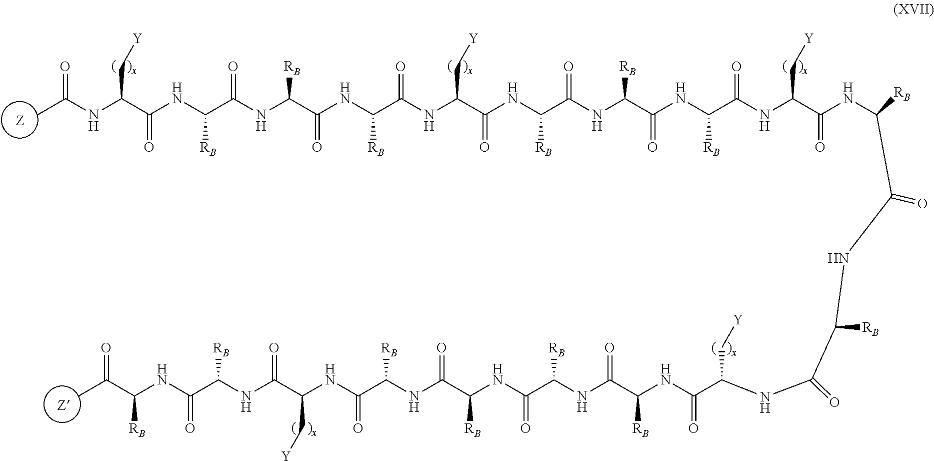

wherein each of Y, R^{B}, R^{E}, x, s, Z, and Z′ are as defined for formula (XVI).

In some embodiments, the polypeptide is represented by formula (XVIII)

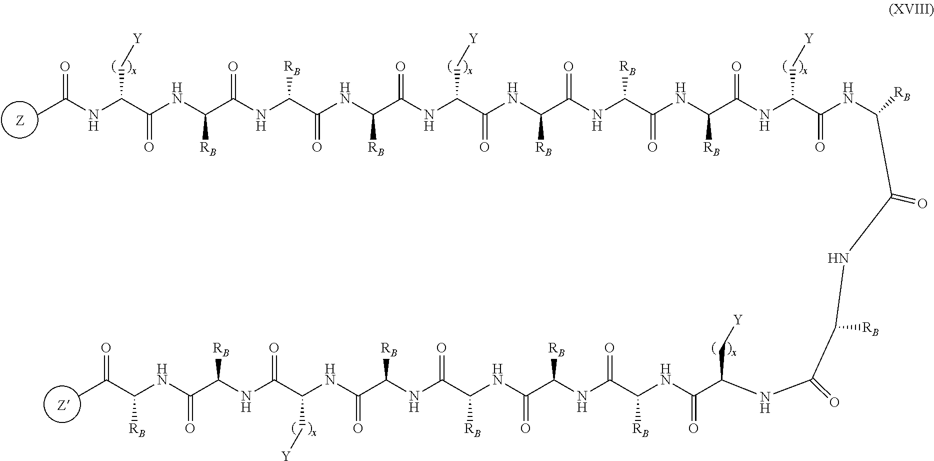

wherein each of Y, R^{B}, R^{E}, x, s, Z, and Z′ are as defined for formula (XVI).

In some embodiments, the polypeptide is represented by formula (XIX)

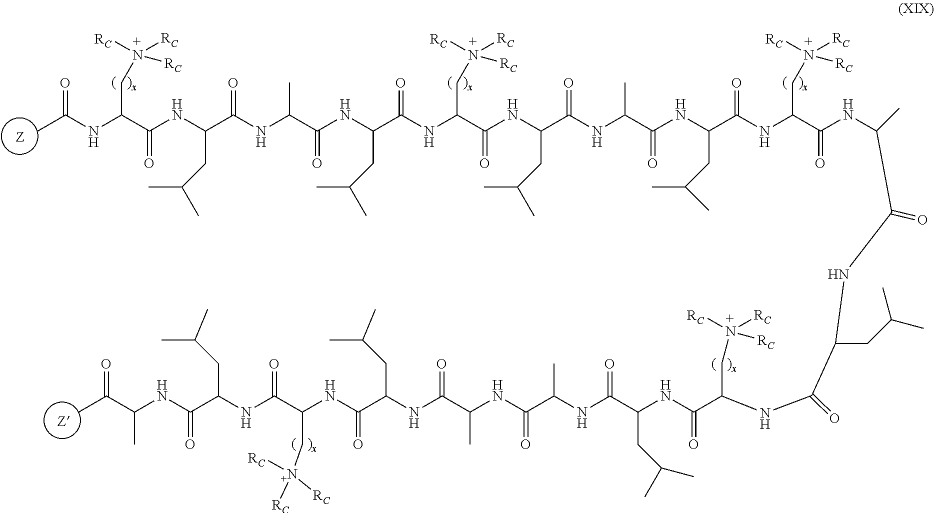

wherein each c is independently hydrogen or optionally substituted 1- 6 alkyl;

x is an integer from 3 to 5; such as 4;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (XX)

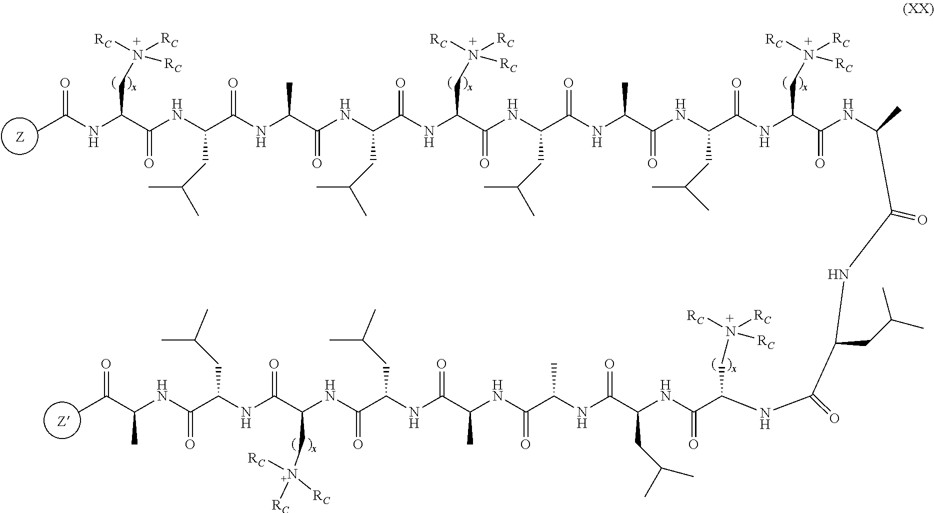

wherein R_{C}, x, Z, and Z′ are as defined for formula (XIX).

In some embodiments, the polypeptide is represented by formula (XXI)

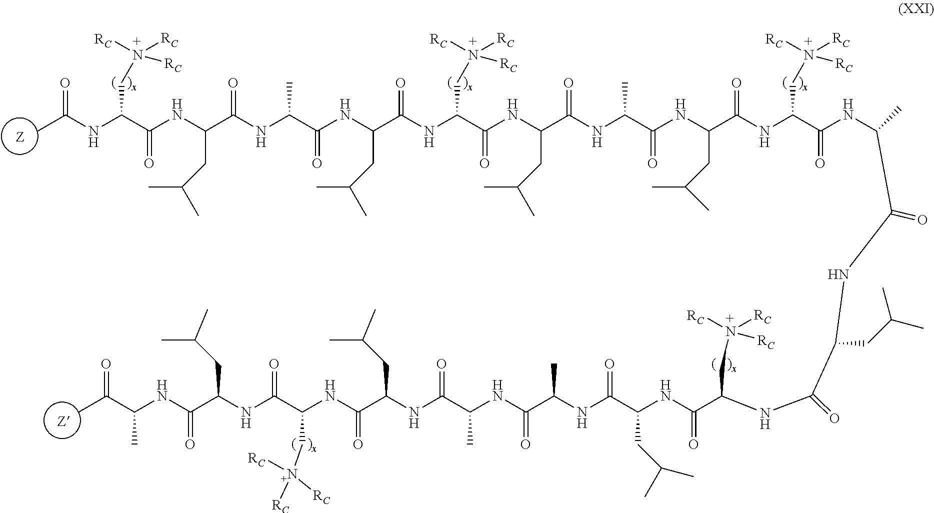

wherein R_{C}, x, Z, and Z′ are as defined for formula (XIX).

In some embodiments, the polypeptide is represented by formula (XXII)

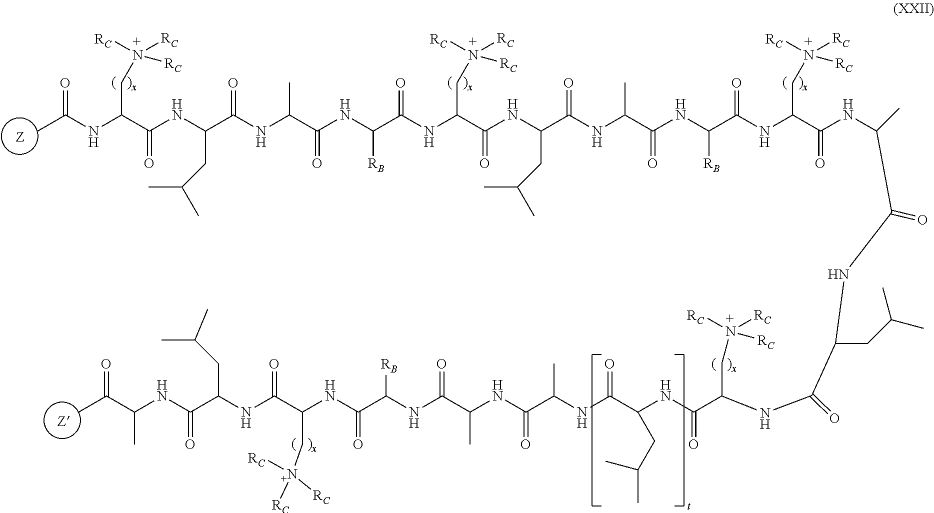

wherein each R_{C} is independently hydrogen or optionally substituted C₁-C₆ alkyl;

x is an integer from 3 to 5; such as 4;

t is 0 or 1;

Z is hydrogen or an optionally substituted C₁-C₆ acyl group, such as an acetyl group; and

Z′ is optionally substituted C₁-C₆ alkoxy, optionally substituted C₁-C₆ alkylamino, —OH, or —NH₂.

In some embodiments, the polypeptide is represented by formula (XXIII)

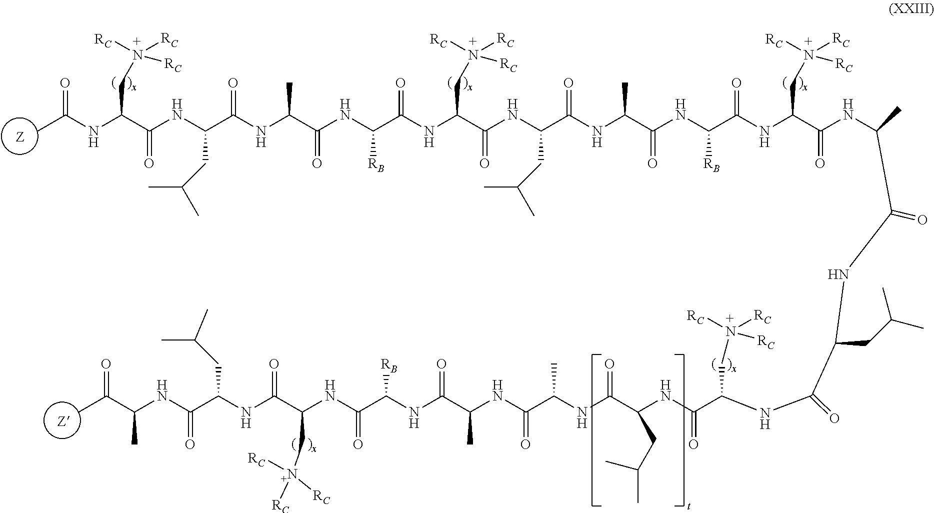

wherein R_{C}, x, t, Z, and Z′ are as defined for formula (XXII).

In some embodiments, the polypeptide is represented by formula (XXIV)

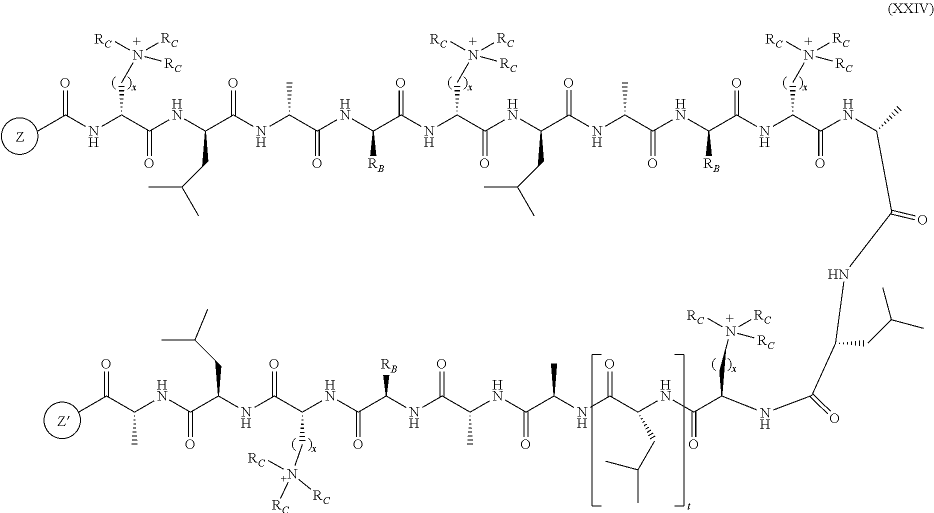

wherein R_{C}, x, t, Z, and Z′ are as defined for formula (XXII).

In some embodiments, the polypeptide is represented by formula (XXV)

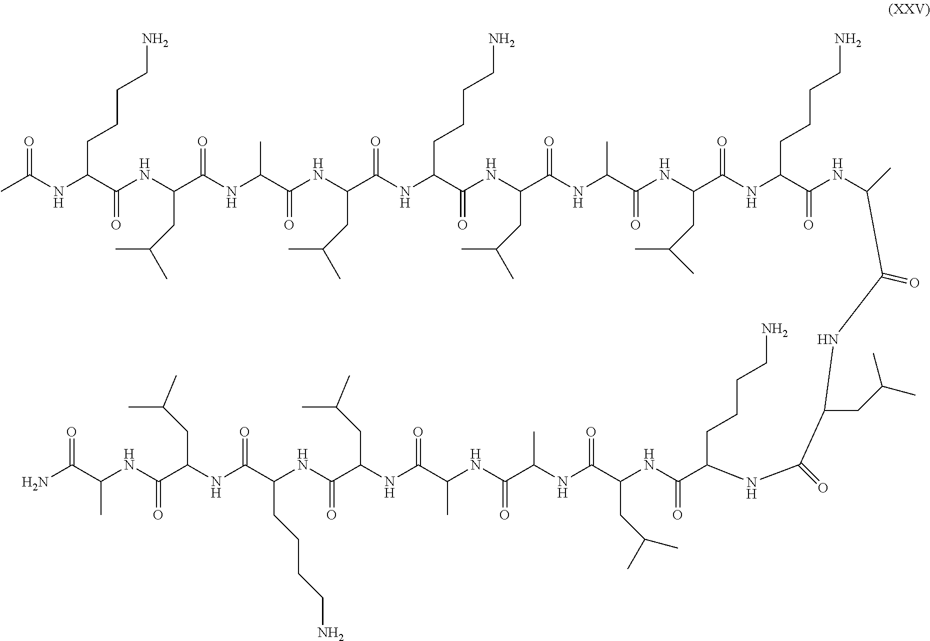 [SMILES: CC(=O)NC(CCCCN)C(=O)NC(CC(C)C)C(=O)NC(C)C(=O)NC(CC(C)C)C(=O)NC(CCCCN)C(=O)NC(CC(C)C)C(=O)NC(C)C(=O)NC(CC(C)C)C(=O)NC(CCCCN)C(=O)NC(C)C(=O)NC(CC(C)C)C(=O)NC(CCCCN)C(=O)NC(CC(C)C)C(=O)NC(C)C(=O)NC(C)C(=O)NC(CC(C)C)C(=O)NC(CCCCN)C(=O)NC(CC(C)C)C(=O)NC(C)C(N)=O]

or a pharmaceutically acceptable salt thereof.

In some embodiments, the polypeptide is represented by formula (XXVI)

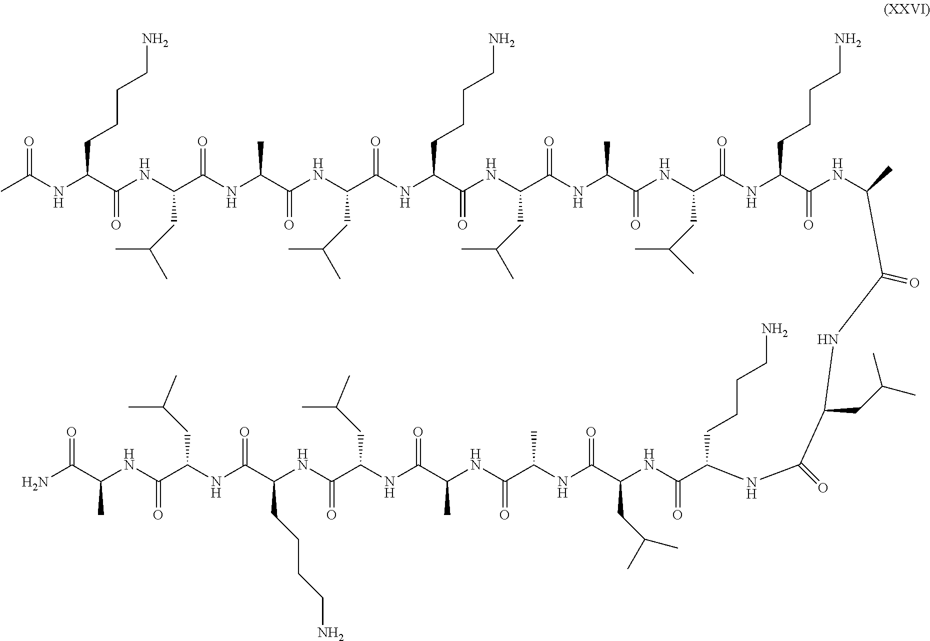 [SMILES: CC(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](C)C(=O)N[C@@H](CC(C)C)C(=O)N[C@@H](CCCCN)C(=O)N[C@@H](CC(C)C)C(=O)N[C@@H](C)C(N)=O]

or a pharmaceutically acceptable salt thereof.

In some embodiments, the polypeptide is represented by formula (XXVII)

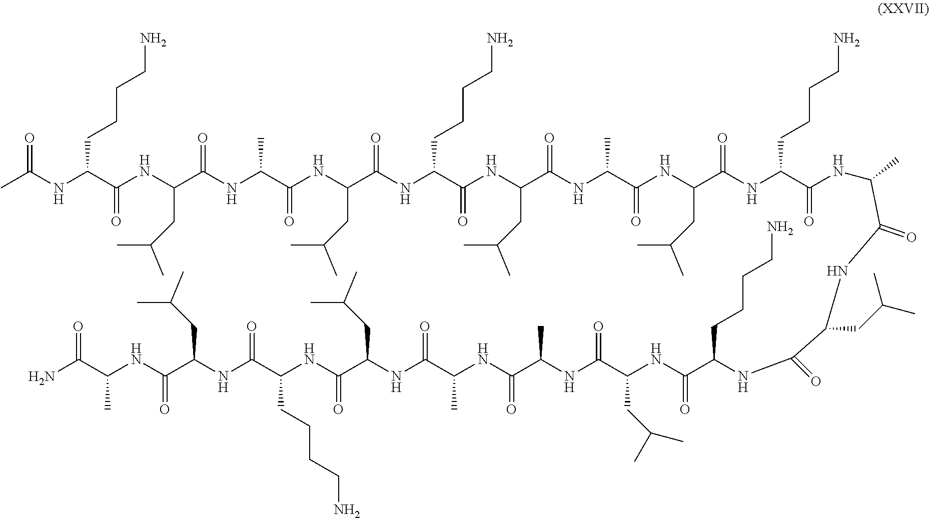 [SMILES: CC(=O)N[C@H](CCCCN)C(=O)NC(CC(C)C)C(=O)N[C@H](C)C(=O)NC(CC(C)C)C(=O)N[C@H](CCCCN)C(=O)NC(CC(C)C)C(=O)N[C@H](C)C(=O)NC(CC(C)C)C(=O)N[C@H](CCCCN)C(=O)N[C@H](C)C(=O)N[C@H](CC(C)C)C(=O)N[C@H](CCCCN)C(=O)N[C@H](CC(C)C)C(=O)N[C@H](C)C(=O)N[C@H](C)C(=O)N[C@H](CC(C)C)C(=O)N[C@H](CCCCN)C(=O)N[C@H](CC(C)C)C(=O)N[C@H](C)C(N)=O]

or a pharmaceutically acceptable salt thereof.

In some embodiments, the polypeptide contains a region having an amino acid sequence that is at least 85% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, polypeptide has an amino acid sequence that is at least 90% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, the polypeptide has an amino acid sequence that is at least 95% identical to an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

In some embodiments, the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to five amino acid substitutions (e.g., by one amino acid substitution, by two amino acid substitutions, by three amino acid substitutions, by four amino acid substitutions, by five amino acid substitutions, or by zero amino acid substitutions).

In some embodiments, the polypeptide has an amino acid sequence that differs from the amino acid sequence of any one of KLALKLALKALKLAALKLA (SEQ ID NO: 1), KLALKLALKALKAALKLA (SEQ ID NO: 2), klalklalkalkaalkla (SEQ ID NO: 3), alklaaklaklalklalk (SEQ ID NO: 4), LKILKkLIkKLLkLL (SEQ ID NO: 5), KLALKLALKALKAALK (SEQ ID NO: 6), KLALKLALKALKAALKLALK (SEQ ID NO: 7), KLAWKLALKALKAALKLA (SEQ ID NO: 8), KLAWKLALKALKAAWKLA (SEQ ID NO: 9), KLAWKLAWKALKAAWKLA (SEQ ID NO: 10), LKLLKKLLKKLLKLL (SEQ ID NO: 11), LKILKkLIkKLLkLL (SEQ ID NO: 12), KALAALLKKAAKLLAALK (SEQ ID NO: 13), and KALAALLKKLAKLLAALK (SEQ ID NO: 14) by up to three amino acid substitutions.

In some embodiments, the amino acid substitutions are conservative amino acid substitutions.

In some embodiments, the polypeptide has an amino acid sequence selected from KLALKLALKALKLAALKLA (SEQ ID NO: 1); KLALKLALKALKAALKLA (SEQ ID NO: 2); klalklalkalkaalkla (SEQ ID NO: 3); alklaaklaklalklalk (SEQ ID NO: 4); LKILKkLIkKLLkLL (SEQ ID NO: 5); KLALKLALKALKAALK (SEQ ID NO: 6); KLALKLALKALKAALKLALK (SEQ ID NO: 7); KLAWKLALKALKAALKLA (SEQ ID NO: 8); KLAWKLALKALKAAWKLA (SEQ ID NO: 9); KLAWKLAWKALKAAWKLA (SEQ ID NO: 10); LKLLKKLLKKLLKLL (SEQ ID NO: 11); LKILKkLIkKLLkLL (SEQ ID NO: 12); KALAALLKKAAKLLAALK (SEQ ID NO: 13); and KALAALLKKLAKLLAALK (SEQ ID NO: 14).

### Alpha-Helicity

Permeation-enhancing peptides that may be used in conjunction with the compositions and methods described herein include those that have an alpha-helical secondary structure. For example, in some embodiments, the polypeptide has an alpha-helicity of at least about 50%. The polypeptide may have, for example, an alpha-helicity of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

Particularly, in some embodiments, the polypeptide has an alpha-helicity of at least about 55%, at least about 60%, at least about 65%, at least about 70%, at least about 75%, or more. In some embodiments, the polypeptide has an alpha-helicity of from about 55% to about 85%, from about 51% to about 84%, from about 52% to about 83%, from about 53% to about 82%, from about 54% to about 81%, from about 55% to about 80%, from about 60% to about 75%, of from about 60% to about 70%. In some embodiments, the polypeptide has an alpha-helicity of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the polypeptide has an alpha-helicity of from about 61% to about 68%, such as an alpha-helicity of 61%, 62%, 63%, 64%, 65%, 66%, 67%, or 68%.

Alpha-helicity can be assessed, for example, using circular dichroism (CD) techniques described herein and known in the art. CD spectra are generally collected in high transparency quartz cuvettes. Both rectangular and cylindrical cells are available, with path lengths ranging from 0.01 to 1 cm. Water jacketed cylindrical cells are available for CD machines that do not have temperature regulated cell holders. Most cylindrical cells and 0.1 cm rectangular cells have relatively low birefringence and give reasonably straight baselines, but all cells are different and baseline spectra must always be collected. Rectangular cells with path lengths greater than 0.2 cm may have high birefringence due to strain. Cells designed for fluorescence, in which all four sides are made of the same material usually have lower birefringence than cells where two sides are frosted. Rectangular cells with path lengths less than 0.1 cm often have a very small total sample volume and a very small surface area facing the light beam of the CD machine. It is important that the light beam be very tightly focused if these cells are used, since large artifacts are produced if the light does not go directly through the sample. Exemplary protocols for using CD to assess peptide secondary structure are described, e.g., in Greenfield, Nature Protocols 1:2876-2890 (2006), the disclosure of which is incorporated herein by reference in its entirety.

### Intramolecular Crosslinks

Permeation-enhancing peptides described herein may contain one or more intramolecular crosslinks, for example, in order to stabilize an alpha-helical secondary structure. Exemplary intramolecular crosslinks that may be used in conjunction with the compositions and methods described herein to stabilize an alpha-helical secondary structure are described, e.g., in Li et al., Chinese Chemical Letters 29:1088-1092 (2018), the disclosure of which is incorporated herein by reference. Particular examples of intramolecular crosslinks that may be incorporated into the permeation-enhancing peptides described herein so as to stabilize an alpha-helical secondary structure are:

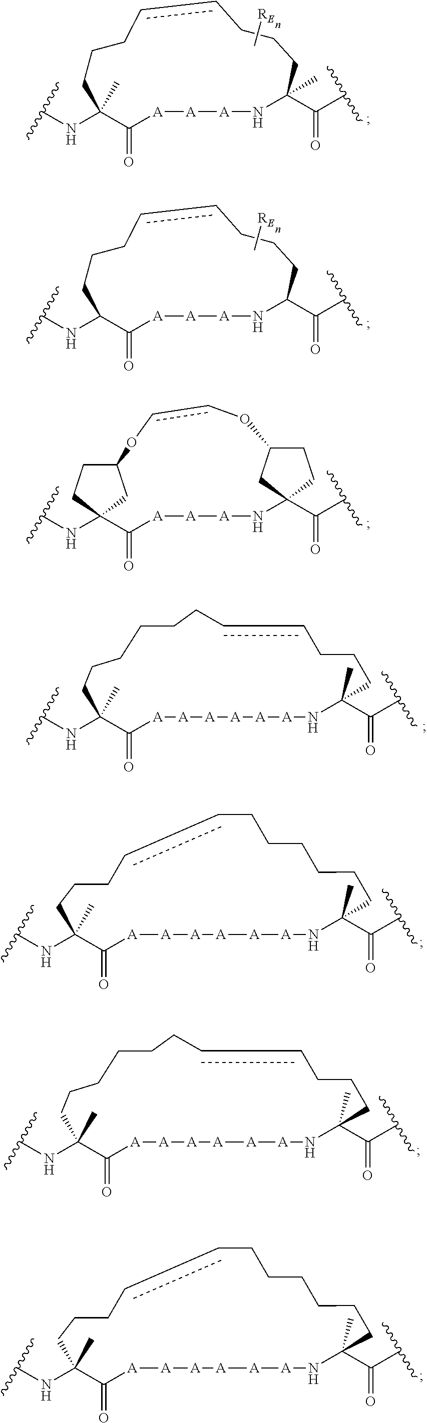

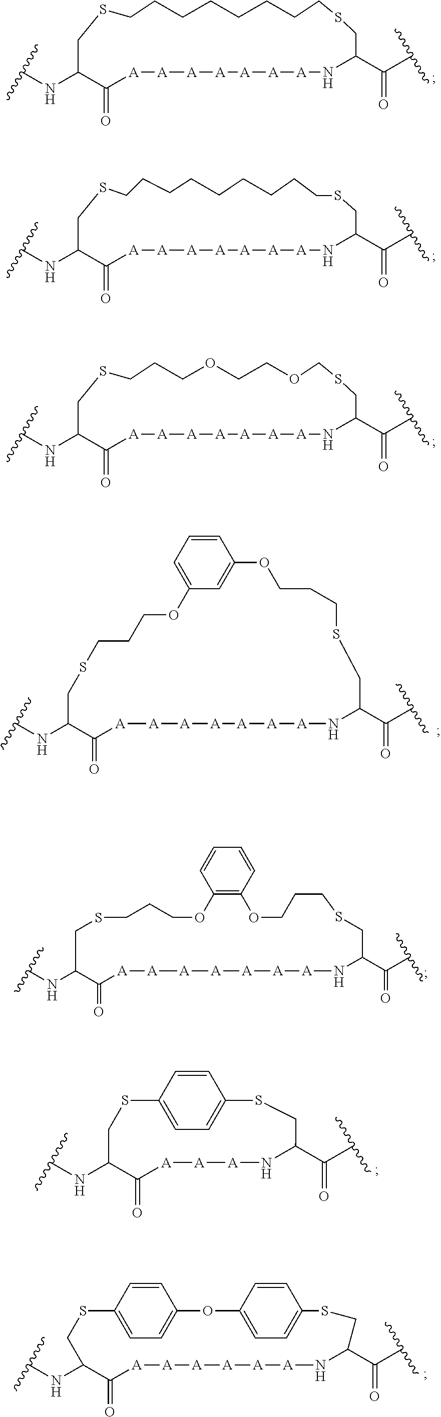

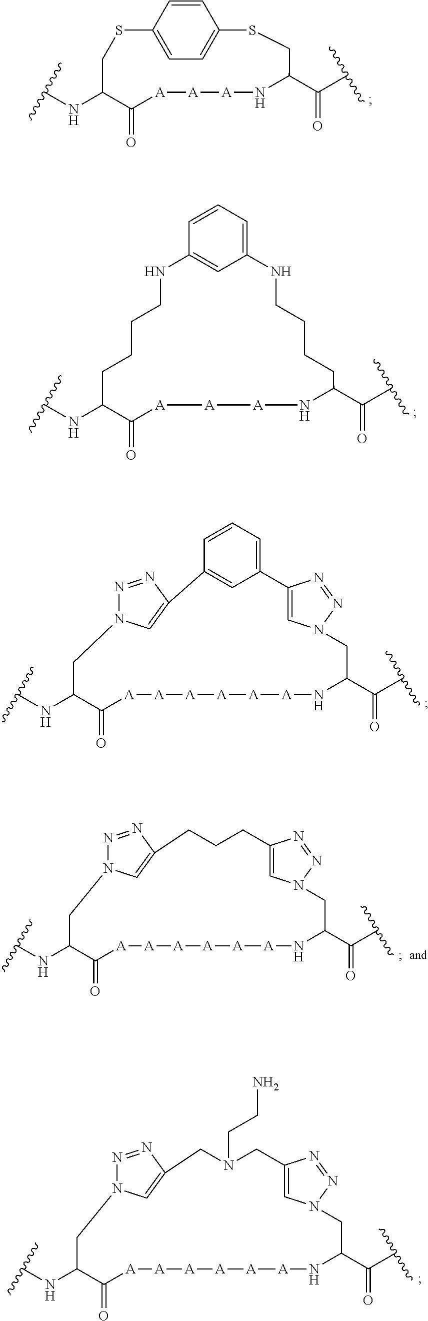

wherein each “A” represents an individual amino acid residue within the polypeptide. As used herein, each dashed line represents an optional double bond.

### Peptide Bond Isosteres

In some embodiments, in lieu of a formal —NH—C(O)— amide bond, amino acid monomers may be covalently bound to one another by way of an amino acid isostere. Exemplary amino acid isosteres that may be used in conjunction with the compositions and methods described herein are described, e.g., in Choudhary et al., Chembiochem 12:1801-1807 (2011), the disclosure of which is incorporated herein by reference. Examples of peptide bond isosteres that may be incorporated into the permeation enhancers described herein are:

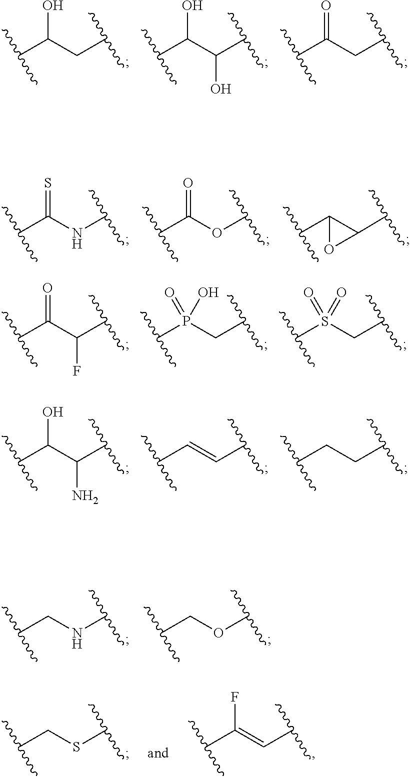

among others.

### Electrostatic Charge

Permeation-enhancing peptides that may be used in conjunction with the compositions and methods described herein include those having a positive charge at physiological pH. One way to quantify the electrostatic charge of a polypeptide is by determining the isoelectric point (pl) of the peptide, which is the pH at which the polypeptide exists primarily in a neutral zwitterionic form. In some embodiments, the permeation-enhancing peptides described herein have an isoelectric point (pl) of from about 8 to about 13. For example, the polypeptide may have an isoelectric point of about 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, or 13. Particularly, in some embodiments, the polypeptide has an isoelectric point of about 8.0, 8.01, 8.02, 8.03, 8.04, 8.05, 8.06, 8.07, 8.08, 8.09, 8.10, 8.11, 8.12, 8.13, 8.14, 8.15, 8.16, 8.17, 8.18, 8.19, 8.20, 8.21, 8.22, 8.23, 8.24, 8.25, 8.26, 8.27, 8.28, 8.29, 8.30, 8.31, 8.32, 8.33, 8.34, 8.35, 8.36, 8.37, 8.38, 8.39, 8.40, 8.41, 8.42, 8.43, 8.44, 8.45, 8.46, 8.47, 8.48, 8.49, 8.50, 8.51, 8.52, 8.53, 8.54, 8.55, 8.56, 8.57, 8.58, 8.59, 8.60, 8.61, 8.62, 8.63, 8.64, 8.65, 8.66, 8.67, 8.68, 8.69, 8.70, 8.71, 8.72, 8.73, 8.74, 8.75, 8.76, 8.77, 8.78, 8.79, 8.80, 8.81, 8.82, 8.83, 8.84, 8.85, 8.86, 8.87, 8.88, 8.89, 8.90, 8.91, 8.92, 8.93, 8.94, 8.95, 8.96, 8.97, 8.98, 8.99, 9.0, 9.01, 9.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, 12.0, 12.01, 8.02, 12.03, 12.04, 12.05, 12.06, 12.07, 12.08, 12.09, 12.10, 12.11, 12.12, 12.13, 12.14, 12.15, 12.16, 12.17, 12.18, 12.19, 12.20, 12.21, 12.22, 12.23, 12.24, 12.25, 12.26, 12.27, 12.28, 12.29, 12.30, 12.31, 12.32, 12.33, 12.34, 12.35, 12.36, 12.37, 12.38, 12.39, 12.40, 12.41, 12.42, 12.43, 12.44, 12.45, 12.46, 12.47, 12.48, 12.49, 12.50, 12.51, 12.52, 12.53, 12.54, 12.55, 12.56, 12.57, 12.58, 12.59, 12.60, 12.61, 12.62, 12.63, 12.64, 12.65, 12.66, 12.67, 12.68, 12.69, 12.70, 12.71, 12.72, 12.73, 12.74, 12.75, 12.76, 12.77, 12.78, 12.79, 12.80, 12.81, 12.82, 12.83, 12.84, 12.85, 12.86, 12.87, 12.88, 12.89, 12.90, 12.91, 12.92, 12.93, 12.94, 12.95, 12.96, 12.97, 12.98, 12.99, or 13.0.

In some embodiments, the polypeptide has a pl of from about 8.5 to about 12.5, such as a pl of 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, or 12.5 (e.g., a pl of 8.50, 8.51, 8.52, 8.53, 8.54, 8.55, 8.56, 8.57, 8.58, 8.59, 8.60, 8.61, 8.62, 8.63, 8.64, 8.65, 8.66, 8.67, 8.68, 8.69, 8.70, 8.71, 8.72, 8.73, 8.74, 8.75, 8.76, 8.77, 8.78, 8.79, 8.80, 8.81, 8.82, 8.83, 8.84, 8.85, 8.86, 8.87, 8.88, 8.89, 8.90, 8.91, 8.92, 8.93, 8.94, 8.95, 8.96, 8.97, 8.98, 8.99, 9.0, 9.01, 8.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, 12.0, 12.01, 8.02, 12.03, 12.04, 12.05, 12.06, 12.07, 12.08, 12.09, 12.10, 12.11, 12.12, 12.13, 12.14, 12.15, 12.16, 12.17, 12.18, 12.19, 12.20, 12.21, 12.22, 12.23, 12.24, 12.25, 12.26, 12.27, 12.28, 12.29, 12.30, 12.31, 12.32, 12.33, 12.34, 12.35, 12.36, 12.37, 12.38, 12.39, 12.40, 12.41, 12.42, 12.43, 12.44, 12.45, 12.46, 12.47, 12.48, 12.49, or 12.50).

In some embodiments, the polypeptide has a pl of from about 9 to about 12, such as a pl of 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, or 12.5 (e.g., a pl of 9.0, 9.01, 8.02, 9.03, 9.04, 9.05, 9.06, 9.07, 9.08, 9.09, 9.10, 9.11, 9.12, 9.13, 9.14, 9.15, 9.16, 9.17, 9.18, 9.19, 9.20, 9.21, 9.22, 9.23, 9.24, 9.25, 9.26, 9.27, 9.28, 9.29, 9.30, 9.31, 9.32, 9.33, 9.34, 9.35, 9.36, 9.37, 9.38, 9.39, 9.40, 9.41, 9.42, 9.43, 9.44, 9.45, 9.46, 9.47, 9.48, 9.49, 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17, 11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, 11.50, 11.51, 11.52, 11.53, 11.54, 11.55, 11.56, 11.57, 11.58, 11.59, 11.60, 11.61, 11.62, 11.63, 11.64, 11.65, 11.66, 11.67, 11.68, 11.69, 11.70, 11.71, 11.72, 11.73, 11.74, 11.75, 11.76, 11.77, 11.78, 11.79, 11.80, 11.81, 11.82, 11.83, 11.84, 11.85, 11.86, 11.87, 11.88, 11.89, 11.90, 11.91, 11.92, 11.93, 11.94, 11.95, 11.96, 11.97, 11.98, 11.99, or 12.0).

In some embodiments, the polypeptide has a pl of from about to about 9.5 to about 11.5, such as a pl of 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, or 11.5 (e.g., a pl of 9.50, 9.51, 9.52, 9.53, 9.54, 9.55, 9.56, 9.57, 9.58, 9.59, 9.60, 9.61, 9.62, 9.63, 9.64, 9.65, 9.66, 9.67, 9.68, 9.69, 9.70, 9.71, 9.72, 9.73, 9.74, 9.75, 9.76, 9.77, 9.78, 9.79, 9.80, 9.81, 9.82, 9.83, 9.84, 9.85, 9.86, 9.87, 9.88, 9.89, 9.90, 9.91, 9.92, 9.93, 9.94, 9.95, 9.96, 9.97, 9.98, 9.99, 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, 11.0, 11.01, 8.02, 11.03, 11.04, 11.05, 11.06, 11.07, 11.08, 11.09, 11.10, 11.11, 11.12, 11.13, 11.14, 11.15, 11.16, 11.17,11.18, 11.19, 11.20, 11.21, 11.22, 11.23, 11.24, 11.25, 11.26, 11.27, 11.28, 11.29, 11.30, 11.31, 11.32, 11.33, 11.34, 11.35, 11.36, 11.37, 11.38, 11.39, 11.40, 11.41, 11.42, 11.43, 11.44, 11.45, 11.46, 11.47, 11.48, 11.49, or 11.50).

In some embodiments, the polypeptide has a pl of from about 10 to about 11, such as a pl of 10, 10.1, 10.2, 10.3, 10.4, 10.5, 10.6, 10.7, 10.8, 10.9, or 11 (e.g., a pl of 10.0, 10.01, 8.02, 10.03, 10.04, 10.05, 10.06, 10.07, 10.08, 10.09, 10.10, 10.11, 10.12, 10.13, 10.14, 10.15, 10.16, 10.17, 10.18, 10.19, 10.20, 10.21, 10.22, 10.23, 10.24, 10.25, 10.26, 10.27, 10.28, 10.29, 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, 10.90, 10.91, 10.92, 10.93, 10.94, 10.95, 10.96, 10.97, 10.98, 10.99, or 11.0).

In some embodiments, the polypeptide has a pl of from about 10.3 to about 10.9, such as a pl of 10.30, 10.31, 10.32, 10.33, 10.34, 10.35, 10.36, 10.37, 10.38, 10.39, 10.40, 10.41, 10.42, 10.43, 10.44, 10.45, 10.46, 10.47, 10.48, 10.49, 10.50, 10.51, 10.52, 10.53, 10.54, 10.55, 10.56, 10.57, 10.58, 10.59, 10.60, 10.61, 10.62, 10.63, 10.64, 10.65, 10.66, 10.67, 10.68, 10.69, 10.70, 10.71, 10.72, 10.73, 10.74, 10.75, 10.76, 10.77, 10.78, 10.79, 10.80, 10.81, 10.82, 10.83, 10.84, 10.85, 10.86, 10.87, 10.88, 10.89, or 10.90. In some embodiments, the polypeptide has a pl of about 10.6.

### Therapeutic Agents

Examples of therapeutic agents that may be incorporated into the pharmaceutical compositions described herein are neurotrophins, immunomodulating agents, aural pressure modulating agents, corticosteroids, antimicrobial agents, antagonists of truncated TrkC or truncated TrkB, non-natural TrkB or TrkC agonists, TrkB receptor agonist antibodies or antigen-binding fragments thereof, TrkB receptor agonist compounds, TrkC receptor agonist antibodies or antigen-binding fragments thereof, TrkC receptor agonist compounds, neuroprotection agents, Atoh1 modulators (e.g., an Atoh1 polypeptide or a nucleic acid vector engineered to express Atoh1, e.g., human Atoh1 (Hath1)), and WNT modulators.

### Neurotrophins

The therapeutic agent may be, for example, a neurotrophin selected from neurotrophin-3 (NT-3), nerve growth factor (NGF), brain-derived neurotrophic factor (BDNF), ciliary neurotrophic factor (CNTF), glial cell-line derived neurotrophic factor (GDNF), neurotrophin-4 (NT-4), fibroblast growth factor (FGF), insulin-like growth factor (IGF), epidermal growth factor (EGF), platelet-derived growth factor (PGF), mesencephalic astrocyte-derived neurotrophic factor (MANF), cerebral dopamine neurotrophic factor (CDNF), a pan-neurotrophin (e.g., PNT-1), a chimeric neurotrophin (e.g., an NGF/BDNF chimera), and combinations thereof.

In some embodiments, the neurotrophic factor is NT-3. The NT-3 may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NOs: 15, 21, or 22. The NT-3 may have, for example, an amino acid sequence that differs from the amino acid sequence of any one of SEQ ID NOs: 15, 21, or 22 by only one or more conservative amino acid substitutions, such as by up to 25, up to 20, up to 15, up to 10, or up to 5 conservative amino acid substitutions. The NT-3 may be an NT-3 variant having an amino acid sequence with one or more (e.g., 1, 2, 3, 4, 5, 6, 7, 8, 9, 10, or more) amino acid substitutions relative to the amino acid sequence of any one of SEQ ID NOs: 15, 21, or 22. The NT-3 variant may have, for example, the sequence of any one of SEQ ID NOs: 48-60. In some embodiments, the NT-3 variant has the sequence of SEQ ID NO: 49. In some embodiments, the NT-3 variant has the sequence of SEQ ID NO: 57. In some embodiments, the NT-3 variant has the sequence of SEQ ID NO: 58. The NT-3 may be encoded by a nucleic acid having the sequence of SEQ ID NO: 16 or SEQ ID NO: 70.

In some embodiments, the neurotrophic factor is NGF. The NGF may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NOs: 19 or 20. The NGF may have, for example, an amino acid sequence that differs from the amino acid sequence of SEQ ID NOs: 19 or 20 by only one or more conservative amino acid substitutions, such as by up to 25, up to 20, up to 15, up to 10, or up to 5 conservative amino acid substitutions. The NGF may be an NGF variant having an amino acid sequence with one or more (e.g., 1, 2, 3, 4, 5, 6, 7, 8, 9, 10, or more) amino acid substitutions relative to the amino acid sequence of SEQ ID NOs: 19 or 20. In some embodiments, the NGF variant has the sequence any one of SEQ ID NOs: 34-37. In some embodiments, the NGF may be an NGF variant having an amino acid sequence with one or more (e.g., 1, 2, 3, 4, 5, 6, 7, 8, 9, 10 or more) amino acid substitutions relative to the amino acid sequence of SEQ ID NOs: 19 or 20 and one or more (e.g., 1, 2, 3, 4, 5, 6, 7, 8, 9, 10 or more) deleted amino acids relative to the amino acid sequence of SEQ ID NOs: 19 or 20. In some embodiments, the NGF variant has the sequence of SEQ ID NO: 38. The NGF may be encoded by a nucleic acid having the sequence of SEQ ID NO: 69 or SEQ ID NO: 76.

In some embodiments, the neurotrophic factor is NT-4. The NT-4 may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NOs: 23 or 24. The NT-4 may have, for example, an amino acid sequence that differs from the amino acid sequence of SEQ ID NOs: 23 or 24 by only one or more conservative amino acid substitutions, such as by up to 25, up to 20, up to 15, up to 10, or up to 5 conservative amino acid substitutions. The NT-4 may be an NT-4 variant having an amino acid sequence with one or more (e.g., 1, 2, 3, 4, 5, 6, 7, 8, 9, 10, or more) amino acid substitutions relative to the amino acid sequence of SEQ ID NOs: 23 or 24. The NT-4 may be encoded by a nucleic acid having the sequence of SEQ ID NO: 71.

In some embodiments, the neurotrophic factor is BDNF. The BDNF may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of any one of SEQ ID NOs: 25-30. The BDNF may have, for example, an amino acid sequence that differs from the amino acid sequence of any one of SEQ ID NOs: 25-30 by only one or more conservative amino acid substitutions, such as by up to 25, up to 20, up to 15, up to 10, or up to 5 conservative amino acid substitutions. The BDNF may be a BDNF variant having an amino acid sequence with one or more (e.g., 1, 2, 3, 4, 5, 6, 7, 8, 9, 10, or more) amino acid substitutions relative to the amino acid sequence of any one of SEQ ID NOs: 25-30. The BDNF may be encoded by a nucleic acid having the sequence of any one of SEQ ID NOs: 72-75.

In some embodiments, the neurotrophin is a pan-neurotrophin, such as PNT-1. The PNT-1 may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 31. The PNT-1 may have, for example, an amino acid sequence that differs from the amino acid sequence of SEQ ID NO: 31 by only one or more conservative amino acid substitutions, such as by up to 25, up to 20, up to 15, up to 10, or up to 5 conservative amino acid substitutions. The PNT-1 may be a PNT-1 variant having an amino acid sequence with one or more (e.g., 1, 2, 3, 4, 5, 6, 7, 8, 9, 10, or more) amino acid substitutions relative to the amino acid sequence of SEQ ID NO: 31. In some embodiments, the PNT-1 variant has the sequence any one of SEQ ID NOs: 39-42 or 45-47.

In some embodiments, the neurotrophin is a chimeric neurotrophin. In some embodiments, the chimeric neurotrophin is an NGF/BDNF chimera. The chimeric neurotrophin may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of any one of SEQ ID NOs: 32, 33, 43, or 44. The chimeric neurotrophin may have, for example, an amino acid sequence that differs from the amino acid sequence of any one of SEQ ID NOs: 32, 33, 43, or 44 by only one or more conservative amino acid substitutions, such as by up to 25, up to 20, up to 15, up to 10, or up to 5 conservative amino acid substitutions. The chimeric neurotrophin may be a chimeric neurotrophin variant having an amino acid sequence with one or more (e.g., 1, 2, 3, 4, 5, 6, 7, 8, 9, 10, or more) amino acid substitutions relative to the amino acid sequence of any one of SEQ ID NOs: 32, 33, 43, or 44.

In some embodiments, the neurotrophic factor is CNTF. The CNTF may have an amino acid sequence having at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of SEQ ID NO: 61. The CNTF may have, for example, an amino acid sequence that differs from the amino acid sequence of SEQ ID NO: 61 by only one or more conservative amino acid substitutions, such as by up to 25, up to 20, up to 15, up to 10, or up to 5 conservative amino acid substitutions. The CNTF may be a CNTF variant having an amino acid sequence with one or more (e.g., 1, 2, 3, 4, 5, 6, 7, 8, 9, 10, or more) amino acid substitutions relative to the amino acid sequence of SEQ ID NO: 61. The CNTF may be encoded by a nucleic acid having the sequence of SEQ ID NO: 77.

In some embodiments, the neurotrophic factor is IGF. The IGF may have an amino acid sequence of IGF1 and have at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of any one of SEQ ID NOs: 62-65. The IGF may have an amino acid sequence of IGF2 and have at least 85% sequence identity (e.g., at least 85%, 86%, 87%, 88%, 89%, 90%, 91%, 92%, 93%, 94%, 95%, 96%, 97%, 98%, 99%, 99.9%, 99.99%, or 100% sequence identity) to the amino acid sequence of any one of SEQ ID NOs: 66-68. The IGF (e.g., IGF1) may have, for example, an amino acid sequence that differs from the amino acid sequence of any one of SEQ ID NOs: 62-65 by only one or more conservative amino acid substitutions, such as by up to 25, up to 20, up to 15, up to 10, or up to 5 conservative amino acid substitutions. The IGF (e.g., IGF2) may have, for example, an amino acid sequence that differs from the amino acid sequence of any one of SEQ ID NOs: 66-68 by only one or more conservative amino acid substitutions, such as by up to 25, up to 20, up to 15, up to 10, or up to 5 conservative amino acid substitutions. The IGF may be an IGF1 variant having an amino acid sequence with one or more (e.g., 1, 2, 3, 4, 5, 6, 7, 8, 9, 10, or more) amino acid substitutions relative to the amino acid sequence of any one of SEQ ID NOs: 62-65. The IGF may be an IGF2 variant having an amino acid sequence with one or more (e.g., 1, 2, 3, 4, 5, 6, 7, 8, 9, 10, or more) amino acid substitutions relative to the amino acid sequence of any one of SEQ ID NOs: 66-68. The IGF may be IGF1 and may be encoded by a nucleic acid having the sequence of any one of SEQ ID NOs: 78-80. The IGF may be IGF2 and may be encoded by a nucleic acid having the sequence of SEQ ID NO: 81 or SEQ ID NO: 82.

In some embodiments, the therapeutic agent is a glial cell line-derived neurotrophic factor family ligand, a neuropoietic cytokine, an anti-inflammatory cytokine, a neuroprotection agent, growth differentiation factor 11, erythropoietin (EPO), granulocyte-colony stimulating factor, granulocyte-macrophage colony stimulating factor, growth differentiation factor-9, thrombopoietin, transforming growth factor alpha (TGF-α), stromal cell-derived factor 1, myostatin (growth differentiation factor 8), parathyroid hormone, parathyroid hormone related peptide, interleukin 1 receptor antagonist, fibroblast growth factor 18, high-mobility group protein 2, glucocorticoid receptor, fibroblast growth factor 9, hepatocyte growth factor, or a TGFβ3-superfamily protein.

In some embodiments, the therapeutic agent is a glial cell line-derived neurotrophic factor family ligand selected from glial cell line-derived neurotrophic factor (GDNF), neurturin, artemin, and persephin.

Exemplary neurotrophin sequences are provided in Table 3, below.

TABLE 3
Neurotrophin Sequences

| SEQ | | |
|---|---|---|
| ID NO. | Sequence Name | Sequence |
| 19 | Nerve growth factor | MSMLFYTLITAFLIGIQAEPHSESNVPAGHTIPQAHWTK |
| | (protein sequence, | LQHSLDTALRRARSAPAAAIAARVAGQTRNITVDPRLFK |
| | UniProt P01138) | KRRLRSPRVLFSTQPPREAADTQDLDFEVGGAAPFNRTH |
| | | RSKRSSSHPIFHRGEFSVCDSVSVWVGDKTTATDIKGKE |
| | | VMVLGEVNINNSVFKQYFFETKCRDPNPVDSGCRGIDSK |
| | | HWNSYCTTTHTFVKALTMDGKQAAWRFIRIDTACVCVLS |
| | | RKAVRRA |
| 20 | Nerve growth | SSSHPIFHRGEFSVCDSVSVWVGDKTTATDIKGKEVMVL |
| | factor | GEVNINNSVFKQYFFETKCRDPNPVDSGCRGIDSKHWNS |
| | (protein sequence, | YCTTTHTFVKALTMDGKQAAWRFIRIDTACVCVLSRKAV |
| | mature form) | RRA |
| 21 | Neurotrophin 3 | MVTFATILQVNKVMSILFYVIFLAYLRGIQGNNMDQRSL |
| | (Isoform 2 | PEDSLNSLIIKLIQADILKNKLSKQMVDVKENYQSTLPK |
| | protein sequence, | AEAPREPERGGPAKSAFQPVIAMDTELLRQQRRYNSPRV |
| | UniProt P20783-2) | LLSDSTPLEPPPLYLMEDYVGSPVVANRTSRRKRYAEHK |
| | | SHRGEYSVCDSESLWVTDKSSAIDIRGHQVTVLGEIKTG |
| | | NSPVKQYFYETRCKEARPVKNGCRGIDDKHWNSQCKTSQ |
| | | TYVRALTSENNKLVGWRWIRIDTSCVCALSRKIGRT |
| 22 | Neurotrophin 3 | YAEHKSHRGEYSVCDSESLWVTDKSSAIDIRGHQVTVLG |
| | (protein sequence, | EIKTGNSPVKQYFYETRCKEARPVKNGCRGIDDKHWNSQ |
| | mature form) | CKTSQTYVRALTSENNKLVGWRWIRIDTSCVCALSRKIG |
| | | RT |
| 23 | Neurotrophin 4 | MLPLPSCSLPILLLFLLPSVPIESQPPPSTLPPFLAPEW |
| | (protein sequence, | DLLSPRVVLSRGAPAGPPLLFLLEAGAFRESAGAPANRS |
| | UniProt P34130) | RRGVSETAPASRRGELAVCDAVSGWVTDRRTAVDLRGRE |
| | | VEVLGEVPAAGGSPLRQYFFETRCKADNAEEGGPGAGGG |
| | | GCRGVDRRHWVSECKAKQSYVRALTADAQGRVGWRWIRI |
| | | DTACVCTLLSRTGRA |
| 24 | Neurotrophin 4 | GVSETAPASRRGELAVCDAVSGWVTDRRTAVDLRGREVE |
| | (protein sequence, | VLGEVPAAGGSPLRQYFFETRCKADNAEEGGPGAGGGGC |
| | mature form) | RGVDRRHWVSECKAKQSYVRALTADAQGRVGWRWIRIDT |
| | | ACVCTLLSRTGRA |
| 25 | Brain-derived | MTILFLTMVISYFGCMKAAPMKEANIRGQGGLAYPGVRT |
| | neurotrophic factor | HGTLESVNGPKAGSRGLTSLADTFEHVIEELLDEDQKVR |
| | (Isoform 1 | PNEENNKDADLYTSRVMLSSQVPLEPPLLFLLEEYKNYL |
| | protein sequence, | DAANMSMRVRRHSDPARRGELSVCDSISEWVTAADKKTA |
| | UniProt P23560) | VDMSGGTVTVLEKVPVSKGQLKQYFYETKCNPMGYTKEG |
| | | CRGIDKRHWNSQCRTTQSYVRALTMDSKKRIGWRFIRID |
| | | TSCVCTLTIKRGR |
| 26 | Brain-derived | MQSREEEWFHQVRRVMTILFLTMVISYFGCMKAAPMKEA |
| | neurotrophic factor | NIRGQGGLAYPGVRTHGTLESVNGPKAGSRGLTSLADTF |
| | (Isoform 3 | EHVIEELLDEDQKVRPNEENNKDADLYTSRVMLSSQVPL |
| | protein sequence, | EPPLLFLLEEYKNYLDAANMSMRVRRHSDPARRGELSVC |
| | UniProt P23560-3) | DSISEWVTAADKKTAVDMSGGTVTVLEKVPVSKGQLKQY |
| | | FYETKCNPMGYTKEGCRGIDKRHWNSQCRTTQSYVRALT |
| | | MDSKKRIGWRFIRIDTSCVCTLTIKRGR |
| 27 | Brain-derived | MCGATSFLHECTRLILVTTQNAEFLQKGLQVHTCFGVYP |
| | neurotrophic factor | HASVWHDCASQKKGCAVYLHVSVEFNKLIPENGFIKFHQ |
| | (Isoform 4 | VRRVMTILFLTMVISYFGCMKAAPMKEANIRGQGGLAYP |
| | protein sequence, | GVRTHGTLESVNGPKAGSRGLTSLADTFEHVIEELLDED |
| | UniProt P23560-4) | QKVRPNEENNKDADLYTSRVMLSSQVPLEPPLLFLLEEY |
| | | KNYLDAANMSMRVRRHSDPARRGELSVCDSISEWVTAAD |
| | | KKTAVDMSGGTVTVLEKVPVSKGQLKQYFYETKCNPMGY |
| | | TKEGCRGIDKRHWNSQCRTTQSYVRALTMDSKKRIGWRF |
| | | IRIDTSCVCTLTIKRGR |
| 28 | Brain-derived | MFHQVRRVMTILFLTMVISYFGCMKAAPMKEANIRGQGG |
| | neurotrophic factor | LAYPGVRTHGTLESVNGPKAGSRGLTSLADTFEHVIEEL |
| | (Isoform 2 | LDEDQKVRPNEENNKDADLYTSRVMLSSQVPLEPPLLFL |
| | protein sequence, | LEEYKNYLDAANMSMRVRRHSDPARRGELSVCDSISEWV |
| | UniProt P23560-2) | TAADKKTAVDMSGGTVTVLEKVPVSKGQLKQYFYETKCN |
| | | PMGYTKEGCRGIDKRHWNSQCRTTQSYVRALTMDSKKRI |
| | | GWRFIRIDTSCVCTLTIKRGR |
| 29 | Brain-derived | MLCAISLCARVRKLRSAGRCGKFHQVRRVMTILFLTMVI |
| | neurotrophic factor | SYFGCMKAAPMKEANIRGQGGLAYPGVRTHGTLESVNGP |
| | (Isoform 5 | KAGSRGLTSLADTFEHVIEELLDEDQKVRPNEENNKDAD |
| | protein sequence, | LYTSRVMLSSQVPLEPPLLFLLEEYKNYLDAANMSMRVR |
| | UniProt P23560-5) | RHSDPARRGELSVCDSISEWVTAADKKTAVDMSGGTVTV |
| | | LEKVPVSKGQLKQYFYETKCNPMGYTKEGCRGIDKRHWN |
| | | SQCRTTQSYVRALTMDSKKRIGWRFIRIDTSCVCTLTIK |
| | | RGR |
| 30 | Brain-derived | HSDPARRGELSVCDSISEWVTAADKKTAVDMSGGTVTVL |
| | neurotrophic factor | EKVPVSKGQLKQYFYETKCNPMGYTKEGCRGIDKRHWNS |
| | (protein sequence, | QCRTTQSYVRALTMDSKKRIGWRFIRIDTSCVCTLTIKR |
| | mature form) | GR |
| 31 | PNT-1 | SSSHPIFHRGEYSVCDSESLWVTDKSSAIDIRGHQVTVL |
| | | GEIKTGNSPVKQYFYETRCKEARPVKNGCRGIDDKHWNS |
| | | QCKTSQTYVRALTSESKKRIGWRWIRIDTSCVCALSRKI |
| | | GRT |
| 32 | NGF/BDNF | SSSHPIFHRGEFSVCDSVSVWVGDKTTATDIKGKEVMVL |
| | chimera 1 | GEVNINNSVFKQYFFETKCNPMGYTKEGCRGIDSKHWNS |
| | (3 + 4 + 5) | QCRTTQSYVRALTMDSKKRIGWRFIRIDTACVCVLSRKA |
| | | VRRA |
| 33 | NGF/BDNF | SSSHPIFHRGEFSVCDSVSVWVTAADKKTAVDMSGGTVM |
| | chimera 2 | VLGEVNINNSVFKQYFFETKCNPMGYTKEGCRGIDSKHW |
| | (1 + 3 + 4 + 5) | NSQCRTTQSYVRALTMDSKKRIGWRFIRIDTACVCVLSR |
| | | KAVRRA |
| 34 | NGF variant 1 | SSSHPIAHRGEFSVCDSVSVWVGDKTTATDIKGKEVMVL |
| | (protein sequence | GEVNINNSVFKQYFFETKCRDPNPVDSGCRGIDSKHWNS |
| | of mature NGF, | YCTTTATFVKALTMDGKQAAWRFIAIDTACVCVLSRKAV |
| | with amino acid | RRA |
| | substitutions | |
| | F7A/H84A/R103A) | |
| 35 | NGF variant 2 | SSSHPIAHRGEFSVCDSVSVWVGDKTTATDIKGKEVMVL |
| | (protein sequence | GEVNIANSVFKQYFFETKCRDPNPVDSGCRGIDSKHWNS |
| | of mature NGF, | YCTTTHTFVKALTMDGKQAAWRFIAIDTACVCVLSRKAV |
| | with amino acid | RRA |
| | substitutions | |
| | F7A/N45A/R103A) | |
| 36 | NGF variant 3 | SSSHPIAHRGEFSVCDSVSVWVGDKTTATDIAGAAVMVL |
| | (protein sequence | GEVNINNSVFKQYFFETKCRDPNPVDSGCRGIDSKHWNS |
| | of mature NGF, | YCTTTATFVKALTMDGKQAAWRFIAIDTACVCVLSRKAV |
| | with amino acid | RRA |
| | substitutions | |
| | K32A/K34A/E35A | |
| | F7A/H84A/R103A) | |
| 37 | NGF variant 4 | SSSHPIAHRGEFSVCDSVSVWVGDKTTATDIAGAAVMVL |
| | (protein sequence | GEVNIANSVFKQYFFETKCRDPNPVDSGCRGIDSKHWNS |
| | of mature NGF, | YCTTTHTFVKALTMDGKQAAWRFIAIDTACVCVLSRKAV |
| | with amino acid | RRA |
| | substitutions | |
| | K32A/K34A/E35A | |
| | F7A/N45A/R103A) | |
| 38 | NGF variant 5 | SSSHPIFHVCDSVSVWVGDKTTATDIAGAAVMVLGEVNI |
| | (protein sequence | NNSVFKQYFFETKCRDPNPVDSGCRGIDSKHWNSYCTTT |
| | of mature NGF, | HTFVKALTMDGKQAAWRFIRIDTACVCVLSRKAVRRA |
| | with amino acid | |
| | changes | |
| | K32A/K34A/E35A | |
| | Δ9-13) | |
| 39 | PNT-1 variant 1 | SSSHPIFHRGEYSVCDSESLWVTDKSSAIDIAGAQVTVL |
| | (protein sequence | GEIKTGNSPVKQYFYETRCKEARPVKNGCRGIDDKHWNS |
| | of PNT-1, | QCKTSQTYVRALTSESKKRIGWRWIRIDTSCVCALSRKI |
| | with amino acid | GRT |
| | substitutions | |
| | R32A/H34A) | |
| 40 | PNT-1 variant 2 | SSSHPIFHRGEYSVCDSESLWVTDKSSAIDIRGHQVTVL |
| | (protein sequence | GEIKTGNSPVKQYFYETRCKEARPVKNGCRGIDDAAWAS |
| | of PNT-1, | QCKTSQTYVRALTSESKKRIGWRWIRIDTSCVCALSRKI |
| | with amino acid | GRT |
| | substitutions | |
| | K74A/H75A/N77A) | |
| 41 | PNT-1 variant 3 | SSSHPIFHRGEYSVCDSESLWVTDKSSAIDIRGHQVTVL |
| | (protein sequence | GEIKTGNSPVKQYFYETRCKEARPVKNGCRGIDDKHWNS |
| | of PNT-1, | QCKTSQTYVRALTSESKKRIGWRWIRIDTSCVCALSAAI |
| | with amino acid | GRT |
| | substitutions | |
| | R115A/K116A) | |
| 42 | PNT-1 variant 4 | SSSHPIFHRGEYSVCDSESLWVTDKSSAIDIAGAQVTVL |
| | (protein sequence | GEIKTGNSPVKQYFYETRCKEARPVKNGCRGIDDAAWAS |
| | of PNT-1, | QCKTSQTYVRALTSESKKRIGWRWIRIDTSCVCALSAAI |
| | with amino acid | GRT |
| | substitutions | |
| | R32A/H34A/K74A/ | |
| | H75A/N77A/ | |
| | R115A/K116A) | |
| 43 | NGF/BDNF chimera 3 | SSSHPIFHRGEFSVCDSVSVWVGDKTTATDIAGAAVMVL |
| | (3 + 4 + 5 | GEVNINNSVFKQYFFETKCNPMGYTKEGCRGIDSKHWNS |
| | and KKE, | QCRTTQSYVRALTMDSKKRIGWRFIRIDTACVCVLSRKA |
| | K32A/K34A/E35A) | VRRA |
| 44 | NGF/BDF chimera 4 | SSSHPIFHRGEFSVCDSVSVWVTAADKKTAVDMAGAAVM |
| | (1 + 3 + 4 + 5 | VLGEVNINNSVFKQYFFETKCNPMGYTKEGCRGIDSKHW |
| | and KKE, | NSQCRTTQSYVRALTMDSKKRIGWRFIRIDTACVCVLSR |
| | K32A/K34A/E35A) | KAVRRA |
| 45 | PNT1 + KKE | SSSHPIFHRGEYSVCDSESLWVTDKSSAIDIAGAAVTVL |
| | (protein sequence | GEIKTGNSPVKQYFYETRCKEARPVKNGCRGIDDKHWNS |
| | of PNT-1, | QCKTSQTYVRALTSESKKRIGWRWIRIDTSCVCALSRKI |
| | with amino acid | GRT |
| | substitutions | |
| | R32A/H34A/Q35A) | |
| 46 | PNT-1 variant 5 | SSSHPIFHRGEYSVCDSESLWVTDKSSAIDIRGHQVTVL |
| | (protein sequence | GEIKTGNSPVKQYFYETRCKESRPVKNGCRGIDDKHWNS |
| | of PNT-1, | QCKTSQTYVRALTSESKKRIGWEWIRIDTSCVCALSRKI |
| | with amino acid | GRT |
| | substitutions | |
| | A61S/R101E) | |
| 47 | PNT-1 variant 6 | SSSHPIFHRGEYSVCDSESLWVTDKSSAIDIRGHQVTVL |
| | (protein sequence | GEIKTGNSPVKQYFYETRCKEARPVKNGCRGIDDKHWNS |
| | of PNT-1, | QCKTSQTYVRALTSESKKRIGWRWIRIDTSCVCALSRKI |
| | with amino acid | ART |
| | substitution | |
| | G118A) | |
| 48 | NT-3 variant 1 | YAEHKSHRGEYSVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIAIDTSCVCALSRKIG |
| | with amino acid | RT |
| | substitutions | |
| | D15A/R103A) | |
| 49 | NT-3 variant 2 | YAEHKSHRGEYSVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIRIDTSCVCALSAAIG |
| | with amino acid | RT |
| | substitutions | |
| | D15A/R114A/K115A) | |
| 50 | NT-3 variant 3 | YAEHKSHRGEASVCASESLWVTDKSSAIDIRGHQVTVLG |
| | (protein sequence | EIKTGNSPVKQYFYETRCKEARPVKNGCAGIDDKHWNSQ |
| | of mature NT3, | CKTSQTYVAALTSENNKLVGWRWIRIDTSCVCALSAAIG |
| | with amino acid | RT |
| | substitutions | |
| | Y11A/D15A/R68A/ | |
| | R87A/R114A/K115A | |
| 51 | NT-3 variant 4 | YAEHKSHRGEYSVCASESLWVTDKSSAIDIRGHQVTVLG |
| | (protein sequence | EIKTGNSPVKQYFYETRCKEARPVKNGCAGIDDKHWNSQ |
| | of mature NT3, | CKTSQTYVAALTSENNKLVGWRWIRIDTSCVCALSAAIG |
| | with amino acid | RT |
| | substitutions | |
| | D15A/R68A/R87A/ | |
| | R114A/K115A) | |
| 52 | NT-3 variant 5 | YAEHKSHRGEASVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVAALTSENNKLVGWRWIRIDTSCVCALSAAIG |
| | with amino acid | RT |
| | substitutions | |
| | Y11A/D15A/R87A/ | |
| | R114A/K115A) | |
| 53 | NT-3 variant 6 | YAEHKSHRGEYSVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVAALTSENNKLVGWRWIRIDTSCVCALSAAIG |
| | with amino acid | RT |
| | substitutions | |
| | D15A/R87A/ | |
| | R114A/K115A) | |
| 54 | NT-3 variant 7 | YAEHKSHRGEASVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIRIDTSCVCALSAAIG |
| | with amino acid | RT |
| | substitutions | |
| | Y11A/D15A/ | |
| | R114A/K115A) | |
| 55 | NT-3 variant 8 | YAEHKSHRGEYSVCASESLWVTDKSSAIDIRGHQVTVLG |
| | (protein sequence | EIKTGNSPVKQAFYETRCKEARPVKNGCRGIDDKHWNSQ |
| | of mature NT3, | CKTSQTYVRALTSENNKLVGWRWIAIDTSCVCALSRKIG |
| | with amino acid | RT |
| | substitutions | |
| | D15A/R103A/Y51A) | |
| 56 | NT-3 variant 9 | YAEHKSHRGEYSVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIAIDTSCVCALSAAIG |
| | with amino acid | RT |
| | substitutions | |
| | D15A/R103A/ | |
| | R114A/K115A) | |
| 57 | NT-3 variant 10 | YAEHKSHRGEYSVCASESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIRIDTSCVCALSRKIG |
| | with amino acid | RT |
| | substitution | |
| | D15A) | |
| 58 | NT-3 variant 11 | YAEHKSHRGEYSVCDSESLWVTDKSSAIDIRGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIRIDTSCVCALSAAIG |
| | with amino acid | RT |
| | substitutions | |
| | R114A/K115A) | |
| 59 | NT-3 variant 12 | YAEHKSHRGEYSVCASESLWVTDKSSAIDIAGHQVTVLG |
| | (protein sequence | EIKTGNSPVKQYFYETRCKEARPVKNGCRGIDDKHWNSQ |
| | of mature NT3, | CKTSQTYVRALTSENNKLVGWRWIRIDTSCVCALSRKIG |
| | with amino acid | RT |
| | substitution | |
| | R31A) | |
| 60 | NT-3 variant 13 | YAEHKSHRGEYSVCASESLWVTDKSSAIDIAGHQVTVLG |
| | (protein sequence | AIKTGNSPVKQYFYETRCKEARPVKNGCRGIDDKHWNSQ |
| | of mature NT3, | CKTSQTYVRALTSENNKLVGWRWIRIDTSCVCALSRKIG |
| | with amino acid | RT |
| | substitution | |
| | E40A) | |
| 61 | Ciliary neurotrophic | MAFTEHSPLTPHRRDLCSRSIWLARKIRSDLTALTESYV |
| | factor (CNTF) | KHQGLNKNINLDSADGMPVASTDQWSELTEAERLQENLQ |
| | (protein sequence, | AYRTFHVLLARLLEDQQVHFTPTEGDFHQAIHTLLLQVA |
| | UniProt P26441) | AFAYQIEELMILLEYKIPRNEADGMPINVGDGGLFEKKL |
| | | WGLKVLQELSQWTVRSIHDLRFISSHQTGIPARGSHYIA |
| | | NNKKM |
| 62 | Insulin like growth | MGKISSLPTQLFKCCFCDFLKVKMHTMSSSHLFYLALCL |
| | factor 1 (IGF1) | LTFTSSATAGPETLCGAELVDALQFVCGDRGFYFNKPTG |
| | (protein sequence, | YGSSSRRAPQTGIVDECCFRSCDLRRLEMYCAPLKPAKS |
| | Isoform 1- | ARSVRAQRHTDMPKTQKYQPPSTNKNTKSQRRKGWPKTH |
| | also known | PGGEQKEGTEASLQIRGKKKEQRREIGSRNAECRGKKGK |
| | as IGF-1B, | |
| | UniProt P05019-1) | |
| 63 | Insulin like growth | MGKISSLPTQLFKCCFCDFLKVKMHTMSSSHLFYLALCL |
| | factor 1 (IGF1) | LTFTSSATAGPETLCGAELVDALQFVCGDRGFYFNKPTG |
| | (protein sequence, | YGSSSRRAPQTGIVDECCFRSCDLRRLEMYCAPLKPAKS |
| | Isoform 2- | ARSVRAQRHTDMPKTQKEVHLKNASRGSAGNKNYRM |
| | also known | |
| | as IGF-1A, | |
| | UniProt P05019-2) | |
| 64 | Insulin like growth | MITPTVKMHTMSSSHLFYLALCLLTFTSSATAGPETLCG |
| | factor 1 (IGF1) | AELVDALQFVCGDRGFYFNKPTGYGSSSRRAPQTGIVDE |
| | (protein sequence, | CCFRSCDLRRLEMYCAPLKPAKSARSVRAQRHTDMPKTQ |
| | Isoform 3, | KEVHLKNASRGSAGNKNYRM |
| | UniProt P05019-3) | |
| 65 | Insulin like growth | MGKISSLPTQLFKCCFCDFLKVKMHTMSSSHLFYLALCL |
| | factor 1 (IGF1) | LTFTSSATAGPETLCGAELVDALQFVCGDRGFYFNKPTG |
| | (protein sequence, | YGSSSRRAPQTGIVDECCFRSCDLRRLEMYCAPLKPAKS |
| | Isoform 4, | ARSVRAQRHTDMPKTQKYQPPSTNKNTKSQRRKGSTFEE |
| | UniProt P05019-4) | RK |
| 66 | Insulin like growth | MGIPMGKSMLVLLTFLAFASCCIAAYRPSETLCGGELVD |
| | factor 2 (IGF2) | TLQFVCGDRGFYFSRPASRVSRRSRGIVEECCFRSCDLA |
| | (protein sequence, | LLETYCATPAKSERDVSTPPTVLPDNFPRYPVGKFFQYD |
| | Isoform 1, | TWKQSTQRLRRGLPALLRARRGHVLAKELEAFREAKRHR |
| | UniProt P01344-1) | PLIALPTQDPAHGGAPPEMASNRK |
| 67 | Insulin like growth | MGIPMGKSMLVLLTFLAFASCCIAAYRPSETLCGGELVD |
| | factor 2 (IGF2) | TLQFVCGDRGFYFRLPGRPASRVSRRSRGIVEECCFRSC |
| | (protein sequence, | DLALLETYCATPAKSERDVSTPPTVLPDNFPRYPVGKFF |
| | Isoform 2, | QYDTWKQSTQRLRRGLPALLRARRGHVLAKELEAFREAK |
| | UniProt P01344-2) | RHRPLIALPTQDPAHGGAPPEMASNRK |
| 68 | Insulin like growth | MVSPDPQIIVVAPETELASMQVQRTEDGVTIIQIFWVGR |
| | factor 2 (IGF2) | KGELLRRTPVSSAMQTPMGIPMGKSMLVLLTFLAFASCC |
| | (protein sequence, | IAAYRPSETLCGGELVDTLQFVCGDRGFYFSRPASRVSR |
| | Isoform 3, | RSRGIVEECCFRSCDLALLETYCATPAKSERDVSTPPTV |
| | UniProt P01344-3) | LPDNFPRYPVGKFFQYDTWKQSTQRLRRGLPALLRARRG |
| | | HVLAKELEAFREAKRHRPLIALPTQDPAHGGAPPEMASN |
| | | RK |
| 69 | Nerve Growth | GATCTTCCTCCCACCCAATCTTCCACAGAGGTGAATTCT |
| | Factor (nucleic | CTGTCTGTGACTCTGTCTCTGTCTGGGTTGGTGATAAGA |
| | acid sequence, | CCACTGCTACCGACATCAAGGGTAAGGAAGTCATGGTTT |
| | mature form) | TGGGTGAAGTTAACATTAACAACTCCGTTTTCAAGCAAT |
| | | ACTTCTTCGAAACTAAGTGTAGAGATCCAAACCCAGTTG |
| | | ACTCTGGTTGTAGAGGTATCGATTCCAAGCACTGGAACT |
| | | CTTACTGTACCACTACCCACACTTTCGTCAAGGCTTTGA |
| | | CTATGGACGGTAAGCAAGCTGCCTGGAGATTCATCAGAA |
| | | TTGACACCGCTTGTGTCTGTGTTTTGTCTAGAAAGGCTG |
| | | TTAGAAGAGCTTGATAA |
| 70 | Neurotrophin 3 | ATGGTTACTTTTGCCACGATCTTACAGGTGAACAAGGTG |
| | (Isoform 2 nucleic | ATGTCCATCTTGTTTTATGTGATATTTCTCGCTTATCTC |
| | acid sequence, | CGTGGCATCCAAGGTAACAACATGGATCAAAGGAGTTTG |
| | CCDS 44806.1) | CCAGAAGACTCGCTCAATTCCCTCATTATTAAGCTGATC |
| | | CAGGCAGATATTTTGAAAAACAAGCTCTCCAAGCAGATG |
| | | GTGGACGTTAAGGAAAATTACCAGAGCACCCTGCCCAAA |
| | | GCTGAGGCTCCCCGAGAGCCGGAGCGGGGAGGGCCCGCC |
| | | AAGTCAGCATTCCAGCCGGTGATTGCAATGGACACCGAA |
| | | CTGCTGCGACAACAGAGACGCTACAACTCACCGCGGGTC |
| | | CTGCTGAGCGACAGCACCCCCTTGGAGCCCCCGCCCTTG |
| | | TATCTCATGGAGGATTACGTGGGCAGCCCCGTGGTGGCG |
| | | AACAGAACATCACGGCGGAAACGGTACGCGGAGCATAAG |
| | | AGTCACCGAGGGGAGTACTCGGTATGTGACAGTGAGAGT |
| | | CTGTGGGTGACCGACAAGTCATCGGCCATCGACATTCGG |
| | | GGACACCAGGTCACGGTGCTGGGGGAGATCAAAACGGGC |
| | | AACTCTCCCGTCAAACAATATTTTTATGAAACGCGATGT |
| | | AAGGAAGCCAGGCCGGTCAAAAACGGTTGCAGGGGTATT |
| | | GATGATAAACACTGGAACTCTCAGTGCAAAACATCCCAA |
| | | ACCTACGTCCGAGCACTGACTTCAGAGAACAATAAACTC |
| | | GTGGGCTGGCGGTGGATACGGATAGACACGTCCTGTGTG |
| | | TGTGCCTTGTCGAGAAAAATCGGAAGAACAT |
| 71 | Neurotrophin 4 | ATGCTCCCTCTCCCCTCATGCTCCCTCCCCATCCTCCTC |
| | (nucleic | CTTTTCCTCCTCCCCAGTGTGCCAATTGAGTCCCAACCC |
| | acid sequence, | CCACCCTCAACATTGCCCCCTTTTCTGGCCCCTGAGTGG |
| | CCDS 12754.1) | GACCTTCTCTCCCCCCGAGTAGTCCTGTCTAGGGGTGCC |
| | | CCTGCTGGGCCCCCTCTGCTCTTCCTGCTGGAGGCTGGG |
| | | GCCTTTCGGGAGTCAGCAGGTGCCCCGGCCAACCGCAGC |
| | | CGGCGTGGGGTGAGCGAAACTGCACCAGCGAGTCGTCGG |
| | | GGTGAGCTGGCTGTGTGCGATGCAGTCAGTGGCTGGGTG |
| | | ACAGACCGCCGGACCGCTGTGGACTTGCGTGGGCGCGAG |
| | | GTGGAGGTGTTGGGCGAGGTGCCTGCAGCTGGCGGCAGT |
| | | CCCCTCCGCCAGTACTTCTTTGAAACCCGCTGCAAGGCT |
| | | GATAACGCTGAGGAAGGTGGCCCGGGGGCAGGTGGAGGG |
| | | GGCTGCCGGGGAGTGGACAGGAGGCACTGGGTATCTGAG |
| | | TGCAAGGCCAAGCAGTCCTATGTGCGGGCATTGACCGCT |
| | | GATGCCCAGGGCCGTGTGGGCTGGCGATGGATTCGAATT |
| | | GACACTGCCTGCGTCTGCACACTCCTCAGCCGGACTGGC |
| | | CGGGCCT |
| 72 | Brain-derived | ACAGAGCCGGGAAGAGGAATGGTTCCACCAGGTGAGAAG |
| | neurotrophic factor | AGTGATGACCATCCTTTTCCTTACTATGGTTATTTCATA |
| | (Isoform 3 nucleic | CTTTGGTTGCATGAAGGCTGCCCCCATGAAAGAAGCAAA |
| | acid sequence, | CATCCGAGGACAAGGTGGCTTGGCCTACCCAGGTGTGCG |
| | CCDS 41628.1) | GACCCATGGGACTCTGGAGAGCGTGAATGGGCCCAAGGC |
| | | AGGTTCAAGAGGCTTGACATCATTGGCTGACACTTTCGA |
| | | ACACGTGATAGAAGAGCTGTTGGATGAGGACCAGAAAGT |
| | | TCGGCCCAATGAAGAAAACAATAAGGACGCAGACTTGTA |
| | | CACGTCCAGGGTGATGCTCAGTAGTCAAGTGCCTTTGGA |
| | | GCCTCCTCTTCTCTTTCTGCTGGAGGAATACAAAAATTA |
| | | CCTAGATGCTGCAAACATGTCCATGAGGGTCCGGCGCCA |
| | | CTCTGACCCTGCCCGCCGAGGGGAGCTGAGCGTGTGTGA |
| | | CAGTATTAGTGAGTGGGTAACGGCGGCAGACAAAAAGAC |
| | | TGCAGTGGACATGTCGGGCGGGACGGTCACAGTCCTTGA |
| | | AAAGGTCCCTGTATCAAAAGGCCAACTGAAGCAATACTT |
| | | CTACGAGACCAAGTGCAATCCCATGGGTTACACAAAAGA |
| | | AGGCTGCAGGGGCATAGACAAAAGGCATTGGAACTCCCA |
| | | GTGCCGAACTACCCAGTCGTACGTGCGGGCCCTTACCAT |
| | | GGATAGCAAAAAGAGAATTGGCTGGCGATTCATAAGGAT |
| | | AGACACTTCTTGTGTATGTACATTGACCATTAAAAGGGG |
| | | AAGA |
| 73 | Brain-derived | ATGTGTGGAGCCACCAGTTTTCTCCATGAGTGCACAAGG |
| | neurotrophic factor | TTAATCCTTGTTACTACTCAGAATGCTGAGTTTCTACAG |
| | (Isoform 4 nucleic | AAAGGGTTGCAGGTCCACACATGTTTTGGCGTCTACCCA |
| | acid sequence, | CACGCTTCTGTATGGCATGACTGTGCATCCCAGAAGAAG |
| | CCDS 44558.1) | GGCTGTGCTGTGTACCTCCACGTTTCAGTGGAATTTAAC |
| | | AAACTGATCCCTGAAAATGGTTTCATAAAGTTCCACCAG |
| | | GTGAGAAGAGTGATGACCATCCTTTTCCTTACTATGGTT |
| | | ATTTCATACTTTGGTTGCATGAAGGCTGCCCCCATGAAA |
| | | GAAGCAAACATCCGAGGACAAGGTGGCTTGGCCTACCCA |
| | | GGTGTGCGGACCCATGGGACTCTGGAGAGCGTGAATGGG |
| | | CCCAAGGCAGGTTCAAGAGGCTTGACATCATTGGCTGAC |
| | | ACTTTCGAACACGTGATAGAAGAGCTGTTGGATGAGGAC |
| | | CAGAAAGTTCGGCCCAATGAAGAAAACAATAAGGACGCA |
| | | GACTTGTACACGTCCAGGGTGATGCTCAGTAGTCAAGTG |
| | | CCTTTGGAGCCTCCTCTTCTCTTTCTGCTGGAGGAATAC |
| | | AAAAATTACCTAGATGCTGCAAACATGTCCATGAGGGTC |
| | | CGGCGCCACTCTGACCCTGCCCGCCGAGGGGAGCTGAGC |
| | | GTGTGTGACAGTATTAGTGAGTGGGTAACGGCGGCAGAC |
| | | AAAAAGACTGCAGTGGACATGTCGGGCGGGACGGTCACA |
| | | GTCCTTGAAAAGGTCCCTGTATCAAAAGGCCAACTGAAG |
| | | CAATACTTCTACGAGACCAAGTGCAATCCCATGGGTTAC |
| | | ACAAAAGAAGGCTGCAGGGGCATAGACAAAAGGCATTGG |
| | | AACTCCCAGTGCCGAACTACCCAGTCGTACGTGCGGGCC |
| | | CTTACCATGGATAGCAAAAAGAGAATTGGCTGGCGATTC |
| | | ATAAGGATAGACACTTCTTGTGTATGTACATTGACCATT |
| | | AAAAGGGGAAGA |
| 74 | Brain-derived | ATGTTCCACCAGGTGAGAAGAGTGATGACCATCCTTTTC |
| | neurotrophic factor | CTTACTATGGTTATTTCATACTTTGGTTGCATGAAGGCT |
| | (Isoform 2 nucleic | GCCCCCATGAAAGAAGCAAACATCCGAGGACAAGGTGGC |
| | acid sequence, | TTGGCCTACCCAGGTGTGCGGACCCATGGGACTCTGGAG |
| | CCDS 7865.1) | AGCGTGAATGGGCCCAAGGCAGGTTCAAGAGGCTTGACA |
| | | TCATTGGCTGACACTTTCGAACACGTGATAGAAGAGCTG |
| | | TTGGATGAGGACCAGAAAGTTCGGCCCAATGAAGAAAAC |
| | | AATAAGGACGCAGACTTGTACACGTCCAGGGTGATGCTC |
| | | AGTAGTCAAGTGCCTTTGGAGCCTCCTCTTCTCTTTCTG |
| | | CTGGAGGAATACAAAAATTACCTAGATGCTGCAAACATG |
| | | TCCATGAGGGTCCGGCGCCACTCTGACCCTGCCCGCCGA |
| | | GGGGAGCTGAGCGTGTGTGACAGTATTAGTGAGTGGGTA |
| | | ACGGCGGCAGACAAAAAGACTGCAGTGGACATGTCGGGC |
| | | GGGACGGTCACAGTCCTTGAAAAGGTCCCTGTATCAAAA |
| | | GGCCAACTGAAGCAATACTTCTACGAGACCAAGTGCAAT |
| | | CCCATGGGTTACACAAAAGAAGGCTGCAGGGGCATAGAC |
| | | AAAAGGCATTGGAACTCCCAGTGCCGAACTACCCAGTCG |
| | | TACGTGCGGGCCCTTACCATGGATAGCAAAAAGAGAATT |
| | | GGCTGGCGATTCATAAGGATAGACACTTCTTGTGTATGT |
| | | ACATTGACCATTAAAAGGGGAAGA |
| 75 | Brain-derived | ATGACCATCCTTTTCCTTACTATGGTTATTTCATACTTT |
| | neurotrophic factor | GGTTGCATGAAGGCTGCCCCCATGAAAGAAGCAAACATC |
| | (Isoform 1 nucleic | CGAGGACAAGGTGGCTTGGCCTACCCAGGTGTGCGGACC |
| | acid sequence, | CATGGGACTCTGGAGAGCGTGAATGGGCCCAAGGCAGGT |
| | CCDS 7866.1) | TCAAGAGGCTTGACATCATTGGCTGACACTTTCGAACAC |
| | | GTGATAGAAGAGCTGTTGGATGAGGACCAGAAAGTTCGG |
| | | CCCAATGAAGAAAACAATAAGGACGCAGACTTGTACACG |
| | | TCCAGGGTGATGCTCAGTAGTCAAGTGCCTTTGGAGCCT |
| | | CCTCTTCTCTTTCTGCTGGAGGAATACAAAAATTACCTA |
| | | GATGCTGCAAACATGTCCATGAGGGTCCGGCGCCACTCT |
| | | GACCCTGCCCGCCGAGGGGAGCTGAGCGTGTGTGACAGT |
| | | ATTAGTGAGTGGGTAACGGCGGCAGACAAAAAGACTGCA |
| | | GTGGACATGTCGGGCGGGACGGTCACAGTCCTTGAAAAG |
| | | GTCCCTGTACAAAAGGCCAACTGAAGCAATACTTCTACG |
| | | AGACCAAGTGCAATCCCATGGGTTACACAAAAGAAGGCT |
| | | GCAGGGGCATAGACAAAAGGCATTGGAACTCCCAGTGCC |
| | | GAACTACCCAGTCGTACGTGCGGGCCCTTACCATGGATA |
| | | GCAAAAAGAGAATTGGCTGGCGATTCATAAGGATAGACA |
| | | CTTCTTGTGTATGTACATTGACCATTAAAAGGGGAAGA |
| 76 | Nerve Growth | ATGTCCATGTTGTTCTACACTCTGATCACAGCTTTTCTG |
| | Factor (nucleic | ATCGGCATACAGGCGGAACCACACTCAGAGAGCAATGTC |
| | acid sequence, | CCTGCAGGACACACCATCCCCCAAGCCCACTGGACTAAA |
| | CCDS882.1) | CTTCAGCATTCCCTTGACACTGCCCTTCGCAGAGCCCGC |
| | | AGCGCCCCGGCAGCGGCGATAGCTGCACGCGTGGCGGGG |
| | | CAGACCCGCAACATTACTGTGGACCCCAGGCTGTTTAAA |
| | | AAGCGGCGACTCCGTTCACCCCGTGTGCTGTTTAGCACC |
| | | CAGCCTCCCCGTGAAGCTGCAGACACTCAGGATCTGGAC |
| | | TTCGAGGTCGGTGGTGCTGCCCCCTTCAACAGGACTCAC |
| | | AGGAGCAAGCGGTCATCATCCCATCCCATCTTCCACAGG |
| | | GGCGAATTCTCGGTGTGTGACAGTGTCAGCGTGTGGGTT |
| | | GGGGATAAGACCACCGCCACAGACATCAAGGGCAAGGAG |
| | | GTGATGGTGTTGGGAGAGGTGAACATTAACAACAGTGTA |
| | | TTCAAACAGTACTTTTTTGAGACCAAGTGCCGGGACCCA |
| | | AATCCCGTTGACAGCGGGTGCCGGGGCATTGACTCAAAG |
| | | CACTGGAACTCATATTGTACCACGACTCACACCTTTGTC |
| | | AAGGCGCTGACCATGGATGGCAAGCAGGCTGCCTGGCGG |
| | | TTTATCCGGATAGATACGGCCTGTGTGTGTGTGCTCAGC |
| | | AGGAAGGCTGTGAGAAGAGCC |
| 77 | Ciliary neurotrophic | ATGGCTTTCACAGAGCATTCACCGCTGACCCCTCACCGT |
| | factor (CNTF) | CGGGACCTCTGTAGCCGCTCTATCTGGCTAGCAAGGAAG |
| | (nucleic acid | ATTCGTTCAGACCTGACTGCTCTTACGGAATCCTATGTG |
| | sequence, | AAGCATCAGGGCCTGAACAAGAACATCAACCTGGACTCT |
| | CCDS31554.1) | GCGGATGGGATGCCAGTGGCAAGCACTGATCAGTGGAGT |
| | | GAGCTGACCGAGGCAGAGCGACTCCAAGAGAACCTTCAA |
| | | GCTTATCGTACCTTCCATGTTTTGTTGGCCAGGCTCTTA |
| | | GAAGACCAGCAGGTGCATTTTACCCCAACCGAAGGTGAC |
| | | TTCCATCAAGCTATACATACCCTTCTTCTCCAAGTCGCT |
| | | GCCTTTGCATACCAGATAGAGGAGTTAATGATACTCCTG |
| | | GAATACAAGATCCCCCGCAATGAGGCTGATGGGATGCCT |
| | | ATTAATGTTGGAGATGGTGGTCTCTTTGAGAAGAAGCTG |
| | | TGGGGCCTAAAGGTGCTGCAGGAGCTTTCACAGTGGACA |
| | | GTAAGGTCCATCCATGACCTTCGTTTCATTTCTTCTCAT |
| | | CAGACTGGGATCCCAGCACGTGGGAGCCATTATATTGCT |
| | | AACAACAAGAAAATG |
| 78 | Insulin like growth | ATGGGAAAAATCAGCAGTCTTCCAACCCAATTATTTAAG |
| | factor 1 (IGF1) | TGCTGCTTTTGTGATTTCTTGAAGGTGAAGATGCACACC |
| | (nucleic acid | ATGTCCTCCTCGCATCTCTTCTACCTGGCGCTGTGCCTG |
| | sequence, | CTCACCTTCACCAGCTCTGCCACGGCTGGACCGGAGACG |
| | Isoform 2-also | CTCTGCGGGGCTGAGCTGGTGGATGCTCTTCAGTTCGTG |
| | known as IGF-1A, | TGTGGAGACAGGGGCTTTTATTTCAACAAGCCCACAGGG |
| | CCDS9091.1) | TATGGCTCCAGCAGTCGGAGGGCGCCTCAGACAGGCATC |
| | | GTGGATGAGTGCTGCTTCCGGAGCTGTGATCTAAGGAGG |
| | | CTGGAGATGTATTGCGCACCCCTCAAGCCTGCCAAGTCA |
| | | GCTCGCTCTGTCCGTGCCCAGCGCCACACCGACATGCCC |
| | | AAGACCCAGAAGGAAGTACATTTGAAGAACGCAAGTAGA |
| | | GGGAGTGCAGGAAACAAGAACTACAGGATG |
| 79 | Insulin like growth | ATGATTACACCTACAGTGAAGATGCACACCATGTCCTCC |
| | factor 1 (IGF1) | TCGCATCTCTTCTACCTGGCGCTGTGCCTGCTCACCTTC |
| | (nucleic acid | ACCAGCTCTGCCACGGCTGGACCGGAGACGCTCTGCGGG |
| | sequence, | GCTGAGCTGGTGGATGCTCTTCAGTTCGTGTGTGGAGAC |
| | Isoform 3, | AGGGGCTTTTATTTCAACAAGCCCACAGGGTATGGCTCC |
| | CCDS44960.1) | AGCAGTCGGAGGGCGCCTCAGACAGGCATCGTGGATGAG |
| | | TGCTGCTTCCGGAGCTGTGATCTAAGGAGGCTGGAGATG |
| | | TATTGCGCACCCCTCAAGCCTGCCAAGTCAGCTCGCTCT |
| | | GTCCGTGCCCAGCGCCACACCGACATGCCCAAGACCCAG |
| | | AAGGAAGTACATTTGAAGAACGCAAGTAGAGGGAGTGCA |
| | | GGAAACAAGAACTACAGGATG |
| 80 | Insulin like growth | ATGGGAAAAATCAGCAGTCTTCCAACCCAATTATTTAAG |
| | factor 1 (IGF1) | TGCTGCTTTTGTGATTTCTTGAAGGTGAAGATGCACACC |
| | (nucleic acid | ATGTCCTCCTCGCATCTCTTCTACCTGGCGCTGTGCCTG |
| | sequence, | CTCACCTTCACCAGCTCTGCCACGGCTGGACCGGAGACG |
| | Isoform 4, | CTCTGCGGGGCTGAGCTGGTGGATGCTCTTCAGTTCGTG |
| | CCDS44961.1) | TGTGGAGACAGGGGCTTTTATTTCAACAAGCCCACAGGG |
| | | TATGGCTCCAGCAGTCGGAGGGCGCCTCAGACAGGCATC |
| | | GTGGATGAGTGCTGCTTCCGGAGCTGTGATCTAAGGAGG |
| | | CTGGAGATGTATTGCGCACCCCTCAAGCCTGCCAAGTCA |
| | | GCTCGCTCTGTCCGTGCCCAGCGCCACACCGACATGCCC |
| | | AAGACCCAGAAGTATCAGCCCCCATCTACCAACAAGAAC |
| | | ACGAAGTCTCAGAGAAGGAAAGGAAGTACATTTGAAGAA |
| | | CGCAAG |
| 81 | Insulin like growth | ATGGGAATCCCAATGGGGAAGTCGATGCTGGTGCTTCTC |
| | factor 2 (IGF2) | ACCTTCTTGGCCTTCGCCTCGTGCTGCATTGCTGCTTAC |
| | (nucleic acid | CGCCCCAGTGAGACCCTGTGCGGCGGGGAGCTGGTGGAC |
| | sequence, | ACCCTCCAGTTCGTCTGTGGGGACCGCGGCTTCTACTTC |
| | Isoform 1, | AGCAGGCCCGCAAGCCGTGTGAGCCGTCGCAGCCGTGGC |
| | CCDS7728.1) | ATCGTTGAGGAGTGCTGTTTCCGCAGCTGTGACCTGGCC |
| | | CTCCTGGAGACGTACTGTGCTACCCCCGCCAAGTCCGAG |
| | | AGGGACGTGTCGACCCCTCCGACCGTGCTTCCGGACAAC |
| | | TTCCCCAGATACCCCGTGGGCAAGTTCTTCCAATATGAC |
| | | ACCTGGAAGCAGTCCACCCAGCGCCTGCGCAGGGGCCTG |
| | | CCTGCCCTCCTGCGTGCCCGCCGGGGTCACGTGCTCGCC |
| | | AAGGAGCTCGAGGCGTTCAGGGAGGCCAAACGTCACCGT |
| | | CCCCTGATTGCTCTACCCACCCAAGACCCCGCCCACGGG |
| | | GGCGCCCCCCCAGAGATGGCCAGCAATCGGAAG |
| 82 | Insulin like growth | ATGGTTTCCCCAGACCCCCAAATTATCGTGGTGGCCCCC |
| | factor 2 (IGF2) | GAGACCGAACTCGCGTCTATGCAAGTCCAACGCACTGAG |
| | (nucleic acid | GACGGGGTAACCATTATCCAGATATTTTGGGTGGGCCGC |
| | sequence, | AAAGGCGAGCTACTTAGACGCACCCCGGTGAGCTCGGCC |
| | Isoform 3, | ATGCAGACACCAATGGGAATCCCAATGGGGAAGTCGATG |
| | CCDS44517.1) | CTGGTGCTTCTCACCTTCTTGGCCTTCGCCTCGTGCTGC |
| | | ATTGCTGCTTACCGCCCCAGTGAGACCCTGTGCGGCGGG |
| | | GAGCTGGTGGACACCCTCCAGTTCGTCTGTGGGGACCGC |
| | | GGCTTCTACTTCAGCAGGCCCGCAAGCCGTGTGAGCCGT |
| | | CGCAGCCGTGGCATCGTTGAGGAGTGCTGTTTCCGCAGC |
| | | TGTGACCTGGCCCTCCTGGAGACGTACTGTGCTACCCCC |
| | | GCCAAGTCCGAGAGGGACGTGTCGACCCCTCCGACCGTG |
| | | CTTCCGGACAACTTCCCCAGATACCCCGTGGGCAAGTTC |
| | | TTCCAATATGACACCTGGAAGCAGTCCACCCAGCGCCTG |
| | | CGCAGGGGCCTGCCTGCCCTCCTGCGTGCCCGCCGGGGT |
| | | CACGTGCTCGCCAAGGAGCTCGAGGCGTTCAGGGAGGCC |
| | | AAACGTCACCGTCCCCTGATTGCTCTACCCACCCAAGAC |
| | | CCCGCCCACGGGGGCGCCCCCCCAGAGATGGCCAGCAAT |
| | | CGGAAG |

### Neuropoietic Cytokines

Therapeutic agents that may be incorporated into the compositions and methods described herein include neuropoietic cytokines, such as interleukin-6, interleukin-11, inteleukin-27, leukemia inhibitory factor, CNTF, cardiotrophin 1, neuropoietin, card iotrophin-like cytokine, and fibroblast growth factor 2.

### Anti-Inflammatory Cytokines

Therapeutic agents that may be incorporated into the compositions and methods described herein include anti-inflammatory cytokines. Examples of anti-inflammatory cytokines useful in conjunction with the compositions and methods described herein are interleukin-4 and interleukin-10.

### Neuroprotection Agents

Therapeutic agents that may be incorporated into the compositions and methods described herein include neuroprotection agents. For example, the pharmaceutical compositions described herein may contain a neuroprotection agent selected from neuregulin-1, vascular endothelial growth factor (VEGF), sodium thiosulfate, and N-acetyl cysteine.

### TGFβ Superfamily Member Proteins

Therapeutic agents that may be incorporated into the compositions and methods described herein include TGFβ superfamily member proteins. For example, the pharmaceutical compositions described herein may contain a TGFβ-superfamily protein selected from TGFβ, TGFβ3, BMP2, and BMP7.

### Immunomodulating Agents

Therapeutic agents that may be incorporated into the compositions and methods described herein include immunomodulating agents. For example the pharmaceutical compositions described herein may contain an immunomodulating agent selected from the group including anti-TNF agents (e.g., anti-TNF antibodies, such as infliximab. adalimumab, and golimumab; fusion proteins, such as etanercept; TACE inhibitors; IKK inhibitors; or calcineurin inhibitors) and toll-like receptor inhibitors.

### Viral Vectors

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors that may be incorporated into the pharmaceutical compositions described herein are adeno-associated viral (AAV) vectors (e.g., AAV1, AAV2, AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, and PHP.S), retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other examples of viral vectors that may be used in conjunction with the compositions and methods described herein are Norwalk virus, togavirus, flavivirus, reoviruses, papovavirus, hepadnavirus, and hepatitis virus, among others. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### Antibodies and Antigen-Binding Fragments Thereof.

In some embodiments, the therapeutic agent is an antibody or antigen-binding fragment thereof, such as an antibody or antigen-binding fragment thereof that binds TrkB, TrkC, or a receptor thereof. The antibody or antigen-binding fragment thereof may be, for example, a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFv.

Exemplary TrkB- and TrkC-binding antibodies useful in conjunction with the compositions and methods described herein are those produced by the cell lines shown in Table 4, below. Other TrkB- and TrkC-binding antibodies that may be used in conjunction with the compositions and methods described herein are recited in WO 2017/019907.

TABLE 4
Examples of hybridoma lines producing TrkB- and
TrkC-binding antibodies

| Antibody | ATCC Deposit No. |
|---|---|
| 38B8 | PTA-8766 |
| 2B7 | 090310-02 |
| A5, light chain | PTA-5682 |
| A5, heavy chain | PTA-5683 |
| 6.1.2 | PTA-2148 |
| 6.4.2 | PTA-2150 |
| 2345 | PTA-2146 |
| 2349 | PTA-2153 |
| 2.5.1 | PTA-2151 |
| 2344 | PTA-2144 |
| 2248 | PTA-2147 |
| 2250 | PTA-2149 |
| 2253 | PTA-2145 |
| 2256 | PTA-2152 |

### Additional Agents

Therapeutic agents that may be incorporated into the pharmaceutical compositions described herein further include liposomes, vesicles, synthetic vesicles, exosomes, synthetic exosomes, dendrimers, and nanoparticles. Additional examples of therapeutic agents that may be used in conjunction with the compositions and methods described herein are small molecules, such as those that are not naturally round window membrane-penetrant. Further examples of such therapeutic agents are interfering RNA molecules, such as a short interfering RNA (siRNA), a short hairpin RNA (shRNA), and a micro RNA (miRNA). Exemplary interfering RNA molecules are those at are at least 85% complementary to, and/or that anneal to, a target nucleic acid of interest, thereby suppressing the expression of the target nucleic acid.

Additional examples of therapeutic agents that may be incorporated into the compositions and methods described herein are, without limitation, antimicrobial agents, an arylcycloalkylamine, an elipticine derivative, anti-apoptotic agents, c-JNK inhibitors, antioxidants, NSAIDs, analgesics, neuroprotection agents, glutamate modulators, interleukin 1 modulators, interleukin-1 antagonists, corticosteroids, anti-TNF agents, calcineurin Inhibitors, IKK inhibitors, Interleukin inhibitors, platelet activating factor antagonists, TNF-α converting enzyme (TACE) inhibitors, Toll-like receptor inhibitors, autoimmune agents, IL-1 modulators, RNA interference agents, aquaporin modulators, estrogen-related receptor beta modulators, GAP junction proteins, vasopressin receptor modulators, NMDA receptor modulators, ENaC receptor modulators, osmotic diuretics, progesterone receptors, prostaglandins, cytotoxic agents, cytoprotective agents, anti-intercellular adhesion molecule-1 antibody, Atohi modulators (e.g., an Atohi polypeptide or a nucleic acid vector engineered to express Atohi, e.g., human Atohi (Hathi)), Mathi modulators, BRN-3 modulators, carbamates, estrogen receptors, fatty acids, gamma-secretase inhibitors, glutamate-receptor modulators, a neurotrophic agent, salicylic acid, nicotine, retinoblastoma protein modulators, ion channel blockers, thyroid hormone receptor modulators, TRPV modulators, adenosine modulators, KCNQ modulators, P2X modulators, CNS modulating agents, anticholinergics, antihistamines, GABA receptor modulators, neurotransmitter reuptake inhibitors, thyrotropin-releasing hormones, free radical modulators, metal atom chelators, mitochondrial modulators, nitric oxide synthase modulators, sirtuin modulators, purinergic receptor modulators, nucleic acid polymer antagonists, small molecule antagonists, polypeptide antagonists, a neurotrophin variant, a WNT modulator, a protein kinase C beta modulator, a repulsive guidance molecule a (RGMa) inhibitor, a neogenin inhibitor, a SK2 channel activator, a BK channel activator, a sphingosine-1-phosphate receptor modulator, a stemness driver, a differentiation inhibitor, an N-Methyl-D-Aspartate (NMDA) receptor antagonist, a histone deacetylase (HDAC) inhibitor, a glycogen synthase kinase inhibitor (e.g., GSK3β and/or GSK3α inhibitor), a proteasome inhibitor, an EZH2/HMT inhibitor, a notch inhibitor, ebselen, ancrod, an α-amino-3-hydroxy-5-methyl-4-isoxazolepropionic acid (AMPA) glutamate-positive allosteric modulator, D-methionine, an antagonist of histamine type 4 receptors, a chemotherapeutic accumulation reducer, choline ester, plant alkaloid, reversible cholinesterase inhibitor, acetylcholine release promoter, anti-adrenergy, a sympathomimetic, an antineoplastic agent, R(+)-N-propargyl-1-aminoindan, and R-azasetron besylate.

Other therapeutic agents that may be used in conjunction with the compositions and methods described herein are recited in WO 2017/019907 and WO 2018/005830, the disclosures of which are incorporated herein by reference.

### Methods of Treatment

The pharmaceutical compositions described herein may be administered to a subject (e.g., a mammalian subject, such as a human patient) in order to treat and/or prevent one or more of a variety of pathologies. For example, the pharmaceutical compositions described herein may be used to deliver a therapeutic agent across the round window membrane of a subject (e.g., a mammalian subject, such as a human patient) so as to treat an otic disease, such as ceruminosis or ceruminosis associated with an otic disease or condition, ear pruritus, otitis externa, otalgia, tinnitus, vestibular dysfunction (e.g., vertigo, dizziness, or loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), auto immune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

Subjects that may be treated as described herein are subjects having or at risk of developing sensorineural hearing loss, deafness, auditory neuropathy, tinnitus, and/or vestibular dysfunction (e.g., subjects having or at risk of developing hearing loss, vestibular dysfunction, or both). The pharmaceutical compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing damage to spiral ganglion neurons (SGNs) (e.g., damage related to acoustic trauma, disease or infection, head trauma, ototoxic drugs, or aging), subjects having or at risk of developing SGN degeneration, subjects having or at risk of developing sensorineural hearing loss, deafness, auditory neuropathy, or tinnitus, subjects having or at risk of developing vestibular dysfunction (e.g., dizziness, vertigo, or imbalance), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the subject has hearing loss and/or vestibular dysfunction that is associated with or results from damage to or loss of hair cells (e.g., cochlear or vestibular hair cells) or SGNs. The methods described herein may include a step of screening a subject for one or more mutations in genes known to be associated with hearing loss or vestibular dysfunction prior to treatment with or administration of the pharmaceutical compositions described herein. A subject can be screened for a genetic mutation using standard methods known to those of skill in the art (e.g., genetic testing). The methods described herein may also include a step of assessing hearing and/or vestibular function in a subject prior to treatment with or administration of the pharmaceutical compositions described herein. Hearing can be assessed using standard tests, such as audiometry, auditory brainstem response (ABR), electrochocleography (ECOG), and otoacoustic emissions. Vestibular function may be assessed using standard tests, such as eye movement testing (e.g., electronystagmogram (ENG) or videonystagmogram (VNG)), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the pharmaceutical compositions described herein. The pharmaceutical compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction or patients exposed to risk factors for acquired hearing loss (e.g., acoustic trauma, disease or infection, head trauma, ototoxic drugs, or aging) or vestibular dysfunction (e.g., disease or infection, head trauma, ototoxic drugs, or aging).

The pharmaceutical compositions and methods described herein can be used to promote or induce hair cell regeneration in a subject (e.g., cochlear and/or vestibular hair cell regeneration) and/or SGN regeneration in a subject. The pharmaceutical compositions and methods described herein can also be used to increase the number of supporting cells in subject (e.g., cochlear and/or vestibular supporting cells, e.g., increase supporting cell proliferation). Subjects that may benefit from pharmaceutical compositions that promote or induce hair cell regeneration and/or SGN regeneration, or that increase the number of supporting cells, include subjects suffering from hearing loss or vestibular dysfunction as a result of loss of hair cells and/or SGNs (e.g., loss of hair cells and/or SGNs related to trauma (e.g., acoustic trauma or head trauma), disease or infection, ototoxic drugs, or aging), and subjects with abnormal hair cells and/or SGNs (e.g., hair cells and/or SGNs that do not function properly when compared to normal hair cells), damaged hair cells and/or SGNs (e.g., hair cell and/or SGN damage related to trauma (e.g., acoustic trauma or head trauma), disease or infection, ototoxic drugs, or aging), or reduced hair cell and/or SGN numbers due to genetic mutations or congenital abnormalities. The pharmaceutical compositions and methods described herein can also be used to promote or increase hair cell survival and/or SGN survival (e.g., increase survival of damaged hair cells and/or SGNs, promote repair of damaged hair cells and/or SGNs, or preserve hair cells and/or SGNs in a subject at risk of loss of hair cells and/or SGNs (e.g., loss of hair cells and/or SGNs due to age, exposure to loud noise, disease or infection, head trauma, or ototoxic drugs)).

The pharmaceutical compositions and methods described herein can also be used to prevent or reduce hair cell and/or SGN damage or death (e.g., cochlear hair cell and/or vestibular hair cell damage or death and/or SGN damage or death). In some embodiments, the methods prevent or reduce ototoxic drug-induced hair cell and/or SGN damage or death. In some embodiments, the methods prevent or reduce ototoxic drug-induced hair cell and/or SGN damage or death in subjects who have been treated with ototoxic drugs, or who are currently undergoing or soon to begin treatment with ototoxic drugs. Ototoxic drugs are toxic to the cells of the inner ear (e.g., hair cells and SGNs), and can cause sensorineural hearing loss, vestibular dysfunction (e.g., vertigo, dizziness, or imbalance), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods described herein prevent or reduce hair cell and/or SGN damage or death (e.g., cochlear hair cell and/or vestibular hair cell damage or death and/or SGN damage or death) related to acoustic trauma, disease or infection, head trauma, or aging.

Particular embodiments of the pharmaceutical composition that can be used to treat subjects having or at risk of developing sensorineural hearing loss, tinnitus, deafness, auditory neuropathy, and/or vestibular dysfunction, promote or induce hair cell and/or SGN regeneration, or prevent or reduce hair cell and/or SGN damage or death (e.g., ototoxic drug-induced hair cell and/or SGN damage or death, noise-induced hair cell and/or SGN damage or death, or age-related hair cell and/or SGN damage or death), promote hair cell regeneration, or increase the number of supporting cells, include those in which the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, a neuroprotection agent, an Atoh1 modulator (e.g., an Atoh1 polypeptide or a nucleic acid vector engineered to express Atoh1, e.g., human Atoh1 (Hath1)), or a WNT modulator. For example, pharmaceutical compositions that are particularly suited for these indications are those in which the therapeutic agent is a neurotrophin selected from NT-3, NGF, BDNF, CNTF, GDNF, NT-4, FGF, IGF, EGF, PGF, MANF, CDNF, a pan-neurotrophin (e.g., PNT-1), a chimeric neurotrophin (e.g., an NGF/BDNF chimera), or a combination thereof.

The pharmaceutical compositions described herein are administered in an amount sufficient to improve hearing, improve vestibular function (e.g., improve balance or reduce dizziness or vertigo), treat, prevent, reduce or slow the development of tinnitus, treat, prevent, reduce, or delay the development of hearing loss, slow the progression of hearing loss, treat, prevent, reduce, or delay the development of vestibular dysfunction, slow the progression of vestibular dysfunction, prevent or reduce hair cell damage (e.g., hair cell damage related to acoustic trauma, head trauma, ototoxic drugs, disease or infection, or aging), prevent, slow, or reduce hair cell death (e.g., ototoxic drug-induced hair cell death, noise-related hair cell death, age-related hair cell death, disease or infection-related hair cell death, or head trauma-related hair cell death), promote or increase hair cell development, increase hair cell numbers (e.g., promote or induce hair cell regeneration), increase supporting cell numbers (e.g., promote supporting cell proliferation), promote or increase hair cell survival, improve hair cell function, prevent or reduce SGN damage, prevent, slow, or reduce SGN death (e.g., ototoxic drug-induced SGN death or age-related SGN death), promote or increase SGN development, increase SGN numbers (e.g., promote or induce SGN) regeneration, increase or promote SGN survival, promote SGN repair, improve SGN function, preserve ribbon synapses, promote or increase ribbon synapse formation, maintain the connections (e.g., synaptic connections) between hair cells and SGNs, or increase or restore the connections (e.g., synaptic connections) between hair cells and SGNs. Hearing may be evaluated using standard hearing tests (e.g., audiometry, ABR, electrochocleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. Vestibular function may be evaluated using standard tests for balance and vertigo (e.g., eye movement testing (e.g., ENG or VNG), posturography, rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. In some embodiments, the pharmaceutical compositions are administered in an amount sufficient to improve the subject's ability to understand speech. The pharmaceutical compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, disease or infection, or acoustic trauma) but do not exhibit hearing impairment or vestibular dysfunction (e.g., vertigo, dizziness, or imbalance), or in subjects exhibiting mild to moderate hearing loss or vestibular dysfunction). Hair cell numbers, hair cell function, SGN numbers, or SGN function may be evaluated indirectly based on hearing tests or tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell numbers, hair cell function, SGN numbers, or SGN function prior to administration of the pharmaceutical compositions described herein. Hair cell damage or death and/or SGN damage or death may be reduced by 5% or more (e.g., 5%, 10%, 15%, 20%, 30%, 40%, 50%, 60%, 70%, 80%, 90%, 100%, 125%, 150%, 200% or more) compared to hair cell damage or death and/or SGN damage or death typically observed in untreated subjects, and can be evaluated indirectly based on standard hearing tests. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the pharmaceutical compositions described herein. The patient may be evaluated 1 month, 2 months, 3 months, 4 months, 5 months, 6 months or more following administration of the pharmaceutical composition depending on the dose and route of administration used for treatment. Depending on the outcome of the evaluation, the patient may receive additional treatments.

### Routes of Administration and Dosing

The pharmaceutical compositions described herein may be administered to a subject (e.g., a mammalian subject, such as a human) by way of one or more of a variety of routes. Examples of routes of administration that may be used in conjunction with the compositions and methods described herein are local administration to the inner ear (e.g., administration into the perilymph or endolymph, e.g., through or to the oval window, round window, semicircular canal, or horizontal canal), as well as intratympanic and transtympanic administration. Additional examples of routes of administration that may be used in conjunction with the compositions and methods described herein are intravenous administration, parenteral administration, intradermal administration, transdermal administration, intramuscular administration, intranasal administration, subcutaneous administration, percutaneous administration, intratracheal administration, intraperitoneal administration, intraarterial administration, intravascular, inhalation, perfusion, lavage, and oral administration. The chosen route of administration will depend on the particular composition administered, the subject, pharmaceutical formulation methods, administration methods (e.g., administration time and administration route), the subject's age, body weight, gender, severity of the disease being treated, the subject's diet, and the subject's metabolism. Compositions may be administered once, or more than once (e.g., once annually, twice annually, three times annually, bi-monthly, or monthly).

In particular embodiments, the pharmaceutical composition is administered intratympanically or transtympanically. Transtympanic administration may include injection or infusion of an effective amount of the pharmaceutical composition of the invention through the tympanic membrane into the tympanic cavity using a hypodermic needle, thereby providing compound to the round window. In some cases, in order to prevent pressure-based damage to the round window, the round window may first be punctured, e.g., using a hypodermic needle or surgical laser, to vent any gas buildup behind the round window.

In some embodiments, at least 50 μL (preferably, at least 100 μL; more preferably, at least 200 μL) of the pharmaceutical composition are administered to the round window of the subject. In particular embodiments, 1 mL or less (e.g., 0.8 mL or less or 0.5 mL or less) of the pharmaceutical composition are administered to the round window of the subject. In certain embodiments, 50 μL to 1 mL (e.g., 100 μL to 1 mL, 200 μL to 1 mL, 100 μL to 0.8 mL, 200 μL to 0.8 mL, 100 μL to 0.5 mL, 200 μL to 0.5 mL, 0.5 mL to 1.0 mL, 0.5 mL to 0.8 mL, or 0.8 mL to 1.0 mL) of the pharmaceutical composition are administered to the round window of the subject.

In some embodiments, the pharmaceutical composition is configured to control the release profile of the therapeutic agent. In general, the release profile of the therapeutic agent is controlled in part by the physical and chemical interactions of the agent with the other components of the composition, e.g., pH, solubility, hydration, complexation, and diffusivity. The pharmaceutical composition may allow for the immediate release of the therapeutic agent. In some embodiments, the pharmaceutical composition may allow for sustained release of the therapeutic agent. For example, the release of the therapeutic agent may be sustained from about 1 day to about 6 weeks, or more (e.g., for about 1 day, about 2 days, about 3 days, about 4 days, about 5 days, about 6 days, about 7 days, about 8 days, about 9 days, about 10 days, about 11 days, about 12 days, about 13 days, 14 days, 21 days, 28 days, 35 days, 42 days, 48 days, or more). The sustained release of the therapeutic agent may occur in a continuous matter, pulsatile manner, or a combination of both. In addition, the pharmaceutical composition may be configured for both an immediate release and controlled release of the therapeutic agent, and may further be dependent on the environmental or physiological conditions of the administration.

### Other Components

The pharmaceutical compositions described herein may contain pharmaceutically acceptable diluents, carriers, and/or excipients. For example, the pharmaceutical compositions described herein may contain, e.g., liquid solvents, buffering agents, viscosity agents, and/or coloring agents. Certain excipients may perform multiple roles. For example, a liquid solvent, in addition to its function as a carrier, may be used as a buffering agent. Such solvents are known in the art, e.g., salines (e.g., hypertonic saline, hypotonic saline, isotonic saline, or phosphate-buffered saline) and artificial perilymph.

Liquid solvents that may be used in conjunction with the pharmaceutical compositions described herein include water, mineral oil, salines (e.g., hypertonic saline, hypotonic saline, isotonic saline, or phosphate-buffered saline), artificial perilymph, and tris buffer. Artificial perilymph is an aqueous solution containing NaCl (120-130 mM), KCl (3.5 mM), CaCl₂ (1.3-1.5 mM), MgCl₂ (1.2 mM), glucose (5.0-11 mM), and buffering agents (e.g., NaHCO₃(25 mM) and NaH₂PO₄ (0.75 mM), or HEPES (20 mM) and NaOH (adjusted to pH of about 7.5)).

Buffering agents may be used to adjust the pH of a pharmaceutical composition (e.g., a pharmaceutical dosage form) of the invention a substantially neutral pH level. Examples of buffering agents that may be used in conjunction with the compositions and methods described herein are known in the art, and include, without limitation, phosphate buffers and Good's buffers (e.g., tris, MES, MOPS, TES, HEPES, HEPPS, tricine, and bicine). In addition to regulating pH, buffering agents may be used to control the osmolarity of the pharmaceutical composition.

Viscosity agents can be used to increase or decrease the dynamic viscosity of the pharmaceutical composition prior to administration or to control the dynamic viscosity post administration. Viscosity agents may further control the release profile of the compound of the pharmaceutical composition. Examples of viscosity agents that may be used in conjunction with the compositions and methods described herein are known in the art and include, without limitation, sodium stearate, bladderwrack, bentonite, eratonia, chondrus, dextrose, furcellaran, Ghatti gum, hectorite, lactose, sucrose, sucralose, maltodextrin, mannitol, sorbitol, honey, cellulose and its derivatives (e.g., ethyl cellulose, ethylhydroxyethyl cellulose, ethylmethyl cellulose, methyl cellulose, hydroxyethyl cellulose, hydroxyethylmethyl cellulose, hydroxypropyl cellulose, hydroxypropyl cellulose, hydroxypropylmethyl-cellulose (HPMC), sodium carboxymethyl-cellulose (CMC)), pectin, chondroitin sulfate, or a combination thereof.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. Identification of a Class of Round Window Membrane-Penetrant Peptides

The delivery of therapeutic agents across the round window membrane poses a challenge, particularly for the passage of high-molecular weight substances. This challenge is evidenced, for example, by the difficulty involved in transporting neurotrophin-3 (NT-3) across the round window membrane. NT-3 is a non-covalent homodimer protein of about 27 kDa. Each chain of NT-3 has three disulfide bonds. The protein has a pl of 9.5, and is positively charged at physiological pH. Although the protein has an EC₅₀ of less than 20 ng/ml, the protein experiences difficulty in penetrating the round window membrane. The experiments described in this example illustrate the identification of a permeation-enhancing peptide, represented by formula (XVI), capable of inducing the passage of NT-3 across the round window membrane and into the inner ear.

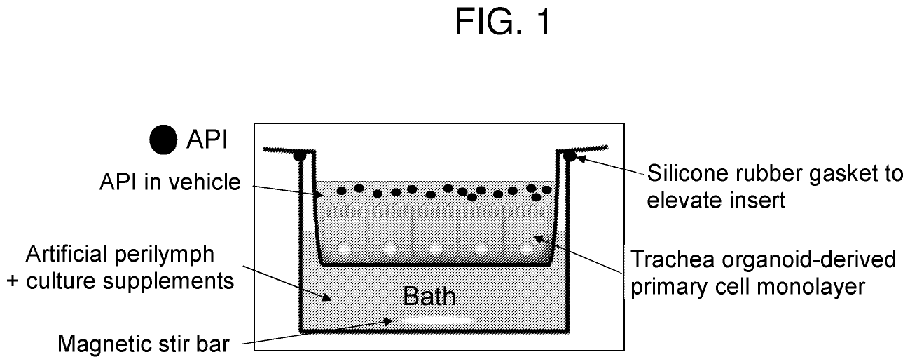

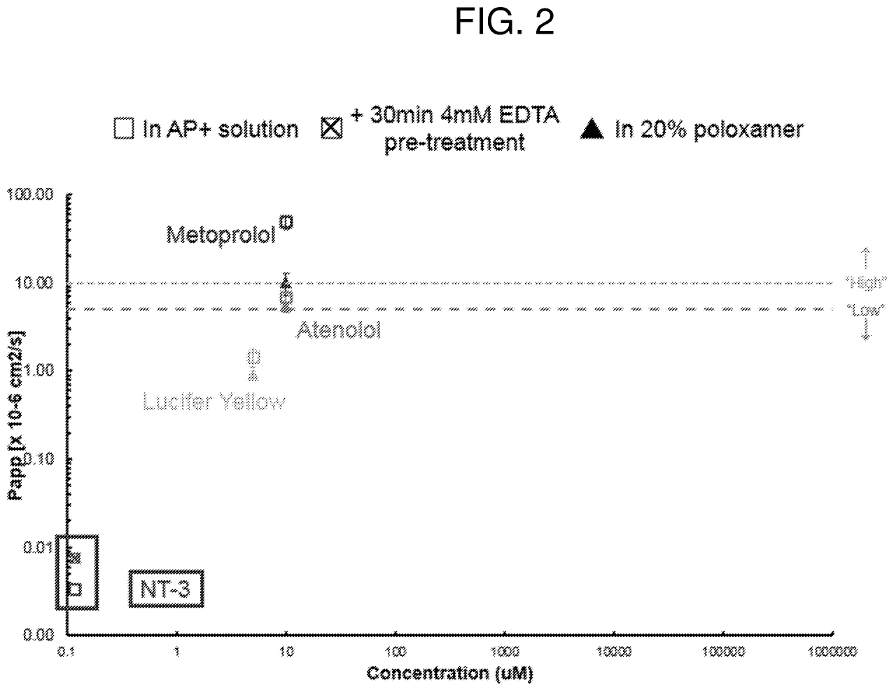

To this end, an in vitro permeability assay was designed to test the apparent permeability of NT-3 against that of other compounds, the experimental design of which is shown in FIG. 1. After the initial formation of tight junctions, which was evaluated using transepithelial electrical resistance (TEER), 100 μL artificial perilymph (AP+) with API was added to the insert, and 500 μL AP+ was added to the bath. Cultures were incubated at 37° C. and 20 μL samples were collected at various time points. As evidenced by the results shown in FIG. 2, NT-3 is poorly round window membrane-penetrant.

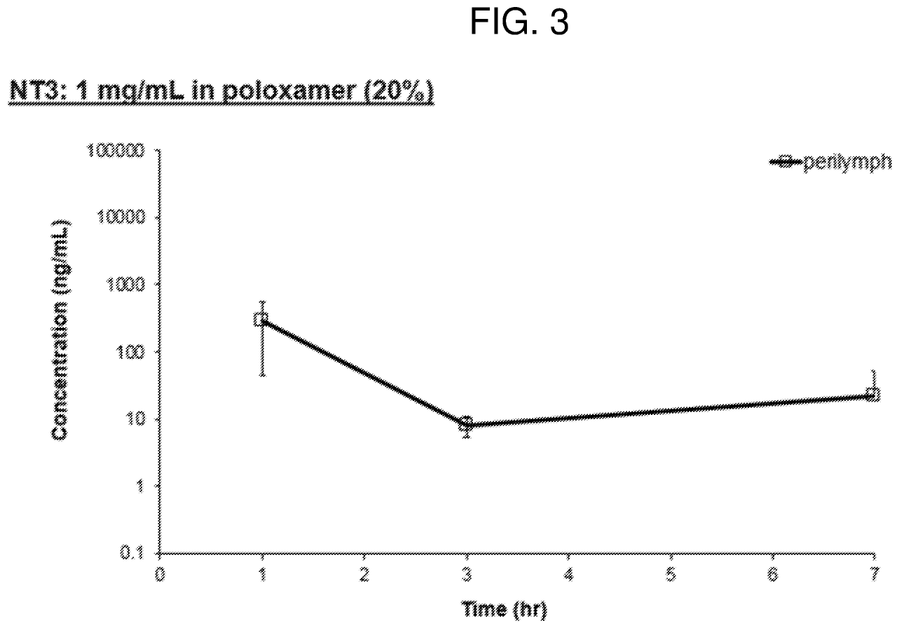

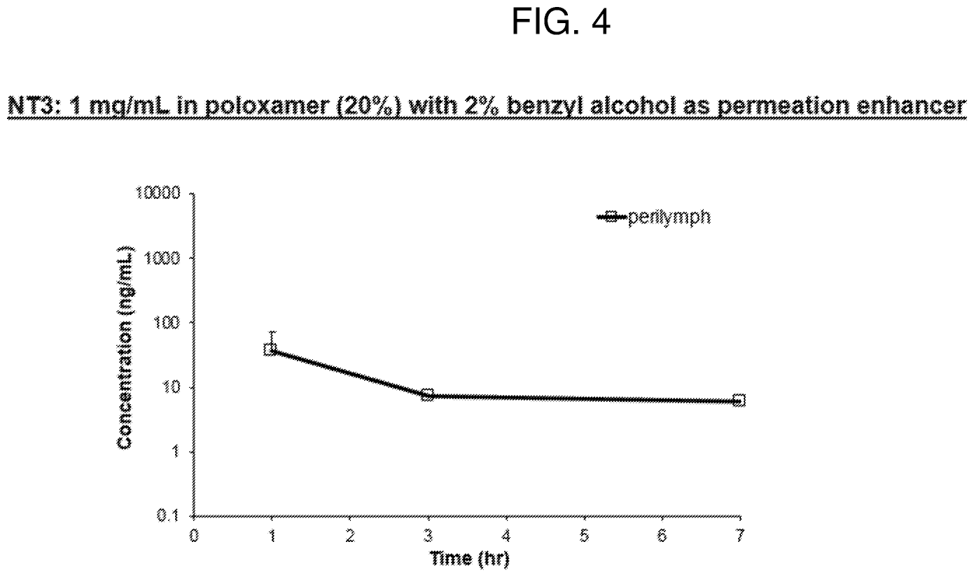

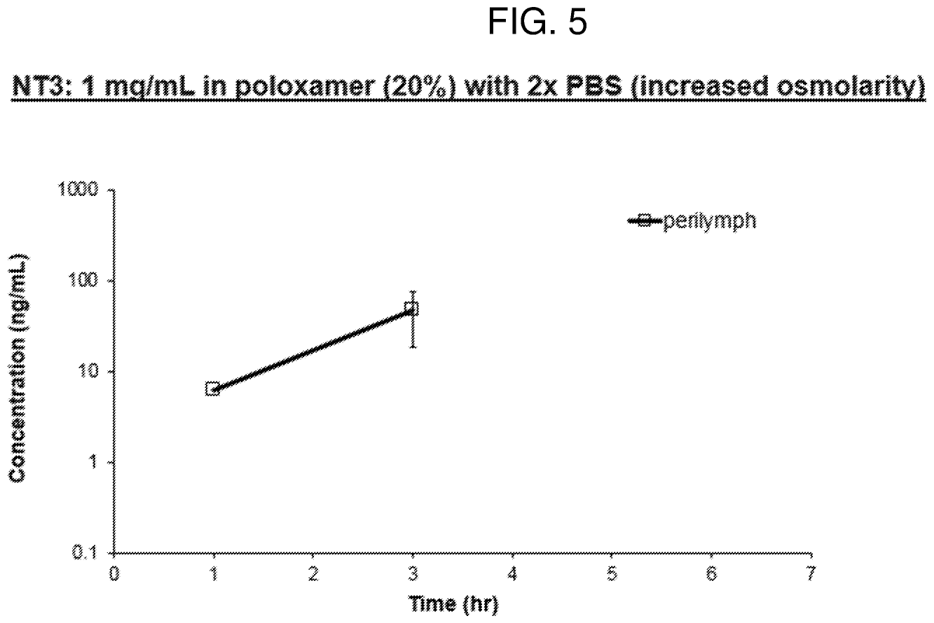

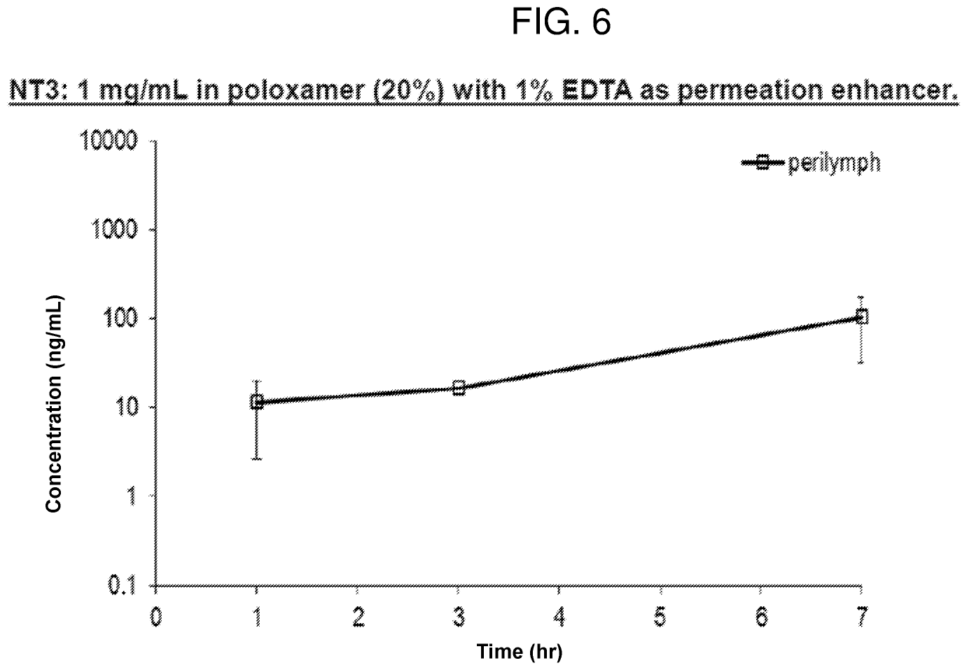

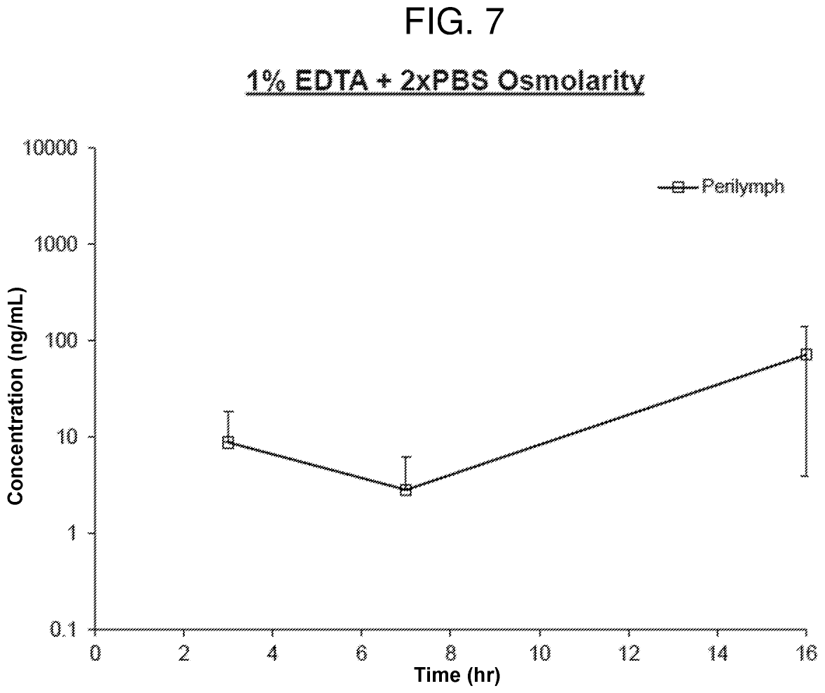

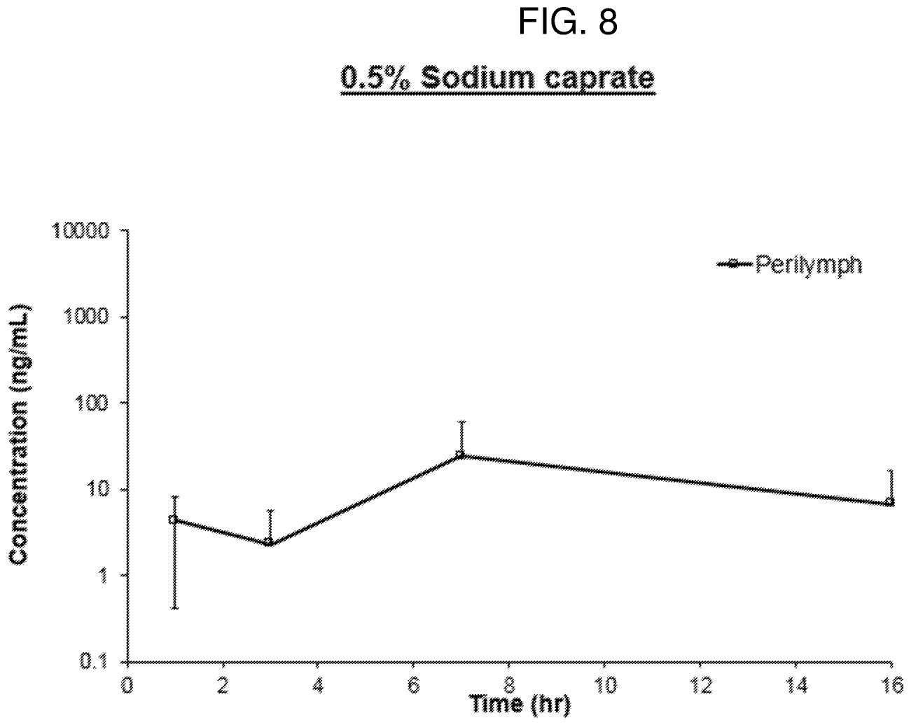

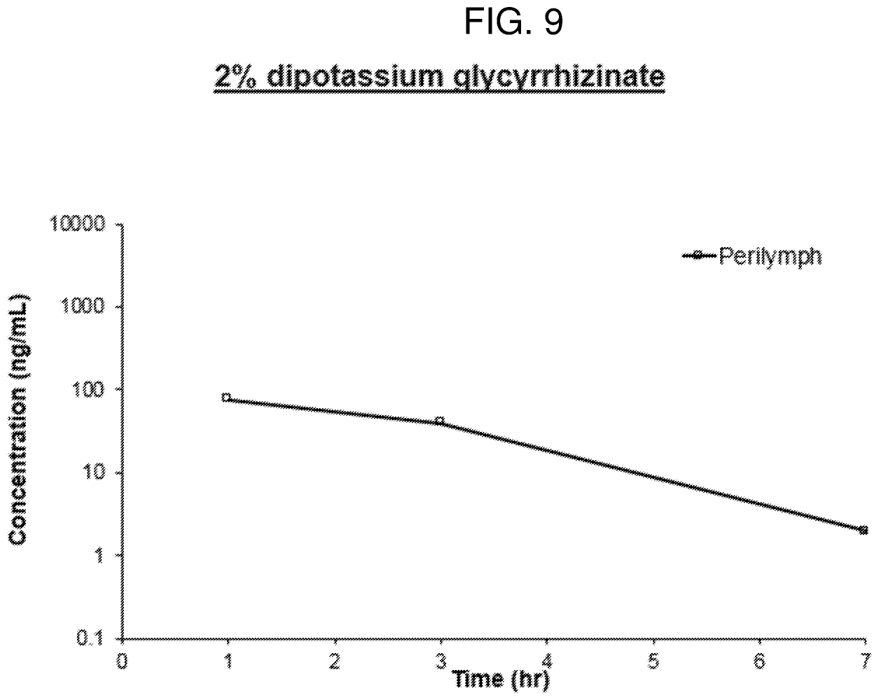

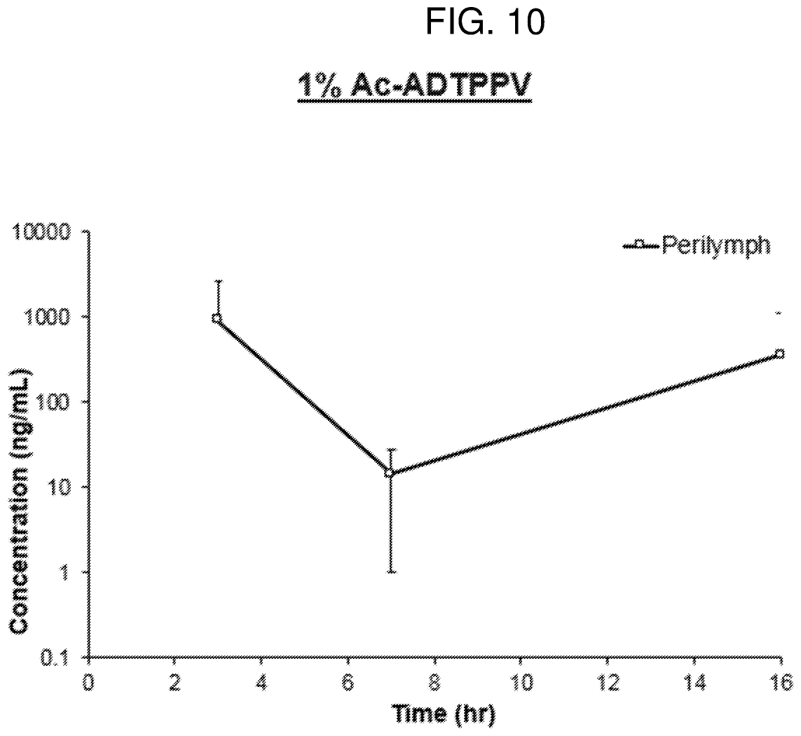

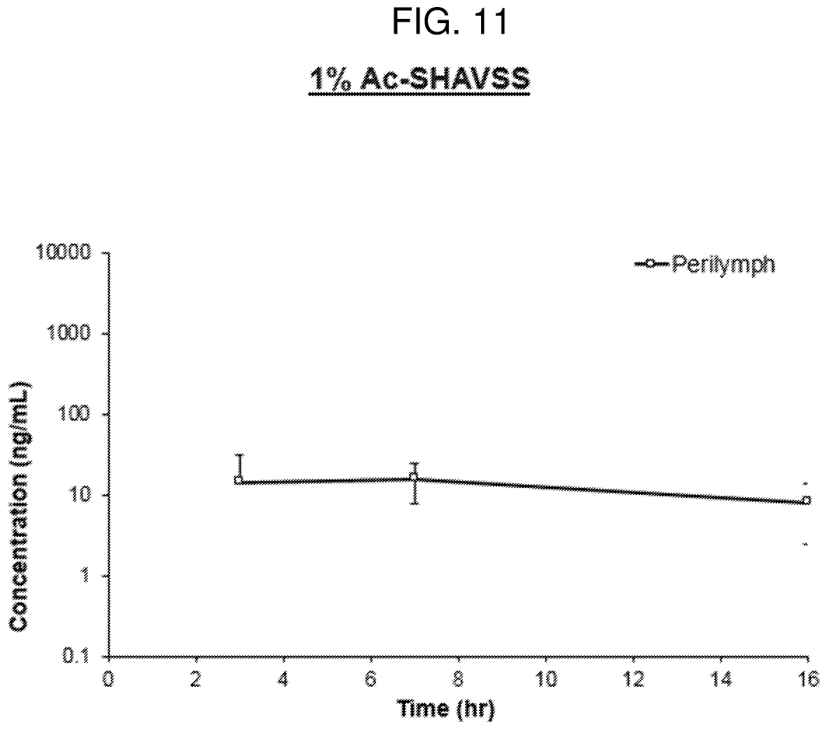

As shown in FIGS. 3A and 3B, only trace cochlear exposure of NT-3 was observed in the perilymph of n-5 guinea pig subjects upon intratympanic administration of 1 mg/ml NT-3 in poloxamer (20% w/v). Moreover, a wide variety of permeation enhancers, including benzyl alcohol, sodium caprate, dipotassium glycyrrhizinate, elevated osmolarity, and short polypeptides were incapable of substantially improving NT-3 delivery across the round window membrane upon intratympanic administration to this model organism (FIGS. 4-11).

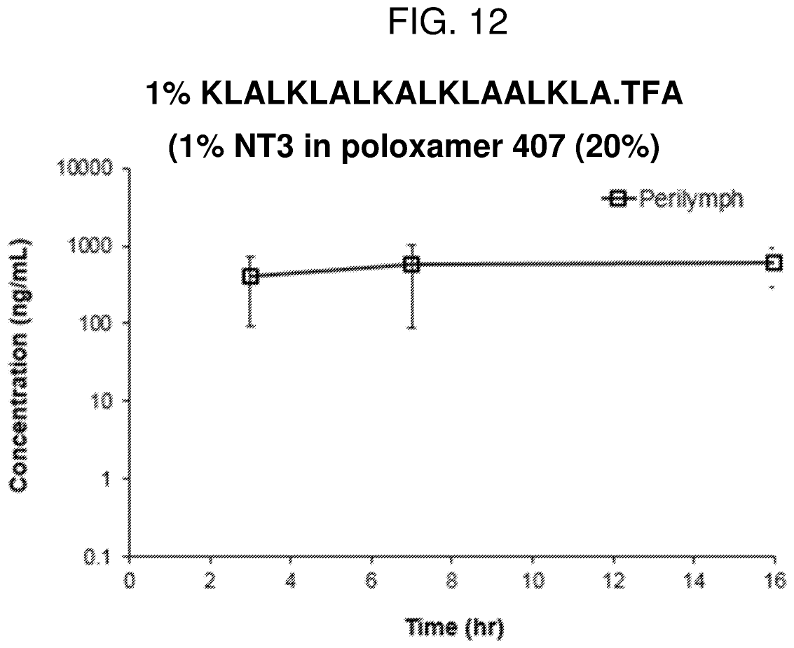

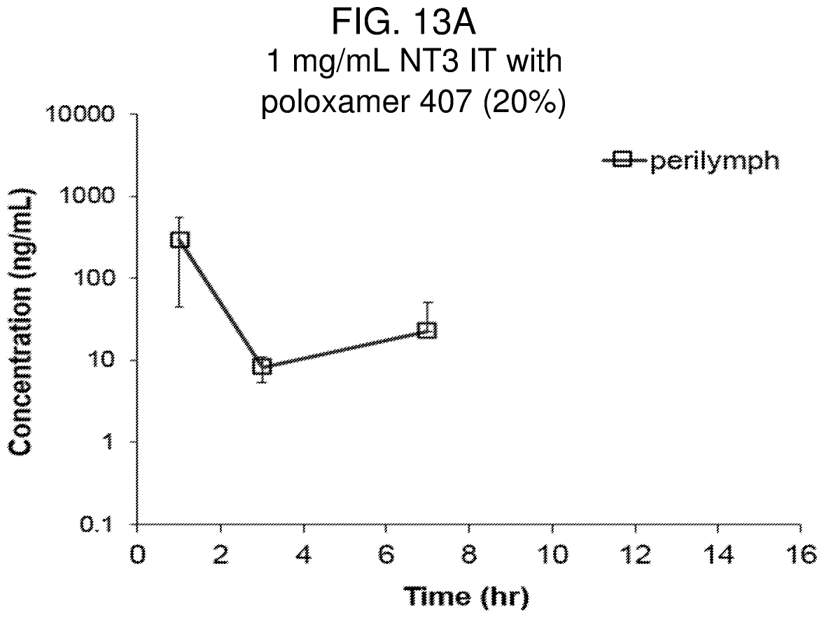

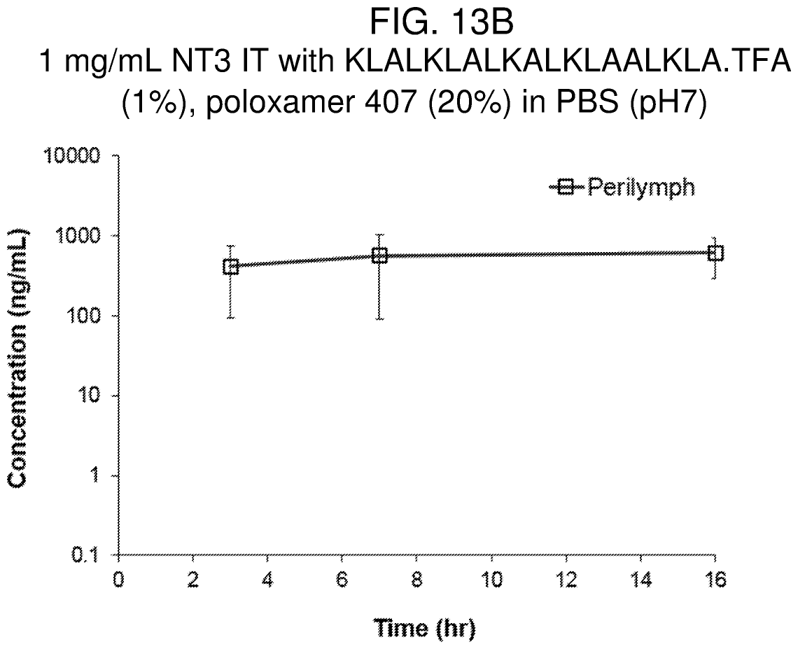

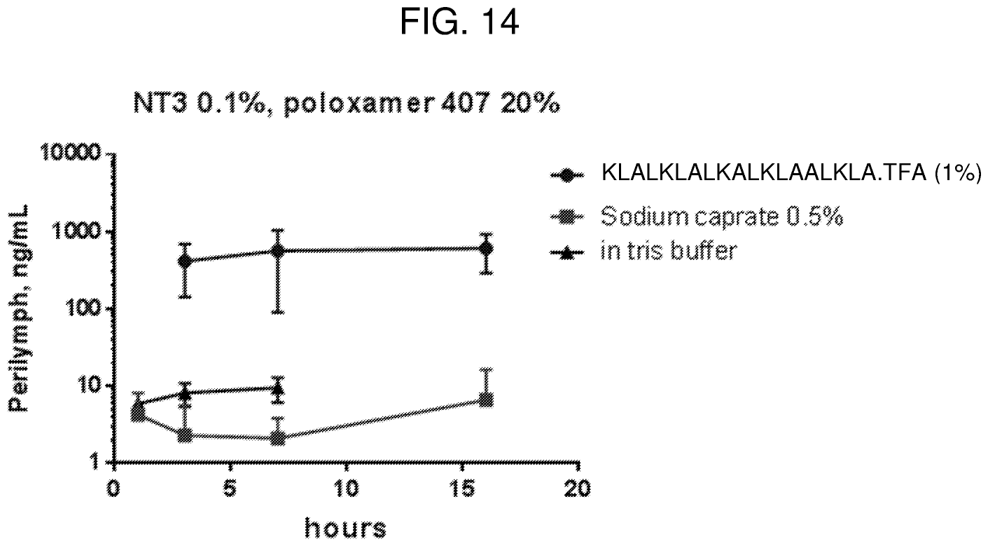

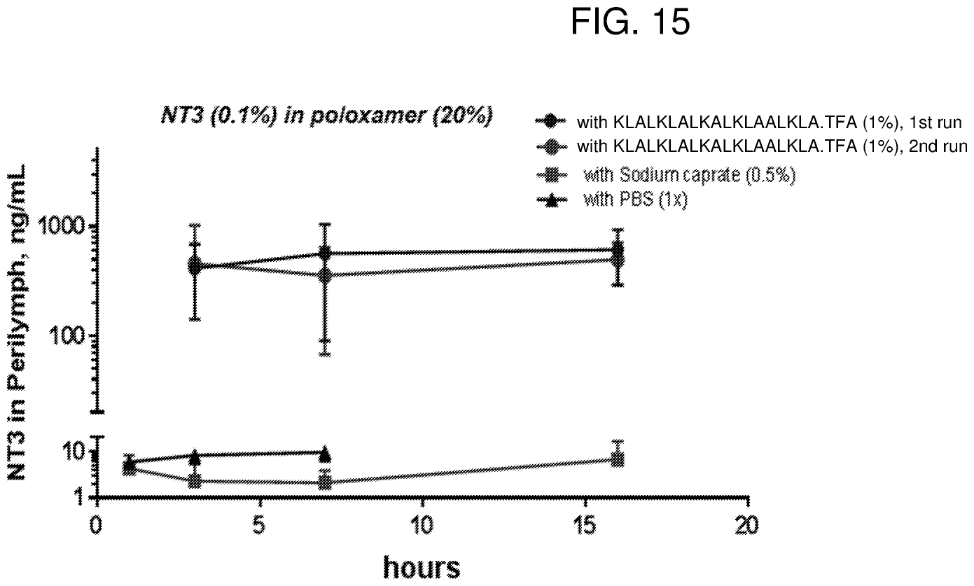

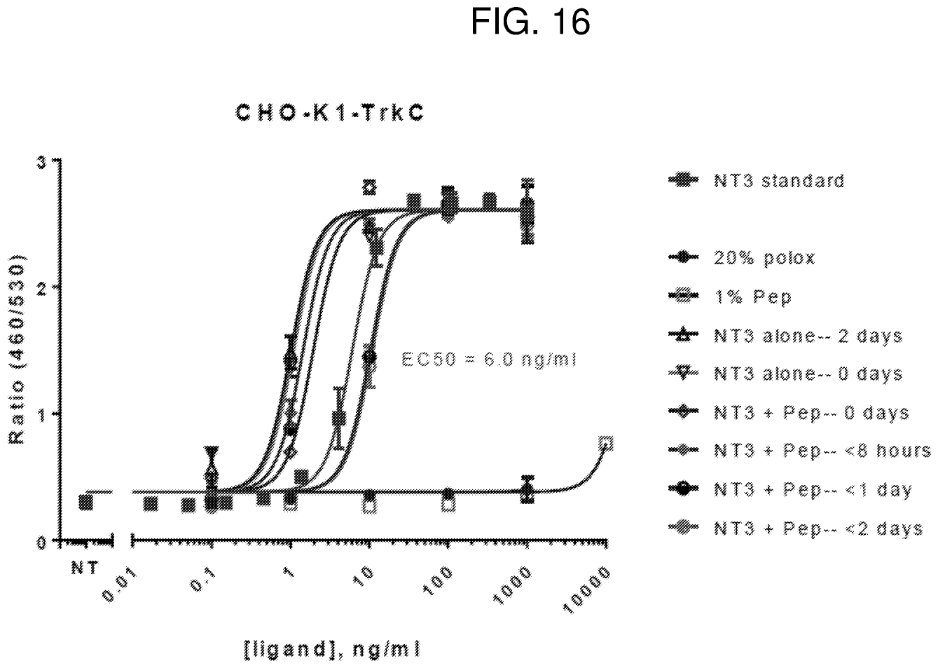

Surprisingly, intratympanic administration of peptide (XVI) was capable of not only significantly elevating cochlear NT-3 exposure, but was also able to effectuate a sustained residence of NT-3 in guinea pig perilymph for an extended period of time (FIGS. 12, 13A, and 13B). This peptide was substantially superior to other permeation enhancers tested, which had little to no effect on NT-3 round window membrane penetration (see, e.g., FIGS. 14 and 15). The peptide (XVI) formulation also exhibited a remarkable storage stability profile, maintaining TrkC-binding activity after 48 hours in storage (FIG. 16).

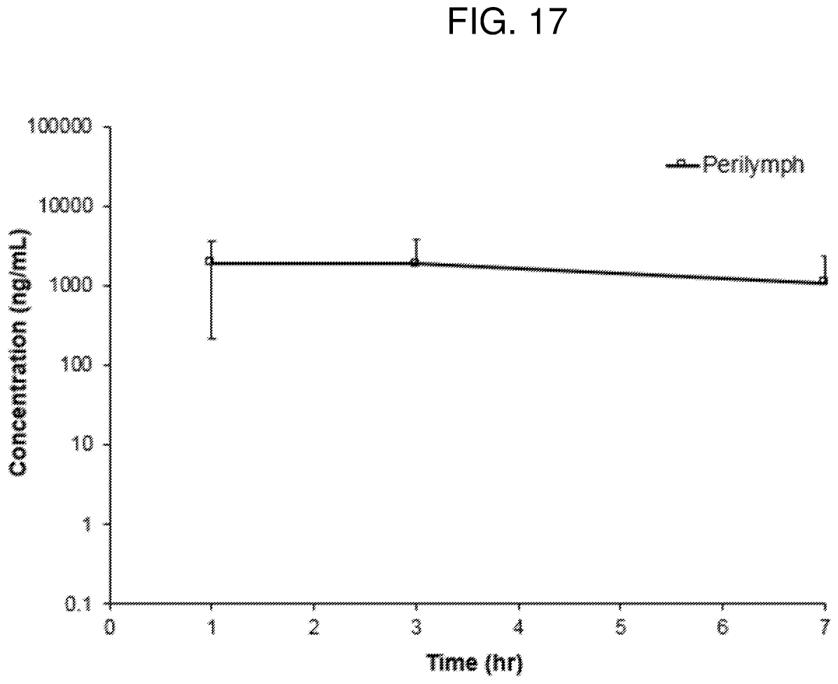

As shown in FIG. 17, the permeation-enhancing effects of peptide (XVI) can be extended to other high-molecular weight compounds, as an IgG1 antibody delivered in the background of this peptide was additionally observed to be capable of penetrating the round window membrane and maintaining residence for extended periods of time.

Taken together, these data demonstrate that a wide array of permeation enhancers failed to achieve significant cochlear NT-3 exposure, and yet peptide (XVI) not only imparted the test subjects with a substantial in vivo concentration of NT-3 in the inner ear, but was capable of promoting NT-3 inner ear residence for extended periods of time. These effects can be extended to other large therapeutic agents, and provide a mode by which high molecular-weight therapeutics may be delivered to the inner ear of a subject in need thereof.

To further characterize the effects of peptide (XVI), the in vitro permeability assay was used to evaluate administration of NT-3 in either vehicle or 1% of peptide (XVI), as depicted in FIG. 18. In this experiment, cultures were incubated at 37° C. and 50 μL samples were collected at various time points. After 3.5 hours of incubation, the top and bottom media were collected separately and the insert was fixed for immunohistochemical staining to evaluate tight and adherens junctions.

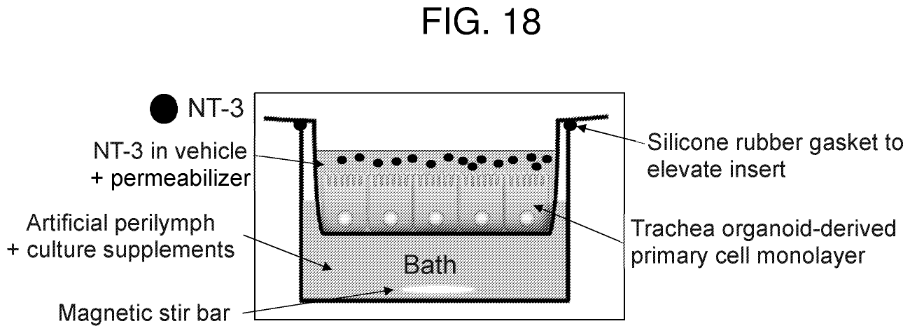

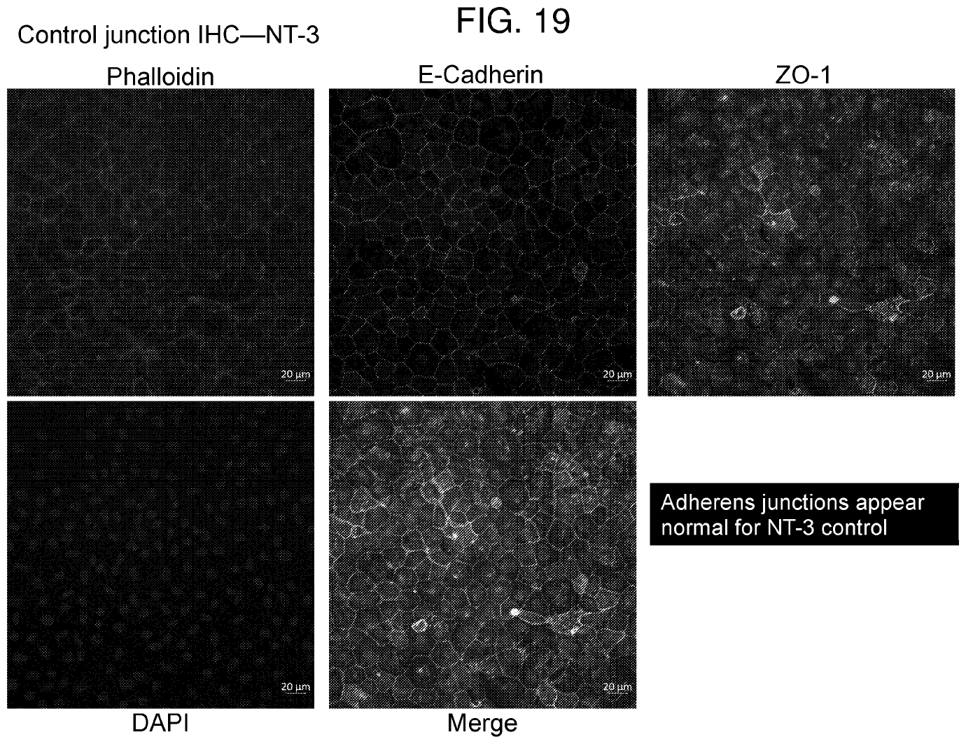

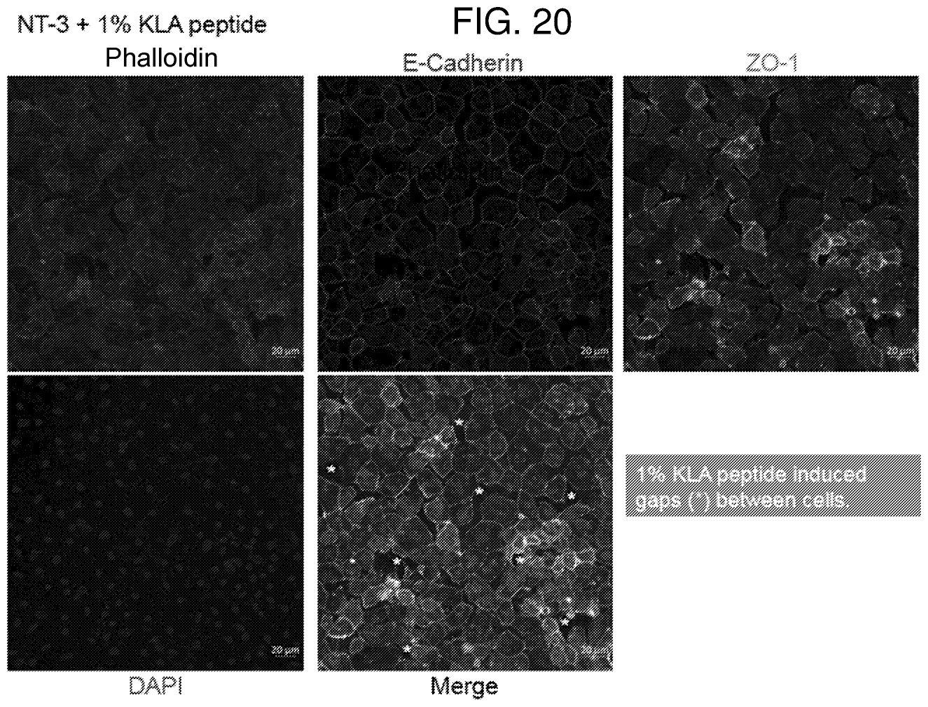

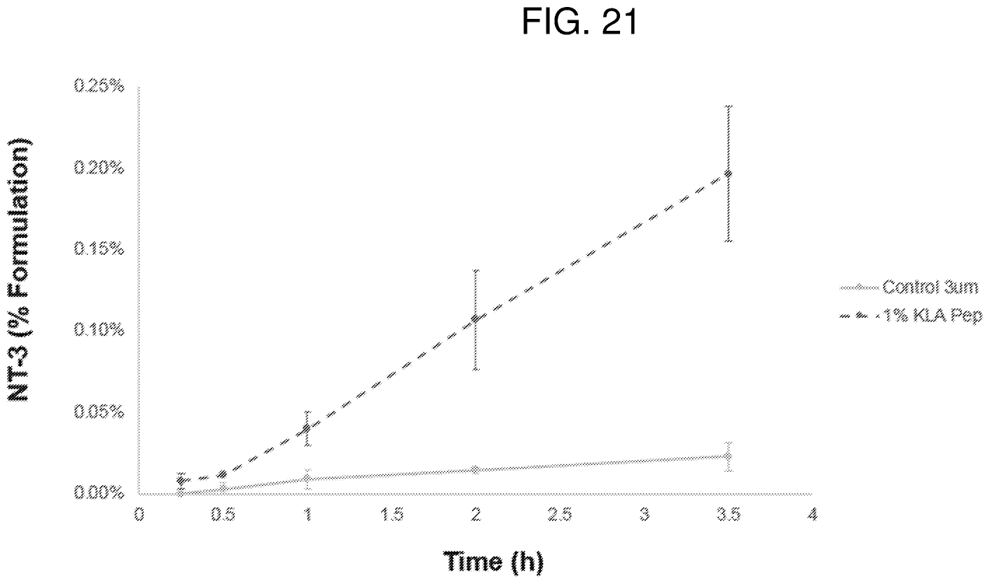

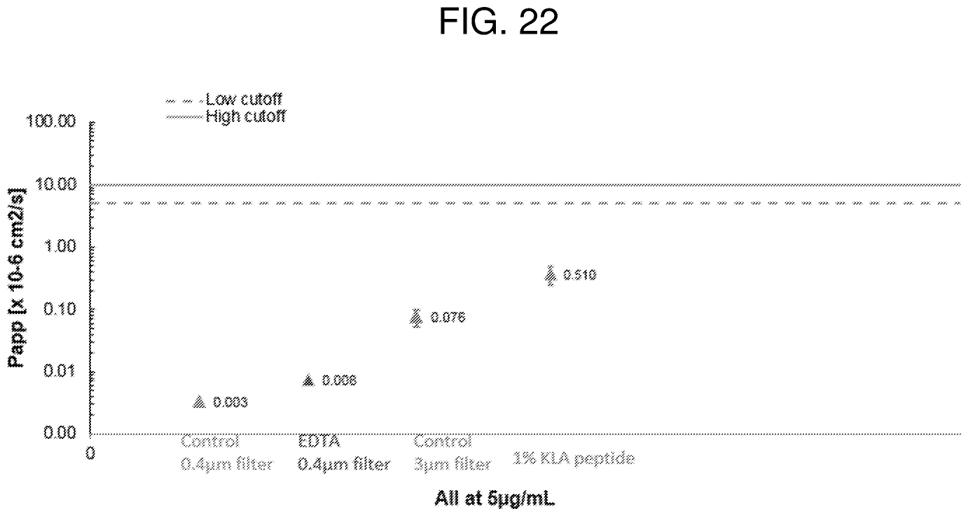

As shown in FIG. 19, adherens junctions appeared normal after incubation with NT-3 and vehicle, exhibiting an organized honeycomb network. After incubation with NT-3 in 1% of peptide (XVI), gaps could be observed between cells, denoted using asterisks in the merged image in FIG. 20. NT-3 also exhibited greater flux (FIG. 21) and apparent permeability (FIG. 22) when incubated with 1% of peptide (XVI) compared to control.

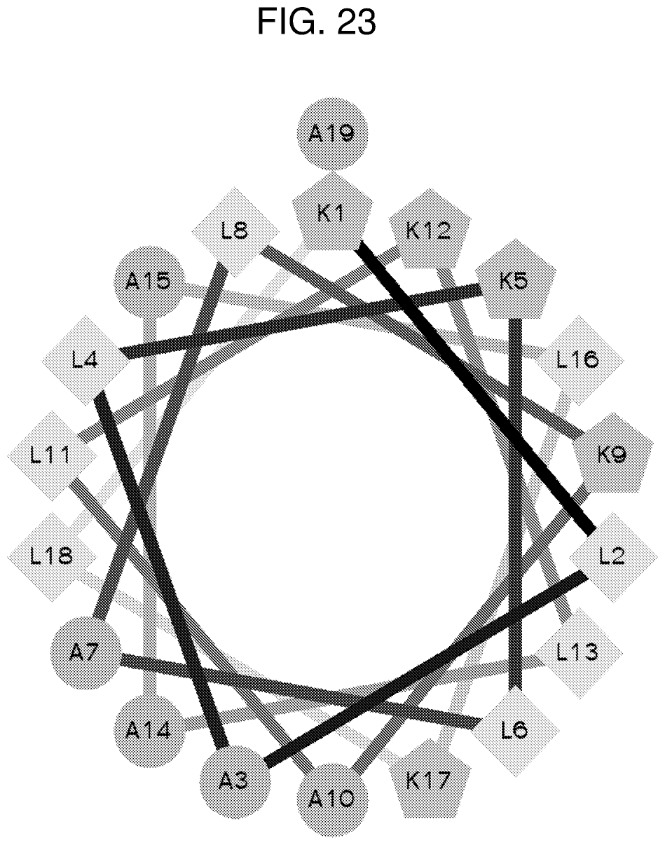

As shown in FIG. 23, peptide (XVI) is a facially amphipathic peptide that exhibits an alpha-helical structure. Peptide (XVI) contains a hydrophobic face, comprised primarily of leucine and alanine residues, as well as a hydrophilic face, have an abundance of positively charged lysine residues. In accordance with the present disclosure, other facially amphipathic, alpha helical peptides described herein can be admixed with a therapeutic agent, such as NT-3, so as to achieve delivery of the therapeutic agent across the round window membrane and to promote sustained, elevated concentrations of the therapeutic agent for extended periods of time.

### Example 2. Delivery of a Therapeutic Agent Across the Round Window Membrane of a Human Patient

Using the compositions and methods described herein, a physician can deliver a therapeutic agent into the inner ear of a subject (e.g., a mammalian subject, such as a human). For example, a physician may administer a pharmaceutical composition containing a permeation-enhancing peptide described herein to a human patient so as to effectuate the passage of a therapeutic agent into the inner ear of the patient (e.g., through the round window membrane). The pharmaceutical composition may be administered so as to treat one or more of a variety of otic disorders, such as ceruminosis or ceruminosis associated with an otic disease or condition, ear pruritus, otitis externa, otalgia, tinnitus, vestibular dysfunction (e.g., vertigo, dizziness, or loss of balance), ear fullness, hearing loss, Meniere's disease, sensorineural hearing loss (e.g., noise-induced hearing loss, age-related hearing loss (presbycusis), ototoxic drug-induced hearing loss, hearing loss related to head trauma, hearing loss related to infection), autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

Upon determining that the patient has one or more of the above conditions and/or is a candidate for therapy, the physician may administer the pharmaceutical composition to the patient by way of one or more routes of administration described herein. For example, the physician may administer the pharmaceutical compositions to the patient by local administration to the inner ear (e.g., administration into the perilymph or endolymph, e.g., through the oval window, round window, semicircular canal, or horizontal canal), and/or by intratympanic or transtympanic administration. Additionally or alternatively, the physician may administer the pharmaceutical composition to the patient by intravenous administration, parenteral administration, intradermal administration, transdermal administration, intramuscular administration, intranasal administration, subcutaneous administration, percutaneous administration, intratracheal administration, intraperitoneal administration, intraarterial administration, intravascular, inhalation, perfusion, lavage, and/or oral administration. The chosen route of administration may depend, e.g., on the particular composition administered, the subject, pharmaceutical formulation methods, administration methods (e.g., administration time and administration route), the subject's age, body weight, gender, severity of the disease being treated, the subject's diet, and the subject's metabolism.

The physician may determine the optimal quantity of the pharmaceutical composition to administer to the patient. For example, the physician may choose to administer at least 50 μL of the pharmaceutical composition to the round window of the subject. Exemplary amounts of the pharmaceutical composition that may be administered to the subject are, without limitation, 50 μL to 1 mL (e.g., 100 μL to 1 mL, 200 μL to 1 mL, 100 μL to 0.8 mL, 200 μL to 0.8 mL, 100 μL to 0.5 mL, 200 μL to 0.5 mL, 0.5 mL to 1.0 mL, 0.5 mL to 0.8 mL, or 0.8 mL to 1.0 mL).

After a period of time, such as from about 1 day to about 6 weeks, or more, the physician may conduct an analysis to determine whether the therapeutic agent is still present in vivo at an effective level. The physician may obtain a sample from the patient, such as a blood sample or perilymph sample, and may determine whether the subject is a candidate for a subsequent administration of the therapeutic agent based, for example, on the concentration of the therapeutic agent remaining in the sample and the time from the initial administration at which the sample was obtained.

### Example 3. Delivery of a Neurotrophin Across the Round Window Membrane of a Human Patient to Prevent or Reduce Sensory Hair Cell Damage or Death

A physician may administer a pharmaceutical composition containing one or more of the permeation-enhancing peptides described herein to a human patient in order to treat, prevent, or reduce sensory hair cell damage or death in the subject. For example, the neurotrophin (e.g., neurotrophin-3 (NT-3), nerve growth factor (NGF), brain-derived neurotrophic factor (BDNF), ciliary neurotrophic factor (CNTF), glial cell-line derived neurotrophic factor (GDNF), neurotrophin-4 (NT-4), fibroblast growth factor (FGF), insulin-like growth factor (IGF), epidermal growth factor (EGF), platelet-derived growth factor (PGF), mesencephalic astrocyte-derived neurotrophic factor (MANF), cerebral dopamine neurotrophic factor (CDNF), a pan-neurotrophic factor (e.g., PNT-1), a chimeric neurotrophin (e.g., a NGF/BDNF chimera), or combinations thereof) may be admixed with a permeation-enhancing peptide described herein, optionally in combination with poloxamer (e.g., poloxamer 407) and administered to the patient so as to treat, prevent, or reduce sensory hair cell damage or death. The resulting pharmaceutical composition may be administered to the patient by way, for example, of local administration to the inner ear (e.g., administration into the perilymph or endolymph, e.g., through the oval window, round window, semicircular canal, or horizontal canal), and/or by intratympanic or transtympanic administration.

The physician may determine the optimal quantity of the pharmaceutical composition to administer to the patient. For example, the physician may choose to administer at least 50 μL of the pharmaceutical composition to the round window of the subject. Exemplary amounts of the pharmaceutical composition that may be administered to the subject are, without limitation, 50 μL to 1 mL (e.g., 100 μL to 1 mL, 200 μL to 1 mL, 100 μL to 0.8 mL, 200 μL to 0.8 mL, 100 μL to 0.5 mL, 200 μL to 0.5 mL, 0.5 mL to 1.0 mL, 0.5 mL to 0.8 mL, or 0.8 mL to 1.0 mL).

After a period of time, such as from about 1 day to about 6 weeks, or more, the physician may conduct an analysis to determine whether the therapeutic agent is still present in vivo at an effective level. The physician may obtain a sample from the patient, such as a blood sample or perilymph sample, and may determine whether the subject is a candidate for a subsequent administration of the therapeutic agent based, for example, on the concentration of the therapeutic agent remaining in the sample and the time from the initial administration at which the sample was obtained.

### Example 4. Delivery of Sodium Thiosulfate Across the Round Window Membrane of a Guinea Pig

Hyaluronan (15.22 mg; Pharma Grade 80, Kikkoman Biochemifa company) was added to distilled water (1.50 mL), and the resulting mixture was stirred for 30 min at 4° C. Peptide KLALKLALKALKLAALKLA (15.10 mg) was added to the solution, and the mixture was stirred for 20 min at 4° C. Sodium thiosulfate pentahydrate (373.05 mg) was added to the solution. The pH was adjusted to pH 7.10 by addition of NaOH (0.1M, 9 uL). The resulting solution was filtered through 0.22 μm Millex-GV sterile filter with 1902 mOsmol/kg to produce a KLALKLALKALKLAALKLA/hyaluronan gel.

Pharmacokinetics of the KLALKLALKALKLAALKLA/hyaluronan gel was then assessed in guinea pigs. Albino guinea pigs (Hartley), body weight at 250-350 g, were used for the studies. For transtympanic dosing, the animal was placed on its shoulder with the surgery ear up and auditory bulla was first exposed using retroauricular approach. A hole of 2-3 mm in diameter was drilled on the bulla to provide direct visualization of the round window niche. Then, 10 μL of an aqueous composition of 0.5 M sodium thiosulfate/2% (w/v) hyaluronan (STS Composition) were applied onto the RWM using a 10 μL Hamilton syringe and a 26-gauge needle. After application, guinea pigs remained at this position for 30 min to allow compound to diffuse into the cochlea. The bulla opening was sealed with a muscle graft and the incision closed with sutures.

Sampling procedures are as follows, in brief. All sampling procedures were terminal. Animals were euthanized with CO₂. 0.5 mL samples of blood were collected by cardiac puncture. Plasma was separated by centrifugation at 5,000 rpm at 4° C. for 10 min and collected in a separate tube. 50 μL of cerebrospinal fluid were collected through the cisterna magma. Perilymph was collected ex vivo to avoid contamination from the cerebrospinal fluid influx via the cochlear aqueduct. The temporal bone was rapidly isolated, and the bulla was removed to expose the cochlea. Any visible remaining dosed compositions were carefully removed with absorbent points under the surgical microscope before perilymph sampling. A small hole was made at the apex, and then 5-7 μL of perilymph was sampled using a pulled glass pipette. All samples were frozen immediately on dry ice and stored in −80° C. until analysis. The concentrations of thiosulfate in the samples were measured using the method disclosed in Togawa et al. Chem. Pharm. Bull., 40:3000-3004, 1992, the disclosure of which is incorporated herein by reference. The results of this study are shown in Table 5. The KLALKLALKALKLAALKLA/hyaluronan gel was found to deliver sodium thiosulfate to produce the following pharmacokinetic parameters.

TABLE 5

| Tₘₐₓ | Cₘₐₓ | Terminal | AUC_{IFN.} | AUCₗₐₛₜ |
|---|---|---|---|---|
| (h) | (ng/mL) | T_{1/2} | (hr * ng/g) | (hr * ng/g) |
| 1 | 1072200 | 2.84 | 4848026 | 4761412 |

### Example 5. Study of the Reversibility of the Tight Junction Opening (Ex Vivo)

To evaluate the proposed mechanism of action of the KLALKLALKALKLAALKLA peptide on the round window membrane (RWM) and the capacity for the RWM to recover after application of this peptide we investigate tight junction integrity before and after application of KLALKLALKALKLAALKLA.HCl in both ex vivo and in vivo settings. Additionally, to evaluate safety of this peptide, we evaluate auditory brain stem responses before and after application of this compound to the guinea pig RWM.

a. Evaluation of KLALKLALKALKLAALKLA Peptide Mechanism on the RWM: Ex Vivo Culture

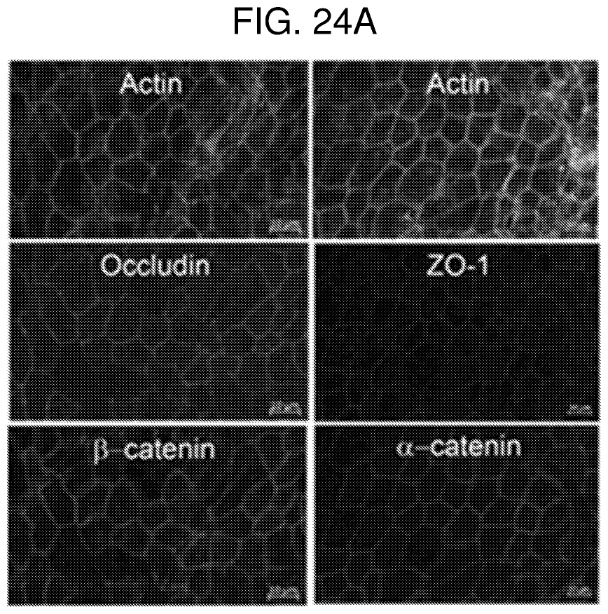

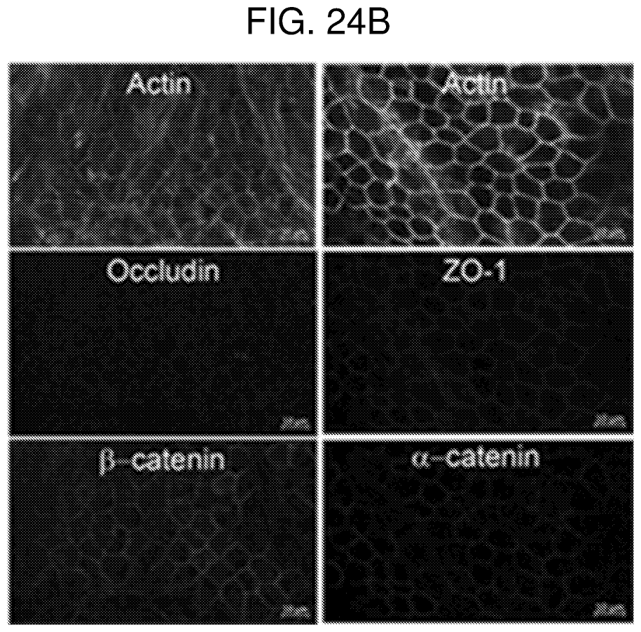

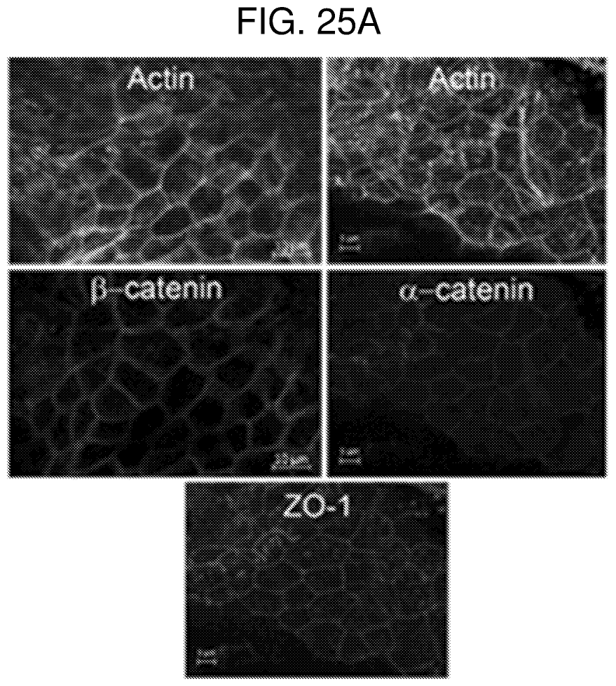

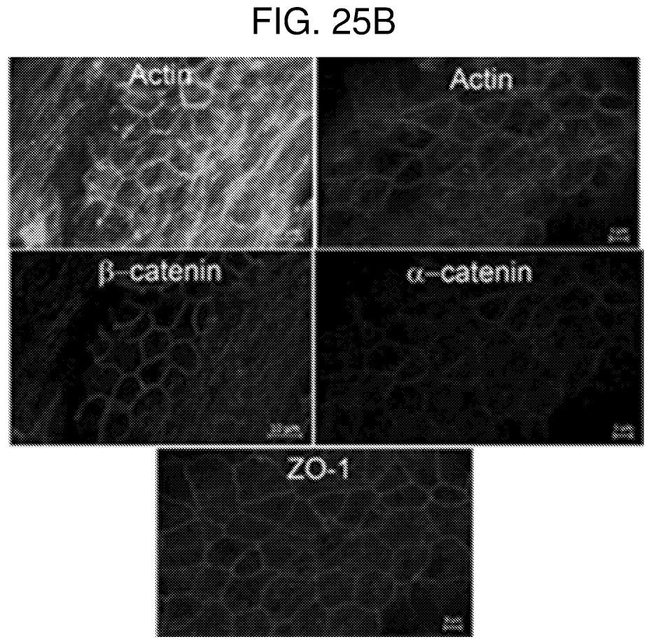

The KLALKLALKALKLAALKLA peptide is hypothesized to interfere with cellular tight junctions. To assess if this mechanism is relevant to the RWM, we excise the RWM from adult guinea pigs (Elm Hill Labs, Chelmsford, Mass.) and stain for a variety of adherens and tight junction proteins. To extract the guinea pig RWM, the temporal bone was removed from freshly sacrificed guinea pig tissue and placed in PBS. An incision was then made in the roof of the bulla to expose the cochlea, followed by trimming of the temporal bone, bulla, and middle ear bones to expose the RWM. The round window niche was then removed from the cochlea and then cultured in DMEM with and without 1% KLALKLALKALKLAALKLA.TFA for 1 hour. This tissue was then fixed in a 4% PFA solution for 30 minutes followed by wash with PBS (3 times, 5-minute incubations). Fixed RWMs were then blocked in goat blocking serum (5% in PBS) then stained with primary antibodies against occludin (1:250, OcIn Antibody. Life Technologies Corporation, Chicago Ill.; Part #331500; mouse, IgG1, K), zona occludin-1 (ZO-1) (1:100, ZO-1/TJP1 Antibody (ZO1-1A12). Life Technologies Corporation, Chicago Ill.; Part #339100; mouse, IgG1), beta-catenin (1:250, Rabbit monoclonal [E247] to CTNNB1; Abcam, Inc., Boston, Mass. Part #ab32572), or alpha-catenin Catenin (1:100, Rabbit monoclonal [EP1793Y] to CTNNA1; Abcam, Inc., Boston, Mass. Part #ab51032) and then washed with PBS (3 times, 5-minute incubations). RWMs were then incubated in secondary antibodies (1:500, goat anti-mouse, IgG1, 647 or 1:500, goat anti-rabbit IgG, 568) and/or Alexa Fluor 488 phalloidin (1:200; Thermo Fisher Scientific) for two hours followed by wash with PBS (3 times, 5-minute incubations). After incubation in secondary antibodies (Thermo Fisher Scientific), the round window tissue was extracted from the round window niche, transferred to a microscopy slide, and a droplet of imaging solution (Flouromount) was added on top of the round window tissue. Next, a #1.5 coverslip was placed on top of the round window tissue and imaging solution, and the resulting tissue preparation was imaged as a z-stack through the thickness of the tissue using a Zeiss LSM 800 confocal microscope. We observe that 1% KLALKLALKALKLAALKLA.TFA disrupts occludin, ZO-1, and alpha-catenin, but not actin (as imaged via phalloidin), or beta-catenin structure (FIGS. 24A and 24B). At a lower dose of the peptide (0.1% KLALKLALKALKLAALKLA.HCL for 1 hour in DMEM), alpha-catenin appeared to be disrupted, but not beta-catenin or ZO-1 structure (FIGS. 25A and 25B).

b. Evaluation of RWM Recovery Capacity after Application of KLALKLALKALKLAALKLA Peptide: Ex Vivo Culture

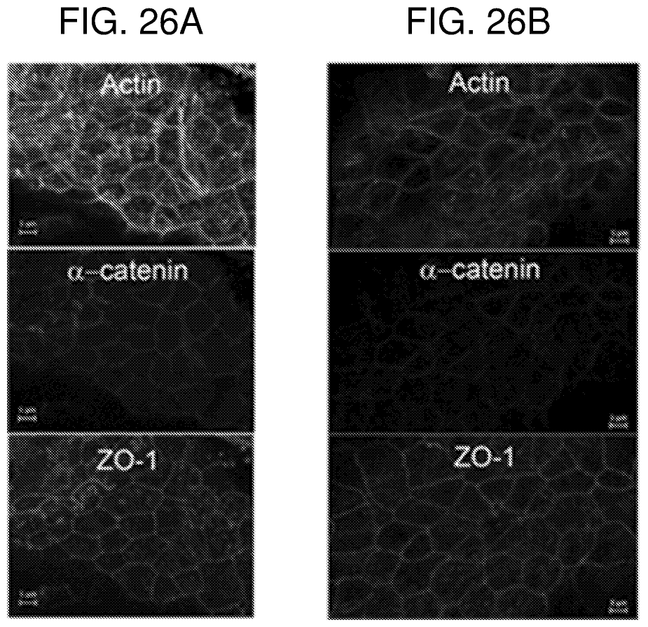

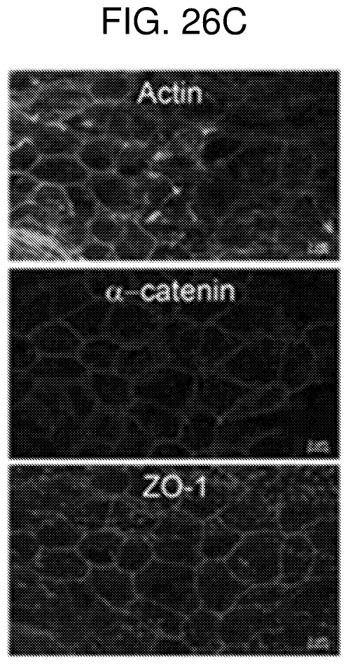

To assess the recovery capacity of the RWM after exposure to the KLALKLALKALKLAALKLA peptide we excise the guinea pig RWM as is described in section (a) and apply 0.1% KLALKLALKALKLAALKLA.HCl for 1 hour and then either immediately fix the tissue or allow for 75 minutes of recovery in DMEM culture media. We then perform the same immunohistochemistry procedure as described in Example 5, section (a), staining for ZO-1 and alpha-catenin as both proteins were indicated as disrupted after exposure to 1% KLALKLALKALKLAALKLA.TFA. We observe that immediately after fixation of the RWM tissue after exposure to 0.1% KLALKLALKALKLAALKLA.HCl for 1 hour, alpha-catenin was disrupted, but ZO-1, beta-catenin, and actin were not. With 75 minutes of recovery in DMEM culture media, alpha-catenin junctions return to normal intensity levels (FIGS. 26A, 26B, and 26C).

c. Evaluation of RWM Integrity after In Vivo Application of KLALKLALKALKLAALKLA Peptide

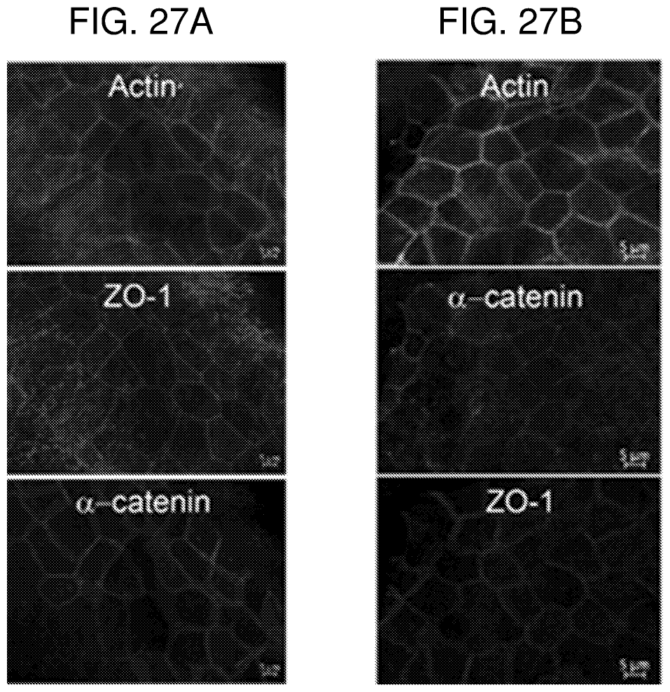

To assess the capacity for the RWM to recover after application of the KLALKLALKALKLAALKLA peptide we record ABR thresholds with and without application of a 50 μL formulation including 0.1% KLALKLALKALKLAALKLA.HCL to the guinea pig RWM via trans-tympanic injection. After application of this peptide, ABR thresholds were recorded after 7 and 14 days, and no noticeable threshold shift was observed. Post-injection (21 days), the guinea pigs in both the KLALKLALKALKLAALKLA exposed and control groups were sacrificed, and the temporal bone was removed as described in Example 5, section (a). The RWM tissue from the guinea pig cochlea was fixed and stained as described in Example 5, section (a) for ZO-1, alpha-catenin, and phalloidin (FIGS. 27A and 27B), given previous demonstration of alpha-catenin being disrupted after a 1-hour exposure to 0.1% KLALKLALKALKLAALKLA.HCL, see Example 5, section (a). Tight junction proteins (actin, ZO-1, and alpha-catenin) are intact 21 days post injection of KLALKLALKALKLAALKLA.HCL in an in vivo setting (FIGS. 27A and 27B) despite depletion of alpha-catenin after a 1-hour exposure to 0.1% KLALKLALKALKLAALKLA.HCL (FIGS. 25A and 25B).

### Example 6. Pharmacokinetics (PK) Studies

### Pharmacokinetics and Auditory Brainstem Response in Rodent

Pre-Operation:

Male guinea pigs weighing 200-300 g of approximately 5-7 weeks of age served as subjects (N=5 per group). Prior to any procedures, animals were anesthetized using Zoletil 50 (20 mg/kg) 10 minutes before surgery via the intramuscular route.

Trans-Tympanic Injection:

Each animal's head was shaved around left ear with an electric razor, and the skin was cleaned with 75% ethanol. A sterile surgical field was established, and sterile draping cloth was applied. A small hole over the upper left side of tympanic membrane was created with a metal syringe. 50 μL of formulations was delivered through the right side of tympanic membrane using a sterile glass Hamilton syringe with 25-26 G blunt needle. The solution was injected at a set rate. The animals were fixed and kept lying on the side for more than 30 min.

Intra-Tympanic Injection:

Under microscopic magnification, a 0.5-1.5 cm post-auricular skin incision was made using sharp scissors, the incision was approximately 6-8 mm caudal to the auriculo-cephalic crease. Deep cuts were avoided to preserve underlying vascular structures. Subcutaneous fat layer, muscles, and tissues were carefully dissected with forceps. The cleidomastoideus muscle body was gently retracted to reveal the shiny dome of the tympanic bulla periosteum. At the caudal aspect of the bulla, the insertion of a deeper cervical muscle, the sternomastoideus, was observed. The facial nerve, which becomes visible at the dorsal and rostral aspect of the bulla dome, was preserved.

Prior to drilling a small hole (0.5 mm diameter) in the posterior part of the bulla, a self-retaining retractor was placed into the soft tissue around the incision. The bulla bone was uncapped in a dorsal and caudal direction using a pair of jeweler's tip forceps. The bone was removed in pieces under high magnification. Throughout this procedure, care was exercised not puncture the stapedial artery, which lies directly beneath the bulla cap, as bleeding from this artery may compromise the procedure. To prevent excessive fluid entry to the middle ear, the amount of the bone removed was minimal but sufficient to allow visualization and access to the round window niche. 10 or 90 μL of formulations were delivered to the round window niche using a sterile glass Hamilton syringe with 25-26 G blunt needle. The delivered agent was allowed to rest within the round window niche for up to 30 min. The small hole was covered with muscular tissue and tissue glue. The incision was closed with sutures (4-0 non-absorbable monofilament or 5-0 non-absorbable nylon) and tissue glue or wound clips. The entire procedure took approximately 3-5 minutes depending on agent specifications. During the procedure and until recovery, animals were placed on a temperature controlled (38° C.) heating pad until consciousness was regained, at which time they were returned to the home-cage.

ABR/Hearing Testing

Prior to ABR recording, the tympanic membrane was inspected for otitis media using a surgical microscope. Animals with otitis media were excluded from the analysis. A TDT RZ6 system was used to record the auditory brainstem response (ABR). Animals were anaesthetized with zoletil (20 mg/kg, I.M.) and placed in a soundproof chamber. Three stainless needle recording electrodes were inserted just under the skin and connected to the appropriate input on the PA4LI low impedance headstage: Channel 1: Ipsilateral ear (same side as stimulus delivery); Reference: contralateral ear; Ground: Vertex (base of skull). A computer-controlled TDT RZ6 System was used to generate the auditory pure tone and power amplified before fed to a closed-field speaker to deliver sound stimuli. The same pure tone stimuli were repeated 512 times, the sound pressure levels were increased in a step of 10 dB SPL (5 dB around threshold) from 20 dB SPL to 90 dB SPL after every 512 repeated trials. The lowest sound level which induced a brainstem response was regarded as the threshold. The hearing thresholds were measured at 4, 24, and 32 kHz. ABR recording was performed before surgery or dosing and on the 7th and 14th day after the formulation dosing. The ABR was recorded first from the left ear and then from the right ear.

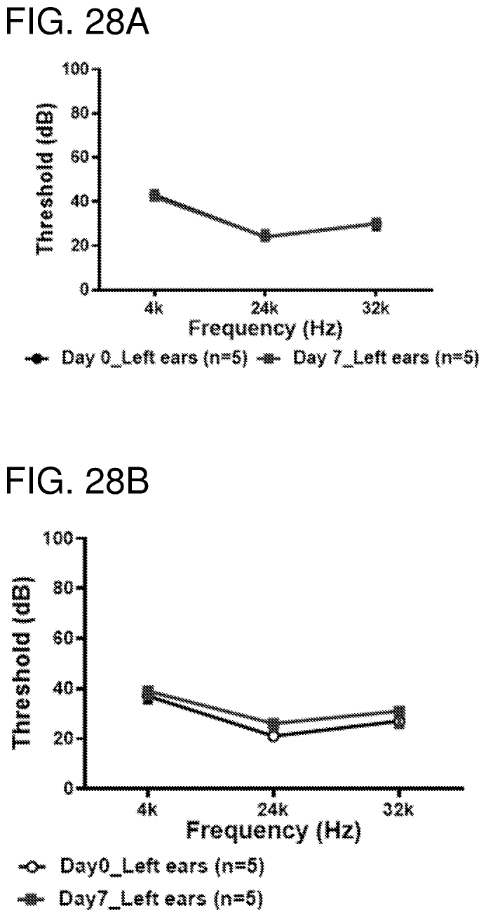

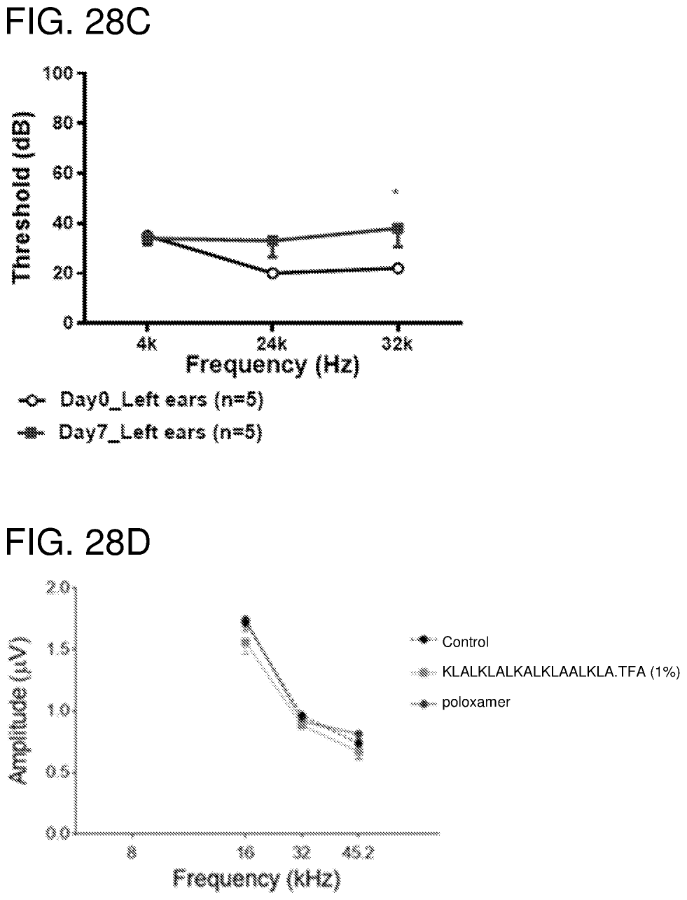

The results of the ABR/hearing testing are summarized in FIGS. 28A, 28B, 28C, and 28D.

Sampling Collection:

Blood Collection:

Without preinflating the euthanasia box, the guinea pig was placed in a box, and 100% carbon dioxide was introduced to render the animal unconscious and reduce animal suffering. The carbon dioxide flow was maintained for at least 1 minute after breathing stopped. The guinea pig was removed from the euthanasia box after death was confirmed. Blood was collected immediately after euthanasia. After the operator fixed the animal's back position, the needle was inserted at the front of the sternal ridge at 4-6 or slightly forward. Then, the needle was pulled back and the blood was returned. For each blood collection, approx. 1 mL of blood were collected.

Csf Collection:

CSF was collected after euthanasia. A 0.5×20 intravenous infusion needle was slowly lowered from 900 to the foramen magnum, reaching a distance of 4.5-5 mm under the skin, and slowly withdrawn to obtain 50-200 μL of clear tissue fluid.

Perilymph Collection:

After euthanasia, the animal was stripped excess skin and muscle tissue to obtain a complete auditory bulla, and the bulla wall was cut with small forceps to expose the cochlea. The basal turn of bulla was cleaned using small cotton ball. The cochlear bottom circle and the round window were coated with bioglue. After drying, a microhole was drilled in the top circle of the cochlea. 2 μL of perilymph were then collected using a microcapillary inserted into the cochlear scala tympani. Perilymph samples were stored at −80° C. until analysis.

PK Study in NHP:

NT3 (0.5%, 5 mg/mL) in formulation A [KLALKLALKALKLAALKLA.TFA (1%), poloxamer 407 (20%), and PBS (pH7)] was administered intratympanically to two cynomolgus monkeys as follows. The animals were anesthetized with isoflurane. A post-auricular incision was made, soft tissue was separated, and the mastoid was drilled to expose the facial recess. The round window was accessed through the facial recess. 100 μL of 0.5% NT3 in 20% poloxamer with 1% KLALKLALKALKLAALKLA were dosed onto the round window membrane into the left ear. The facial recess was packed with a piece of muscle, and the same procedure and dosing were performed on the right ear. The staggered dosing was one hour between two ears. The animals were sacrificed at three or six hours after dosing the second ear, the remaining NT3 was removed from the round window, and the cochleas were exacted. A small opening was made on the cochlear capsule at the apex. 10 μL of perilymph were sampled. The perilymph sample was immediately frozen on dry ice and stored at −80° C. until analysis. The concentration of NT3 was qualified using an ELISA assay.

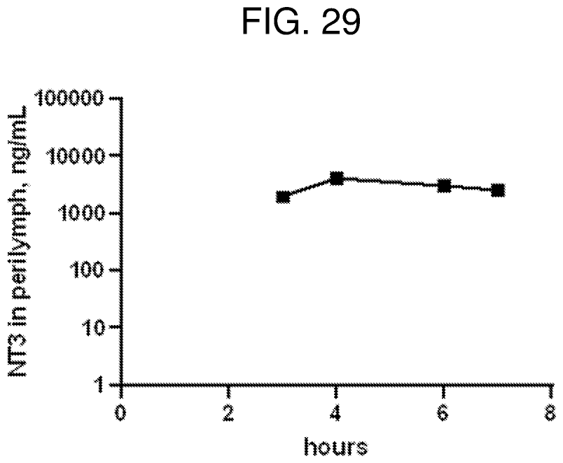

The results of the pharmacokinetic studies are summarized in Table 6 and FIG. 29.

TABLE 6

| | | Dose | | Exposure @ | Exposure @ |
|---|---|---|---|---|---|
| | Species | volume (uL) | Formulation | 3 h (ng/mL) | 7 h (ng/mL) |
| 1 | mice | 5 | NT3 (0.5%), | 7902 ± 3163 | 5618 ± 2188 |
| | | | KLALKLALKALKLAALKLA•HCl | | |
| | | | (1%), P407 (20%) in PBS (pH7) | | |
| 2 | mice | 5 | mAb (0.5%), | 3585 ± 2732 | 3890 ± 2614 |
| | | | KLALKLALKALKLAALKLA•HCl | | |
| | | | (0.25%), HA (1%) in His (pH6) | | |
| 3 | mice | 5 | NT3 (0.5%), | 1622 ± 379 | 1270 ± 506 |
| | | | KLALKLALKALKLAALKLA•HCl | | |
| | | | (0.1%), HA (1%) in PBS (pH7) | | |
| 4 | mice | 5 | mAb (2.5%), | 278325 ± 218738 | 172081 ± 172146 |
| | | | KLALKLALKALKLAALKLA•HCl | | |
| | | | (0.25%), HA (1%) in His (pH6) | | |
| 5 | mice | 5 | NT3 (0.5%), | 6985 ± 3027 | 3892 ± 2126 |
| | | | KLALKLALKALKLAALKLA•HCl | | |
| | | | (0.25%), P407 (20%) in PBS | | |
| | | | (pH7) | | |
| 6 | mice | 5 | NT3 (0.5%), | 7043 ± 6184 | 3556 ± 2399 |
| | | | KLALKLALKALKLAALKLA•HCl | | |
| | | | (0.25%), HA (1%) in PBS (pH7) | | |
| 7 | mice | 5 | NT3 (0.1%), | 1546 ± 1075 | 1811 ± 1484 |
| | | | KLALKLALKALKLAALKLA•HCl | | |
| | | | (0.25%), P407 (20%) in PBS | | |
| | | | (pH7) | | |
| 8 | Guinea | 10 | NT3 (0.1%), P407 (20%) in Tris | 8.13 ± 2.73 | 22.2 ± 28.6 |
| | pig | | | | |
| 9 | Guinea | 10 | NT3 (0.1%), | 456 ± 647 | 720 ± 566 |
| | pig | | KLALKLALKALKLAALKLA•HCl | | |
| | | | (0.25%), HA (1%) His (pH6) | | |
| 10 | Guinea | 10 | NT3 (0.5%), | 4236 ± 4256 | 41125 ± 3607 |
| | pig | | KLALKLALKALKLAALKLA•HCl | | |
| | | | (0.25%), P407 (20%), PBS | | |
| | | | (pH7) | | |
| 11 | Guinea | 10 | mAb (0.5%), | 9498 ± 17253 | 1673 ± 1255 |
| | pig | | KLALKLALKALKLAALKLA•TFA | | |
| | | | (0.25%), HA (1%), His (pH6) | | |

### Example 7. Preparation of NT3 Formulations

Formulation 1: NT3 (0.5%) in 20% poloxamer 407 (P407) in PBS (pH7) with KLALKLALKALKLAALKLA.HCl (0.1%) 1001.25 mg of poloxamer 407 (P407) was placed into a vial, and 4 mL of PBS was added into the vial. The resulting mixture was stirred gently until a clear solution at ˜4° C. ice bath. 3.06 mg of NT-3 and 0.62 mg of KLALKLALKALKLAALKLA.HCl were added into a plastic vial. 0.6 mL of poloxamer solution in PBS was added to the vial. The resulting mixture was gently stirred in ice bath until fully clear.

Formulation 2: NT3 (0.5%) with KLALKLALKALKLAALKLA.HCl (0.1%) in sodium hyaluronate (HA) (1%) in PBS (pH7)

29.58 mg of sodium hyaluronate (Pharma Grade 80, Kikkoman Biochemifa company; 0.6-1.2 mDa) was placed a vial, and 3.0 mL of PBS (10 mM) was added into the vial. The resulting mixture was stirred at ˜4° C. in an ice bath until fully dissolution. 0.6 mL of the resulting solution was added to a vial containing 3.14 mg of NT-3 and 0.62 mg of KLALKLALKALKLAALKLA.HCl. The resulting mixture was gently stirred, and a clear solution was obtained.

Formulation 3: mAb (0.5%) with KLALKLALKALKLAALKLA.HCl (0.25%) in sodium hyaluronate (HA) (1%) in His buffer (pH6)

40.25 mg of sodium hyaluronate (Pharma Grade 80, Kikkoman Biochemifa company; 0.6-1.2 mDa) and 9.96 mg of KLALKLALKALKLAALKLA.HCl were placed in a vial. 2.00 mL of His buffer (pH6.0, 10 mM) was added, and the resulting mixture was stirred in ice bath to a clear solution. 0.5 mL of the resulting solution was added into a vial containing 0.500 mL of mAb (10.3 mg/mL). After gent shaking, the desired formulation was obtained.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A pharmaceutical composition formulated for otic administration to a human patient, the pharmaceutical composition comprising a therapeutic agent and a permeation enhancer, wherein the permeation enhancer is an alpha-helical, facially amphipathic polypeptide having a molecular weight of from about 1,000 Da to about 3,500 Da and a pl of at least 7.0.

2. The pharmaceutical composition of claim 1, wherein polypeptide comprises one or more regions represented, from N-terminus to C-terminus, by formula (I)
X¹—X²—X² (I)
wherein each X¹ independently represents an amino acid comprising a cationic side chain at physiological pH;
each X² independently represents an amino acid comprising a hydrophobic side chain; and
each “—” independently represents a peptide bond or a peptide bond isostere.

3. The pharmaceutical composition of claim 2, wherein:
each X¹ independently represents an amino acid comprising a lysine side chain; and
each X² independently represents an amino acid comprising an alanine, leucine, or tryptophan side chain.

4. The pharmaceutical composition of any one of claims 1-3, wherein the polypeptide comprises from 2 to 10 of the regions represented by formula (I).

5. The pharmaceutical composition of any one of claims 1-4, wherein each of the regions represented by formula (I) are consecutive or separated by up to two amino acid residues.

6. The pharmaceutical composition of claim 1, wherein the polypeptide comprises one or more regions represented, from N-terminus to C-terminus, by formula (II)
X³—X⁴—X⁵—X⁴ (II)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain; and
each X⁵ independently represents an amino acid comprising an alanine side chain.

7. The pharmaceutical composition of claim 6, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

8. The pharmaceutical composition of claim 6 or 7, wherein the polypeptide comprises from 1 to 10 of the regions represented by formula (II).

9. The pharmaceutical composition of claim 1, wherein the polypeptide comprises one or more regions represented, from N-terminus to C-terminus, by formula (III)
X³—X⁴—X⁵—X⁴—X³—X⁴—X⁵—X⁴ (III)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain; and
each “—” independently represents a peptide bond or a peptide bond isostere.

10. The pharmaceutical composition of claim 9, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

11. The pharmaceutical composition of claim 9 or 10, wherein the polypeptide comprises from 1 to 5 of the regions represented by formula (III).

12. The pharmaceutical composition of claim 1, wherein the polypeptide comprises a region represented, from N-terminus to C-terminus, by formula (IV)
[X³—X⁴— X⁵— X⁴]ₙ—[X³—X⁴—X⁵]ₘ (IV)
wherein each X³ independently represents an amino acid comprising a lysine or arginine side chain;
each X⁴ independently represents an amino acid comprising a leucine, isoleucine, valine, methionine, tryptophan, phenylalanine, or tyrosine side chain;
each X⁵ independently represents an amino acid comprising an alanine side chain;
n represents an integer from 1 to 5;
m represents an integer from 1 to 5; and
each “—” independently represents a peptide bond or a peptide bond isostere.

13. The pharmaceutical composition of claim 12, wherein:
each X³ independently represents an amino acid comprising a lysine side chain;
each X⁴ independently represents an amino acid comprising a leucine or tryptophan side chain; and
each X⁵ independently represents an amino acid comprising an alanine side chain.

14. The pharmaceutical composition of claim 12 or 13, wherein n represents an integer from 2 to 4.

15. The pharmaceutical composition of any one of claims 12-14, wherein m represents an integer from 1 to 3.

16. The pharmaceutical composition of any one of claims 1-15, wherein the polypeptide comprises one or more intramolecular crosslinks.

17. The pharmaceutical composition of claim 16, wherein the one or more intramolecular crosslinks stabilize an alpha-helical structure.

18. The pharmaceutical composition of claim 16 or 17, wherein the polypeptide comprises one or more electrostatic intramolecular crosslinks.

19. The pharmaceutical composition of claim 16 or 17, wherein the polypeptide comprises one or more covalent intramolecular crosslinks.

20. The pharmaceutical composition of any one of claims 1-19, wherein the polypeptide is cyclized from N-terminus to C-terminus.

21. The pharmaceutical composition of any one of claims 1-20, wherein the polypeptide is from 10 to 30 amino acid residues in length.

22. The pharmaceutical composition of claim 21, wherein the polypeptide is 19 amino acid residues in length.

23. The pharmaceutical composition of any one of claims 1-22, wherein the polypeptide has an

24. The pharmaceutical composition of claim 23, wherein the polypeptide has a pl of about 10.6.

25. The pharmaceutical composition of any one of claims 1-24, wherein the polypeptide has a molecular weight of from about 1,400 Da to about 2,800 Da.

26. The pharmaceutical composition of claim 25, wherein the polypeptide has a molecular weight of about 1,990 Da.

27. The pharmaceutical composition of any one of claims 1-26, wherein the polypeptide has at least about 50% alpha-helicity as assessed by circular dichroism.

28. The pharmaceutical composition of claim 27, wherein the polypeptide has from about 61% to about 68% alpha-helicity as assessed by circular dichroism.

29. The pharmaceutical composition of claim 1, wherein the polypeptide is represented by formula (VII) 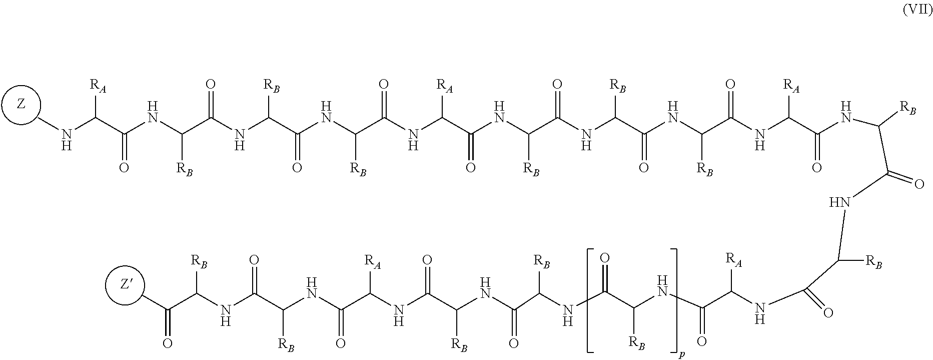
wherein each R_{A} is independently selected from: 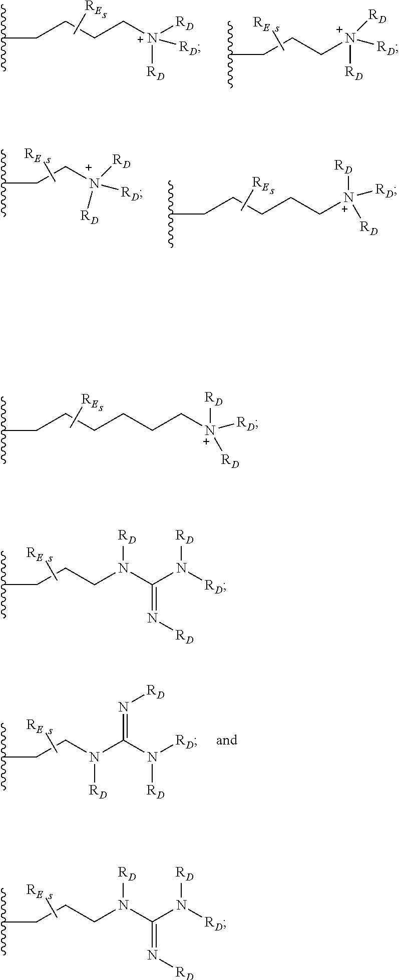
each R_{B} is independently selected from: 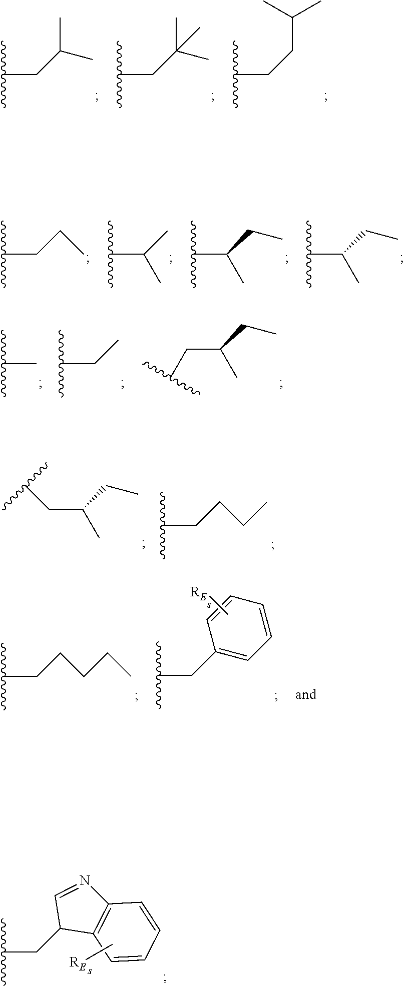
each R_{D} is independently hydrogen or an optionally substituted C₁-C₆ alkyl group;
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
p is an integer from 0 to 3;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

30. The pharmaceutical composition of claim 29, wherein the polypeptide is represented by formula (VIII) 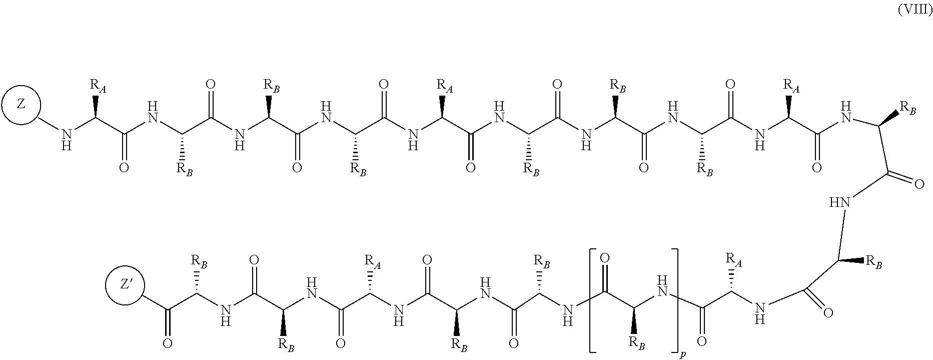
wherein each of R^{A}, R^{B}, R^{D}, R^{E}, p, s, Z, and Z′ are as defined for formula (VII).

31. The pharmaceutical composition of claim 29, wherein the polypeptide is represented by formula (IX) 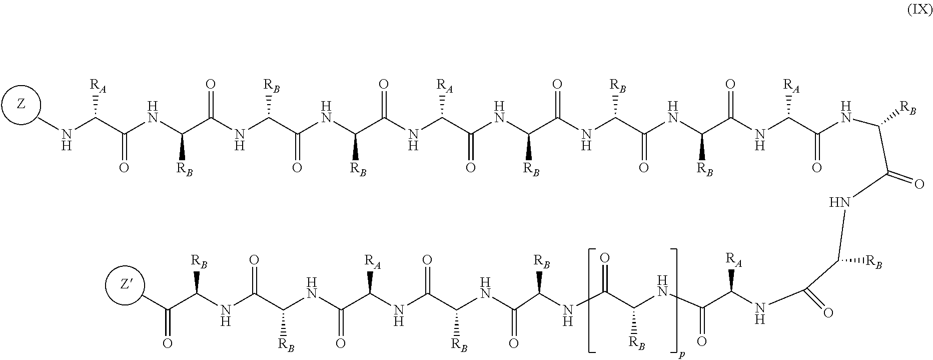
wherein each of R^{A}, R^{B}, R^{D}, R^{E}, p, s, Z, and Z′ are as defined for formula (VII).

32. The pharmaceutical composition of claim 1, wherein the polypeptide is represented by formula (X) 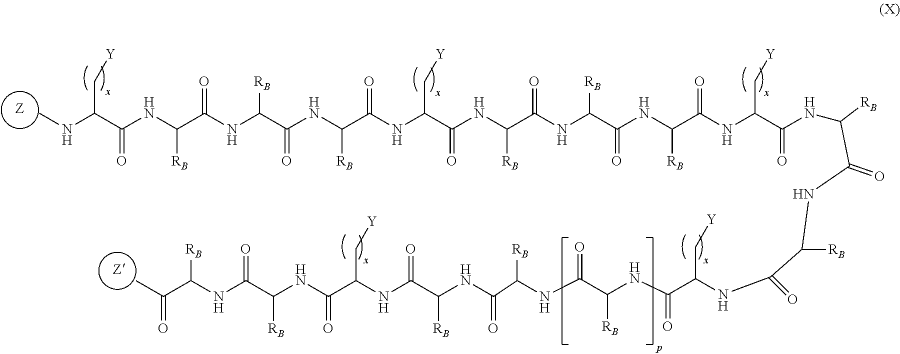
wherein each Y is independently an optionally substituted amino group or an optionally substituted guanidinium group;
each R_{B} is independently selected from: 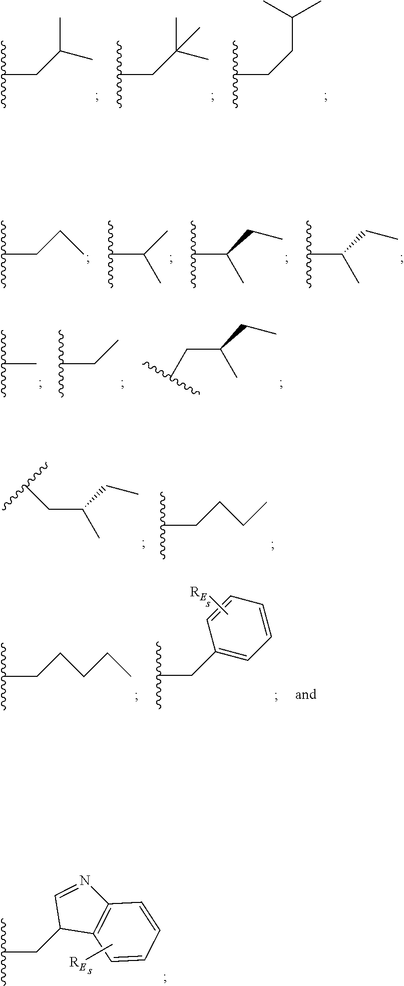
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
x is an integer from 3 to 5;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

33. The pharmaceutical composition of claim 32, wherein the polypeptide is represented by formula (XI) 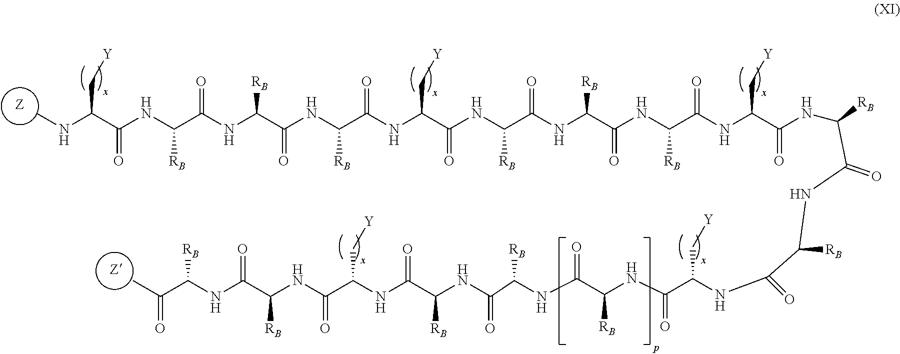
wherein each of Y, R^{B}, R^{E}, x, s, Z, and Z′ are as defined for formula (X).

34. The pharmaceutical composition of claim 32, wherein the polypeptide is represented by formula (XII) 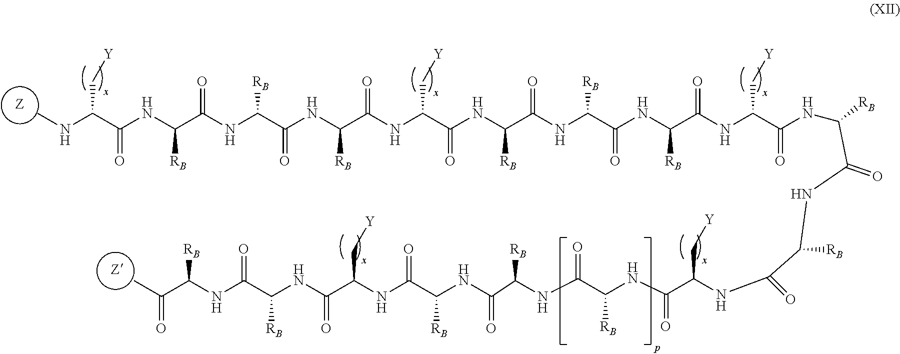
wherein each of Y, R^{B}, R^{E}, x, s, Z, and Z′ are as defined for formula (X).

35. The pharmaceutical composition of claim 1, wherein the polypeptide is represented by formula (XIII) 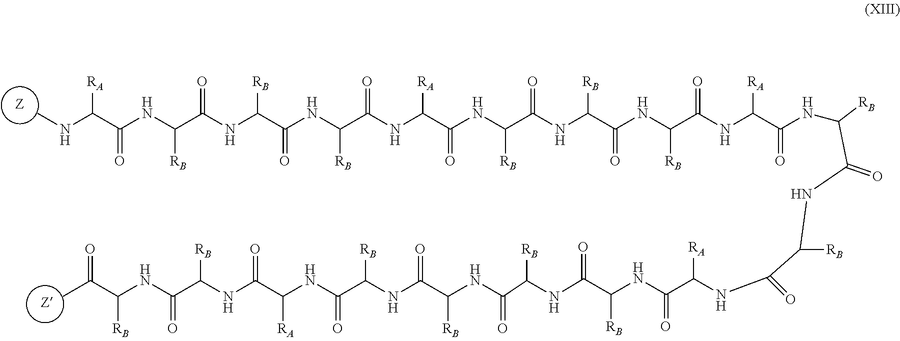
wherein each R_{A} is independently selected from: 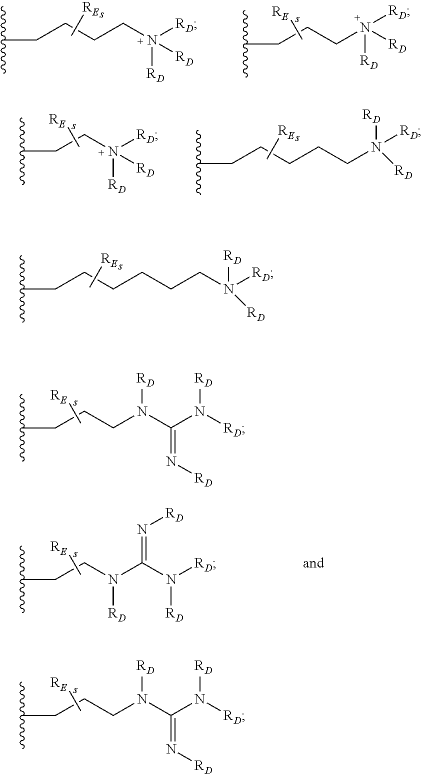
each R_{B} is independently selected from: 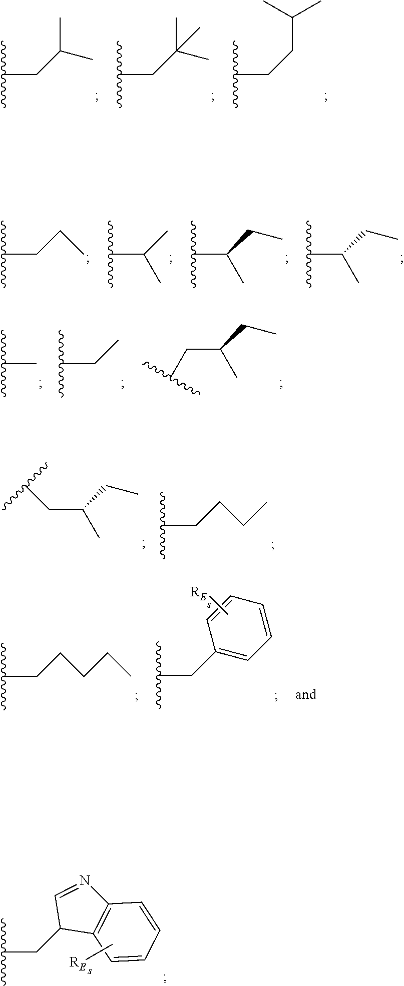
each R_{D} is independently hydrogen or an optionally substituted C₁-C₆ alkyl group;
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

36. The pharmaceutical composition of claim 35, wherein the polypeptide is represented by formula (XIV) 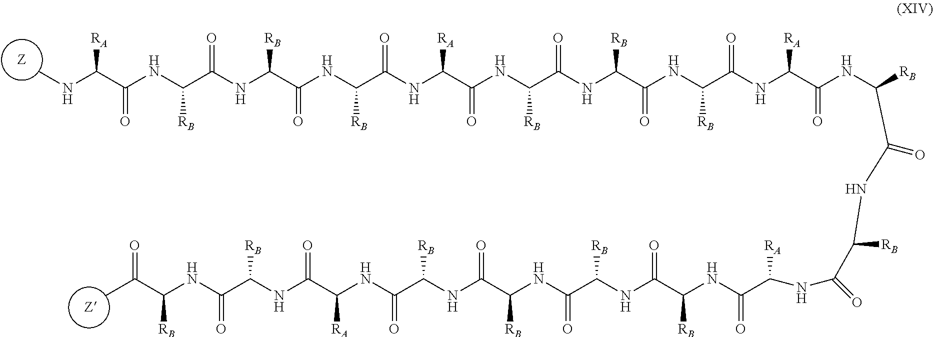
wherein each of R^{A}, R^{B}, R^{D}, R^{E}, s, Z, and Z′ are as defined for formula (XIII).

37. The pharmaceutical composition of claim 35, wherein the polypeptide is represented by formula (XV) 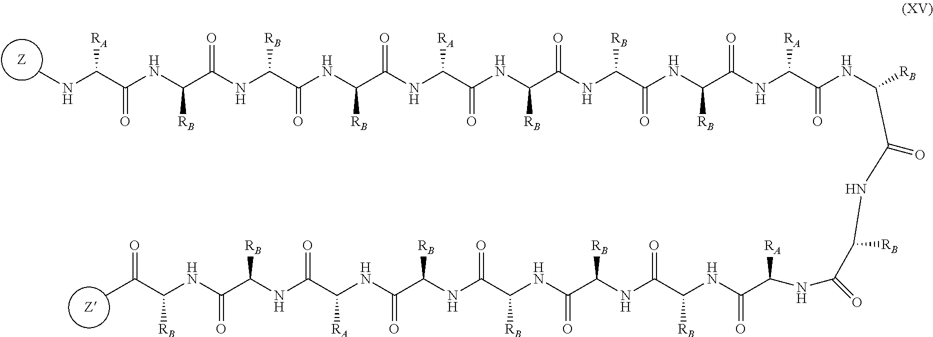
wherein each of R^{A}, R^{B}, R^{D}, R^{E}, s, Z, and Z′ are as defined for formula (XIII).

38. The pharmaceutical composition of claim 1, wherein the polypeptide is represented by formula (XVI) 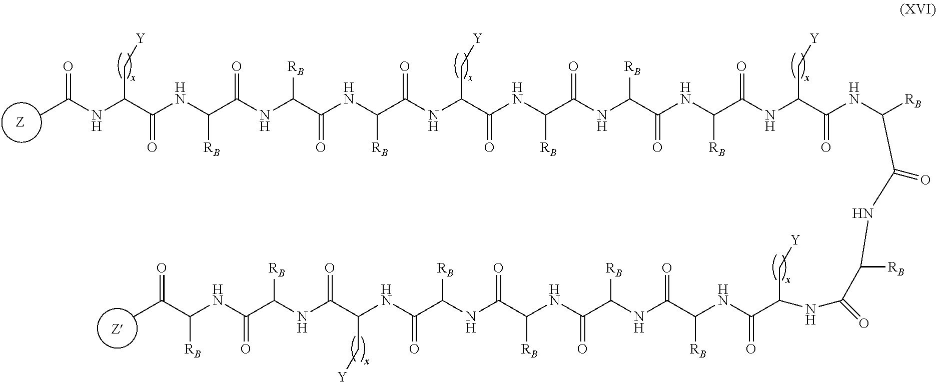
wherein each Y is independently an optionally substituted amino group or an optionally substituted guanidinium group;
each R_{B} is independently selected from: 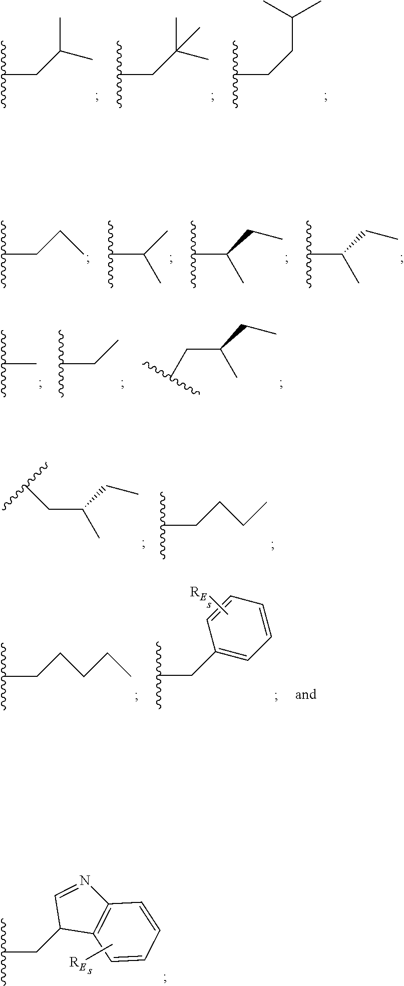
each R_{E}, if present, is independently an optionally substituted C₁-C₆ alkyl group, an optionally substituted C₁-C₆ acyl group; a halogen; an optionally substituted C₁-C₆ alkoxy group; an optionally substituted C₁-C₆ alkylamino group; or an optionally substituted C₁-C₆ alkylthio group;
x is an integer from 3 to 5;
s is an integer from 0 to 5;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

39. The pharmaceutical composition of claim 38, wherein the polypeptide is represented by formula (XVII) 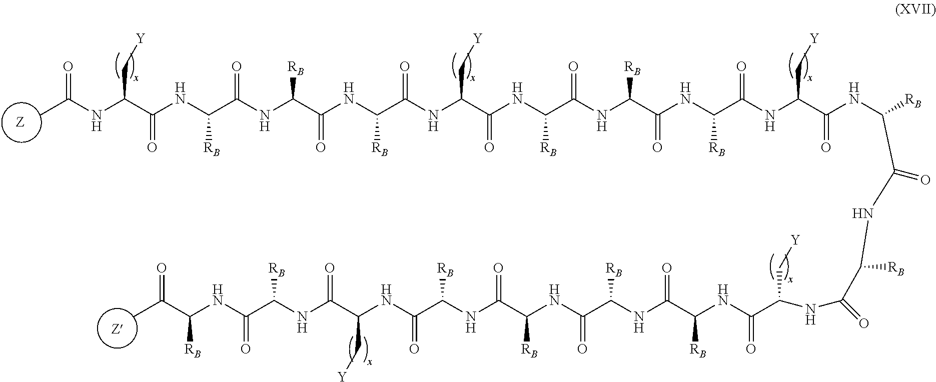
wherein each of Y, R^{B}, R^{E}, x, s, Z, and Z′ are as defined for formula (XVI).

40. The pharmaceutical composition of claim 38, wherein the polypeptide is represented by formula (XVIII) 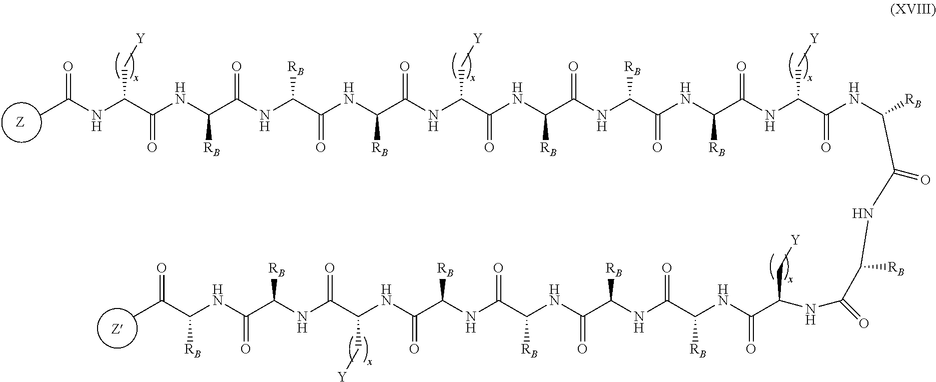
wherein each of Y, R^{B}, R^{E}, x, s, Z, and Z′ are as defined for formula (XVI).

41. The pharmaceutical composition of claim 1, wherein the polypeptide is represented by formula (XIX) 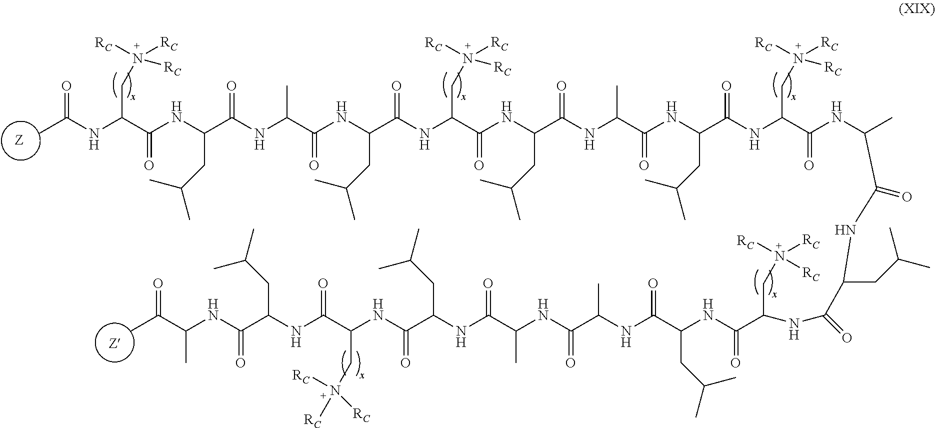
wherein each R_{C} is independently hydrogen or optionally substituted C₁-C₆ alkyl;
x is an integer from 3 to 5; optionally wherein x is 4;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

42. The pharmaceutical composition of claim 41, wherein the polypeptide is represented by formula (XX) 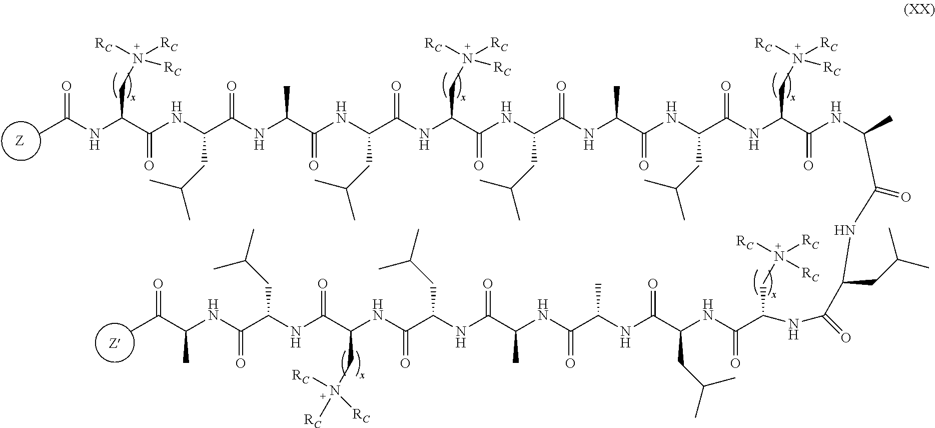
wherein R_{C}, x, Z, and Z′ are as defined for formula (XIX).

43. The pharmaceutical composition of claim 41, wherein the polypeptide is represented by formula (XXI) 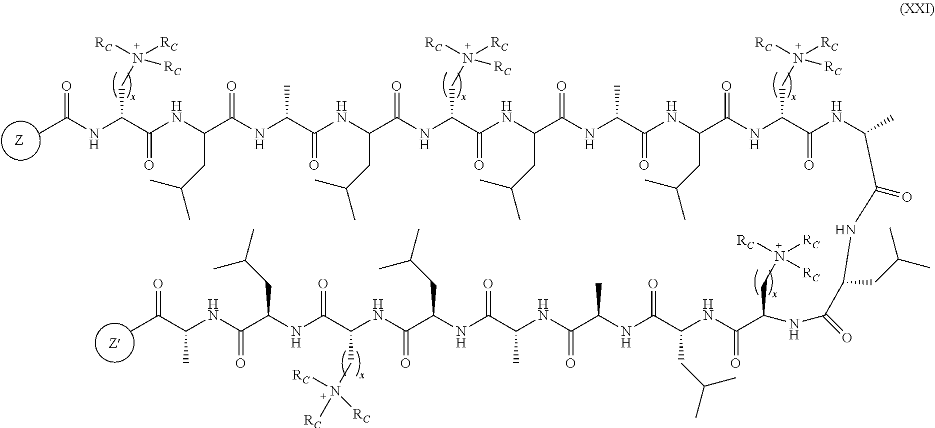
wherein R_{C}, x, Z, and Z′ are as defined for formula (XIX).

44. The pharmaceutical composition of claim 1, wherein the polypeptide is represented by formula (XXII) 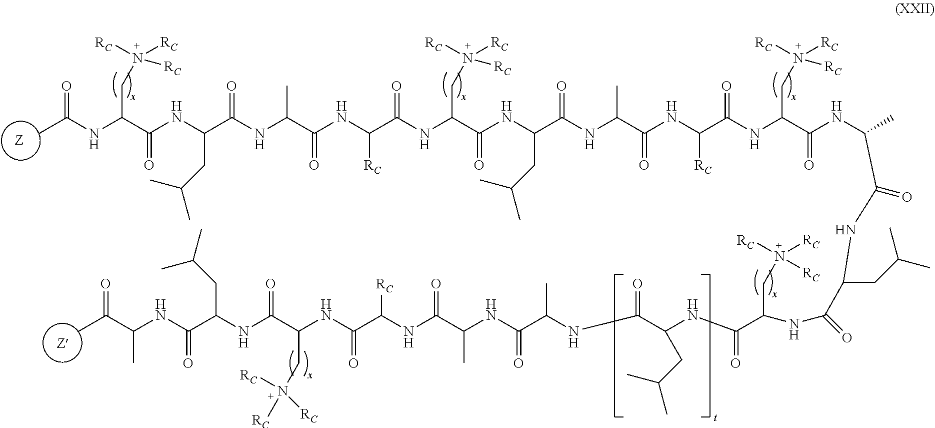
wherein each R_{C} is independently hydrogen or optionally substituted C₁-C₆ alkyl;
x is an integer from 3 to 5; optionally wherein x is 4;
t is 0 or 1;
Z is hydrogen or an optionally substituted C₁-C₆ acyl group, optionally wherein Z is an acetyl group; and
Z′ is optionally substituted C₁-C₆ alkoxy, optionally substituted C₁-C₆ alkylamino, —OH, or —NH₂.

45. The pharmaceutical composition of claim 44, wherein the polypeptide is represented by formula (XXIII) 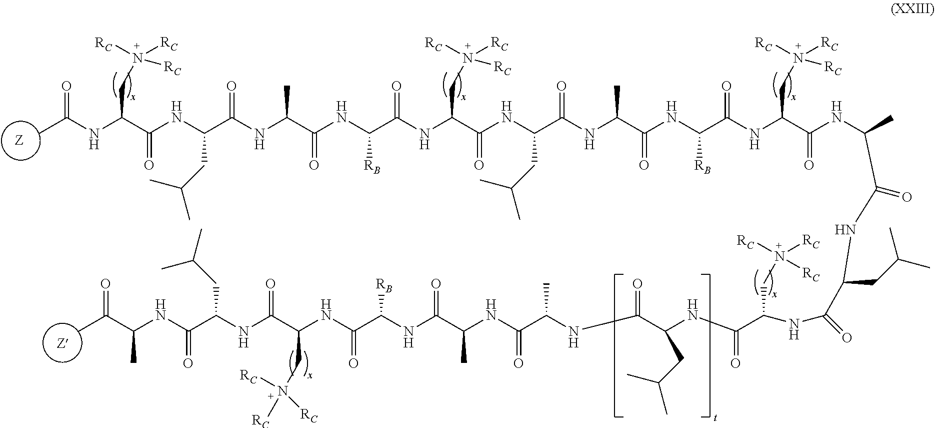
wherein R_{C}, x, t, Z, and Z′ are as defined for formula (XXII).

46. The pharmaceutical composition of claim 44, wherein the polypeptide is represented by formula (XXIV) 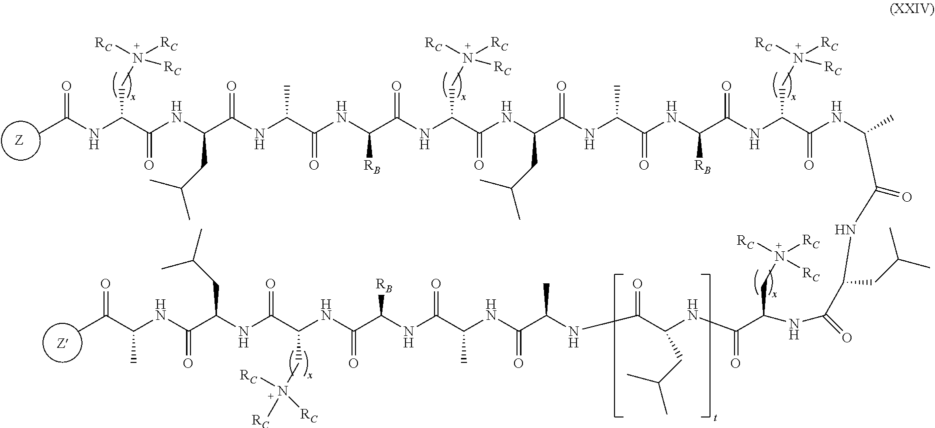
wherein R_{C}, x, t, Z, and Z′ are as defined for formula (XXII).

47. The pharmaceutical composition of claim 1, wherein the polypeptide is represented by formula (XXV) 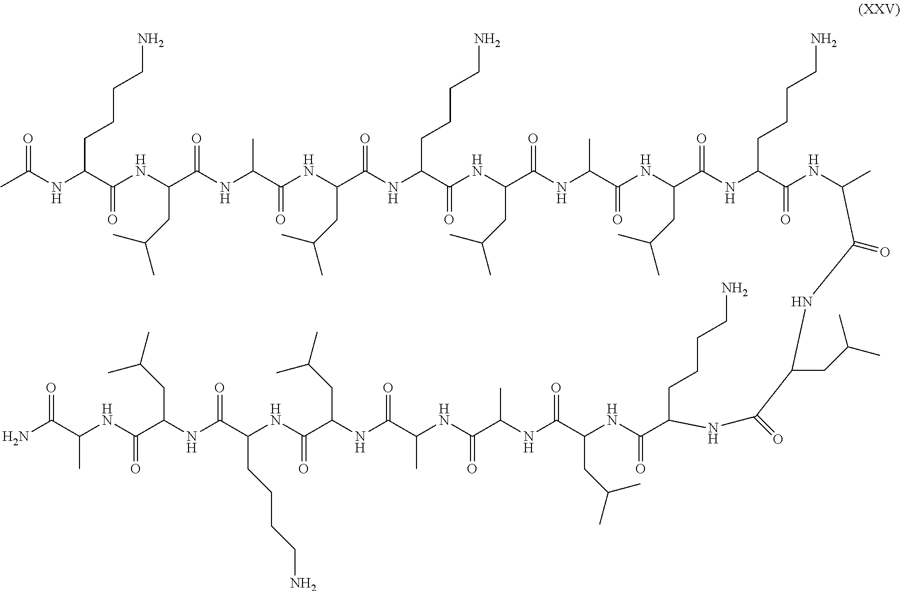 [SMILES: CC(=O)NC(CCCCN)C(=O)NC(CC(C)C)C(=O)NC(C)C(=O)NC(CC(C)C)C(=O)NC(CCCCN)C(=O)NC(CC(C)C)C(=O)NC(C)C(=O)NC(CC(C)C)C(=O)NC(CCCCN)C(=O)NC(C)C(=O)NC(CC(C)C)C(=O)NC(CCCCN)C(=O)NC(CC(C)C)C(=O)NC(C)C(=O)NC(C)C(=O)NC(CC(C)C)C(=O)NC(CCCCN)C(=O)NC(CC(C)C)C(=O)NC(C)C(N)=O]
or a pharmaceutically acceptable salt thereof.

48. The pharmaceutical composition of claim 47, wherein the polypeptide is represented by formula (XXVI) 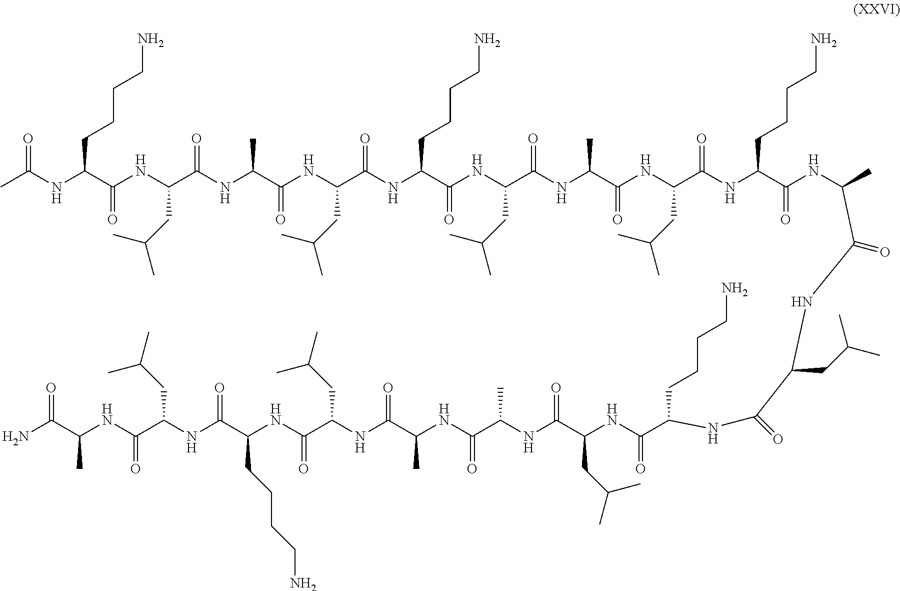 [SMILES: CC(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](CC(C)C)C(=O)N[C@@H](CCCCN)C(=O)N[C@@H](C)C(=O)N[C@@H](CC(C)C)C(=O)N[C@@H](CCCCN)C(=O)N[C@@H](CC(C)C)C(=O)N[C@@H](C)C(=O)N[C@@H](C)C(=O)N[C@@H](CC(C)C)C(=O)N[C@@H](CCCCN)C(=O)N[C@@H](CC(C)C)C(=O)N[C@@H](C)C(N)=O]
or a pharmaceutically acceptable salt thereof.

49. The pharmaceutical composition of claim 47, wherein the polypeptide is represented by formula (XXVII) 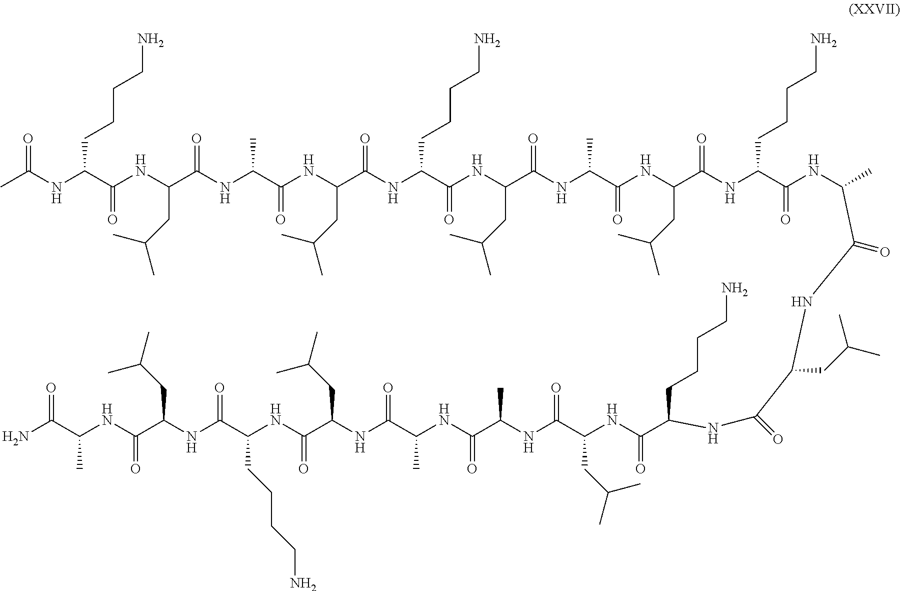 [SMILES: CC(=O)N[C@H](CCCCN)C(=O)NC(CC(C)C)C(=O)N[C@H](C)C(=O)NC(CC(C)C)C(=O)N[C@H](CCCCN)C(=O)NC(CC(C)C)C(=O)N[C@H](C)C(=O)NC(CC(C)C)C(=O)N[C@H](CCCCN)C(=O)N[C@H](C)C(=O)N[C@H](CC(C)C)C(=O)N[C@H](CCCCN)C(=O)N[C@H](CC(C)C)C(=O)N[C@H](C)C(=O)N[C@H](C)C(=O)N[C@H](CC(C)C)C(=O)N[C@H](CCCCN)C(=O)N[C@H](CC(C)C)C(=O)N[C@H](C)C(N)=O]
or a pharmaceutically acceptable salt thereof.

50. The pharmaceutical composition of claim 1, wherein the polypeptide comprises a region having an amino acid sequence that is at least 85% identical to an amino acid sequence selected from:
(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

51. The pharmaceutical composition of claim 1, wherein the polypeptide has an amino acid sequence selected from:
(SEQ ID NO: 1)
(i) KLALKLALKALKLAALKLA;
(SEQ ID NO: 2)
(ii) KLALKLALKALKAALKLA;
(SEQ ID NO: 3)
(iii) klalklalkalkaalkla;
(SEQ ID NO: 4)
(iv) alklaaklaklalklalk;
(SEQ ID NO: 5)
(v) LKlLKkLlkKLLkLL;
(SEQ ID NO: 6)
(vi) KLALKLALKALKAALK;
(SEQ ID NO: 7)
(vii) KLALKLALKALKAALKLALK;
(SEQ ID NO: 8)
(viii) KLAWKLALKALKAALKLA;
(SEQ ID NO: 9)
(ix) KLAWKLALKALKAAWKLA;
(SEQ ID NO: 10)
(x) KLAWKLAWKALKAAWKLA;
(SEQ ID NO: 11)
(xi) LKLLKKLLKKLLKLL;
(SEQ ID NO: 12)
(xii) LKlLKkLlkKLLkLL;
(SEQ ID NO: 13)
(xiii) KALAALLKKAAKLLAALK;
and
(SEQ ID NO: 14)
(xiv) KALAALLKKLAKLLAALK.

52. The pharmaceutical composition of any one of claims 1-51, wherein the polypeptide is present within the pharmaceutical composition at a concentration of from about 0.001% w/v to about 50% w/v.

53. The pharmaceutical composition of claim 52, wherein the polypeptide is present within the pharmaceutical composition at a concentration of from about 0.1% w/v to about 5% w/v.

54. The pharmaceutical composition of claim 52, wherein the polypeptide is present within the pharmaceutical composition at a concentration of about 0.1% w/v to about 1% w/v.

55. The pharmaceutical composition of any one of claims 1-54, wherein the therapeutic agent is present within the pharmaceutical composition at a concentration of from about 0.001% w/v to about 50% w/v.

56. The pharmaceutical composition of claim 55, wherein the therapeutic agent is present within the pharmaceutical composition at a concentration of from about 0.1% w/v to about 1% w/v.

57. The pharmaceutical composition of any one of claims 1-56, wherein the pharmaceutical composition further comprises a gelling agent.

58. The pharmaceutical composition of claim 57, wherein the gelling agent is selected from the group consisting of hyaluronan, hyaluronic acid, a polyoxyethylene-polyoxypropylene block copolymer, poly(lactic-co-glycolic) acid, polylactic acid, polycaprolactone, alginic acid or a salt thereof, polyethylene glycol, a cellulose, a cellulose ether, agar-agar, gelatin, glucomannan, galactomannan (e.g., locust bean gum or tara gum), xanthan gum, guar gum, chitosan, pectin, starch, tragacanth, carrageenan, polyvinylpyrrolidone, polyvinyl alcohol, paraffin, polyethoxylated sorbitan monolaurate, petrolatum, silicates, fibroin, gellan, CARBOPOL 940®, polyoxamines, lecithin gels, polysorbate-80, (poly)aniline derivatives, xyloglucane, collagen, silicon dioxide, tyloxapol, Cremophor, aluminum magnesium silicate, sodium stearate, bladderwrack, bentonite, eratonia, chondrus, dextrose, furcellaran, Ghatti gum, hectorite, lactose, sucrose, sucralose, maltodextrin, mannitol, sorbitol, honey, maize starch, wheat starch, rice starch, potato starch, oxypolygelatin, polygeline, sterculia gum, propylene carbonate, methyl vinyl ether/maleic anhydride copolymer, poly(methoxyethyl methacrylate), and poly(methoxyethoxyethyl methacrylate), and combinations thereof.

59. The pharmaceutical composition of claim 57, wherein the gelling agent is selected from the group consisting of a polyoxyethylene-polyoxypropylene block copolymer, alginic acid or a pharmaceutically acceptable salt thereof, collagen, hyaluronic acid or a pharmaceutically acceptable salt thereof, gelatin, and fibroin.

60. The pharmaceutical composition of claim 59, wherein the polyoxyethylene-polyoxypropylene block copolymer is poloxamer 407.

61. The pharmaceutical composition of claim 59, wherein the polyoxyethylene-polyoxypropylene block copolymer is poloxamer 188.

62. The pharmaceutical composition of any one of claims 58-61, wherein the polyoxyethylene-polyoxypropylene block copolymer is present within the pharmaceutical composition at a concentration of from about 0.001% w/v to about 50% w/v.

63. The pharmaceutical composition of claim 62, wherein the polyoxyethylene-polyoxypropylene block copolymer is present within the pharmaceutical composition at a concentration of about 20% w/v.

64. The pharmaceutical composition of any one of claims 1-63, wherein upon intratympanic or transtympanic administration to a mammalian subject, the therapeutic agent is delivered across the round window membrane of the subject.

65. The pharmaceutical composition of claim 64, wherein upon intratympanic or transtympanic administration to a mammalian subject, the therapeutic agent remains present within perilymph of the subject for at least from about 1 hour to about 6 weeks following the administration to the subject.

66. The pharmaceutical composition of claim 65, wherein upon intratympanic or transtympanic administration to a mammalian subject, the therapeutic agent remains present within perilymph of the subject for about 16 hours following the administration to the subject.

67. The pharmaceutical composition of any one of claims 1-66, wherein the pharmaceutical composition comprises a unit dosage form having a volume of from about 50 μL to about 1 mL.

68. The pharmaceutical composition of any one of claims 1-67, wherein the therapeutic agent is a neurotrophin, an immunomodulating agent, an aural pressure modulating agent, a corticosteroid, an antimicrobial agent, an antagonist of truncated TrkC or truncated TrkB, a non-natural TrkB or TrkC agonist, a TrkB receptor agonist antibody, a TrkB receptor agonist compound, a TrkC receptor agonist antibody, a TrkC receptor agonist compound, an Atoh1 modulator, or a WNT modulator.

69. The pharmaceutical composition of claim 68, wherein the therapeutic agent is a neurotrophin selected from neurotrophin-3 (NT-3), nerve growth factor (NGF), brain-derived neurotrophic factor (BDNF), ciliary neurotrophic factor (CNTF), glial cell-line derived neurotrophic factor (GDNF), neurotrophin-4 (NT-4), fibroblast growth factor (FGF), insulin-like growth factor (IGF), epidermal growth factor (EGF), platelet-derived growth factor (PGF), mesencephalic astrocyte-derived neurotrophic factor (MANF), cerebral dopamine neurotrophic factor (CDNF), a pan-neurotrophic factor, a chimeric neurotrophic factor, and combinations thereof.

70. The pharmaceutical composition of any one of claims 1-68, wherein the therapeutic agent is a glial cell line-derived neurotrophic factor family ligand, a neuropoietic cytokine, an anti-inflammatory cytokine, a neuroprotection agent, growth differentiation factor 11, erythropoietin (EPO), granulocyte-colony stimulating factor, granulocyte-macrophage colony stimulating factor, growth differentiation factor-9, thrombopoietin, transforming growth factor alpha (TGF-α), stromal cell-derived factor 1, myostatin, parathyroid hormone, parathyroid hormone related peptide, interleukin 1 receptor antagonist, fibroblast growth factor 18, high-mobility group protein 2, glucocorticoid receptor, fibroblast growth factor 9, hepatocyte growth factor, or a TGFβ-superfamily protein.

71. The pharmaceutical composition of any one of claims 1-68, wherein the therapeutic agent is a nucleic acid vector.

72. The pharmaceutical composition of any one of claims 1-71, wherein the therapeutic agent is an antibody or antigen-binding fragment thereof.

73. The pharmaceutical composition of claim 72, wherein the antibody or antigen-binding fragment thereof is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFv.

74. The pharmaceutical composition of any one of claims 1-68, wherein the therapeutic agent is encapsulated within a liposome, vesicle, synthetic vesicle, exosome, synthetic exosome, dendrimer, or nanoparticle.

75. The pharmaceutical composition of any one of claims 1-68, wherein the therapeutic agent is a small molecule, optionally wherein the small molecule is one that is not naturally round window membrane-penetrant.

76. The pharmaceutical composition of any one of claims 1-68, wherein the therapeutic agent is an interfering RNA.

77. The pharmaceutical composition of claim 76, wherein the interfering RNA is a short interfering RNA (siRNA), a short hairpin RNA (shRNA), or a micro RNA (miRNA).

78. The pharmaceutical composition of any one of claims 1-77, wherein the pharmaceutical composition is a gel at normal human body temperature.

79. The pharmaceutical composition of claim 78, wherein the gel has a dynamic viscosity of at about 100 cP to about 1,000,000 cP.

80. The pharmaceutical composition of any one of claims 1-79, wherein the pharmaceutical composition further comprises a pharmaceutically acceptable liquid solvent.

81. The pharmaceutical composition of claim 80, wherein the pharmaceutically acceptable liquid solvent is water.

82. The pharmaceutical composition of any one of claims 1-81, wherein the pharmaceutical composition comprises one or more agents selected from an antimicrobial agent, an arylcycloalkylamine, an elipticine derivative, an anti-apoptotic agent, a c-JNK inhibitor, an antioxidant, an NSAID, an analgesic, a neuroprotection agent, a glutamate modulator, an interleukin 1 modulator, an interleukin-1 antagonist, a corticosteroid, an anti-TNF agent, a calcineurin inhibitor, an IKK inhibitor, an interleukin inhibitor, a platelet activating factor antagonist, a TNF-α converting enzyme (TACE) inhibitor, a Toll-like receptor inhibitor, an autoimmune agent, an IL-1 modulator, an RNA interference agent, an aquaporin modulator, an estrogen-related receptor beta modulator, a GAP junction protein, a vasopressin receptor modulator, an NMDA receptor modulator, an ENaC receptor modulator, an osmotic diuretic, a progesterone receptor, a prostaglandin, a cytotoxic agent, a cytoprotective agent, an anti-intercellular adhesion molecule-1 antibody, an Atoh1 modulator, a Math1 modulator, a BRN-3 modulator, a carbamate, an estrogen receptor, a fatty acid, a gamma-secretase inhibitor, a glutamate-receptor modulator, a neurotrophic agent, salicylic acid, nicotine, a retinoblastoma protein modulator, an ion channel blocker, a thyroid hormone receptor modulator, a TRPV modulator, an adenosine modulator, a KCNQ modulator, a P2X modulator, a CNS modulating agent, an anticholinergic, an antihistamine, a GABA receptor modulator, a neurotransmitter reuptake inhibitor, a thyrotropin-releasing hormone, a free radical modulator, a metal atom chelator, a mitochondrial modulator, a nitric oxide synthase modulator, a sirtuin modulator, a purinergic receptor modulator, a truncated TrkC or TrkB antagonist, a truncated TrkC or TrkB isoform, a nucleic acid polymer antagonist, a small molecule antagonist, a polypeptide antagonist, a non-natural TrkC or TrkB agonist, a neurotrophin variant, a WNT modulator, a glycogen synthase kinase inhibitor, a protein kinase C beta modulator, a repulsive guidance molecule a (RGMa) inhibitor, a neogenin inhibitor, a SK2 channel activator, a BK channel activator, a sphingosine-1-phosphate receptor modulator, a stemness driver, a differentiation inhibitor, an N-Methyl-D-Aspartate (NMDA) receptor antagonist, a histone deacetylase (HDAC) inhibitor, a proteasome inhibitor, an EZH2/HMT inhibitor, a notch inhibitor, ebselen, ancrod, an α-amino-3-hydroxy-5-methyl-4-isoxazolepropionic acid (AMPA) glutamate-positive allosteric modulator, D-methionine, an antagonist of histamine type 4 receptors, a chemotherapeutic accumulation reducer, choline ester, plant alkaloid, reversible cholinesterase inhibitor, acetylcholine release promoter, anti-adrenergy, a sympathomimetic, an antineoplastic agent, R(+)-N-propargyl-1-aminoindan, and R-azasetron besylate.

83. A method of delivering a therapeutic agent across the round window membrane of a subject, the method comprising administering to the subject a therapeutically effective amount of the pharmaceutical composition of any one of claims 1-82.

84. The method of claim 83, wherein the pharmaceutical composition is administered to or near the round window membrane.

85. The method of claim 83 or 84, wherein the pharmaceutical composition is administered intratympanically ortranstympanically.

86. The method of any one of claims 83-85, wherein the method is used to treat an otic disease.

87. The method of claim 86, wherein the otic disease is ceruminosis or ceruminosis associated with an otic disease or condition, ear pruritus, otitis externa, otalgia, tinnitus, vestibular dysfunction, vertigo, dizziness, loss of balance, ear fullness, hearing loss, Meniere's disease, sensorineural hearing loss, noise-induced hearing loss, age-related hearing loss (presbycusis), ototoxic drug-induced hearing loss, hearing loss related to head trauma, hearing loss related to infection, autoimmune ear disease, ototoxicity, excitotoxicity, hidden hearing loss, cochlear synaptopathy, endolymphatic hydrops, labyrinthitis, Ramsay Hunt's Syndrome, vestibular neuronitis, or microvascular compression syndrome, hyperacusis, presbystasis, central auditory processing disorder, auditory neuropathy, improvement of cochlea implant performance, or a combination thereof.

88. A method of treating a subject having or at risk of developing hearing loss, comprising administering to the subject a therapeutically effective amount of the pharmaceutical composition of any one of claims 1-82.

89. The method of claim 88, wherein the hearing loss is genetic hearing loss.

90. The method of claim 89, wherein the genetic hearing loss is autosomal dominant hearing loss, autosomal recessive hearing loss, or X-linked hearing loss.

91. The method of claim 89, wherein the hearing loss is acquired hearing loss.

92. The method of claim 91, wherein the acquired hearing loss is noise-induced hearing loss, age-related hearing loss, disease or infection-related hearing loss, head trauma-related hearing loss, or ototoxic drug-induced hearing loss.

93. A method of treating a subject having or at risk of developing vestibular dysfunction, comprising administering to the subject a therapeutically effective amount of the pharmaceutical composition of any one of claims 1-82.

94. The method of claim 93, wherein the vestibular dysfunction is vertigo, dizziness, or imbalance.

95. A method of promoting hair cell regeneration in a subject in need thereof, comprising administering to the subject a therapeutically effective amount of the pharmaceutical composition of any one of claims 1-82.

96. A method of promoting SGN regeneration in a subject in need thereof, comprising administering to the subject a therapeutically effective amount of the pharmaceutical composition of any one of claims 1-82.

97. A method of preventing or reducing ototoxic drug-induced hair cell damage or death in a subject in need thereof, comprising administering to the subject a therapeutically effective amount of the pharmaceutical composition of any one of claims 1-82.

98. A method of preventing or reducing ototoxic drug-induced SGN damage or death, comprising administering to the subject a therapeutically effective amount of the pharmaceutical composition of any one of claims 1-82.

99. The method of claim 93, 97, or 98, wherein the ototoxic drug is selected from the group consisting of aminoglycosides, antineoplastic drugs, ethacrynic acid, furosemide, salicylates, and quinine.

100. A method of treating a subject having or at risk of developing tinnitus, comprising administering to the subject a therapeutically effective amount of the pharmaceutical composition of any one of claims 1-82.

101. A method of preventing or reducing hair cell damage or death in a subject in need thereof, comprising administering to the subject a therapeutically effective amount of the pharmaceutical composition of any one of claims 1-82.

102. A method of preventing or reducing SGN damage or death in a subject in need thereof, comprising administering to the subject a therapeutically effective amount of the pharmaceutical composition of any one of claims 1-82.

103. A method of increasing hair cell survival in a subject in need thereof, comprising administering to the subject a therapeutically effective amount of the pharmaceutical composition of any one of claims 1-82.

104. A method of increasing SGN survival in a subject in need thereof, comprising administering to the subject a therapeutically effective amount of the pharmaceutical composition of any one of claims 1-82.

105. A method of increasing the number of supporting cells subject in need thereof, comprising administering to the subject a therapeutically effective amount of the pharmaceutical composition of any one of claims 1-82.

106. The method of any one of claims 83-105, wherein the pharmaceutical composition is locally administered.

107. The method of any one of claims 87-106, wherein the pharmaceutical composition is administered in an amount sufficient to prevent or reduce hearing loss, prevent or reduce vestibular dysfunction, prevent or reduce tinnitus, delay the development of hearing loss, delay the development of vestibular dysfunction, slow the progression of hearing loss, slow the progression of vestibular dysfunction, improve hearing, improve vestibular function, improve hair cell function, prevent or reduce hair cell damage, prevent, slow, or reduce hair cell death, promote or increase hair cell survival, increase supporting cell numbers, increase hair cell numbers, promote or induce hair cell regeneration, improve SGN function, prevent or reduce SGN damage, prevent, slow, or reduce SGN death, promote or increase SGN survival, increase SGN numbers, promote or induce SGN regeneration, preserve ribbon synapses, promote or increase ribbon synapse formation, maintain the connections between hair cells and SGNs, or increase or restore the connections between hair cells and SGNs.

108. The method of any one of claims 83-107, wherein the subject is a mammalian subject.

109. The method of claim 108, wherein the mammalian subject is a human subject.

110. A kit comprising the pharmaceutical composition of any one of claims 1-82.

111. The kit of claim 110, wherein the kit further comprises a package insert instructing a user of the kit to administer the pharmaceutical composition to a subject in need thereof.

112. The kit of claim 111, wherein the subject is a mammalian subject.

113. The kit of claim 112, wherein the mammalian subject is a human subject.
